SEQUENCE LISTING (840 sequences)

SEQ ID NO: 1 - protein, 5 aa, synthetic construct
DYYMS

SEQ ID NO: 2 - protein, 17 aa, synthetic construct
YISSSGSTIYYADSVKG

SEQ ID NO: 3 - protein, 8 aa, synthetic construct
PLRWGMDV

SEQ ID NO: 4 - protein, 30 aa, synthetic construct
QVQLVESGGGLVKPGGSLRLSCAASGFTFS

SEQ ID NO: 5 - protein, 14 aa, synthetic construct
WIRQAPGKGLEWVS

SEQ ID NO: 6 - protein, 32 aa, synthetic construct
RFTISRDNAKNSLYLQMNSLRAEDTAVYYCAR

SEQ ID NO: 7 - protein, 11 aa, synthetic construct
WGQGTTVTVSS

SEQ ID NO: 8 - protein, 117 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSS

SEQ ID NO: 9 - protein, 11 aa, synthetic construct
RASQSISSYLN

SEQ ID NO: 10 - protein, 7 aa, synthetic construct
AASSLQS

SEQ ID NO: 11 - protein, 9 aa, synthetic construct
HQSSSLPLT

SEQ ID NO: 12 - protein, 23 aa, synthetic construct
DIQMTQSPSSLSASVGDRVTITC

SEQ ID NO: 13 - protein, 15 aa, synthetic construct
WYQQKPGKAPKLLIY

SEQ ID NO: 14 - protein, 32 aa, synthetic construct
GVPSRFSGSGSGTDFTLTISSLQPEDAATYYC

SEQ ID NO: 15 - protein, 10 aa, synthetic construct
FGGGTKVEIK

SEQ ID NO: 16 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIK

SEQ ID NO: 17 - protein, 5 aa, synthetic construct
DYYMS

SEQ ID NO: 18 - protein, 17 aa, synthetic construct
YISSSGSTIYYADSVKG

SEQ ID NO: 19 - protein, 8 aa, synthetic construct
PLRWGMDV

SEQ ID NO: 20 - protein, 30 aa, synthetic construct; identical to SEQ ID NO: 4

SEQ ID NO: 21 - protein, 14 aa, synthetic construct
WIRQAPGKGLEWVS

SEQ ID NO: 22 - protein, 32 aa, synthetic construct; identical to SEQ ID NO: 6

SEQ ID NO: 23 - protein, 11 aa, synthetic construct
WGQGTTVTVSS

SEQ ID NO: 24 - protein, 117 aa, synthetic construct; identical to SEQ ID NO: 8

SEQ ID NO: 25 - protein, 11 aa, synthetic construct
RASQSISSYLN

SEQ ID NO: 26 - protein, 7 aa, synthetic construct
AASSLQS

SEQ ID NO: 27 - protein, 9 aa, synthetic construct
HQSSSLPLT

SEQ ID NO: 28 - protein, 23 aa, synthetic construct
DIQMTQSPSSLSASVGDRVTITC

SEQ ID NO: 29 - protein, 15 aa, synthetic construct
WYQQKPGKAPKLLIY

SEQ ID NO: 30 - protein, 32 aa, synthetic construct
GVPSRFSGSGSGTDFTLTISSLQPEDFAVYYC

SEQ ID NO: 31 - protein, 10 aa, synthetic construct
FGGGTKLEIK

SEQ ID NO: 32 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFAVYYCHQSSSLPLTFGGGTKLEIK

SEQ ID NO: 33 - protein, 5 aa, synthetic construct
DYYMS

SEQ ID NO: 34 - protein, 17 aa, synthetic construct
YISSSGSTIYYADSVKG

SEQ ID NO: 35 - protein, 8 aa, synthetic construct
PLRWGMDV

SEQ ID NO: 36 - protein, 30 aa, synthetic construct; identical to SEQ ID NO: 4

SEQ ID NO: 37 - protein, 14 aa, synthetic construct
WIRQAPGKGLEWVS

SEQ ID NO: 38 - protein, 32 aa, synthetic construct; identical to SEQ ID NO: 6

SEQ ID NO: 39 - protein, 11 aa, synthetic construct
WGQGTTVTVSS

SEQ ID NO: 40 - protein, 117 aa, synthetic construct; identical to SEQ ID NO: 8

SEQ ID NO: 41 - protein, 11 aa, synthetic construct
RASQSISSYLN

SEQ ID NO: 42 - protein, 7 aa, synthetic construct
AASSLQS

SEQ ID NO: 43 - protein, 9 aa, synthetic construct
HQSNSLPLT

SEQ ID NO: 44 - protein, 23 aa, synthetic construct
DIQMTQSPSSLSASVGDRVTITC

SEQ ID NO: 45 - protein, 15 aa, synthetic construct
WYQQKPGKAPKLLIY

SEQ ID NO: 46 - protein, 32 aa, synthetic construct
GVPSRFSGSGSGTDFTLTISSLQPEDVAVYYC

SEQ ID NO: 47 - protein, 10 aa, synthetic construct
FGGGTKVEIK

SEQ ID NO: 48 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCHQSNSLPLTFGGGTKVEIK

SEQ ID NO: 49 - protein, 5 aa, synthetic construct
DYYMS

SEQ ID NO: 50 - protein, 17 aa, synthetic construct
YISSSGSTIVYADSVKG

SEQ ID NO: 51 - protein, 8 aa, synthetic construct
PIRRGMDV

SEQ ID NO: 52 - protein, 30 aa, synthetic construct; identical to SEQ ID NO: 4

SEQ ID NO: 53 - protein, 14 aa, synthetic construct
WIRQAPGKGLEWVS

SEQ ID NO: 54 - protein, 32 aa, synthetic construct; identical to SEQ ID NO: 6

SEQ ID NO: 55 - protein, 11 aa, synthetic construct
WGQGTTVTVSS

SEQ ID NO: 56 - protein, 117 aa, synthetic construct
QVQLVESGGGLVKPGGSLRLSCAASGFTFSDYYMSWIRQAPGKGLEWVSYISSSGSTIVYADSVKGRFTISRDNAKNSLYLQMNSLRAEDTAVYYCARPIRRGMDVWGQGTTVTVSS

SEQ ID NO: 57 - protein, 11 aa, synthetic construct
RASQSISSYLN

SEQ ID NO: 58 - protein, 7 aa, synthetic construct
AASSLQS

SEQ ID NO: 59 - protein, 9 aa, synthetic construct
HQSSSLPLT

SEQ ID NO: 60 - protein, 23 aa, synthetic construct
DIQMTQSPSSLSASVGDRVTITC

SEQ ID NO: 61 - protein, 15 aa, synthetic construct
WYQQKPGKAPKLLIY

SEQ ID NO: 62 - protein, 32 aa, synthetic construct; identical to SEQ ID NO: 14

SEQ ID NO: 63 - protein, 10 aa, synthetic construct
FGGGTKVEIK

SEQ ID NO: 64 - protein, 107 aa, synthetic construct; identical to SEQ ID NO: 16

SEQ ID NO: 65 - protein, 5 aa, synthetic construct
DYYMS

SEQ ID NO: 66 - protein, 17 aa, synthetic construct
YISSSGSTINYADSVKG

SEQ ID NO: 67 - protein, 8 aa, synthetic construct
PIRWGMDV

SEQ ID NO: 68 - protein, 30 aa, synthetic construct; identical to SEQ ID NO: 4

SEQ ID NO: 69 - protein, 14 aa, synthetic construct
WIRQAPGKGLEWVS

SEQ ID NO: 70 - protein, 32 aa, synthetic construct; identical to SEQ ID NO: 6

SEQ ID NO: 71 - protein, 11 aa, synthetic construct
WGQGTTVTVSS

SEQ ID NO: 72 - protein, 117 aa, synthetic construct
QVQLVESGGGLVKPGGSLRLSCAASGFTFSDYYMSWIRQAPGKGLEWVSYISSSGSTINYADSVKGRFTISRDNAKNSLYLQMNSLRAEDTAVYYCARPIRWGMDVWGQGTTVTVSS

SEQ ID NO: 73 - protein, 11 aa, synthetic construct
RASQSISSYLN

SEQ ID NO: 74 - protein, 7 aa, synthetic construct
AASSLQS

SEQ ID NO: 75 - protein, 9 aa, synthetic construct
HQSSSLPLT

SEQ ID NO: 76 - protein, 23 aa, synthetic construct
DIQMTQSPSSLSASVGDRVTITC

SEQ ID NO: 77 - protein, 15 aa, synthetic construct
WYQQKPGKAPKLLIY

SEQ ID NO: 78 - protein, 32 aa, synthetic construct; identical to SEQ ID NO: 14

SEQ ID NO: 79 - protein, 10 aa, synthetic construct
FGGGTKVEIK

SEQ ID NO: 80 - protein, 107 aa, synthetic construct; identical to SEQ ID NO: 16

SEQ ID NO: 81 - protein, 5 aa, synthetic construct
SYAIS

SEQ ID NO: 82 - protein, 17 aa, synthetic construct
GIIPIFGTANYAQKFQG

SEQ ID NO: 83 - protein, 16 aa, synthetic construct
ERIAVAGTNYYYGMDV

SEQ ID NO: 84 - protein, 30 aa, synthetic construct
EVQLVQSGAEVKKPGSSVKVSCKASGGTFS

SEQ ID NO: 85 - protein, 14 aa, synthetic construct
WVRQAPGQGLEWMG

SEQ ID NO: 86 - protein, 32 aa, synthetic construct
RVTITADESTSTAYMELSSLRSDDTAVYYCAR

SEQ ID NO: 87 - protein, 11 aa, synthetic construct
WGQGTLVTVSS

SEQ ID NO: 88 - protein, 125 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSDDTAVYYCARERIAVAGTNYYYGMDVWGQGTLVTVSS

SEQ ID NO: 89 - protein, 12 aa, synthetic construct
RASQSVSSSNLA

SEQ ID NO: 90 - protein, 7 aa, synthetic construct
GASTRAT

SEQ ID NO: 91 - protein, 8 aa, synthetic construct
QQYGSSIT

SEQ ID NO: 92 - protein, 23 aa, synthetic construct
EIVLTQSPATLSVSPGERATLSC

SEQ ID NO: 93 - protein, 15 aa, synthetic construct
WYQQKPGQAPRLLIY

SEQ ID NO: 94 - protein, 32 aa, synthetic construct
GIPARFSGTGSGTDFTLTISRLEPEDFAVYYC

SEQ ID NO: 95 - protein, 10 aa, synthetic construct
FGQGTRLEIK

SEQ ID NO: 96 - protein, 107 aa, synthetic construct
EIVLTQSPATLSVSPGERATLSCRASQSVSSSNLAWYQQKPGQAPRLLIYGASTRATGIPARFSGTGSGTDFTLTISRLEPEDFAVYYCQQYGSSITFGQGTRLEIK

SEQ ID NO: 97 - protein, 5 aa, synthetic construct
GYYMH

SEQ ID NO: 98 - protein, 17 aa, synthetic construct
RINPNSGGTNYAQKFQG

SEQ ID NO: 99 - protein, 8 aa, synthetic construct
GTRHAFDI

SEQ ID NO: 100 - protein, 30 aa, synthetic construct
EVQLVESGAEVKKPGASVKVSCKASGYTFT

SEQ ID NO: 101 - protein, 14 aa, synthetic construct
WVRQAPGQGLVWMG

SEQ ID NO: 102 - protein, 32 aa, synthetic construct
RVTMTRDTSISTAYMELSRLRSDDTAVYYCAF

SEQ ID NO: 103 - protein, 11 aa, synthetic construct
WGQGTMVTVSS

SEQ ID NO: 104 - protein, 117 aa, synthetic construct
EVQLVESGAEVKKPGASVKVSCKASGYTFTGYYMHWVRQAPGQGLVWMGRINPNSGGTNYAQKFQGRVTMTRDTSISTAYMELSRLRSDDTAVYYCAFGTRHAFDIWGQGTMVTVSS

SEQ ID NO: 105 - protein, 13 aa, synthetic construct
TRSSGSIASNYVQ

SEQ ID NO: 106 - protein, 7 aa, synthetic construct
EDNQRPS

SEQ ID NO: 107 - protein, 10 aa, synthetic construct
QSYDSIVHVV

SEQ ID NO: 108 - protein, 22 aa, synthetic construct
NFMLTQPHSVSESPGKTVTISC

SEQ ID NO: 109 - protein, 15 aa, synthetic construct
WYQQRPGSAPTTVIY

SEQ ID NO: 110 - protein, 34 aa, synthetic construct
GVPDRFSGSIDSSSNSASLTISGLKTEDEADYYC

SEQ ID NO: 111 - protein, 11 aa, synthetic construct
FGGGTKVTVLG

SEQ ID NO: 112 - protein, 112 aa, synthetic construct
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDSIVHVVFGGGTKVTVLG

SEQ ID NO: 113 - protein, 5 aa, synthetic construct
SYAIS

SEQ ID NO: 114 - protein, 17 aa, synthetic construct
GIIPIFGTANYAQKFQG

SEQ ID NO: 115 - protein, 13 aa, synthetic construct
DVIVGATSSGMDV

SEQ ID NO: 116 - protein, 30 aa, synthetic construct
EVQLVQSGAEVKKPGASVKVSCKASGGTFS

SEQ ID NO: 117 - protein, 14 aa, synthetic construct
WVRQAPGQGLEWMG

SEQ ID NO: 118 - protein, 32 aa, synthetic construct
RVTITADKSTSTAYMELSSLRSEDTAVYYCAR

SEQ ID NO: 119 - protein, 11 aa, synthetic construct
WGQGTTVTVSS

SEQ ID NO: 120 - protein, 122 aa, synthetic construct
EVQLVQSGAEVKKPGASVKVSCKASGGTFSSYAISWVRQAPGQGLEWMGGIIPIFGTANYAQKFQGRVTITADKSTSTAYMELSSLRSEDTAVYYCARDVIVGATSSGMDVWGQGTTVTVSS

SEQ ID NO: 121 - protein, 11 aa, synthetic construct
RASQSISRYLN

SEQ ID NO: 122 - protein, 7 aa, synthetic construct
GASSLQS

SEQ ID NO: 123 - protein, 9 aa, synthetic construct
QQSYSTPLT

SEQ ID NO: 124 - protein, 23 aa, synthetic construct
DIQMTQSPSSVSASVGDRVTITC

SEQ ID NO: 125 - protein, 15 aa, synthetic construct
WYQQKPGKAPKLLIY

SEQ ID NO: 126 - protein, 32 aa, synthetic construct
GVPSRFSGSGFGTDFTLTISSLQPEDFATYYC

SEQ ID NO: 127 - protein, 10 aa, synthetic construct
FGQGTKLEIK

SEQ ID NO: 128 - protein, 107 aa, synthetic construct
DIQMTQSPSSVSASVGDRVTITCRASQSISRYLNWYQQKPGKAPKLLIYGASSLQSGVPSRFSGSGFGTDFTLTISSLQPEDFATYYCQQSYSTPLTFGQGTKLEIK

SEQ ID NO: 129 - protein, 114 aa, Homo sapiens
MAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 130 - DNA, 345 bp, Homo sapiens
  source 1..345: mol_type = unassigned DNA
atggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgctgccatccttgcgctgctccctgcactcggcctgctgctctggggacccggccagctctag

SEQ ID NO: 131 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 132 - protein, 5 aa, Homo sapiens
EPKSC

SEQ ID NO: 133 - protein, 107 aa, Homo sapiens [sequence omitted]

SEQ ID NO: 134 - protein, 98 aa, Homo sapiens
ASTRGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 135 - protein, 15 aa, synthetic construct
GGGGSGGGGSGGGGS

SEQ ID NO: 136 - DNA, 660 bp, synthetic construct
  source 1..660: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcgagacccctccggtggggtatggacgtctggggccaagggaccacggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 137 - DNA, 642 bp, synthetic construct
  source 1..642: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcatcagagtagtagtttaccgctcactttcggcggagggaccaaggtggaaatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 138 - DNA, 660 bp, synthetic construct
  source 1..660: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagacccctccggtggggtatggacgtctggggccaagggaccacggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 139 - DNA, 642 bp, synthetic construct
  source 1..642: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagattttgcagtgtattactgtcatcagagtagtagtttacccctcactttcggcggagggaccaaactggagatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 140 - DNA, 660 bp, synthetic construct
  source 1..660: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcgagacccctccggtggggtatggacgtctggggccaagggaccacggtcactgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 141 - DNA, 642 bp, synthetic construct
  source 1..642: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgtcatcagagtaatagtttaccgctcactttcggcggagggaccaaagtggagatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 142 - DNA, 660 bp, synthetic construct
  source 1..660: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatcgtctatgcagattccgtgaaagggcgcttcaccatctctcgtgacaatgcaaagaacagtttatacttacaaatgaactcgctgcgtgcggaagatacggctgtgtattactgtgcgagacccatccggaggggtatggacgtctggggccaagggaccacggtcaccgtctcctcagcgtcgaccagagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 143 - DNA, 642 bp, synthetic construct; identical to SEQ ID NO: 137
  source 1..642: mol_type = other DNA

SEQ ID NO: 144 - DNA, 660 bp, synthetic construct
  source 1..660: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatcaactatgcagattccgtgaaagggcgcttcaccatctctcgtgacaatgcaaagaacagtttatacttacaaatgaactcgctgcgtgcggaagatacggctgtgtattactgtgcgagacccatccggtggggtatggacgtctggggccaagggaccacggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 145 - DNA, 642 bp, synthetic construct; identical to SEQ ID NO: 137
  source 1..642: mol_type = other DNA

SEQ ID NO: 146 - DNA, 741 bp, synthetic construct
  source 1..741: mol_type = other DNA
gaggtccagctggtgcagtctggggctgaggtgaagaagcctgggtcctcggtgaaggtctcttgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgacgacacggccgtgtattactgtgcgagagagcgtatagcagtggctggtactaactactactacggtatggacgtctggggccaaggcaccctggtcaccgtctcctcaggaggtggcgggtctggtgggggcggaagcggtggtggcggatccgaaattgtgttgacacagtctccagccaccctgtctgtgtctccaggggaaagagccaccctctcctgcagggccagtcagagtgttagcagcagcaacttagcctggtaccagcagaaacctggccaggctcccaggctcctcatctatggtgcatccaccagggccactggtatcccagccaggttcagtggcactgggtctgggacagacttcactctcaccatcagcaggctggagcctgaagattttgcagtgtattactgtcagcagtatggtagctcgatcaccttcggccaagggacacgactagagattaaa

SEQ ID NO: 147 - DNA, 732 bp, synthetic construct
  source 1..732: mol_type = other DNA
gaggtgcagctggtggagtccggggctgaggtgaagaagcctggggcctcagtgaaggtctcctgcaaggcttctggatacaccttcaccggctactatatgcactgggtgcgacaggcccctggacaagggcttgtgtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgttcgggacgcgccatgcttttgatatctggggccaagggacaatggtcaccgtctcttcaggaggtggcgggtctggtgggggcggaagcggtggtggcggatccaattttatgctgactcagccccactctgtgtcggagtccccgggaaagacggtaaccatctcctgcacccgcagtagtggctccattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcatcgtccatgtggtgttcggcggagggaccaaggtcaccgtcctaggt

SEQ ID NO: 148 - DNA, 732 bp, synthetic construct
  source 1..732: mol_type = other DNA
gaggtgcagctggtgcagtctggggctgaggtgaagaagcctggggcctcagtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagagacgttatagtgggagctactagctcgggtatggacgtctggggccaagggaccacggtcaccgtctcctcaggaggtggcgggtctggtgggggcggaagcggtggtggcggatccgacatccagatgacccagtctccatcttccgtgtctgcatctgtaggagacagagtcaccatcacttgccgggcaagtcagagcattagcagatatttaaattggtatcagcagaaaccagggaaagcccctaagctcctgatctatggtgcatccagtttgcaaagtggggtcccatcaaggttcagtggcagtggatttgggacagatttcactctcaccatcagcagtctgcaacctgaagattttgcaacttactactgtcaacagagttacagtacccctctgacgttcggccaagggaccaagctggaaatcaaa

SEQ ID NO: 149 - protein, 15 aa, Homo sapiens
EPKSCDKTHTCPPCP

SEQ ID NO: 150 - DNA, 45 bp, Homo sapiens
  source 1..45: mol_type = unassigned DNA
gagcccaaatcttgtgacaaaactcacacatgcccaccgtgccca

SEQ ID NO: 151 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - DNA, 651 bp, Homo sapiens
  source 1..651: mol_type = unassigned DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 153 - skipped

SEQ ID NO: 154 - protein, 30 aa, synthetic construct
EVQLVESGGGLVQPGGSLRLSCAASAFYFT

SEQ ID NO: 155 - protein, 14 aa, synthetic construct
WVRQAPGQGLEWVS

SEQ ID NO: 156 - protein, 32 aa, synthetic construct
RFTISRDNSKNTLYLQMNSLRAEDTAMYYCAR

SEQ ID NO: 157 - protein, 11 aa, synthetic construct
WGQGTLVTVSS

SEQ ID NO: 158 - skipped

SEQ ID NO: 159 - protein, 25 aa, synthetic construct
VPVLTQPHSVSESPGKTVTISCTRS

SEQ ID NO: 160 - protein, 17 aa, synthetic construct
VQWYQQRPGSSPTTVIY

SEQ ID NO: 161 - protein, 38 aa, synthetic construct
QRPSGVPDRFSGSIDMSSNSASLTISGLRTEDEGDYYC

SEQ ID NO: 162 - protein, 11 aa, synthetic construct
FGGGTQLTVLG

SEQ ID NO: 163 - skipped

SEQ ID NO: 164 - protein, 30 aa, synthetic construct; identical to SEQ ID NO: 154

SEQ ID NO: 165 - protein, 14 aa, synthetic construct
WVRQAPGQGLERVS

SEQ ID NO: 166 - protein, 32 aa, synthetic construct
RFTISRDNSKNTLYLEMNSLRAEDTAMYYCAR

SEQ ID NO: 167 - protein, 11 aa, synthetic construct
WGQGTLVTVSS

SEQ ID NO: 168 - skipped

SEQ ID NO: 169 - protein, 25 aa, synthetic construct
QSALTQPASVSGSPGQSITISCTRA

SEQ ID NO: 170 - protein, 17 aa, synthetic construct
VSWYQQHPGKAPKLMIY

SEQ ID NO: 171 - protein, 36 aa, synthetic construct
NRPSGVSNRFSGSKSGNTASLTISGLQAEDEADYYC

SEQ ID NO: 172 - protein, 11 aa, synthetic construct
FGGGTQLTVLG

SEQ ID NO: 173 - skipped

SEQ ID NO: 174 - protein, 30 aa, synthetic construct
EVQLVESGGGLVQPGGSLRLSCAASYFDFD

SEQ ID NO: 175 - protein, 14 aa, synthetic construct
WVRQAPGQGLEWVS

SEQ ID NO: 176 - protein, 32 aa, synthetic construct
RFTISRDNSKNTLYLQMNSLRAEDTAVYYCAR

SEQ ID NO: 177 - protein, 11 aa, synthetic construct
WGQGTLVTVSS

SEQ ID NO: 178 - skipped

SEQ ID NO: 179 - protein, 23 aa, synthetic construct
DIQMTQSPSSLSASVGDRVTITC

SEQ ID NO: 180 - protein, 15 aa, synthetic construct
WYQQKPGKAPKLLIY

SEQ ID NO: 181 - protein, 32 aa, synthetic construct
GVPSRFSGSGSGTDFTLTISSLQPEDFGVYYC

SEQ ID NO: 182 - protein, 10 aa, synthetic construct
FGQGTRLEIK

SEQ ID NO: 183 - skipped

SEQ ID NO: 184 - protein, 30 aa, synthetic construct
EVQLVESGGGLVQPGGSLRLSCAASYLDFD

SEQ ID NO: 185 - protein, 14 aa, synthetic construct
WFRQAPGQGLEWVS

SEQ ID NO: 186 - protein, 32 aa, synthetic construct
RFTISRDNSQNTLYLQMNSLRAEDTAVYYCAR

SEQ ID NO: 187 - protein, 11 aa, synthetic construct
WGQGTLVTVSS

SEQ ID NO: 188 - skipped

SEQ ID NO: 189 - protein, 23 aa, synthetic construct
DIQMTQSPSSLSASVGDRVTITC

SEQ ID NO: 190 - protein, 15 aa, synthetic construct
WYQQKPGKAPKLLIY

SEQ ID NO: 191 - protein, 32 aa, synthetic construct
GVPSRFSGSGSGTDFTLTISSLQPEDVGVYYC

SEQ ID NO: 192 - protein, 10 aa, synthetic construct
FGGGTKLEIK

SEQ ID NO: 193-202 - skipped (10 entries)

SEQ ID NO: 203 - protein, 15 aa, synthetic construct
GGGGSGGSGSGGGGS

SEQ ID NO: 204 - protein, 15 aa, synthetic construct
GGGGSGGSGSSGGGS

SEQ ID NO: 205 - protein, 15 aa, synthetic construct
GGGGSGSGASSGGGS

SEQ ID NO: 206 - protein, 15 aa, synthetic construct
GGGGSGSGASGGGGS

SEQ ID NO: 207 - protein, 15 aa, synthetic construct
SGGGSGGGGSGGGGS

SEQ ID NO: 208 - protein, 18 aa, synthetic construct
GSTSGSGKPGSGEGSTKG

SEQ ID NO: 209 - protein, 19 aa, synthetic construct
PNGASQSSSASHTGSAPGS

SEQ ID NO: 210 - protein, 19 aa, synthetic construct
PNGASNSGSAPDTSSAPGS

SEQ ID NO: 211 - protein, 19 aa, synthetic construct
PNGASHSGSAPNTSSAPGS

SEQ ID NO: 212 - protein, 19 aa, synthetic construct
PNGASESGSASKTSSASGS

SEQ ID NO: 213 - protein, 19 aa, synthetic construct
PNGASNSGSAPKTGSASGS

SEQ ID NO: 214 - protein, 19 aa, synthetic construct
PNGASKSGSASQTSSAPGS

SEQ ID NO: 215 - protein, 19 aa, synthetic construct
PNGASHSSSASQTGSAPGS

SEQ ID NO: 216 - protein, 12 aa, synthetic construct
PNGASKRSAPGS

SEQ ID NO: 217 - protein, 19 aa, synthetic construct
PNGASHSGSAPHTSSASGS

SEQ ID NO: 218 - protein, 15 aa, synthetic construct
RGRGRGRGRSRGGGS

SEQ ID NO: 219 - protein, 15 aa, synthetic construct
SHGGSHGGGSGGGGS

SEQ ID NO: 220 - protein, 5 aa, synthetic construct
GQAGR

SEQ ID NO: 221 - protein, 8 aa, synthetic construct
GGGSGGGG

SEQ ID NO: 222 - protein, 5 aa, synthetic construct
SGGGG

SEQ ID NO: 223 - protein, 5 aa, synthetic construct
GGGGS

SEQ ID NO: 224 - protein, 30 aa, synthetic construct
GGGGSGGGGSGGGGSGGGGSGGGGSGGGGS

SEQ ID NO: 225 - protein, 16 aa, synthetic construct
GGGGGSGGGGSGGGGS

SEQ ID NO: 226 - protein, 26 aa, synthetic construct
GGGGGSGGGGSGGGGSGGGGSGGGGS

SEQ ID NO: 227 - protein, 25 aa, synthetic construct
GGGGSGGGGSGGGGSGGGGSGGGGS

SEQ ID NO: 228 - skipped

SEQ ID NO: 229 - protein, 18 aa, synthetic construct
VEGGSGGSGGSGGSGGVD

SEQ ID NO: 230 - protein, 22 aa, synthetic construct
MDMRVPAQLLGLLLLWLRGARC

SEQ ID NO: 231 - DNA, 66 bp, synthetic construct
  source 1..66: mol_type = other DNA
atggatatgagggtccccgcacaattgctcggtctccttctgctttggctccggggcgcgcggtgc

SEQ ID NO: 232 - protein, 22 aa, synthetic construct
MDMRVLAQLLGLLLLCFPGARC

SEQ ID NO: 233 - protein, 20 aa, synthetic construct
MEAPAQLLFLLLLWLPDTTG

SEQ ID NO: 234 - protein, 20 aa, synthetic construct
MVLQTQVFISLLLWISGAYG

SEQ ID NO: 235 - protein, 19 aa, synthetic construct
MLPSQLIGFLLLWVPASRG

SEQ ID NO: 236 - protein, 22 aa, synthetic construct
MDMRVPAQLLGLLLLWLSGARC

SEQ ID NO: 237 - protein, 15 aa, synthetic construct
EPKSCDKTHTCPPCP

SEQ ID NO: 238 - protein, 15 aa, synthetic construct
DKTHTCPPCPAPPVA

SEQ ID NO: 239 - protein, 12 aa, synthetic construct
ESKYGPPCPPCP

SEQ ID NO: 240 - protein, 12 aa, synthetic construct
ESKYGPPCPSCP

SEQ ID NO: 241 - protein, 49 aa, synthetic construct
KPTTTPAPRPPTPAPTIASQPLSLRPEACRPAAGGAVHTRGLDFACDIY

SEQ ID NO: 242 - protein, 47 aa, synthetic construct
TTTPAPRPPTPAPTIASQPLSLRPEACRPAAGGAVHTRGLDFACDIY

SEQ ID NO: 243 - protein, 45 aa, synthetic construct
TTTPAPRPPTPAPTIASQPLSLRPEACRPAAGGAVHTRGLDFACD

SEQ ID NO: 244 - protein, 39 aa, synthetic construct
IEVMYPPPYLDNERSNGTIIHVKGKHLCPSPLFPGPSKP

SEQ ID NO: 245 - protein, 43 aa, synthetic construct
GAAAIEVMYPPPYLDNEKSNGTIIHVKGKHLCPSPLFPGPSKP

SEQ ID NO: 246 - protein, 21 aa, synthetic construct
LCYLLDGILFIYGVILTALFL

SEQ ID NO: 247 - protein, 22 aa, synthetic construct
MALIVLGGVAGLLLFIGLGIFF

SEQ ID NO: 248 - protein, 8 aa, synthetic construct
EPKSCDKT

SEQ ID NO: 249 - protein, 24 aa, synthetic construct
IYIWAPLAGTCGVLLLSLVITLYC

SEQ ID NO: 250 - protein, 22 aa, synthetic construct
IWAPLAGTCGVLLLSLVITLYC

SEQ ID NO: 251 - protein, 19 aa, synthetic construct
IWAPLAGTCGVLLLSLVIT

SEQ ID NO: 252 - protein, 27 aa, synthetic construct
FWVLVVVGGVLACYSLLVTVAFIIFWV

SEQ ID NO: 253 - protein, 21 aa, synthetic construct
FWLPIGCAAFVVVCILGCILI

SEQ ID NO: 254 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 255 - protein, 113 aa, synthetic construct
RVKFSRSADAPAYKQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 256 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 257 - protein, 42 aa, synthetic construct
KRGRKKLLYIFKQPFMRPVQTTQEEDGCSCRFPEEEEGGCEL

SEQ ID NO: 258 - protein, 37 aa, synthetic construct
KRGRKKLLYIFKQPFMRPVQTTQEEDGCSCRFPEEEE

SEQ ID NO: 259 - protein, 41 aa, synthetic construct
RSKRSRLLHSDYMNMTPRRPGPTRKHYQPYAPPRDFAAYRS

SEQ ID NO: 260 - protein, 42 aa, synthetic construct
ALYLLRRDQRLPPDAHKPPGGGSFRTPIQEEQADAHSTLAKI

SEQ ID NO: 261 - protein, 38 aa, synthetic construct
CWLTKKKYSSSVHDPNGEYMFMRAVNTAKKSRLTDVTL

SEQ ID NO: 262 - protein, 24 aa, synthetic construct
LCARPRRSPAQEDGKVYINMPGRG

SEQ ID NO: 263 - protein, 52 aa, synthetic construct
YFLGRLVPRGRGAAEAATRKQRITETESPYQELQGQRSDVYSDLNTQRPYYK

SEQ ID NO: 264 - protein, 48 aa, synthetic construct
QRRKYRSNKGESPVEPAEPCHYSCPREEEGSTIPIQEDYRKPEPACSP

SEQ ID NO: 265 - protein, 483 aa, synthetic construct
MALPVTALLLPLALLLHAARPQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 266 - protein, 483 aa, synthetic construct
MALPVTALLLPLALLLHAARPQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFAVYYCHQSSSLPLTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 267 - protein, 483 aa, synthetic construct
MALPVTALLLPLALLLHAARPQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDVAVYYCHQSNSLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 268 - protein, 483 aa, synthetic construct
MALPVTALLLPLALLLHAARPQVQLVESGGGLVKPGGSLRLSCAASGFTFSDYYMSWIRQAPGKGLEWVSYISSSGSTIVYADSVKGRFTISRDNAKNSLYLQMNSLRAEDTAVYYCARPIRRGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 269 - protein, 483 aa, synthetic construct
MALPVTALLLPLALLLHAARPQVQLVESGGGLVKPGGSLRLSCAASGFTFSDYYMSWIRQAPGKGLEWVSYISSSGSTINYADSVKGRFTISRDNAKNSLYLQMNSLRAEDTAVYYCARPIRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 270 - protein, 491 aa, synthetic construct
MALPVTALLLPLALLLHAARPEVQLVQSGAEVKKPGSSVKVSCKASGGTFSSYAISWVRQAPGQGLEWMGGIIPIFGTANYAQKFQGRVTITADESTSTAYMELSSLRSDDTAVYYCARERIAVAGTNYYYGMDVWGQGTLVTVSSGGGGSGGGGSGGGGSEIVLTQSPATLSVSPGERATLSCRASQSVSSSNLAWYQQKPGQAPRLLIYGASTRATGIPARFSGTGSGTDFTLTISRLEPEDFAVYYCQQYGSSITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 271 - protein, 488 aa, synthetic construct
MALPVTALLLPLALLLHAARPEVQLVESGAEVKKPGASVKVSCKASGYTFTGYYMHWVRQAPGQGLVWMGRINPNSGGTNYAQKFQGRVTMTRDTSISTAYMELSRLRSDDTAVYYCAFGTRHAFDIWGQGTMVTVSSGGGGSGGGGSGGGGSNFMLTQPHSVSESPGKTVTISCTRSSGSIASNYVQWYQQRPGSAPTTVIYEDNQRPSGVPDRFSGSIDSSSNSASLTISGLKTEDEADYYCQSYDSIVHVVFGGGTKVTVLGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 272 - protein, 488 aa, synthetic construct
MALPVTALLLPLALLLHAARPEVQLVQSGAEVKKPGASVKVSCKASGGTFSSYAISWVRQAPGQGLEWMGGIIPIFGTANYAQKFQGRVTITADKSTSTAYMELSSLRSEDTAVYYCARDVIVGATSSGMDVWGQGTTVTVSSGGGGSGGGGSGGGGSDIQMTQSPSSVSASVGDRVTITCRASQSISRYLNWYQQKPGKAPKLLIYGASSLQSGVPSRFSGSGFGTDFTLTISSLQPEDFATYYCQQSYSTPLTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 273 - protein, 483 aa, synthetic construct
MALPVTALLLPLALLLHAARPQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 274 - protein, 483 aa, synthetic construct
MALPVTALLLPLALLLHAARPQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFAVYYCHQSSSLPLTFGGGTKLEIKGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 275 - protein, 483 aa, synthetic construct
MALPVTALLLPLALLLHAARPQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDVAVYYCHQSNSLPLTFGGGTKVEIKGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 276 - protein, 483 aa, synthetic construct
MALPVTALLLPLALLLHAARPQVQLVESGGGLVKPGGSLRLSCAASGFTFSDYYMSWIRQAPGKGLEWVSYISSSGSTIVYADSVKGRFTISRDNAKNSLYLQMNSLRAEDTAVYYCARPIRRGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 277 - protein, 483 aa, synthetic construct
MALPVTALLLPLALLLHAARPQVQLVESGGGLVKPGGSLRLSCAASGFTFSDYYMSWIRQAPGKGLEWVSYISSSGSTINYADSVKGRFTISRDNAKNSLYLQMNSLRAEDTAVYYCARPIRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 278 - protein, 491 aa, synthetic construct
MALPVTALLLPLALLLHAARPEVQLVQSGAEVKKPGSSVKVSCKASGGTFSSYAISWVRQAPGQGLEWMGGIIPIFGTANYAQKFQGRVTITADESTSTAYMELSSLRSDDTAVYYCARERIAVAGTNYYYGMDVWGQGTLVTVSSGGGGSGGGGSGGGGSEIVLTQSPATLSVSPGERATLSCRASQSVSSSNLAWYQQKPGQAPRLLIYGASTRATGIPARFSGTGSGTDFTLTISRLEPEDFAVYYCQQYGSSITFGQGTRLEIKGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 279 - protein, 488 aa, synthetic construct
MALPVTALLLPLALLLHAARPEVQLVESGAEVKKPGASVKVSCKASGYTFTGYYMHWVRQAPGQGLVWMGRINPNSGGTNYAQKFQGRVTMTRDTSISTAYMELSRLRSDDTAVYYCAFGTRHAFDIWGQGTMVTVSSGGGGSGGGGSGGGGSNFMLTQPHSVSESPGKTVTISCTRSSGSIASNYVQWYQQRPGSAPTTVIYEDNQRPSGVPDRFSGSIDSSSNSASLTISGLKTEDEADYYCQSYDSIVHVVFGGGTKVTVLGGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 280 - protein, 488 aa, synthetic construct
MALPVTALLLPLALLLHAARPEVQLVQSGAEVKKPGASVKVSCKASGGTFSSYAISWVRQAPGQGLEWMGGIIPIFGTANYAQKFQGRVTITADKSTSTAYMELSSLRSEDTAVYYCARDVIVGATSSGMDVWGQGTTVTVSSGGGGSGGGGSGGGGSDIQMTQSPSSVSASVGDRVTITCRASQSISRYLNWYQQKPGKAPKLLIYGASSLQSGVPSRFSGSGFGTDFTLTISSLQPEDFATYYCQQSYSTPLTFGQGTKLEIKGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 281 - protein, 271 aa, synthetic construct
GGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 282 - DNA, 813 bp, synthetic construct
  source 1..813: mol_type = other DNA
ggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactagg

SEQ ID NO: 283 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 284 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 285 - protein, 108 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 286 - protein, 117 aa, synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 287 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 288 - protein, 125 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 289 - protein, 107 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 290 - protein, 119 aa, synthetic construct
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 291 - protein, 107 aa, synthetic construct
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 292 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 293 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 294 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 295 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 296 - protein, 107 aa, synthetic construct [sequence omitted]

SEQ ID NO: 297 - protein, 114 aa, synthetic construct
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 298 - protein, 112 aa, synthetic construct
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 299 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 300 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 301 - protein, 113 aa, synthetic construct
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 302 - protein, 576 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 303 - protein, 447 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 305 - protein, 576 aa, synthetic construct; identical to SEQ ID NO: 302

SEQ ID NO: 306 - protein, 447 aa, synthetic construct; identical to SEQ ID NO: 303

SEQ ID NO: 307 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFAVYYCHQSSSLPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 576 aa, synthetic construct; identical to SEQ ID NO: 302

SEQ ID NO: 309 - protein, 447 aa, synthetic construct; identical to SEQ ID NO: 303

SEQ ID NO: 310 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCHQSNSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 576 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSQVQLVESGGGLVKPGGSLRLSCAASGFTFSDYYMSWIRQAPGKGLEWVSYISSSGSTIVYADSVKGRFTISRDNAKNSLYLQMNSLRAEDTAVYYCARPIRRGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 312 - protein, 447 aa, synthetic construct
QVQLVESGGGLVKPGGSLRLSCAASGFTFSDYYMSWIRQAPGKGLEWVSYISSSGSTIVYADSVKGRFTISRDNAKNSLYLQMNSLRAEDTAVYYCARPIRRGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 214 aa, synthetic construct; identical to SEQ ID NO: 304

SEQ ID NO: 314 - protein, 576 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSQVQLVESGGGLVKPGGSLRLSCAASGFTFSDYYMSWIRQAPGKGLEWVSYISSSGSTINYADSVKGRFTISRDNAKNSLYLQMNSLRAEDTAVYYCARPIRWGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 315 - protein, 447 aa, synthetic construct
QVQLVESGGGLVKPGGSLRLSCAASGFTFSDYYMSWIRQAPGKGLEWVSYISSSGSTINYADSVKGRFTISRDNAKNSLYLQMNSLRAEDTAVYYCARPIRWGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 214 aa, synthetic construct; identical to SEQ ID NO: 304

SEQ ID NO: 317 - DNA, 1728 bp, synthetic construct
  source 1..1728: mol_type = other DNA
gaggtgcagctggtggagtctgggggaggcttggtacagcctggagggtccctgagactctcctgtgcagcctctggattcaccttcagtaactatggtatgagctggatccgccaggctccagggaaggggctagagtggattgggagtatctattacagtgggagcaccatctacaacccctccctcaagagtctagtcaccatctccagagacaattccaagaacacgctgtatctgcaaatgaacagcctgagagccgaggacacagccacgtattactgtgcgagagagagtattgactactggggccagggaaccctggtcaccgtctcctcaagtggagggggtggcagcggtggcgggggaagtggcggtggagggtcgcaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatcaactatgcagattccgtgaaagggcgcttcaccatctctcgtgacaatgcaaagaacagtttatacttacaaatgaactcgctgcgtgcggaagatacggctgtgtattactgtgcgagacccatccggtggggtatggacgtctggggccaagggaccacggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgcttcacgcagaagagcctctccctgtctccgggtaaa

SEQ ID NO: 318 - DNA, 1341 bp, synthetic construct
  source 1..1341: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatcaactatgcagattccgtgaaagggcgcttcaccatctctcgtgacaatgcaaagaacagtttatacttacaaatgaactcgctgcgtgcggaagatacggctgtgtattactgtgcgagacccatccggtggggtatggacgtctggggccaagggaccacggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 319 - DNA, 642 bp, synthetic construct; identical to SEQ ID NO: 137
  source 1..642: mol_type = other DNA

SEQ ID NO: 320 - protein, 584 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSEVQLVQSGAEVKKPGSSVKVSCKASGGTFSSYAISWVRQAPGQGLEWMGGIIPIFGTANYAQKFQGRVTITADESTSTAYMELSSLRSDDTAVYYCARERIAVAGTNYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 321 - protein, 455 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSDDTAVYYCARERIAVAGTNYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 214 aa, synthetic construct
EIVLTQSPATLSVSPGERATLSCRASQSVSSSNLAWYQQKPGQAPRLLIYGASTRATGIPARFSGTGSGTDFTLTISRLEPEDFAVYYCQQYGSSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - protein, 576 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSEVQLVESGAEVKKPGASVKVSCKASGYTFTGYYMHWVRQAPGQGLVWMGRINPNSGGTNYAQKFQGRVTMTRDTSISTAYMELSRLRSDDTAVYYCAFGTRHAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 324 - protein, 447 aa, synthetic construct
EVQLVESGAEVKKPGASVKVSCKASGYTFTGYYMHWVRQAPGQGLVWMGRINPNSGGTNYAQKFQGRVTMTRDTSISTAYMELSRLRSDDTAVYYCAFGTRHAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 217 aa, synthetic construct
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDSIVHVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 326 - protein, 581 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSEVQLVQSGAEVKKPGASVKVSCKASGGTFSSYAISWVRQAPGQGLEWMGGIIPIFGTANYAQKFQGRVTITADKSTSTAYMELSSLRSEDTAVYYCARDVIVGATSSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 327 - protein, 452 aa, synthetic construct
EVQLVQSGAEVKKPGASVKVSCKASGGTFSSYAISWVRQAPGQGLEWMGGIIPIFGTANYAQKFQGRVTITADKSTSTAYMELSSLRSEDTAVYYCARDVIVGATSSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 214 aa, synthetic construct
DIQMTQSPSSVSASVGDRVTITCRASQSISRYLNWYQQKPGKAPKLLIYGASSLQSGVPSRFSGSGFGTDFTLTISSLQPEDFATYYCQQSYSTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 576 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 214 aa, synthetic construct; identical to SEQ ID NO: 304

SEQ ID NO: 331 - protein, 576 aa, synthetic construct; identical to SEQ ID NO: 329

SEQ ID NO: 332 - protein, 214 aa, synthetic construct; identical to SEQ ID NO: 307

SEQ ID NO: 333 - protein, 576 aa, synthetic construct; identical to SEQ ID NO: 329

SEQ ID NO: 334 - protein, 214 aa, synthetic construct; identical to SEQ ID NO: 310

SEQ ID NO: 335 - protein, 576 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSQVQLVESGGGLVKPGGSLRLSCAASGFTFSDYYMSWIRQAPGKGLEWVSYISSSGSTIVYADSVKGRFTISRDNAKNSLYLQMNSLRAEDTAVYYCARPIRRGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 214 aa, synthetic construct; identical to SEQ ID NO: 304

SEQ ID NO: 337 - protein, 576 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSQVQLVESGGGLVKPGGSLRLSCAASGFTFSDYYMSWIRQAPGKGLEWVSYISSSGSTINYADSVKGRFTISRDNAKNSLYLQMNSLRAEDTAVYYCARPIRWGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 214 aa, synthetic construct; identical to SEQ ID NO: 304

SEQ ID NO: 339 - DNA, 1728 bp, synthetic construct
  source 1..1728: mol_type = other DNA
gaggtgcagctggtggagtctgggggaggcttggtacagcctggagggtccctgagactctcctgtgcagcctctggattcaccttcagtaactatggtatgagctggatccgccaggctccagggaaggggctagagtggattgggagtatctattacagtgggagcaccatctacaacccctccctcaagagtctagtcaccatctccagagacaattccaagaacacgctgtatctgcaaatgaacagcctgagagccgaggacacagccacgtattactgtgcgagagagagtattgactactggggccagggaaccctggtcaccgtctcctcaagtggagggggtggcagcggtggcgggggaagtggcggtggagggtcgcaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatcaactatgcagattccgtgaaagggcgcttcaccatctctcgtgacaatgcaaagaacagtttatacttacaaatgaactcgctgcgtgcggaagatacggctgtgtattactgtgcgagacccatccggtggggtatggacgtctggggccaagggaccacggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 340 - DNA, 642 bp, synthetic construct; identical to SEQ ID NO: 137
  source 1..642: mol_type = other DNA

SEQ ID NO: 341 - protein, 584 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSEVQLVQSGAEVKKPGSSVKVSCKASGGTFSSYAISWVRQAPGQGLEWMGGIIPIFGTANYAQKFQGRVTITADESTSTAYMELSSLRSDDTAVYYCARERIAVAGTNYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - protein, 214 aa, synthetic construct; identical to SEQ ID NO: 322

SEQ ID NO: 343 - protein, 576 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSEVQLVESGAEVKKPGASVKVSCKASGYTFTGYYMHWVRQAPGQGLVWMGRINPNSGGTNYAQKFQGRVTMTRDTSISTAYMELSRLRSDDTAVYYCAFGTRHAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 344 - protein, 217 aa, synthetic construct; identical to SEQ ID NO: 325

SEQ ID NO: 345 - protein, 581 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSSSGGGGSGGGGSGGGGSEVQLVQSGAEVKKPGASVKVSCKASGGTFSSYAISWVRQAPGQGLEWMGGIIPIFGTANYAQKFQGRVTITADKSTSTAYMELSSLRSEDTAVYYCARDVIVGATSSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 214 aa, synthetic construct; identical to SEQ ID NO: 328

SEQ ID NO: 347 - protein, 762 aa, synthetic construct
MTRLTVLALLAGLLASSRAQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 762 aa, synthetic construct
MTRLTVLALLAGLLASSRAQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFAVYYCHQSSSLPLTFGGGTKLEIKGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 762 aa, synthetic construct
MTRLTVLALLAGLLASSRAQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARPLRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDVAVYYCHQSNSLPLTFGGGTKVEIKGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 762 aa, synthetic construct
MTRLTVLALLAGLLASSRAQVQLVESGGGLVKPGGSLRLSCAASGFTFSDYYMSWIRQAPGKGLEWVSYISSSGSTIVYADSVKGRFTISRDNAKNSLYLQMNSLRAEDTAVYYCARPIRRGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 762 aa, synthetic construct
MTRLTVLALLAGLLASSRAQVQLVESGGGLVKPGGSLRLSCAASGFTFSDYYMSWIRQAPGKGLEWVSYISSSGSTINYADSVKGRFTISRDNAKNSLYLQMNSLRAEDTAVYYCARPIRWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDAATYYCHQSSSLPLTFGGGTKVEIKGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 770 aa, synthetic construct
MTRLTVLALLAGLLASSRAEVQLVQSGAEVKKPGSSVKVSCKASGGTFSSYAISWVRQAPGQGLEWMGGIIPIFGTANYAQKFQGRVTITADESTSTAYMELSSLRSDDTAVYYCARERIAVAGTNYYYGMDVWGQGTLVTVSSGGGGSGGGGSGGGGSEIVLTQSPATLSVSPGERATLSCRASQSVSSSNLAWYQQKPGQAPRLLIYGASTRATGIPARFSGTGSGTDFTLTISRLEPEDFAVYYCQQYGSSITFGQGTRLEIKGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 767 aa, synthetic construct
MTRLTVLALLAGLLASSRAEVQLVESGAEVKKPGASVKVSCKASGYTFTGYYMHWVRQAPGQGLVWMGRINPNSGGTNYAQKFQGRVTMTRDTSISTAYMELSRLRSDDTAVYYCAFGTRHAFDIWGQGTMVTVSSGGGGSGGGGSGGGGSNFMLTQPHSVSESPGKTVTISCTRSSGSIASNYVQWYQQRPGSAPTTVIYEDNQRPSGVPDRFSGSIDSSSNSASLTISGLKTEDEADYYCQSYDSIVHVVFGGGTKVTVLGGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 767 aa, synthetic construct
MTRLTVLALLAGLLASSRAEVQLVQSGAEVKKPGASVKVSCKASGGTFSSYAISWVRQAPGQGLEWMGGIIPIFGTANYAQKFQGRVTITADKSTSTAYMELSSLRSEDTAVYYCARDVIVGATSSGMDVWGQGTTVTVSSGGGGSGGGGSGGGGSDIQMTQSPSSVSASVGDRVTITCRASQSISRYLNWYQQKPGKAPKLLIYGASSLQSGVPSRFSGSGFGTDFTLTISSLQPEDFATYYCQQSYSTPLTFGQGTKLEIKGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 5 aa, synthetic construct
EYYMS

SEQ ID NO: 356 - protein, 5 aa, synthetic construct
DYYMT

SEQ ID NO: 357 - protein, 5 aa, synthetic construct
EYYMT

SEQ ID NO: 358 - protein, 5 aa, synthetic construct
DYYMH

SEQ ID NO: 359 - protein, 5 aa, synthetic construct
DYYMA

SEQ ID NO: 360 - protein, 5 aa, synthetic construct
EYYMH

SEQ ID NO: 361 - protein, 5 aa, synthetic construct
EYYMA

SEQ ID NO: 362 - protein, 5 aa, synthetic construct
EYYMD

SEQ ID NO: 363 - protein, 5 aa, synthetic construct
DYYMD

SEQ ID NO: 364 - protein, 5 aa, synthetic construct
EYYME

SEQ ID NO: 365 - protein, 17 aa, synthetic construct
YLSSSGSTIYYADSVKG

SEQ ID NO: 366 - protein, 17 aa, synthetic construct
YLTSSGSTIYYADSVKG

SEQ ID NO: 367 - protein, 17 aa, synthetic construct
YISSSGSTIYYAESVKG

SEQ ID NO: 368 - protein, 17 aa, synthetic construct
YISTSGSTIYYAESVKG

SEQ ID NO: 369 - protein, 17 aa, synthetic construct
YISSSGTTIYYAESVKG

SEQ ID NO: 370 - protein, 17 aa, synthetic construct
YISSSGSTIYFAESVKG

SEQ ID NO: 371 - protein, 17 aa, synthetic construct
YISSTGSTIYYADSVKG

SEQ ID NO: 372 - protein, 17 aa, synthetic construct
YISTSGSTIYYADSVKG

SEQ ID NO: 373 - protein, 17 aa, synthetic construct
YISSSGTTIYYADSVKG

SEQ ID NO: 374 - protein, 17 aa, synthetic construct
YISSSGSTIYYADTVKG

SEQ ID NO: 375 - protein, 8 aa, synthetic construct
PLKWGMDV

SEQ ID NO: 376 - protein, 8 aa, synthetic construct
PLRWGMEV

SEQ ID NO: 377 - protein, 8 aa, synthetic construct
PLRWGMDA

SEQ ID NO: 378 - protein, 8 aa, synthetic construct
PLRWSMDV

SEQ ID NO: 379 - protein, 8 aa, synthetic construct
PLKWGMDA

SEQ ID NO: 380 - protein, 8 aa, synthetic construct
PLKWGMEV

SEQ ID NO: 381 - protein, 8 aa, synthetic construct
PLRWGLDV

SEQ ID NO: 382 - protein, 8 aa, synthetic construct
PVRWGMDV

SEQ ID NO: 383 - protein, 8 aa, synthetic construct
PLRFGMDV

SEQ ID NO: 384 - protein, 8 aa, synthetic construct
PVKWGMDV

SEQ ID NO: 385 - protein, 11 aa, synthetic construct
KASQSISSYLN

SEQ ID NO: 386 - protein, 11 aa, synthetic construct
RATQSISSYLN

SEQ ID NO: 387 - protein, 11 aa, synthetic construct
RASQTISSYLN

SEQ ID NO: 388 - protein, 11 aa, synthetic construct
RASQSITSYLN

SEQ ID NO: 389 - protein, 11 aa, synthetic construct
RASQSISTYLN

SEQ ID NO: 390 - protein, 11 aa, synthetic construct
KASQSISSYLD

SEQ ID NO: 391 - protein, 11 aa, synthetic construct
RASESISSYLN

SEQ ID NO: 392 - protein, 11 aa, synthetic construct
RASQSISSYMN

SEQ ID NO: 393 - protein, 11 aa, synthetic construct
RTSQSISSYLN

SEQ ID NO: 394 - protein, 11 aa, synthetic construct
RASQSLSSYLN

SEQ ID NO: 395 - protein, 7 aa, synthetic construct
TASSLQS

SEQ ID NO: 396 - protein, 7 aa, synthetic construct
ATSSLQS

SEQ ID NO: 397 - protein, 7 aa, synthetic construct
AATSLQS

SEQ ID NO: 398 - protein, 7 aa, synthetic construct
AASTLQS

SEQ ID NO: 399 - protein, 7 aa, synthetic construct
AASSLES

SEQ ID NO: 400 - protein, 7 aa, synthetic construct
AASSLQT

SEQ ID NO: 401 - protein, 7 aa, synthetic construct
TASSLQT

SEQ ID NO: 402 - protein, 7 aa, synthetic construct
AASSLQA

SEQ ID NO: 403 - protein, 7 aa, synthetic construct
AASSMQS

SEQ ID NO: 404 - protein, 7 aa, synthetic construct
ADSSLQS

SEQ ID NO: 405 - protein, 9 aa, synthetic construct
HESSSLPLT

SEQ ID NO: 406 - protein, 9 aa, synthetic construct
HQTSSLPLT

SEQ ID NO: 407 - protein, 9 aa, synthetic construct
HQSTSLPLT

SEQ ID NO: 408 - protein, 9 aa, synthetic construct
HQSSTLPLT

SEQ ID NO: 409 - protein, 9 aa, synthetic construct
HQSSSMPLT

SEQ ID NO: 410 - protein, 9 aa, synthetic construct
HQSSSLPMT

SEQ ID NO: 411 - protein, 9 aa, synthetic construct
HQSSSLPLS

SEQ ID NO: 412 - protein, 9 aa, synthetic construct
TQSSSLPLT

SEQ ID NO: 413 - protein, 9 aa, synthetic construct
HQSSSLPLD

SEQ ID NO: 414 - protein, 9 aa, synthetic construct
HQSSSVPLT

SEQ ID NO: 415 - protein, 5 aa, synthetic construct
EYYMS

SEQ ID NO: 416 - protein, 5 aa, synthetic construct
DYYMT

SEQ ID NO: 417 - protein, 5 aa, synthetic construct
EYYMT

SEQ ID NO: 418 - protein, 5 aa, synthetic construct
DYYMH

SEQ ID NO: 419 - protein, 5 aa, synthetic construct
DYYMA

SEQ ID NO: 420 - protein, 5 aa, synthetic construct
EYYMH

SEQ ID NO: 421 - protein, 5 aa, synthetic construct
EYYMA

SEQ ID NO: 422 - protein, 5 aa, synthetic construct
EYYMD

SEQ ID NO: 423 - protein, 5 aa, synthetic construct
DYYMD

SEQ ID NO: 424 - protein, 5 aa, synthetic construct
EYYME

SEQ ID NO: 425 - protein, 17 aa, synthetic construct
YLSSSGSTIYYADSVKG

SEQ ID NO: 426 - protein, 17 aa, synthetic construct
YLTSSGSTIYYADSVKG

SEQ ID NO: 427 - protein, 17 aa, synthetic construct
YISSSGSTIYYAESVKG

SEQ ID NO: 428 - protein, 17 aa, synthetic construct
YISTSGSTIYYAESVKG

SEQ ID NO: 429 - protein, 17 aa, synthetic construct
YISSSGTTIYYAESVKG

SEQ ID NO: 430 - protein, 17 aa, synthetic construct
YISSSGSTIYFAESVKG

SEQ ID NO: 431 - protein, 17 aa, synthetic construct
YISSTGSTIYYADSVKG

SEQ ID NO: 432 - protein, 17 aa, synthetic construct
YISTSGSTIYYADSVKG

SEQ ID NO: 433 - protein, 17 aa, synthetic construct
YISSSGTTIYYADSVKG

SEQ ID NO: 434 - protein, 17 aa, synthetic construct
YISSSGSTIYYADTVKG

SEQ ID NO: 435 - protein, 8 aa, synthetic construct
PLKWGMDV

SEQ ID NO: 436 - protein, 8 aa, synthetic construct
PLRWGMEV

SEQ ID NO: 437 - protein, 8 aa, synthetic construct
PLRWGMDA

SEQ ID NO: 438 - protein, 8 aa, synthetic construct
PLRWSMDV

SEQ ID NO: 439 - protein, 8 aa, synthetic construct
PLKWGMDA

SEQ ID NO: 440 - protein, 8 aa, synthetic construct
PLKWGMEV

SEQ ID NO: 441 - protein, 8 aa, synthetic construct
PLRWGLDV

SEQ ID NO: 442 - protein, 8 aa, synthetic construct
PVRWGMDV

SEQ ID NO: 443 - protein, 8 aa, synthetic construct
PLRFGMDV

SEQ ID NO: 444 - protein, 8 aa, synthetic construct
PVKWGMDV

SEQ ID NO: 445 - protein, 11 aa, synthetic construct
KASQSISSYLN

SEQ ID NO: 446 - protein, 11 aa, synthetic construct
RATQSISSYLN

SEQ ID NO: 447 - protein, 11 aa, synthetic construct
RASQTISSYLN

SEQ ID NO: 448 - protein, 11 aa, synthetic construct
RASQSITSYLN

SEQ ID NO: 449 - protein, 11 aa, synthetic construct
RASQSISTYLN

SEQ ID NO: 450 - protein, 11 aa, synthetic construct
KASQSISSYLD

SEQ ID NO: 451 - protein, 11 aa, synthetic construct
RASESISSYLN

SEQ ID NO: 452 - protein, 11 aa, synthetic construct
RASQSISSYMN

SEQ ID NO: 453 - protein, 11 aa, synthetic construct
RTSQSISSYLN

SEQ ID NO: 454 - protein, 11 aa, synthetic construct
RASQSLSSYLN

SEQ ID NO: 455 - protein, 7 aa, synthetic construct
TASSLQS

SEQ ID NO: 456 - protein, 7 aa, synthetic construct
ATSSLQS

SEQ ID NO: 457 - protein, 7 aa, synthetic construct
AATSLQS

SEQ ID NO: 458 - protein, 7 aa, synthetic construct
AASTLQS

SEQ ID NO: 459 - protein, 7 aa, synthetic construct
AASSLES

SEQ ID NO: 460 - protein, 7 aa, synthetic construct
AASSLQT

SEQ ID NO: 461 - protein, 7 aa, synthetic construct
TASSLQT

SEQ ID NO: 462 - protein, 7 aa, synthetic construct
AASSLQA

SEQ ID NO: 463 - protein, 7 aa, synthetic construct
AASSMQS

SEQ ID NO: 464 - protein, 7 aa, synthetic construct
ADSSLQS

SEQ ID NO: 465 - protein, 9 aa, synthetic construct
HESSSLPLT

SEQ ID NO: 466 - protein, 9 aa, synthetic construct
HQTSSLPLT

SEQ ID NO: 467 - protein, 9 aa, synthetic construct
HQSTSLPLT

SEQ ID NO: 468 - protein, 9 aa, synthetic construct
HQSSTLPLT

SEQ ID NO: 469 - protein, 9 aa, synthetic construct
HQSSSMPLT

SEQ ID NO: 470 - protein, 9 aa, synthetic construct
HQSSSLPMT

SEQ ID NO: 471 - protein, 9 aa, synthetic construct
HQSSSLPLS

SEQ ID NO: 472 - protein, 9 aa, synthetic construct
TQSSSLPLT

SEQ ID NO: 473 - protein, 9 aa, synthetic construct
HQSSSLPLD

SEQ ID NO: 474 - protein, 9 aa, synthetic construct
HQSSSVPLT

SEQ ID NO: 475 - protein, 5 aa, synthetic construct
EYYMS

SEQ ID NO: 476 - protein, 5 aa, synthetic construct
DYYMT

SEQ ID NO: 477 - protein, 5 aa, synthetic construct
EYYMT

SEQ ID NO: 478 - protein, 5 aa, synthetic construct
DYYMH

SEQ ID NO: 479 - protein, 5 aa, synthetic construct
DYYMA

SEQ ID NO: 480 - protein, 5 aa, synthetic construct
EYYMH

SEQ ID NO: 481 - protein, 5 aa, synthetic construct
EYYMA

SEQ ID NO: 482 - protein, 5 aa, synthetic construct
EYYMD

SEQ ID NO: 483 - protein, 5 aa, synthetic construct
DYYMD

SEQ ID NO: 484 - protein, 5 aa, synthetic construct
EYYME

SEQ ID NO: 485 - protein, 17 aa, synthetic construct
YLSSSGSTIYYADSVKG

SEQ ID NO: 486 - protein, 17 aa, synthetic construct
YLTSSGSTIYYADSVKG

SEQ ID NO: 487 - protein, 17 aa, synthetic construct
YISSSGSTIYYAESVKG

SEQ ID NO: 488 - protein, 17 aa, synthetic construct
YISTSGSTIYYAESVKG

SEQ ID NO: 489 - protein, 17 aa, synthetic construct
YISSSGTTIYYAESVKG

SEQ ID NO: 490 - protein, 17 aa, synthetic construct
YISSSGSTIYFAESVKG

SEQ ID NO: 491 - protein, 17 aa, synthetic construct
YISSTGSTIYYADSVKG

SEQ ID NO: 492 - protein, 17 aa, synthetic construct
YISTSGSTIYYADSVKG

SEQ ID NO: 493 - protein, 17 aa, synthetic construct
YISSSGTTIYYADSVKG

SEQ ID NO: 494 - protein, 17 aa, synthetic construct
YISSSGSTIYYADTVKG

SEQ ID NO: 495 - protein, 8 aa, synthetic construct
PLKWGMDV

SEQ ID NO: 496 - protein, 8 aa, synthetic construct
PLRWGMEV

SEQ ID NO: 497 - protein, 8 aa, synthetic construct
PLRWGMDA

SEQ ID NO: 498 - protein, 8 aa, synthetic construct
PLRWSMDV

SEQ ID NO: 499 - protein, 8 aa, synthetic construct
PLKWGMDA

SEQ ID NO: 500 - protein, 8 aa, synthetic construct
PLKWGMEV

SEQ ID NO: 501 - protein, 8 aa, synthetic construct
PLRWGLDV

SEQ ID NO: 502 - protein, 8 aa, synthetic construct
PVRWGMDV

SEQ ID NO: 503 - protein, 8 aa, synthetic construct
PLRFGMDV

SEQ ID NO: 504 - protein, 8 aa, synthetic construct
PVKWGMDV

SEQ ID NO: 505 - protein, 11 aa, synthetic construct
KASQSISSYLN

SEQ ID NO: 506 - protein, 11 aa, synthetic construct
RATQSISSYLN

SEQ ID NO: 507 - protein, 11 aa, synthetic construct
RASQTISSYLN

SEQ ID NO: 508 - protein, 11 aa, synthetic construct
RASQSITSYLN

SEQ ID NO: 509 - protein, 11 aa, synthetic construct
RASQSISTYLN

SEQ ID NO: 510 - protein, 11 aa, synthetic construct
KASQSISSYLD

SEQ ID NO: 511 - protein, 11 aa, synthetic construct
RASESISSYLN

SEQ ID NO: 512 - protein, 11 aa, synthetic construct
RASQSISSYMN

SEQ ID NO: 513 - protein, 11 aa, synthetic construct
RTSQSISSYLN

SEQ ID NO: 514 - protein, 11 aa, synthetic construct
RASQSLSSYLN

SEQ ID NO: 515 - protein, 7 aa, synthetic construct
TASSLQS

SEQ ID NO: 516 - protein, 7 aa, synthetic construct
ATSSLQS

SEQ ID NO: 517 - protein, 7 aa, synthetic construct
AATSLQS

SEQ ID NO: 518 - protein, 7 aa, synthetic construct
AASTLQS

SEQ ID NO: 519 - protein, 7 aa, synthetic construct
AASSLES

SEQ ID NO: 520 - protein, 7 aa, synthetic construct
AASSLQT

SEQ ID NO: 521 - protein, 7 aa, synthetic construct
TASSLQT

SEQ ID NO: 522 - protein, 7 aa, synthetic construct
AASSLQA

SEQ ID NO: 523 - protein, 7 aa, synthetic construct
AASSMQS

SEQ ID NO: 524 - protein, 7 aa, synthetic construct
ADSSLQS

SEQ ID NO: 525 - protein, 9 aa, synthetic construct
DQSNSLPLT

SEQ ID NO: 526 - protein, 9 aa, synthetic construct
HESNSLPLT

SEQ ID NO: 527 - protein, 9 aa, synthetic construct
HQTNSLPLT

SEQ ID NO: 528 - protein, 9 aa, synthetic construct
HQSESLPLT

SEQ ID NO: 529 - protein, 9 aa, synthetic construct
HQSNTLPLT

SEQ ID NO: 530 - protein, 9 aa, synthetic construct
HQSNSMPLT

SEQ ID NO: 531 - protein, 9 aa, synthetic construct
HQSNSLPMT

SEQ ID NO: 532 - protein, 9 aa, synthetic construct
HQSNSLPLS

SEQ ID NO: 533 - protein, 9 aa, synthetic construct
HQANSLPLT

SEQ ID NO: 534 - protein, 9 aa, synthetic construct
HQSNALPLT

SEQ ID NO: 535 - protein, 5 aa, synthetic construct
EYYMS

SEQ ID NO: 536 - protein, 5 aa, synthetic construct
DYYMT

SEQ ID NO: 537 - protein, 5 aa, synthetic construct
EYYMT

SEQ ID NO: 538 - protein, 5 aa, synthetic construct
DYYMH

SEQ ID NO: 539 - protein, 5 aa, synthetic construct
DYYMA

SEQ ID NO: 540 - protein, 5 aa, synthetic construct
EYYMH

SEQ ID NO: 541 - protein, 5 aa, synthetic construct
EYYMA

SEQ ID NO: 542 - protein, 5 aa, synthetic construct
EYYMD

SEQ ID NO: 543 - protein, 5 aa, synthetic construct
DYYMD

SEQ ID NO: 544 - protein, 5 aa, synthetic construct
EYYME

SEQ ID NO: 545 - protein, 17 aa, synthetic construct
FISSSGSTIVYADSVKG

SEQ ID NO: 546 - protein, 17 aa, synthetic construct
YMSSSGSTIVYADSVKG

SEQ ID NO: 547 - protein, 17 aa, synthetic construct
YITSSGSTIVYADSVKG

SEQ ID NO: 548 - protein, 17 aa, synthetic construct
YISTSGSTIVYADSVKG

SEQ ID NO: 549 - protein, 17 aa, synthetic construct
YISSTGSTIVYADSVKG

SEQ ID NO: 550 - protein, 17 aa, synthetic construct
YISSSGTTIVYADSVKG

SEQ ID NO: 551 - protein, 17 aa, synthetic construct
YISSSGSTLVYADSVKG

SEQ ID NO: 552 - protein, 17 aa, synthetic construct
YISSSGSTIVYAESVKG

SEQ ID NO: 553 - protein, 17 aa, synthetic construct
YISSSGSTIVYADTVKG

SEQ ID NO: 554 - protein, 17 aa, synthetic construct
YISSSGSTIVYADSVRG

SEQ ID NO: 555 - protein, 8 aa, synthetic construct
PLRRGMDV

SEQ ID NO: 556 - protein, 8 aa, synthetic construct
PMRRGMDV

SEQ ID NO: 557 - protein, 8 aa, synthetic construct
PIKRGMDV

SEQ ID NO: 558 - protein, 8 aa, synthetic construct
PIRKGMDV

SEQ ID NO: 559 - protein, 8 aa, synthetic construct
PIRRAMDV

SEQ ID NO: 560 - protein, 8 aa, synthetic construct
PIRRGLDV

SEQ ID NO: 561 - protein, 8 aa, synthetic construct
PIRRGMEV

SEQ ID NO: 562 - protein, 8 aa, synthetic construct
PIRRGMDA

SEQ ID NO: 563 - protein, 8 aa, synthetic construct
PIRRSMDV

SEQ ID NO: 564 - protein, 8 aa, synthetic construct
PIRRGMQV

SEQ ID NO: 565 - protein, 11 aa, synthetic construct
KASQSISSYLN

SEQ ID NO: 566 - protein, 11 aa, synthetic construct
RATQSISSYLN

SEQ ID NO: 567 - protein, 11 aa, synthetic construct
RASQTISSYLN

SEQ ID NO: 568 - protein, 11 aa, synthetic construct
RASQSITSYLN

SEQ ID NO: 569 - protein, 11 aa, synthetic construct
RASQSISTYLN

SEQ ID NO: 570 - protein, 11 aa, synthetic construct
KASQSISSYLD

SEQ ID NO: 571 - protein, 11 aa, synthetic construct
RASESISSYLN

SEQ ID NO: 572 - protein, 11 aa, synthetic construct
RASQSISSYMN

SEQ ID NO: 573 - protein, 11 aa, synthetic construct
RTSQSISSYLN

SEQ ID NO: 574 - protein, 11 aa, synthetic construct
RASQSLSSYLN

SEQ ID NO: 575 - protein, 7 aa, synthetic construct
TASSLQS

SEQ ID NO: 576 - protein, 7 aa, synthetic construct
ATSSLQS

SEQ ID NO: 577 - protein, 7 aa, synthetic construct
AATSLQS

SEQ ID NO: 578 - protein, 7 aa, synthetic construct
AASTLQS

SEQ ID NO: 579 - protein, 7 aa, synthetic construct
AASSLES

SEQ ID NO: 580 - protein, 7 aa, synthetic construct
AASSLQT

SEQ ID NO: 581 - protein, 7 aa, synthetic construct
TASSLQT

SEQ ID NO: 582 - protein, 7 aa, synthetic construct
AASSLQA

SEQ ID NO: 583 - protein, 7 aa, synthetic construct
AASSMQS

SEQ ID NO: 584 - protein, 7 aa, synthetic construct
ADSSLQS

SEQ ID NO: 585 - protein, 9 aa, synthetic construct
HESSSLPLT

SEQ ID NO: 586 - protein, 9 aa, synthetic construct
HQTSSLPLT

SEQ ID NO: 587 - protein, 9 aa, synthetic construct
HQSTSLPLT

SEQ ID NO: 588 - protein, 9 aa, synthetic construct
HQSSTLPLT

SEQ ID NO: 589 - protein, 9 aa, synthetic construct
HQSSSMPLT

SEQ ID NO: 590 - protein, 9 aa, synthetic construct
HQSSSLPMT

SEQ ID NO: 591 - protein, 9 aa, synthetic construct
HQSSSLPLS

SEQ ID NO: 592 - protein, 9 aa, synthetic construct
TQSSSLPLT

SEQ ID NO: 593 - protein, 9 aa, synthetic construct
HQSSSLPLD

SEQ ID NO: 594 - protein, 9 aa, synthetic construct
HQSSSVPLT

SEQ ID NO: 595 - protein, 5 aa, synthetic construct
EYYMS

SEQ ID NO: 596 - protein, 5 aa, synthetic construct
DYYMT

SEQ ID NO: 597 - protein, 5 aa, synthetic construct
EYYMT

SEQ ID NO: 598 - protein, 5 aa, synthetic construct
DYYMH

SEQ ID NO: 599 - protein, 5 aa, synthetic construct
DYYMA

SEQ ID NO: 600 - protein, 5 aa, synthetic construct
EYYMH

SEQ ID NO: 601 - protein, 5 aa, synthetic construct
EYYMA

SEQ ID NO: 602 - protein, 5 aa, synthetic construct
EYYMD

SEQ ID NO: 603 - protein, 5 aa, synthetic construct
DYYMD

SEQ ID NO: 604 - protein, 5 aa, synthetic construct
EYYME

SEQ ID NO: 605 - protein, 17 aa, synthetic construct
FISSSGSTINYADSVKG

SEQ ID NO: 606 - protein, 17 aa, synthetic construct
YLSSSGSTINYADSVKG

SEQ ID NO: 607 - protein, 17 aa, synthetic construct
YITSSGSTINYADSVKG

SEQ ID NO: 608 - protein, 17 aa, synthetic construct
YISTSGSTINYADSVKG

SEQ ID NO: 609 - protein, 17 aa, synthetic construct
YISSTGSTINYADSVKG

SEQ ID NO: 610 - protein, 17 aa, synthetic construct
YISSSASTINYADSVKG

SEQ ID NO: 611 - protein, 17 aa, synthetic construct
YISSSGTTINYADSVKG

SEQ ID NO: 612 - protein, 17 aa, synthetic construct
YISSSGSTINYAESVKG

SEQ ID NO: 613 - protein, 17 aa, synthetic construct
YISSSGSTINYADSVRG

SEQ ID NO: 614 - protein, 17 aa, synthetic construct
YISSSGSTINYADSVKA

SEQ ID NO: 615 - protein, 8 aa, synthetic construct
PLRWGMDV

SEQ ID NO: 616 - protein, 8 aa, synthetic construct
PIKWGMDV

SEQ ID NO: 617 - protein, 8 aa, synthetic construct
PIRFGMDV

SEQ ID NO: 618 - protein, 8 aa, synthetic construct
PIRWAMDV

SEQ ID NO: 619 - protein, 8 aa, synthetic construct
PIRWGLDV

SEQ ID NO: 620 - protein, 8 aa, synthetic construct
PIRWGMEV

SEQ ID NO: 621 - protein, 8 aa, synthetic construct
PIRWGMDA

SEQ ID NO: 622 - protein, 8 aa, synthetic construct
PIRWSMDV

SEQ ID NO: 623 - protein, 8 aa, synthetic construct
PMRWGMDV

SEQ ID NO: 624 - protein, 8 aa, synthetic construct
PIRWGMEA

SEQ ID NO: 625 - protein, 11 aa, synthetic construct
KASQSISSYLN

SEQ ID NO: 626 - protein, 11 aa, synthetic construct
RATQSISSYLN

SEQ ID NO: 627 - protein, 11 aa, synthetic construct
RASQTISSYLN

SEQ ID NO: 628 - protein, 11 aa, synthetic construct
RASQSITSYLN

SEQ ID NO: 629 - protein, 11 aa, synthetic construct
RASQSISTYLN

SEQ ID NO: 630 - protein, 11 aa, synthetic construct
KASQSISSYLD

SEQ ID NO: 631 - protein, 11 aa, synthetic construct
RASESISSYLN

SEQ ID NO: 632 - protein, 11 aa, synthetic construct
RASQSISSYMN

SEQ ID NO: 633 - protein, 11 aa, synthetic construct
RTSQSISSYLN

SEQ ID NO: 634 - protein, 11 aa, synthetic construct
RASQSLSSYLN

SEQ ID NO: 635 - protein, 7 aa, synthetic construct
TASSLQS

SEQ ID NO: 636 - protein, 7 aa, synthetic construct
ATSSLQS

SEQ ID NO: 637 - protein, 7 aa, synthetic construct
AATSLQS

SEQ ID NO: 638 - protein, 7 aa, synthetic construct
AASTLQS

SEQ ID NO: 639 - protein, 7 aa, synthetic construct
AASSLES

SEQ ID NO: 640 - protein, 7 aa, synthetic construct
AASSLQT

SEQ ID NO: 641 - protein, 7 aa, synthetic construct
TASSLQT

SEQ ID NO: 642 - protein, 7 aa, synthetic construct
AASSLQA

SEQ ID NO: 643 - protein, 7 aa, synthetic construct
AASSMQS

SEQ ID NO: 644 - protein, 7 aa, synthetic construct
ADSSLQS

SEQ ID NO: 645 - protein, 9 aa, synthetic construct
HESSSLPLT

SEQ ID NO: 646 - protein, 9 aa, synthetic construct
HQTSSLPLT

SEQ ID NO: 647 - protein, 9 aa, synthetic construct
HQSTSLPLT

SEQ ID NO: 648 - protein, 9 aa, synthetic construct
HQSSTLPLT

SEQ ID NO: 649 - protein, 9 aa, synthetic construct
HQSSSMPLT

SEQ ID NO: 650 - protein, 9 aa, synthetic construct
HQSSSLPMT

SEQ ID NO: 651 - protein, 9 aa, synthetic construct
HQSSSLPLS

SEQ ID NO: 652 - protein, 9 aa, synthetic construct
TQSSSLPLT

SEQ ID NO: 653 - protein, 9 aa, synthetic construct
HQSSSLPLD

SEQ ID NO: 654 - protein, 9 aa, synthetic construct
HQSSSVPLT

SEQ ID NO: 655 - protein, 5 aa, synthetic construct
TYAIS

SEQ ID NO: 656 - protein, 5 aa, synthetic construct
SFAIS

SEQ ID NO: 657 - protein, 5 aa, synthetic construct
SYTIS

SEQ ID NO: 658 - protein, 5 aa, synthetic construct
SYALS

SEQ ID NO: 659 - protein, 5 aa, synthetic construct
SYAMS

SEQ ID NO: 660 - protein, 5 aa, synthetic construct
SYAIT

SEQ ID NO: 661 - protein, 5 aa, synthetic construct
SYAIA

SEQ ID NO: 662 - protein, 5 aa, synthetic construct
SYAID

SEQ ID NO: 663 - protein, 5 aa, synthetic construct
EYAIT

SEQ ID NO: 664 - protein, 5 aa, synthetic construct
SYDIS

SEQ ID NO: 665 - protein, 17 aa, synthetic construct
AIIPIFGTANYAQKFQG

SEQ ID NO: 666 - protein, 17 aa, synthetic construct
GILPIFGTANYAQKFQG

SEQ ID NO: 667 - protein, 17 aa, synthetic construct
GIIPLFGTANYAQKFQG

SEQ ID NO: 668 - protein, 17 aa, synthetic construct
GIIPIFATANYAQKFQG

SEQ ID NO: 669 - protein, 17 aa, synthetic construct
GIIPIFGTAEYAQKFQG

SEQ ID NO: 670 - protein, 17 aa, synthetic construct
GIIPIFGTANYANKFQG

SEQ ID NO: 671 - protein, 17 aa, synthetic construct
GIIPIFGTANYAQRFQG

SEQ ID NO: 672 - protein, 17 aa, synthetic construct
GIIPIFGTANYAQKFNG

SEQ ID NO: 673 - protein, 17 aa, synthetic construct
GIIPIFGTANYAQKFQA

SEQ ID NO: 674 - protein, 17 aa, synthetic construct
GIIPIFGTANYAQKYQG

SEQ ID NO: 675 - protein, 16 aa, synthetic construct
DRIAVAGTNYYYGMDV

SEQ ID NO: 676 - protein, 16 aa, synthetic construct
EKIAVAGTNYYYGMDV

SEQ ID NO: 677 - protein, 16 aa, synthetic construct
ERLAVAGTNYYYGMDV

SEQ ID NO: 678 - protein, 16 aa, synthetic construct
ERIALAGTNYYYGMDV

SEQ ID NO: 679 - protein, 16 aa, synthetic construct
ERIAVAGSNYYYGMDV

SEQ ID NO: 680 - protein, 16 aa, synthetic construct
ERIAVAGTDYYYGMDV

SEQ ID NO: 681 - protein, 16 aa, synthetic construct
ERIAVAGTNYYYAMDV

SEQ ID NO: 682 - protein, 16 aa, synthetic construct
ERIAVAGTNYYYGMEV

SEQ ID NO: 683 - protein, 16 aa, synthetic construct
ERIAVAGTNYYYGMDA

SEQ ID NO: 684 - protein, 16 aa, synthetic construct
ERIAVAGTNYYYGMDS

SEQ ID NO: 685 - protein, 12 aa, synthetic construct
KASQSVSSSNLA

SEQ ID NO: 686 - protein, 12 aa, synthetic construct
RSSQSVSSSNLA

SEQ ID NO: 687 - protein, 12 aa, synthetic construct
RATQSVSSSNLA

SEQ ID NO: 688 - protein, 12 aa, synthetic construct
RASNSVSSSNLA

SEQ ID NO: 689 - protein, 12 aa, synthetic construct
RASQTVSSSNLA

SEQ ID NO: 690 - protein, 12 aa, synthetic construct
RASQSASSSNLA

SEQ ID NO: 691 - protein, 12 aa, synthetic construct
RASQSVTSSNLA

SEQ ID NO: 692 - protein, 12 aa, synthetic construct
RASQSVSSSELA

SEQ ID NO: 693 - protein, 12 aa, synthetic construct
RASQSVSSSNVA

SEQ ID NO: 694 - protein, 12 aa, synthetic construct
RASQSVSSSNLG

SEQ ID NO: 695 - protein, 7 aa, synthetic construct
AASTRAT

SEQ ID NO: 696 - protein, 7 aa, synthetic construct
GGSTRAT

SEQ ID NO: 697 - protein, 7 aa, synthetic construct
GATTRAT

SEQ ID NO: 698 - protein, 7 aa, synthetic construct
GASSRAT

SEQ ID NO: 699 - protein, 7 aa, synthetic construct
GASTKAT

SEQ ID NO: 700 - protein, 7 aa, synthetic construct
GASTRGT

SEQ ID NO: 701 - protein, 7 aa, synthetic construct
GASTRAS

SEQ ID NO: 702 - protein, 7 aa, synthetic construct
GASTRAA

SEQ ID NO: 703 - protein, 7 aa, synthetic construct
GASTRAD

SEQ ID NO: 704 - protein, 7 aa, synthetic construct
GASTKGT

SEQ ID NO: 705 - protein, 8 aa, synthetic construct
EQYGSSIT

SEQ ID NO: 706 - protein, 8 aa, synthetic construct
QEYGSSIT

SEQ ID NO: 707 - protein, 8 aa, synthetic construct
QQFGSSIT

SEQ ID NO: 708 - protein, 8 aa, synthetic construct
QQYASSIT

SEQ ID NO: 709 - protein, 8 aa, synthetic construct
QQYGTSIT

SEQ ID NO: 710 - protein, 8 aa, synthetic construct
QQYGSTIT

SEQ ID NO: 711 - protein, 8 aa, synthetic construct
QQYGSSLT

SEQ ID NO: 712 - protein, 8 aa, synthetic construct
QQYGSSIS

SEQ ID NO: 713 - protein, 8 aa, synthetic construct
QQYGSSIA

SEQ ID NO: 714 - protein, 8 aa, synthetic construct
QNYGSSIT

SEQ ID NO: 715 - protein, 5 aa, synthetic construct
AYYMH

SEQ ID NO: 716 - protein, 5 aa, synthetic construct
GFYMH

SEQ ID NO: 717 - protein, 5 aa, synthetic construct
GYFMH

SEQ ID NO: 718 - protein, 5 aa, synthetic construct
GYYLH

SEQ ID NO: 719 - protein, 5 aa, synthetic construct
GYYVH

SEQ ID NO: 720 - protein, 5 aa, synthetic construct
GYYMD

SEQ ID NO: 721 - protein, 5 aa, synthetic construct
GYYMS

SEQ ID NO: 722 - protein, 5 aa, synthetic construct
GYYME

SEQ ID NO: 723 - protein, 5 aa, synthetic construct
GYYMT

SEQ ID NO: 724 - protein, 5 aa, synthetic construct
GYYMA

SEQ ID NO: 725 - protein, 17 aa, synthetic construct
KINPNSGGTNYAQKFQG

SEQ ID NO: 726 - protein, 17 aa, synthetic construct
RLNPNSGGTNYAQKFQG

SEQ ID NO: 727 - protein, 17 aa, synthetic construct
RIDPNSGGTNYAQKFQG

SEQ ID NO: 728 - protein, 17 aa, synthetic construct
RINPDSGGTNYAQKFQG

SEQ ID NO: 729 - protein, 17 aa, synthetic construct
RINPNTGGTNYAQKFQG

SEQ ID NO: 730 - protein, 17 aa, synthetic construct
RINPNSAGTNYAQKFQG

SEQ ID NO: 731 - protein, 17 aa, synthetic construct
RINPNSGGTEYAQKFQG

SEQ ID NO: 732 - protein, 17 aa, synthetic construct
RINPNSGGTNYSQKFQG

SEQ ID NO: 733 - protein, 17 aa, synthetic construct
RINPNSGGTNYAQRFQG

SEQ ID NO: 734 - protein, 17 aa, synthetic construct
RINPNSGGTNYAQKFNG

SEQ ID NO: 735 - protein, 8 aa, synthetic construct
ATRHAFDI

SEQ ID NO: 736 - protein, 8 aa, synthetic construct
GSRHAFDI

SEQ ID NO: 737 - protein, 8 aa, synthetic construct
GTKHAFDI

SEQ ID NO: 738 - protein, 8 aa, synthetic construct
GTRSAFDI

SEQ ID NO: 739 - protein, 8 aa, synthetic construct
GTRHDFDI

SEQ ID NO: 740 - protein, 8 aa, synthetic construct
GTRHAYDI

SEQ ID NO: 741 - protein, 8 aa, synthetic construct
GTRHAFEI

SEQ ID NO: 742 - protein, 8 aa, synthetic construct
GTRHAFDV

SEQ ID NO: 743 - protein, 8 aa, synthetic construct
GTRHAFDL

SEQ ID NO: 744 - protein, 8 aa, synthetic construct
GARHAFDI

SEQ ID NO: 745 - protein, 13 aa, synthetic construct
SRSSGSIASNYVQ

SEQ ID NO: 746 - protein, 13 aa, synthetic construct
TKSSGSIASNYVQ

SEQ ID NO: 747 - protein, 13 aa, synthetic construct
TRTSGSIASNYVQ

SEQ ID NO: 748 - protein, 13 aa, synthetic construct
TRSTGSIASNYVQ

SEQ ID NO: 749 - protein, 13 aa, synthetic construct
TRSSASIASNYVQ

SEQ ID NO: 750 - protein, 13 aa, synthetic construct
TRSSGTIASNYVQ

SEQ ID NO: 751 - protein, 13 aa, synthetic construct
TRSSGSLASNYVQ

SEQ ID NO: 752 - protein, 13 aa, synthetic construct
TRSSGSIGSNYVQ

SEQ ID NO: 753 - protein, 13 aa, synthetic construct
TRSSGSIASNYLQ

SEQ ID NO: 754 - protein, 13 aa, synthetic construct
TRSSGSIASNYIQ

SEQ ID NO: 755 - protein, 7 aa, synthetic construct
DDNQRPS

SEQ ID NO: 756 - protein, 7 aa, synthetic construct
EENQRPS

SEQ ID NO: 757 - protein, 7 aa, synthetic construct
EDQQRPS

SEQ ID NO: 758 - protein, 7 aa, synthetic construct
EDNNRPS

SEQ ID NO: 759 - protein, 7 aa, synthetic construct
EDNQKPS

SEQ ID NO: 760 - protein, 7 aa, synthetic construct
EDNQRPT

SEQ ID NO: 761 - protein, 7 aa, synthetic construct
EEDQRPS

SEQ ID NO: 762 - protein, 7 aa, synthetic construct
EDNQKPT

SEQ ID NO: 763 - protein, 7 aa, synthetic construct
NDNQRPS

SEQ ID NO: 764 - protein, 7 aa, synthetic construct
EENQKPS

SEQ ID NO: 765 - protein, 10 aa, synthetic construct
NSYDSIVHVV

SEQ ID NO: 766 - protein, 10 aa, synthetic construct
QTYDSIVHVV

SEQ ID NO: 767 - protein, 10 aa, synthetic construct
QSFDSIVHVV

SEQ ID NO: 768 - protein, 10 aa, synthetic construct
QSYESIVHVV

SEQ ID NO: 769 - protein, 10 aa, synthetic construct
QSYDTIVHVV

SEQ ID NO: 770 - protein, 10 aa, synthetic construct
QSYDSLVHVV

SEQ ID NO: 771 - protein, 10 aa, synthetic construct
QSYDSIAHVV

SEQ ID NO: 772 - protein, 10 aa, synthetic construct
QSYDSIVDVV

SEQ ID NO: 773 - protein, 10 aa, synthetic construct
QSYDSIVHIV

SEQ ID NO: 774 - protein, 10 aa, synthetic construct
QSYDSIVHVA

SEQ ID NO: 775 - protein, 5 aa, synthetic construct
TYAIS

SEQ ID NO: 776 - protein, 5 aa, synthetic construct
SFAIS

SEQ ID NO: 777 - protein, 5 aa, synthetic construct
SYTIS

SEQ ID NO: 778 - protein, 5 aa, synthetic construct
SYALS

SEQ ID NO: 779 - protein, 5 aa, synthetic construct
SYAMS

SEQ ID NO: 780 - protein, 5 aa, synthetic construct
SYAIT

SEQ ID NO: 781 - protein, 5 aa, synthetic construct
SYAIA

SEQ ID NO: 782 - protein, 5 aa, synthetic construct
SYAID

SEQ ID NO: 783 - protein, 5 aa, synthetic construct
EYAIT

SEQ ID NO: 784 - protein, 5 aa, synthetic construct
SYDIS

SEQ ID NO: 785 - protein, 17 aa, synthetic construct
AIIPIFGTANYAQKFQG

SEQ ID NO: 786 - protein, 17 aa, synthetic construct
GILPIFGTANYAQKFQG

SEQ ID NO: 787 - protein, 17 aa, synthetic construct
GIIPLFGTANYAQKFQG

SEQ ID NO: 788 - protein, 17 aa, synthetic construct
GIIPIFATANYAQKFQG

SEQ ID NO: 789 - protein, 17 aa, synthetic construct
GIIPIFGTAEYAQKFQG

SEQ ID NO: 790 - protein, 17 aa, synthetic construct
GIIPIFGTANYANKFQG

SEQ ID NO: 791 - protein, 17 aa, synthetic construct
GIIPIFGTANYAQRFQG

SEQ ID NO: 792 - protein, 17 aa, synthetic construct
GIIPIFGTANYAQKFNG

SEQ ID NO: 793 - protein, 17 aa, synthetic construct
GIIPIFGTANYAQKFQA

SEQ ID NO: 794 - protein, 17 aa, synthetic construct
GIIPIFGTANYAQKYQG

SEQ ID NO: 795 - protein, 13 aa, synthetic construct
EVIVGATSSGMDV

SEQ ID NO: 796 - protein, 13 aa, synthetic construct
DIIVGATSSGMDV

SEQ ID NO: 797 - protein, 13 aa, synthetic construct
DVLVGATSSGMDV

SEQ ID NO: 798 - protein, 13 aa, synthetic construct
DVIAGATSSGMDV

SEQ ID NO: 799 - protein, 13 aa, synthetic construct
DVIVGASSSGMDV

SEQ ID NO: 800 - protein, 13 aa, synthetic construct
DVIVGATTSGMDV

SEQ ID NO: 801 - protein, 13 aa, synthetic construct
DVIVGATSSAMDV

SEQ ID NO: 802 - protein, 13 aa, synthetic construct
DVIVGATSSGMEV

SEQ ID NO: 803 - protein, 13 aa, synthetic construct
DVIVGATSSGMDA

SEQ ID NO: 804 - protein, 13 aa, synthetic construct
DVIVGATSSGMDL

SEQ ID NO: 805 - protein, 11 aa, synthetic construct
KASQSISRYLN

SEQ ID NO: 806 - protein, 11 aa, synthetic construct
RGSQSISRYLN

SEQ ID NO: 807 - protein, 11 aa, synthetic construct
RATQSISRYLN

SEQ ID NO: 808 - protein, 11 aa, synthetic construct
RASESISRYLN

SEQ ID NO: 809 - protein, 11 aa, synthetic construct
RASQTISRYLN

SEQ ID NO: 810 - protein, 11 aa, synthetic construct
RASQSITRYLN

SEQ ID NO: 811 - protein, 11 aa, synthetic construct
RASQSISKYLN

SEQ ID NO: 812 - protein, 11 aa, synthetic construct
RASQSISRFLN

SEQ ID NO: 813 - protein, 11 aa, synthetic construct
RASQSISRYIN

SEQ ID NO: 814 - protein, 11 aa, synthetic construct
RASQSISRYLE

SEQ ID NO: 815 - protein, 7 aa, synthetic construct
AASSLQS

SEQ ID NO: 816 - protein, 7 aa, synthetic construct
GGSSLQS

SEQ ID NO: 817 - protein, 7 aa, synthetic construct
GATSLQS

SEQ ID NO: 818 - protein, 7 aa, synthetic construct
GASTLQS

SEQ ID NO: 819 - protein, 7 aa, synthetic construct
GASSIQS

SEQ ID NO: 820 - protein, 7 aa, synthetic construct
GASSAQS

SEQ ID NO: 821 - protein, 7 aa, synthetic construct
GASSLES

SEQ ID NO: 822 - protein, 7 aa, synthetic construct
GASSLQT

SEQ ID NO: 823 - protein, 7 aa, synthetic construct
GASSLQD

SEQ ID NO: 824 - protein, 7 aa, synthetic construct
GASSMQS

SEQ ID NO: 825 - protein, 9 aa, synthetic construct
NQSYSTPLT

SEQ ID NO: 826 - protein, 9 aa, synthetic construct
QNSYSTPLT

SEQ ID NO: 827 - protein, 9 aa, synthetic construct
QQTYSTPLT

SEQ ID NO: 828 - protein, 9 aa, synthetic construct
QQSFSTPLT

SEQ ID NO: 829 - protein, 9 aa, synthetic construct
QQSYTTPLT

SEQ ID NO: 830 - protein, 9 aa, synthetic construct
QQSYSSPLT

SEQ ID NO: 831 - protein, 9 aa, synthetic construct
QQSYSTPIT

SEQ ID NO: 832 - protein, 9 aa, synthetic construct
QQSYSTPVT

SEQ ID NO: 833 - protein, 9 aa, synthetic construct
QQSYSTPLS

SEQ ID NO: 834 - protein, 9 aa, synthetic construct
QQSYSTPLA

SEQ ID NO: 835 - protein, 113 aa, synthetic construct
EVQLVESGGGLVQPGGSLRLSCAASGFTFSNYGMSWIRQAPGKGLEWIGSIYYSGSTIYNPSLKSLVTISRDNSKNTLYLQMNSLRAEDTATYYCARESIDYWGQGTLVTVSS

SEQ ID NO: 836 - protein, 21 aa, synthetic construct
IYIWAPLAGTCGVLLLSLVIT

SEQ ID NO: 837 - protein, 119 aa, synthetic construct [sequence omitted]

SEQ ID NO: 838 - protein, 7 aa, synthetic construct
DDSQDYY

SEQ ID NO: 839 - protein, 15 aa, synthetic construct
GSGSSGSGSSGSGSS

SEQ ID NO: 840 - protein, 6 aa, synthetic construct
GSGSSG

(19) United States
(12) Patent Application Publication
(10) Pub. No.: US 2026/0317442 A1
(43) Pub. Date: Oct. 15, 2026

(54) MOLECULES THAT BIND TO PSCA POLYPEPTIDES

(71) Applicant: UNIVERSITY OF PITTSBURGH - OF THE COMMONWEALTH SYSTEM OF HIGHER EDUCATION, Pittsburgh, PA (US)

(72) Inventors: Dimiter Stanchev Dimitrov, Pittsburgh, PA (US); Dontcho V. Jelev, Pittsburgh, PA (US); Dusan Baek, Pittsburgh, PA (US); John W. Mellors, Pittsburgh, PA (US); Wei Li, Pittsburgh, PA (US); Xiaojie Chu, Pittsburgh, PA (US)

(21) Appl. No.: 19/481,261

(22) PCT Filed: May 8, 2024
(86) PCT No.: PCT/US2024/028372

§ 371 Date: Nov. 4, 2025

Related U.S. Application Data
(60) Provisional application No. 63/465,126, filed on May 9, 2023.

(51) Int. Cl. C07K 16/30; A61K 40/31; A61K 40/42; A61K 45/06; C07K 14/705

(52) U.S. Cl. CPC C07K 16/3069; A61K 40/31; A61K 40/4274; A61K 45/06; C07K 14/7051; C07K 14/70517; C07K 14/70596; A61K 2239/13; A61K 2239/17; A61K 2239/21; A61K 2239/22; A61K 2239/29; A61K 2239/58; C07K 2317/31; C07K 2317/565; C07K 2317/567; C07K 2317/622

(57) ABSTRACT

This document provides methods and materials involved in binding a binder (e.g., an antibody, antigen binding fragment, antibody domain, CAR, cell engager, and/or ADC) to a PSCA polypeptide. For example, binders (e.g., antibodies, antigen binding fragments, antibody domains, CARs, cell engagers, and/or ADCs) that bind to a PSCA polypeptide and methods and materials for using one or more such binding molecules to treat a mammal (e.g., a human) having cancer are provided.

### CROSS-REFERENCE TO RELATED APPLICATIONS

This application claims the benefit of U.S. Patent Application Ser. No. 63/465,126, filed on May 9, 2023. The disclosure of the prior application is considered part of, and is incorporated by reference in, the disclosure of this application.

### SEQUENCE LISTING

This application contains a Sequence Listing that has been submitted electronically as an XML file named “48881-0042WO1 SL.xml.” The XML file, created on May 1, 2024, is 769,610 bytes in size. The material in the XML file is hereby incorporated by reference in its entirety.

### BACKGROUND

### 1. Technical Field

This document relates to methods and materials involved in binding a molecule (e.g., an antibody, a fragment of an antibody, an antibody domain, a chimeric antigen receptor (CAR), a cell engager, or an antibody-drug conjugate (ADC)) to a PSCA polypeptide. For example, this document provides binders (e.g., antibodies, antigen binding fragments, antibody domains, CARs, cell engagers, or ADCs) that bind to a PSCA polypeptide and methods and materials for using such binders to treat cancer. This document also provides cells (e.g., host cells) designed to express one or more binders (e.g., antibodies, antigen binding fragments, antibody domains, CARs, or cell engagers) having the ability to bind to a PSCA polypeptide and methods and materials for using such cells to treat cancer.

### 2. Background Information

Prostate stem cell antigen (PSCA) polypeptides are glycosylphosphatidylinositol (GPI)-anchored cell surface polypeptides belonging to the Thy-1/Ly-6 family. As a GPI-anchored polypeptide, PSCA polypeptides are located in lipid rafts of the plasma membrane and may have a role in regulating cell signaling events through receptor/ligand or other protein-protein binding, although the specific physiological function of PSCA polypeptides remain unclear. PSCA polypeptides are found to be upregulated in various types of solid tumors including prostate, bladder, renal cell, and pancreatic cancers; and its expression positively correlates with tumor grade and disease extent. PSCA polypeptides have been found to be expressed at 100% of bone metastases of prostate cancer (Saeki et al., Clin. Cancer Res., 16(14):3533-8 (2010)). These observations establish PSCA polypeptides as potential immunotherapeutic targets for prostate cancers.

During last decades, several monoclonal antibodies were identified that target human PSCA polypeptides. Most of them were derived from mouse hybridomas, which were obtained by immunization of mice using PSCA polypeptide overexpressing cells lines followed by large-scale B cell screening. These antibodies eradicated PSCA polypeptide expressing xenografts in immunocompromised mice either through antibody-dependent cellular cytotoxicity (ADCC; clone 1G8, which inhibited growth of LAPC9 xenografts), antibody-drug conjugates (ADCs; clone 8D11 from Genentech), or chimeric antigen receptor armed T cells (CAR-T cells; Bellicum GoCAR-T BPX-601) (Foster et al., Mol. Ther., 25 (9): 2176-2188 (2017)). Among them, the Bellicum GoCAR-T BPX-601 was evaluated in phase I/II clinical trials, and showed good tolerability (Stein et al., J. Clin. Oncol., 41 (6): 140-140 (2023)).

### SUMMARY

This document provides methods and materials involved in binding a molecule (e.g., an antibody, an antigen binding fragment, an antibody domain, a CAR, a cell engager, or an ADC) to a PSCA polypeptide. For example, this document provides binders (e.g., antibodies, antigen binding fragments, antibody domains, CARs, cell engagers, or ADCs) that bind to a PSCA polypeptide and methods and materials for using one or more such binders to treat a mammal (e.g., a human) having cancer (e.g., prostate cancer such as metastatic prostate cancer, bladder cancer, renal cell cancer, and pancreatic cancer).

This document also provides cells (e.g., host cells) designed to express one or more binders (e.g., antibodies, antigen binding fragments, antibody domains, CARs, or cell engagers) having the ability to bind to a PSCA polypeptide and methods and materials for using such cells to treat cancer (e.g., prostate cancer such as metastatic prostate cancer, bladder cancer, renal cell cancer, and pancreatic cancer).

As described herein, binders (e.g., one or more antibodies, one or more antigen binding fragments, one or more antibody domains, one or more CARs, one or more cell engagers, and/or one or more ADCs) can be designed to have the ability to bind to a PSCA polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human PSCA polypeptide as set forth in SEQ ID NO: 129 (see, e.g., Example 6).

As described herein, anti-PSCA binders (e.g., Clones #1, #2, #3, #4, #5, #6, #7, and #8) bind to PSCA polypeptides (e.g., human PSCA polypeptides). See, e.g., FIGS. 4 and 6. In addition, BiKEs designed to include the CDRs of Clone #5 exhibited ADCC of cells expressing PSCA polypeptides (FIGS. 9C and D). These results demonstrate that anti-PSCA binders provided herein can be used to treat cancer (e.g., prostate cancer such as metastatic prostate cancer, bladder cancer, renal cell cancer, and pancreatic cancer).

In some cases, two sets of three CDRs of an antibody or antigen binding fragment provided herein (e.g., SEQ ID NOs: 1-3 and 9-11; SEQ ID NOs: 17-19 and SEQ ID NOs: 25-27; SEQ ID NOs: 33-35 and SEQ ID NOs: 41-43; SEQ ID NOs: 49-51 and SEQ ID NOs: 57-59; SEQ ID NOs: 65-67 and SEQ ID NOs: 73-75; SEQ ID NOs: 81-83 and SEQ ID NOs: 89-91; SEQ ID NOs: 97-99 and SEQ ID NOs: 105-107; or SEQ ID NOs: 113-115 and SEQ ID NOs: 121-123) can be engineered into a CAR to create CAR⁺ cells (e.g., CAR⁺ T cells, CAR⁺ stem cells such as CAR⁺ induced pluripotent stem cells, or CAR⁺ NK cells) having the ability to target PSCA⁺ cells (e.g., PSCA⁺ tumor cells and/or PSCA⁺ tumor vasculature), can be engineered into an antibody structure that includes an Fc region to create antibodies having the ability to target PSCA⁺ cells (e.g., PSCA⁺ tumor cells and/or PSCA⁺ tumor vasculature) and induce ADCC against the target PSCA⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target PSCA⁺ cells (e.g., PSCA⁺ tumor cells and/or PSCA⁺ tumor vasculature) and induce one or more immune responses (e.g., T cell immune responses and/or ADCC using a cell engager in the absence of an Fc-containing antibody) against the target PSCA⁺ cells. It is noted that BiKE- and TriKE-mediated killing can be referred to as ADCC even though it is not initiated by an Fc domain in cases when the engager lacks an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, scFv-drug conjugates, and/or antibody domain-drug conjugates can be designed to include an appropriate binder provided herein to create the conjugate. Such conjugates can be used to deliver the drug payload to target cells such as cancer cells (e.g., PSCA⁺ cancer cells) or cancer vasculature (e.g., PSCA⁺ cancer vasculature).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer (e.g., a PSCA⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer (e.g., prostate cancer such as metastatic prostate cancer, bladder cancer, renal cell cancer, and pancreatic cancer).

As also described herein, cells (e.g., host cells) can be designed to express one or more binders (e.g., antibodies, antigen binding fragments, antibody domains, CARs, or cell engagers) having the ability to bind to a PSCA polypeptide. For example, cells such as T cells (e.g., CTLs), stem cells (e.g., induced pluripotent stem cells), or NK cells can be engineered to express one or more CARs having the ability to bind to a PSCA polypeptide. Such cells (e.g., PSCA-specific CAR⁺ T cells or NK cells) can be used to treat cancer (e.g., prostate cancer such as metastatic prostate cancer, bladder cancer, renal cell cancer, and pancreatic cancer)).

In some cases, a binder (e.g., an antibody, antigen binding fragment, and/or antibody domain) provided herein can be used to detect the presence or absence of a PSCA polypeptide. For example, a binder (e.g., an antibody, antigen binding fragment, and/or antibody domain) provided herein can be used to determine whether or not a sample (e.g., a biological sample such tumor biopsy) obtained from a mammal (e.g., a human) contains PSCA⁺ cells (e.g., PSCA⁺ cancer cells). Having the ability to detect the presence or absence of a PSCA polypeptide (e.g., PSCA⁺ cancer cells) can allow clinicians, health professionals, and patients to make better decisions about possible treatment options. For example, detection of PSCA⁺ cancer cells within a mammal can allow clinicians, health professionals, and patients to select an appropriate anti-cancer treatment that targets the PSCA⁺ cancer cells. Such treatments that target the PSCA⁺ cancer cells can include administration of one or more of the binders described herein having the ability to bind to a PSCA polypeptide and/or administration of one or more cells (e.g., PSCA-specific CAR⁺ T cells or NK cells) designed to express a binder described herein.

In general, one aspect of this document features an antibody comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (ii) a heavy chain variable domain or region comprising the amino acid sequences set forth in SEQ ID NO:17 (or SEQ ID NO: 17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO: 33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO: 57 with one, two, or three amino acid additions, deletions, or substitutions), SEQ ID NO: 58 (or SEQ ID NO:58 with one, two, or three amino acid additions, deletions, or substitutions), and SEQ ID NO:59 (or SEQ ID NO:59 with one, two, or three amino acid additions, deletions, or substitutions); (v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions); (vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); (vii) a heavy chain variable domain or region comprising the amino acid sequences set forth in SEQ ID NO: 97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO:106 (or SEQ ID NO:106 with one, two, or three amino acid additions, deletions, or substitutions), and SEQ ID NO: 107 (or SEQ ID NO:107 with one, two, or three amino acid additions, deletions, or substitutions); or (viii) a heavy chain variable domain or region comprising the amino acid sequences set forth in SEQ ID NO:113 (or SEQ ID NO:113 with one, two, or three amino acid additions, deletions, or substitutions), SEQ ID NO:114 (or SEQ ID NO:114 with one, two, or three amino acid additions, deletions, or substitutions), and SEQ ID NO:115 (or SEQ ID NO:115 with one, two, or three amino acid additions, deletions, or substitutions) and a light chain variable domain or region comprising the amino acid sequences set forth in SEQ ID NO: 121 (or SEQ ID NO: 121 with one, two, or three amino acid additions, deletions, or substitutions), SEQ ID NO: 122 (or SEQ ID NO:122 with one, two, or three amino acid additions, deletions, or substitutions), and SEQ ID NO: 123 (or SEQ ID NO:123 with one, two, or three amino acid additions, deletions, or substitutions). The antibody can comprise the ability to bind to SEQ ID NO:129. The antibody can comprise the heavy chain variable domain or region of the (i) and the light chain variable domain or region of the (i). The heavy chain variable domain or region can comprise an amino acid sequence having at least 90 percent identity to the amino acid sequence set forth in SEQ ID NO:8, and the light chain variable domain or region can comprise an amino acid sequence having at least 90 percent identity to the amino acid sequence set forth in SEQ ID NO:16. The antibody can comprise the heavy chain variable domain or region of the (ii) and the light chain variable domain or region of the (ii). The heavy chain variable domain or region comprises an amino acid sequence having at least 90 percent identity to the amino acid sequence set forth in SEQ ID NO:24, and the light chain variable domain or region comprises an amino acid sequence having at least 90 percent identity to the amino acid sequence set forth in SEQ ID NO:32. The antibody can comprise the heavy chain variable domain or region of the (iii) and the light chain variable domain or region of the (iii). The heavy chain variable domain or region can comprise an amino acid sequence having at least 90 percent identity to the amino acid sequence set forth in SEQ ID NO:40, and the light chain variable domain or region can comprise an amino acid sequence having at least 90 percent identity to the amino acid sequence set forth in SEQ ID NO:48. The antibody can comprise the heavy chain variable domain or region of the (iv) and the light chain variable domain or region of the (iv). The heavy chain variable domain or region can comprise an amino acid sequence having at least 90 percent identity to the amino acid sequence set forth in SEQ ID NO:56, and the light chain variable domain or region can comprise an amino acid sequence having at least 90 percent identity to the amino acid sequence set forth in SEQ ID NO:64. The antibody can comprise the heavy chain variable domain or region of the (v) and the light chain variable domain or region of the (v). The heavy chain variable domain or region can comprise an amino acid sequence having at least 90 percent identity to the amino acid sequence set forth in SEQ ID NO: 72, and the light chain variable domain or region can comprise an amino acid sequence having at least 90 percent identity to the amino acid sequence set forth in SEQ ID NO: 80. The antibody can comprise the heavy chain variable domain or region of the (vi) and the light chain variable domain or region of the (vi). The heavy chain variable domain or region can comprise an amino acid sequence having at least 90 percent identity to the amino acid sequence set forth in SEQ ID NO:88 and the light chain variable domain or region can comprise an amino acid sequence having at least 90 percent identity to the amino acid sequence set forth in SEQ ID NO:96. The antibody can comprise the heavy chain variable domain or region of the (vii) and the light chain variable domain or region of the (vii). The heavy chain variable domain or region can comprise an amino acid sequence having at least 90 percent identity to the amino acid sequence set forth in SEQ ID NO:104, and the light chain variable domain or region can comprise an amino acid sequence having at least 90 percent identity to the amino acid sequence set forth in SEQ ID NO:112. The antibody can comprise the heavy chain variable domain or region of the (viii) and the light chain variable domain or region of the (viii). The heavy chain variable domain or region can comprise an amino acid sequence having at least 90 percent identity to the amino acid sequence set forth in SEQ ID NO:120, and the light chain variable domain or region can comprise an amino acid sequence having at least 90 percent identity to the amino acid sequence set forth in SEQ ID NO:128. The antibody can be a monoclonal antibody. The antibody can be an scFv antibody. This paragraph can be referred to as first aspect paragraph.

In another aspect, this document features an antigen binding fragment comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO:19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO: 33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO: 57 with one, two, or three amino acid additions, deletions, or substitutions), SEQ ID NO: 58 (or SEQ ID NO:58 with one, two, or three amino acid additions, deletions, or substitutions), and SEQ ID NO:59 (or SEQ ID NO:59 with one, two, or three amino acid additions, deletions, or substitutions); (v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions); (vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); (vii) a heavy chain variable domain or region comprising the amino acid sequences set forth in SEQ ID NO: 97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO:106 (or SEQ ID NO:106 with one, two, or three amino acid additions, deletions, or substitutions), and SEQ ID NO: 107 (or SEQ ID NO:107 with one, two, or three amino acid additions, deletions, or substitutions); or (viii) a heavy chain variable domain or region comprising the amino acid sequences set forth in SEQ ID NO:113 (or SEQ ID NO:113 with one, two, or three amino acid additions, deletions, or substitutions), SEQ ID NO:114 (or SEQ ID NO:114 with one, two, or three amino acid additions, deletions, or substitutions), and SEQ ID NO:115 (or SEQ ID NO:115 with one, two, or three amino acid additions, deletions, or substitutions) and a light chain variable domain or region comprising the amino acid sequences set forth in SEQ ID NO: 121 (or SEQ ID NO: 121 with one, two, or three amino acid additions, deletions, or substitutions), SEQ ID NO: 122 (or SEQ ID NO:122 with one, two, or three amino acid additions, deletions, or substitutions), and SEQ ID NO:123 (or SEQ ID NO:123 with one, two, or three amino acid additions, deletions, or substitutions). The antigen binding fragment can comprise the ability to bind to SEQ ID NO: 129. The antigen binding fragment can comprise the heavy chain variable domain or region of the (i) and the light chain variable domain or region of the (i). The heavy chain variable domain or region can comprise an amino acid sequence having at least 90 percent identity to the amino acid sequence set forth in SEQ ID NO: 8, and the light chain variable domain or region can comprise an amino acid sequence having at least 90 percent identity to the amino acid sequence set forth in SEQ ID NO: 16. The antigen binding fragment can comprise the heavy chain variable domain or region of the (ii) and the light chain variable domain or region of the (ii). The heavy chain variable domain or region can comprise an amino acid sequence having at least 90 percent identity to the amino acid sequence set forth in SEQ ID NO:24, and the light chain variable domain or region can comprise an amino acid sequence having at least 90 percent identity to the amino acid sequence set forth in SEQ ID NO:32. The antigen binding fragment can comprise the heavy chain variable domain or region of the (iii) and the light chain variable domain or region of the (iii). The heavy chain variable domain or region can comprise an amino acid sequence having at least 90 percent identity to the amino acid sequence set forth in SEQ ID NO:40, and the light chain variable domain or region can comprise an amino acid sequence having at least 90 percent identity to the amino acid sequence set forth in SEQ ID NO:48. The antigen binding fragment can comprise the heavy chain variable domain or region of the (iv) and the light chain variable domain or region of the (iv). The heavy chain variable domain or region can comprise an amino acid sequence having at least 90 percent identity to the amino acid sequence set forth in SEQ ID NO:56, and the light chain variable domain or region can comprise an amino acid sequence having at least 90 percent identity to the amino acid sequence set forth in SEQ ID NO:64. The antigen binding fragment can comprise the heavy chain variable domain or region of the (v) and the light chain variable domain or region of the (v). The heavy chain variable domain or region can comprise an amino acid sequence having at least 90 percent identity to the amino acid sequence set forth in SEQ ID NO: 72, and the light chain variable domain or region can comprise an amino acid sequence having at least 90 percent identity to the amino acid sequence set forth in SEQ ID NO: 80. The antigen binding fragment can comprise the heavy chain variable domain or region of the (vi) and the light chain variable domain or region of the (vi). The heavy chain variable domain or region can comprise an amino acid sequence having at least 90 percent identity to the amino acid sequence set forth in SEQ ID NO:88, and the light chain variable domain or region can comprise an amino acid sequence having at least 90 percent identity to the amino acid sequence set forth in SEQ ID NO:96. The antigen binding fragment can comprise the heavy chain variable domain or region of the (vii) and the light chain variable domain or region of the (vii). The heavy chain variable domain or region can comprise an amino acid sequence having at least 90 percent identity to the amino acid sequence set forth in SEQ ID NO:104, and the light chain variable domain or region can comprise an amino acid sequence having at least 90 percent identity to the amino acid sequence set forth in SEQ ID NO:112. The antigen binding fragment can comprise the heavy chain variable domain or region of the (viii) and the light chain variable domain or region of the (viii). The heavy chain variable domain or region can comprise an amino acid sequence having at least 90 percent identity to the amino acid sequence set forth in SEQ ID NO:120, and the light chain variable domain or region can comprise an amino acid sequence having at least 90 percent identity to the amino acid sequence set forth in SEQ ID NO:128. The antigen binding fragment can be monoclonal. The antigen binding fragment can be a Fab. This paragraph can be referred to as second aspect paragraph.

In another aspect, this document features an antibody domain comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) or a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions) or a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO: 33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) or a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) or a light chain variable domain or region comprising the amino acid sequences set forth in SEQ ID NO:57 (or SEQ ID NO: 57 with one, two, or three amino acid additions, deletions, or substitutions), SEQ ID NO: 58 (or SEQ ID NO:58 with one, two, or three amino acid additions, deletions, or substitutions), and SEQ ID NO:59 (or SEQ ID NO:59 with one, two, or three amino acid additions, deletions, or substitutions); (v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) or a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions); (vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) or a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); (vii) a heavy chain variable domain or region comprising the amino acid sequences set forth in SEQ ID NO: 97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) or a light chain variable domain or region comprising the amino acid sequences set forth in SEQ ID NO: 105 (or SEQ ID NO:105 with one, two, or three amino acid additions, deletions, or substitutions), SEQ ID NO:106 (or SEQ ID NO: 106 with one, two, or three amino acid additions, deletions, or substitutions), and SEQ ID NO: 107 (or SEQ ID NO:107 with one, two, or three amino acid additions, deletions, or substitutions); or (viii) a heavy chain variable domain or region comprising the amino acid sequences set forth in SEQ ID NO:113 (or SEQ ID NO:113 with one, two, or three amino acid additions, deletions, or substitutions), SEQ ID NO:114 (or SEQ ID NO:114 with one, two, or three amino acid additions, deletions, or substitutions), and SEQ ID NO:115 (or SEQ ID NO:115 with one, two, or three amino acid additions, deletions, or substitutions) or a light chain variable domain or region comprising the amino acid sequences set forth in SEQ ID NO: 121 (or SEQ ID NO: 121 with one, two, or three amino acid additions, deletions, or substitutions), SEQ ID NO: 122 (or SEQ ID NO:122 with one, two, or three amino acid additions, deletions, or substitutions), and SEQ ID NO:123 (or SEQ ID NO:123 with one, two, or three amino acid additions, deletions, or substitutions). The antibody domain can comprise the ability to bind to SEQ ID NO:129. The antibody domain can comprise the heavy chain variable domain or region of (i). The antibody domain can comprises an amino acid sequence having at least 90 percent identity to the amino acid sequence set forth in SEQ ID NO:8. The antibody domain can comprise the light chain variable domain or region of (i). The antibody domain can comprises an amino acid sequence having at least 90 percent identity to the amino acid sequence set forth in SEQ ID NO:16. The antibody domain can comprise the heavy chain variable domain or region of (ii). The antibody domain can comprises an amino acid sequence having at least 90 percent identity to the amino acid sequence set forth in SEQ ID NO:24. The antibody domain can comprise the light chain variable domain or region of (ii). The antibody domain can comprises an amino acid sequence having at least 90 percent identity to the amino acid sequence set forth in SEQ ID NO: 32. The antibody domain can comprise the heavy chain variable domain or region of (iii). The antibody domain can comprises an amino acid sequence having at least 90 percent identity to the amino acid sequence set forth in SEQ ID NO:40. The antibody domain can comprise the light chain variable domain or region of (iii). The antibody domain can comprises an amino acid sequence having at least 90 percent identity to the amino acid sequence set forth in SEQ ID NO:48. The antibody domain can comprise the heavy chain variable domain or region of (iv). The antibody domain can comprises an amino acid sequence having at least 90 percent identity to the amino acid sequence set forth in SEQ ID NO:56. The antibody domain can comprise light chain variable domain or region of (iv). The antibody domain can comprises an amino acid sequence having at least 90 percent identity to the amino acid sequence set forth in SEQ ID NO: 64. The antibody domain can comprise the heavy chain variable domain or region of (v). The antibody domain can comprises an amino acid sequence having at least 90 percent identity to the amino acid sequence set forth in SEQ ID NO:72. The antibody domain can comprise the light chain variable domain or region of (v). The antibody domain can comprises an amino acid sequence having at least 90 percent identity to the amino acid sequence set forth in SEQ ID NO:80. The antibody domain can comprise the heavy chain variable domain or region of (vi). The antibody domain can comprises an amino acid sequence having at least 90 percent identity to the amino acid sequence set forth in SEQ ID NO:88. The antibody domain can comprise light chain variable domain or region of (vi). The antibody domain can comprises an amino acid sequence having at least 90 percent identity to the amino acid sequence set forth in SEQ ID NO: 96. The antibody domain can comprise the heavy chain variable domain or region of (vii). The antibody domain can comprises an amino acid sequence having at least 90 percent identity to the amino acid sequence set forth in SEQ ID NO:104. The antibody domain can comprise the light chain variable domain or region of (vii). The antibody domain can comprises an amino acid sequence having at least 90 percent identity to the amino acid sequence set forth in SEQ ID NO:112. The antibody domain can comprise the heavy chain variable domain or region of (viii). The antibody domain can comprises an amino acid sequence having at least 90 percent identity to the amino acid sequence set forth in SEQ ID NO:120. The antibody domain can comprise light chain variable domain or region of (viii). The antibody domain can comprises an amino acid sequence having at least 90 percent identity to the amino acid sequence set forth in SEQ ID NO:128. The antibody domain can be monoclonal. The antibody domain can be a VH domain. The antibody domain can be a VL domain. This paragraph can be referred to as third aspect paragraph.

In another aspect, this document features a chimeric antigen receptor comprising (or consisting essentially of, or consisting of) an antigen binding domain, a hinge, a transmembrane domain, and one or more signaling domains, wherein the antigen binding domain comprises (or consists essentially of, or consists of) any antibody, any antigen-binding fragment, or any antibody domain of any of first aspect paragraph, second aspect paragraph, or third aspect paragraph. The antigen binding domain can comprise a scFv having the ability to bind to a PSCA polypeptide. The antigen binding domain can comprise a VH domain having the ability to bind to a PSCA polypeptide. The hinge can comprise a hinge set forth in Example 15. The transmembrane domain can comprise a transmembrane domain set forth in Example 16. The chimeric antigen receptor can comprise one or more signaling domains set forth in Example 17. This paragraph can be referred to as fourth aspect paragraph.

In another aspect, this document features a cell comprising (or consisting essentially of, or consisting of) any chimeric antigen receptor of the preceding paragraph. The cell can be a T cell, a stem cell, or an NK cell.

In another aspect, this document features a cell engager comprising (or consisting essentially of, or consisting of) a first antigen binding domain, a linker, and a second antigen binding domain, wherein the first antigen binding domain comprises (or consists essentially of, or consists of) any antibody, any antigen-binding fragment, or any antibody domain of any of first aspect paragraph, second aspect paragraph, or third aspect paragraph. The first antigen binding domain can comprise a VH domain having the ability to bind to a PSCA polypeptide. The first antigen binding domain can comprise a VL domain having the ability to bind to a PSCA polypeptide. The first antigen binding domain can comprise an Fab having the ability to bind to a PSCA polypeptide. The first antigen binding domain can be an IgG having the ability to bind to a PSCA polypeptide. The linker can comprise a linker set forth in Example 12 or Example 15. The second antigen binding domain can bind to a polypeptide expressed on the surface of T cells. The polypeptide expressed on the surface of T cells can be a CD3 polypeptide. The second antigen binding domain can be an antigen binding domain set forth in Example 19. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in Example 20. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in Example 20. This paragraph can be referred to as fifth aspect paragraph.

In another aspect, this document features a nucleic acid comprising (or consisting essentially of, or consisting of) a nucleic acid sequence encoding at least part of the antibody, the antigen-binding fragment, or the antibody domain of any of first aspect paragraph, second aspect paragraph, or third aspect paragraph. The nucleic acid sequence can encode the heavy chain variable domain or region of any one of the (i)-(viii) of first aspect paragraph. The nucleic acid sequence can encode the light chain variable domain or region of any one of the (i)-(viii) of first aspect paragraph. The nucleic acid can be a viral vector. The nucleic acid can be a phagemid.

In another aspect, this document features a nucleic acid comprising (or consisting essentially of, or consisting of) a nucleic acid sequence encoding any chimeric antigen receptor of fourth aspect paragraph or any cell engager of fifth aspect paragraph. The nucleic acid can be a viral vector. The nucleic acid can be a phagemid.

In another aspect, this document features a host cell comprising (or consisting essentially of, or consisting of) any nucleic acid of the preceding paragraph. This paragraph can be referred to as sixth aspect paragraph.

In another aspect, this document features a host cell that expresses any chimeric antigen receptor of fourth aspect paragraph or any cell engager of fifth aspect paragraph. The host cell can be a T cell, stem cell, or NK cell. This paragraph can be referred to as seventh aspect paragraph.

In another aspect, this document features an antibody-drug conjugate (ADC) comprising (or consisting essentially of, or consisting of) an antigen binging domain covalently linked to a drug, wherein the antigen binging domain comprises an antibody, an antigen binding fragment, or an antibody domain of any of first aspect paragraph, second aspect paragraph, or third aspect paragraph. The antigen binding domain can comprise a scFv having the ability to bind to a PSCA polypeptide. The antigen binding domain can comprise a Fab having the ability to bind to a PSCA polypeptide. The antigen binding domain can comprise a VH domain having the ability to bind to a PSCA polypeptide. The antigen binding domain can comprise a VL domain having the ability to bind to a PSCA polypeptide. The antigen binding domain can comprise an IgG having the ability to bind to a PSCA polypeptide. The drug can be selected from the group consisting of auristatins, mertansine, or pyrrolobenzodiazepine (PBD) dimers. This paragraph can be referred to as eighth aspect paragraph.

In another aspect, this document features a composition comprising (or consisting essentially of, or consisting of) an antibody, an antigen binding fragment, or an antibody domain of any of first aspect paragraph, second aspect paragraph, or third aspect paragraph. The composition can comprise any antibody of first aspect paragraph. The composition can comprise any antigen binding fragment of second aspect paragraph. The composition can comprise any antibody domain of third aspect paragraph.

In another aspect, this document features a composition comprising (or consisting essentially of, or consisting of) any cell engager of fifth aspect paragraph.

In another aspect, this document features a composition comprising (or consisting essentially of, or consisting of) any cell of sixth aspect paragraph or seventh aspect paragraph.

In another aspect, this document features a composition comprising (or consisting essentially of, or consisting of) any ADC of any one of eighth aspect paragraph. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a method of treating a mammal having cancer. The method comprises (or consists essentially of, or consists of) administering, to the mammal, a composition of any of the preceding four paragraphs. The mammal can be a human. The cancer can be a PSCA⁺ cancer. The PSCA⁺ cancer can be selected from the group consisting of PSCA⁺ prostate cancer, PSCA⁺ metastatic prostate cancer, PSCA⁺ bladder cancer, PSCA⁺ renal cell cancer, and PSCA⁺ pancreatic cancer. The number of cancer cells within the mammal can be reduced following the administering step.

In another aspect, this document features a method of treating a mammal having cancer. The method comprises (or consists essentially of, or consists of) (a) administering, to the mammal, a composition of any of the preceding four paragraphs referred to in the preceding paragraph, and (b) administering, to the mammal, a composition comprising a checkpoint inhibitor. The mammal can be a human. The cancer can be a PSCA⁺ cancer. The PSCA⁺ cancer can be selected from the group consisting of PSCA⁺ prostate cancer, PSCA⁺ metastatic prostate cancer, PSCA⁺ bladder cancer, PSCA⁺ renal cell cancer, and PSCA⁻ pancreatic cancer. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab. The number of cancer cells within the mammal can be reduced following the administering steps (a) and (b).

In another aspect, this document features a method for binding a binding molecule to a PSCA polypeptide. The method comprises (or consists essentially of, or consists of) contacting the PSCA polypeptide with an antibody, an antigen binding fragment, or an antibody domain of any of first aspect paragraph, second aspect paragraph, or third aspect paragraph. The contacting can be performed in vitro. The contacting can be performed in vivo. The contacting can be performed within a mammal by administering the antibody, the antigen binding fragment, or the antibody domain to the mammal. The mammal can be a human.

In another aspect, this document features a method for binding a binding molecule to a PSCA polypeptide. The method comprises (or consists essentially of, or consists of) contacting the PSCA polypeptide with any chimeric antigen receptor of fourth aspect paragraph, any cell engager of fifth aspect paragraph, or any ADC of eighth aspect paragraph. The contacting can be performed in vitro. The contacting can be performed in vivo. The contacting can be performed within a mammal by administering the chimeric antigen receptor, the cell engager, or the ADC to the mammal. The mammal can be a human.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a scFv provided herein, followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3ζ intracellular signaling domain sequence).

FIG. 2 is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a VH domain (or VL domain) provided herein (e.g., a VH domain designed to include one set of three CDRs of a heavy chain provided herein such as SEQ ID NOs: 1-3; SEQ ID NOs: 17-19 SEQ ID NOs: 33-35; SEQ ID NOs: 49-51; SEQ ID NOs: 65-67; SEQ ID NOs: 81-83; SEQ ID NOs: 97-99; or SEQ ID NOs: 113-115; or a VL domain designed to include one set of three CDRs of a light chain provided herein such as SEQ ID NOs: 9-11; SEQ ID NOs: 25-27; SEQ ID NOs: 41-43; SEQ ID NOs: 57-59; SEQ ID NOs: 73-75; SEQ ID NOs: 89-91; SEQ ID NOs: 105-107; or SEQ ID NOs: 121-123), followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3ζ intracellular signaling domain sequence).

FIG. 3 is a schematic of an exemplary BiTE designed using CDR1, CDR2, and CDR3 of a heavy chain and CDR1, CDR2, and CDR3 of a light chain in an Ig format (e.g., an IgG1 format). A humanized anti-CD3 scFv (e.g., an gOKT3-7 scFv set forth in U.S. Pat. No. 6,750,325) can be linked to the C-terminus of the light chain via a linker (e.g., a (G4S) 3 linker (SEQ ID NO: 135)).

FIG. 4 is a graph plotting affinity enhanced clones binding to recombinant human PSCA polypeptides with comparisons to the original clone G7, as tested by ELISA.

FIGS. 5A-5D. Characterization of the affinity enhanced Clone #5 (F12). FIGS. 5A and 5B provide a comparison of binding kinetics of G7 (FIG. 5A) and F12 (FIG. 5B) by BlitZ, which is a bio-interferometry method that measures antibody on-rates and off-rates. The enhanced binding affinity of Clone #5 is mainly ascribed to the decreased Koff rate. FIGS. 5C and 5D show results evaluating binding of G7 and F12 to cell surface PSCA polypeptides on PC3 cells. F12 exhibited substantial binding to PC3-PSCA⁺ cells without binding to PC3-PSCA⁻ cells.

FIGS. 6A-6D. Testing internalization of IgG1 F12 on prostate cancer cell lines using a pH sensitive dye, which was conjugated to the F12 antibody and only illuminates under the low pH environment. FIG. 6A is a graph showing F12 internalized into acid lysosome in a concentration and time dependent manner. The internalization was dependent on PSCA polypeptide expression on the cell surface of PC3 cells. FIG. 6B is a graph showing F12 internalized into acid lysosome in a concentration and time dependent manner. The internalization was dependent on PSCA polypeptide expression on the cell surface of Du145 cells. FIG. 6C is a graph showing that F12 internalization is dependent on temperature. 4° C. did not promote F12 internalization. FIG. 6D is a graph showing that F12 (dye) conjugate internalization can be specifically inhibited by the naked IgG1 antibody in a concentration dependent manner both on PC3 and Du145 cells.

FIGS. 7A-7E. Generation of F12-MMAE ADC and testing on mouse xenograft model. FIG. 7A. The scheme of F12-MMAE ADC that is designed to include an antibody (e.g., F12), and lysine mediated conjugation with the dipeptidyl linker, PAB spacer and MMAE payload. FIG. 7B. Mass spectrum to determine the drug-antibody-ratio (DAR) of 2. FIG. 7C. The in vitro cell killing of F12-MMAE-ADC testing on PC3-PSCA cell lines. The payload MMAE killing efficacy was comparable to the PBD payload. FIG. 7D. The anti-tumor efficacy on PC3-PSCA xenograft NSG mouse model. PC3-PSCA cells (5 million) were implanted into mice flank with mitragel (50%). Two weeks later, both naked IgG1 F12 and F12-ADC were injected into mice through i.p. injection at once weekly with totally four doses. Tumor size and mice body weights were monitored every three days. Results showed that the ADC reduced tumor volumes, while IgG1 F12 alone did not. FIG. 7E. The ADC was safe as no body weight loss was observed.

FIGS. 8A-8D. Generation of BiKEs containing F12 targeting PSCA polypeptides and an anti-human CD16A antibody domain (VH7, shown as cross hatched; FIG. 8A). IgG1 F12 is a typical IgG1 antibody with wild type Fc for implementing ADCC. In 1+2 BiKE, the monovalent VH7 was fused to IgG1 heavy chain N terminal region through a flexible linker. The knob-into-hole technique was used for creating an asymmetric molecule. The LALAPG mutations were introduced into the Fc to silent the Fc mediated effector function. Thus, the ADCC was only triggered by VH7. In 2+2 BiKE, bivalent VH7 domains were introduced to increase CD16A engagement. The Fc in 2+2 BiKE also contained the LALAPG mutation. FIG. 8B. SDS-PAGE for IgG and BiKEs. IgG1 and 2+2 BiKE were produced by co-transfect equal molar ration of HC and LC plasmids, while the 1+2 BiKE was expressed by co-transfected the hole HC, knob HC, and the LC in 1:1:2 ratio. FIG. 8C is a schematic showing how the IgG1 F12 mounts ADCC though the WT Fc. FIG. 8D is a schematic showing how the BiKE evokes ADCC though formation of immune synapse by simultaneously binding tumor antigen on tumor cells and binding to CD16A on NK cells.

FIGS. 9A-9D. Characterization of BiKEs in terms of antigen binding and in vitro ADCC using the Promega ADCC assay kits, Jurkat T cells stably expressing CD16A, and NFAT-RE driven luciferase expression cassette. FIG. 9A shows binding of IgG1 F12 and BiKEs to the recombinant PSCA polypeptides. BiKEs exhibited lower binding affinity to PSCA polypeptides than IgG1 F12. FIG. 9B shows binding of IgG1 F12 and BiKEs to recombinant human CD16A. The lower signal for IgG1 was consistent to the binding of Fc to CD16A, which the anti-CD16A antibody based BiKE showed much higher affinity to CD16A. FIG. 9C shows ADCC of IgG1 F12 and BiKEs using the promega ADCC kit. The potency of 2+2 BiKE was similar as IgG1 F12. IgG1 F12 exhibited the highest efficacy. The higher binding affinity to PSCA polypeptides for IgG1 F12 compared to BikEs may explain the higher efficacy of IgG1 F12. 2+2 BiKE was more potent than 1+2 BiKE, demonstrating the ability of bivalent VH7. The ADCC effect was specific since no ADCC signals were observed in PSCA negative PC3 cells (FIG. 9D), and in groups that did not contain the target cells (Effector cells+Abs).

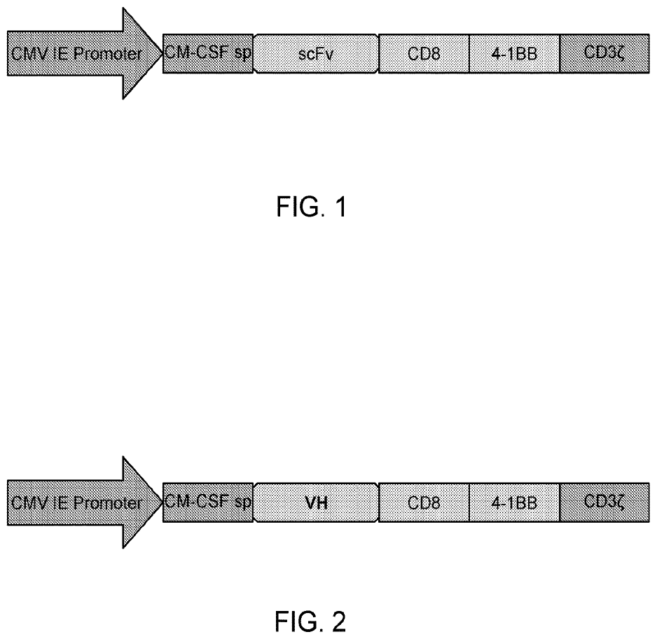

### DETAILED DESCRIPTION

This document provides binders (e.g., antibodies, antigen binding fragments, antibody domains, CARs, cell engagers, and ADCs) that bind (e.g., specifically bind) to a PSCA polypeptide (e.g., a human PSCA polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:129 and/or an extracellular domain thereof (see, e.g., Example 6).

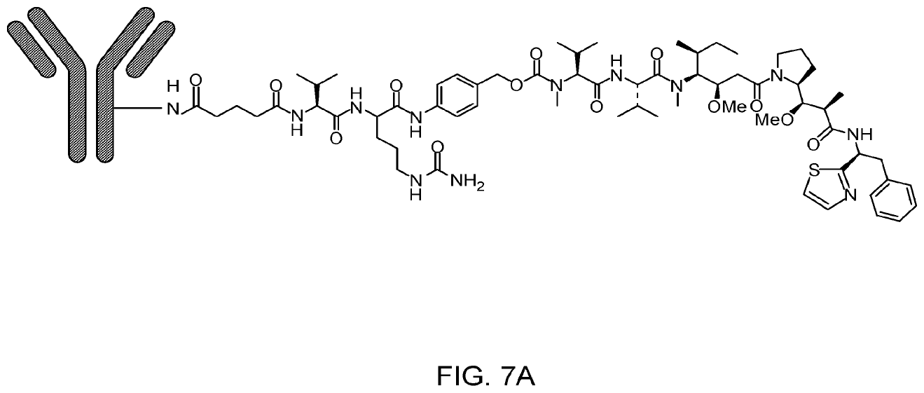

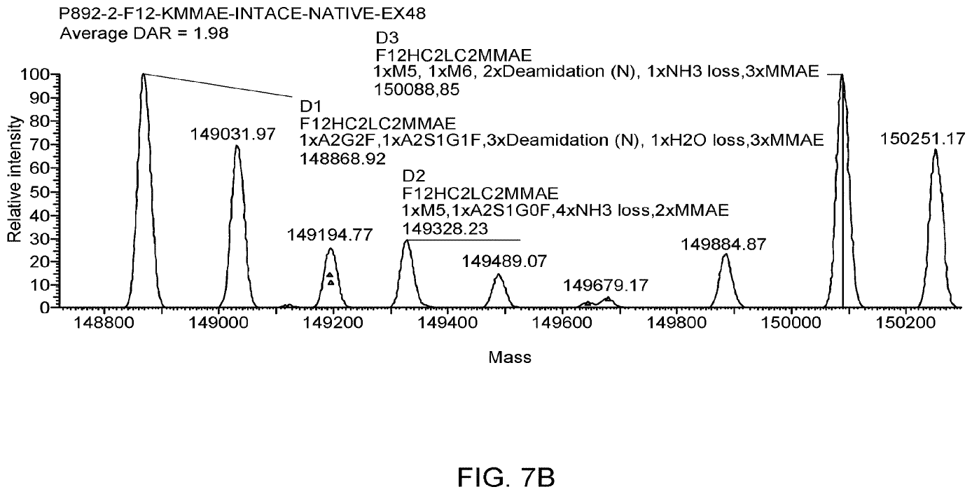

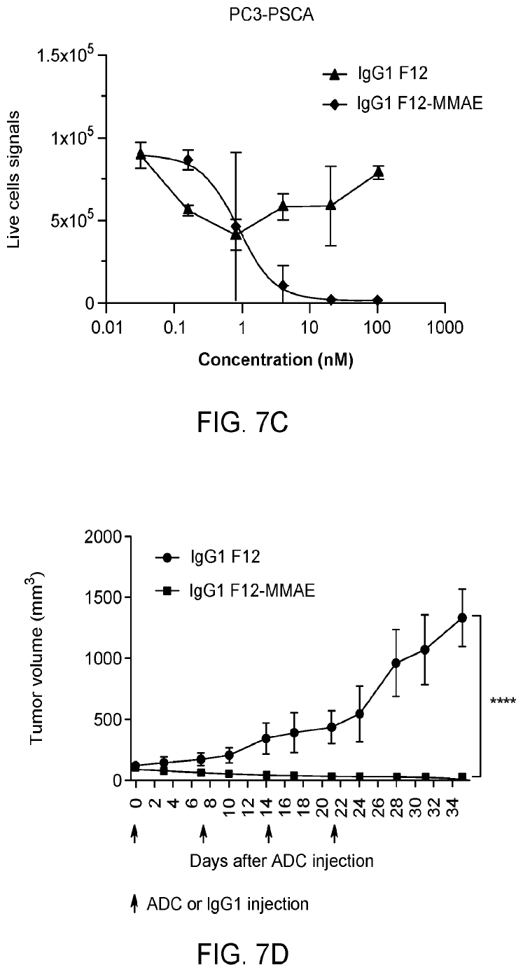

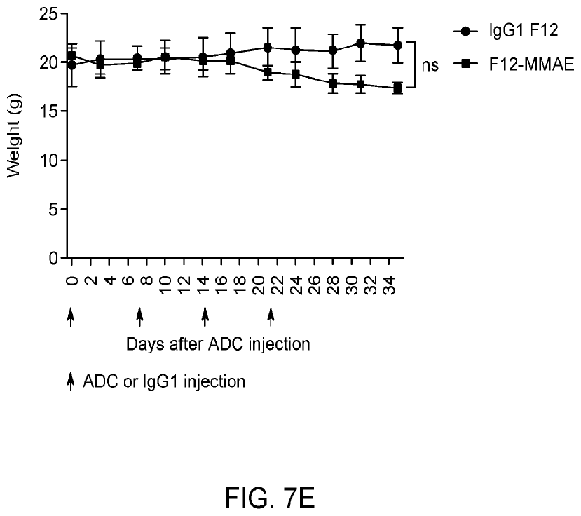

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Färber-Schwarz, Methods Mol. Biol., 907:713-27 (2012); and Brinkmann and Kontermann, MAbs., 9 (2): 182-212 (2017)). The two variable regions can be connected by a polypeptide linker (e.g., a polypeptide linker having five to ten residues in length or a polypeptide linker as set forth in Example 12). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in Example 12). See, also, Chen et al., Adv. Drug Deliv. Rev., 65(10): 1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains. Examples of scFv structures of scFv's provided herein include, without limitation, those structures set forth in FIGS. 7-9, 12, 13A-13B, 14A-14B, 15A-15B, 16A-16B, 17A-17B, 18A-18B, 19A-19B, 20A-20B, and 21A-21B.

An antibody provided herein can include the CDRs as described herein (e.g., as described in Table 49) and can be configured to be a human antibody, a humanized antibody, or a chimeric antibody. In some cases, an antibody provided herein can include the CDRs as described herein (e.g., as described in Table 49) and can be a monoclonal antibody. In some cases, an antibody provided herein can include the CDRs as described herein (e.g., as described in Table 49) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 49) and can be configured to be a human antigen binding fragment, a humanized antigen binding fragment, or a chimeric antigen binding fragment. In some cases, an antigen binding fragment provided herein can include the CDRs as described herein (e.g., as described in Table 49) and can be a monoclonal antigen binding fragment. In some cases, an antigen binding fragment provided herein can include the CDRs as described herein (e.g., as described in Table 49) and can be configured as a Fab antibody. In some cases, a Fab antibody can include a partial hinge sequence (e.g., EPKSCDKT (SEQ ID NO:248)) for disulfide bonding between heavy and light chains of the Fab. Examples of Fab's provided herein include, without limitation, those structures set forth in FIGS. 2-6.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 49) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 49) and can be a monoclonal antibody domain. In some cases, an antibody domain provided herein can include the CDRs as described herein (e.g., as described in Table 49) and can be engineered as a single VH domain or a single VL domain. Examples of VH domain's provided herein include, without limitation, those heavy chain variable domains set forth in FIGS. 2A, 3A, 4A, 5A, 6A, 7A, 8A, and 9A. Examples of VL domain's provided herein include, without limitation, those light chain variable domains set forth in FIGS. 2B, 3B, 4B, 5B, 6B, 7B, 8B, and 9B.

An anti-PSCA antibody, anti-PSCA antigen binding fragment, or anti-PSCA antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-PSCA antibody) can be a scFv antibody. In some cases, an antigen binding fragment provided herein (e.g., an anti-PSCA antibody fragment) can be a Fab. In some cases, an antibody provided herein (e.g., an anti-PSCA antibody) can be a fully intact antibody having the structure set forth in Example 9. In some cases, an antibody domain provided herein (e.g., an anti-PSCA antibody domain) can be a VH domain or a VL domain.

In some cases, an anti-PSCA antibody, anti-PSCA antigen binding fragment, or anti-PSCA antibody domain provided herein can be fully human. In some cases, an anti-PSCA antibody, anti-PSCA antigen binding fragment, or anti-PSCA antibody domain provided herein can have a low risk for inducing immunogenicity within a human.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to a PSCA polypeptide (e.g., a human PSCA polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a PSCA polypeptide (e.g., a human PSCA polypeptide). In some examples, a CAR provided herein can be designed to include an antigen binding domain that includes a single set of three CDRs (e.g., a CDR1, CDR2, and CDR3) of an antibody domain (e.g., a VH domain) provided herein (e.g., a VH domain designed to include one set of three CDRs of a heavy chain provided herein such as SEQ ID NOs: 1-3; SEQ ID NOs: 17-19 SEQ ID NOs: 33-35; SEQ ID NOs: 49-51; SEQ ID NOs: 65-67; SEQ ID NOs: 81-83; SEQ ID NOs: 97-99; or SEQ ID NOs: 113-115).

In some examples, a CAR provided herein can be designed to include an antigen binding domain that includes a single set of three CDRs (e.g., a CDR1, CDR2, and CDR3) of an antibody domain (e.g., a VL domain) provided herein (e.g., a VL domain designed to include one set of three CDRs of a light chain provided herein such as SEQ ID NOs: 9-11; SEQ ID NOs: 25-27; SEQ ID NOs: 41-43; SEQ ID NOs: 57-59; SEQ ID NOs: 73-75; SEQ ID NOs: 89-91; SEQ ID NOs: 105-107; or SEQ ID NOs: 121-123).

In some cases, an antigen binding domain of a CAR targeting a PSCA polypeptide can be designed to include a scFv antibody described herein such as a scFv set forth in Examples 7, 10, or 11.

Examples of CAR structures that can be used to make a CAR provided herein include, without limitation, those set forth in FIGS. 29-44.

In some cases, a CAR provided herein can be designed to include a signal peptide. Any appropriate signal peptide can be used to design a CAR described herein. Examples of signal peptide that can be used to make a CAR described herein include without limitation, a human IGKV1-39-derived signal peptide, IGKV1-16, IGKV1-33, IGKV3-11, IGKV4-1, or IGKV6-21. In some cases, a CAR provided herein can be designed to include a signal peptide that comprises, consists essentially of, or consists of one of the amino acid sequences set forth in Example 14. In some cases, a CAR provided herein can be designed to include a signal peptide that comprises, consists essentially of, or consists of one of the amino acid sequences set forth in Example 14 with one, two, three, four, five, six, seven, eight, nine, or ten amino acid deletions, additions, substitutions, or combinations thereof. In some cases, a CAR provided herein can be designed to include a signal peptide that comprises, consists essentially of, or consists of one of the amino acid sequences set forth in Example 14 with two or less, three or less, four or less, five or less, six or less, seven or less, eight or less, nine or less, or ten or less amino acid deletions, additions, substitutions, or combinations thereof.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD32-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as a hinge to make a CAR described herein. For example, any one of the linker sequences set forth in Example 12 can be used as a hinge of a CAR described herein.

In some cases, a CAR provided herein can be designed to include a hinge that comprises, consists essentially of, or consists of one of the amino acid sequences set forth in Example 12 or Example 15. In some cases, a CAR provided herein can be designed to include a hinge that comprises, consists essentially of, or consists of one of the amino acid sequences set forth in Example 12 or Example 15 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in Example 12 or Example 15 with two or less, three or less, four or less, five or less, six or less, seven or less, eight or less, nine or less, or ten or less amino acid deletions, additions, substitutions, or combinations thereof.

A CAR provided herein can be designed to include any appropriate transmembrane domain. For example, the transmembrane domain of a CAR provided herein can be, without limitation, a CD35 transmembrane domain, a CD4 transmembrane domain, a CD8a transmembrane domain, a CD28 transmembrane domain, and a 4-1BB transmembrane domain. In some cases, a CAR provided herein can be designed to include a transmembrane domain that comprises, consists essentially of, or consists of one of the amino acid sequences set forth in Example 16. In some cases, a CAR provided herein can be designed to include a transmembrane domain that comprises, consists essentially of, or consists of one of the amino acid sequences set forth in Example 16 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in Example 16 with two or less, three or less, four or less, five or less, six or less, seven or less, eight or less, nine or less, or ten or less amino acid deletions, additions, substitutions, or combinations thereof.

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD32 intracellular signaling domains, CD27 intracellular signaling domains, CD2ζ intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ C. intracellular signaling domain. In some cases, a CAR described herein can be designed to be a second generation CAR having a CD28 intracellular signaling domain followed by a CD3ζ C. intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, a CAR provided herein can be designed to include at least one intracellular signaling domain that comprises, consists essentially of, or consists of one of the amino acid sequences set forth in Example 17. In some cases, a CAR provided herein can be designed to include at least one intracellular signaling domain that comprises, consists essentially of, or consists of one of the amino acid sequences set forth in Example 17 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in Example 17 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting a PSCA polypeptide can be designed to include a scFv comprising two sets of three CDRs of an antibody or antigen binding fragment provided herein (e.g., SEQ ID NOs: 1-3 and 9-11; SEQ ID NOs: 17-19 and SEQ ID NOs: 25-27; SEQ ID NOs: 33-35 and SEQ ID NOs: 41-43; SEQ ID NOs: 49-51 and SEQ ID NOs: 57-59; SEQ ID NOs: 65-67 and SEQ ID NOs: 73-75; SEQ ID NOs: 81-83 and SEQ ID NOs: 89-91; SEQ ID NOs: 97-99 and SEQ ID NOs: 105-107; or SEQ ID NOs: 113-115 and SEQ ID NOs: 121-123), followed by a hinge such as a hinge/linker set forth in Example 12 or Example 15 (e.g., an IgG4-derived hinge, a CD8a hinge, or a linker plus IgG4-derived hinge), followed by a transmembrane domain such as a transmembrane domain set forth in Example 16 (e.g., a human CD28 transmembrane domain or a CD8a transmembrane domain), followed by one or more intracellular signaling domains such as one or more intracellular signaling domain set forth in Example 17 (e.g., a human 4-1BB intracellular signaling domain followed by a human CD35 intracellular signaling domain).

In some cases, a CAR targeting a PSCA polypeptide can be designed to include a scFv comprising two sets of three CDRs of an antibody or antigen binding fragment provided herein (e.g., SEQ ID NOs: 1-3 and 9-11; SEQ ID NOs: 17-19 and SEQ ID NOs: 25-27; SEQ ID NOs: 33-35 and SEQ ID NOs: 41-43; SEQ ID NOs: 49-51 and SEQ ID NOs: 57-59; SEQ ID NOs: 65-67 and SEQ ID NOs: 73-75; SEQ ID NOs: 81-83 and SEQ ID NOs: 89-91; SEQ ID NOs: 97-99 and SEQ ID NOs: 105-107; or SEQ ID NOs: 113-115 and SEQ ID NOs: 121-123), followed by a hinge such as a hinge/linker set forth in Example 12 or Example 15 (e.g., an IgG4-derived hinge, a CD8α hinge, or a linker plus IgG4-derived hinge), followed by a transmembrane domain such as a transmembrane domain set forth in Example 16 (e.g., a human CD28 transmembrane domain or a CD8α transmembrane domain), followed by one or more intracellular signaling domains such as one or more intracellular signaling domain set forth in Example 17 (e.g., a human CD28 intracellular signaling domain followed by a human CD3ζ intracellular signaling domain). Examples of such CARs are set forth in FIGS. 29-44.

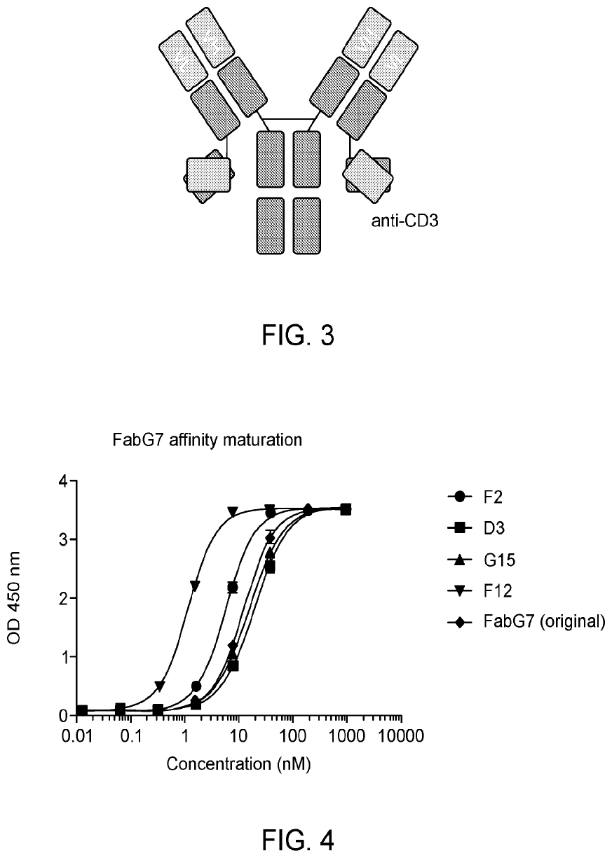

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to a PSCA polypeptide (e.g., a human PSCA polypeptide) and at least one antigen binding domain having the ability to bind to an antigen expressed on the surface of a cell (e.g., a T cell or an NK cell). In some cases, a cell engager described herein can link a PSCA⁺ cell (e.g., a PSCA⁺ cancer cell) to another cell (e.g., a T cell or an NK cell) via the two or more antigen binding domains of the cell engager. An example of a cell engager structure of cell engagers provided herein includes, without limitation, the structure set forth in FIG. 3. In some cases, the anti-CD3 scFv depicted in FIG. 3 can be replaced with a different antigen binding domain having the ability to bind to an antigen expressed on the surface of a cell (e.g., a T cell or an NK cell).

When a cell engager includes an antigen binding domain having the ability to bind to a PSCA polypeptide (e.g., a human PSCA polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to a PSCA polypeptide (e.g., a human PSCA polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to a PSCA polypeptide (e.g., a human PSCA polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a PSCA polypeptide (e.g., a human PSCA polypeptide). In some examples, a cell engager provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment.

In some examples, a cell engager provided herein can be designed to include an antigen binding domain that includes a single set of three CDRs (e.g., a CDR1, CDR2, and CDR3) of an antibody domain (e.g., a VH domain or a VL domain) provided herein (e.g., a VH domain designed to include one set of three CDRs of a heavy chain provided herein such as SEQ ID NOs: 1-3; SEQ ID NOs: 17-19 SEQ ID NOs: 33-35; SEQ ID NOs: 49-51; SEQ ID NOs: 65-67; SEQ ID NOs: 81-83; SEQ ID NOs: 97-99; or SEQ ID NOs: 113-115, or a VL domain designed to include one set of three CDRs of a light chain provided herein such as SEQ ID NOs: 9-11; SEQ ID NOs: 25-27; SEQ ID NOs: 41-43; SEQ ID NOs: 57-59; SEQ ID NOs: 73-75; SEQ ID NOs: 89-91; SEQ ID NOs: 105-107; or SEQ ID NOs: 121-123).

In some cases, an antigen binding domain of a cell engager targeting a PSCA polypeptide can be designed to include a VH domain described herein or a scFv described herein (see, e.g., FIGS. 7-9), or a Fab antibody described herein (see, e.g., FIGS. 2-6). In some cases, an antigen binding domain of a CAR described herein that has the ability to bind to a PSCA polypeptide (e.g., a human PSCA polypeptide) can be used as an antigen binding domain of a cell engager that targets PSCA⁺ cells.

As described herein, a cell engager can be designed to include at least one antigen binding domain having the ability to bind to a PSCA polypeptide (e.g., a human PSCA polypeptide) and at least one other antigen binding domain. That at least one other antigen binding domain can have the ability to bind to any appropriate antigen expressed on the surface of a cell. For example, when designing a cell engager such as a BiTE to link a PSCA⁺ cell and a T cell, the cell engager can include an antigen binding domain having the ability to bind to a PSCA polypeptide (e.g., a human PSCA polypeptide) and an antigen binding domain having the ability to bind to a polypeptide expressed on the surface of a T cell. Examples of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

Examples of BiTE structures that can be used to make a BiTE provided herein include, without limitation, those set forth in Example 22.

In some cases, a cell engager provided herein can be designed to include an antigen binding domain that comprises, consists essentially of, or consists of one of the amino acid sequences set forth in Example 19. In some cases, a cell engager provided herein can be designed to include an antigen binding domain that comprises, consists essentially of, or consists of one of the amino acid sequences set forth in Example 19 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in Example 19 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

When designing a cell engager such as a BiKE or a TriKE to link a PSCA⁺ cell and an NK cell, the cell engager can include an antigen binding domain having the ability to bind to a PSCA polypeptide (e.g., a human PSCA polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, and NKp46 polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36 (7): 433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2A antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

In some cases, a cell engager provided herein can be designed to include an antigen binding domain (e.g., a scFv or VH) that comprises, consists essentially of, or consists of one or more of the amino acid sequences set forth in Example 20. In some cases, a cell engager provided herein can be designed to include an antigen binding domain (e.g., a scFv or VH) that comprises, consists essentially of, or consists of one of the amino acid sequences set forth in Example 20 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in Example 20 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in Example 12. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGSGGGGSGGGGS (SEQ ID NO:135) linker. In some cases, a hinge of a CAR described herein can be used as a linker to make a cell engager described herein. For example, any one of the sequences set forth in Example 15 can be used as a linker of a cell engager described herein.

In some cases, a cell engager provided herein can be designed to include a linker that comprises, consists essentially of, or consists of one of the amino acid sequences set forth in Example 12 or Example 15. In some cases, a cell engager provided herein can be designed to include a linker that comprises, consists essentially of, or consists of one of the amino acid sequences set forth in Example 12 or Example 15 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in Example 12 or Example 15 with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting a PSCA polypeptide can be designed to include an antigen binding domain comprising two sets of three CDRs of an antibody or antigen binding fragment provided herein (e.g., SEQ ID NOs: 1-3 and 9-11; SEQ ID NOs: 17-19 and SEQ ID NOs: 25-27; SEQ ID NOs: 33-35 and SEQ ID NOs: 41-43; SEQ ID NOs: 49-51 and SEQ ID NOs: 57-59; SEQ ID NOs: 65-67 and SEQ ID NOs: 73-75; SEQ ID NOs: 81-83 and SEQ ID NOs: 89-91; SEQ ID NOs: 97-99 and SEQ ID NOs: 105-107; or SEQ ID NOs: 113-115 and SEQ ID NOs: 121-123), followed by a linker such as a linker/hinge set forth in Example 12 or Example 15 (e.g., SEQ ID NO: 135), followed by an antigen binding domain having the ability to bind to a polypeptide expressed on the surface of a T cell (e.g., an anti-human CD3 scFv) (see, e.g., FIGS. 64-71).

In some cases, a cell engager (e.g., a BiTE) targeting a PSCA polypeptide can be designed to include an IgG (e.g., IgG1) configuration having (a) a heavy chain comprising, consisting essentially of, or consisting of a heavy chain variable domain comprising a set of three CDRs provided herein (e.g., SEQ ID NOs: 1-3; SEQ ID NOs: 17-19; SEQ ID NOs: 33-35; SEQ ID NOs: 49-51; SEQ ID NOs: 65-67; SEQ ID NOs: 81-83; SEQ ID NOs: 97-99; or SEQ ID NOs: 113-115), an Ig hinge, and constant domains (e.g., CH1, CH2, and CH3 domains), (b) a light chain comprising, consisting essentially of, or consisting of a light chain variable domain comprising a set of three CDRs provided herein (e.g., SEQ ID NOs: 9-11; SEQ ID NOs: 25-27; SEQ ID NOs: 41-43; SEQ ID NOs: 57-59; SEQ ID NOs: 73-75; SEQ ID NOs: 89-91; SEQ ID NOs: 105-107; or SEQ ID NOs: 121-123), a constant domain (e.g., a kappa or lambda constant domain), and (c) an antigen binding domain having the ability to bind to a polypeptide expressed on the surface of a T cell (e.g., an anti-human CD3 scFv).

In some cases, a cell engager (e.g., a BiKE or a TriKE) targeting a PSCA polypeptide can be designed to include an antigen binding domain comprising two sets of three CDRs of an antibody or antigen binding fragment provided herein (e.g., SEQ ID NOs: 1-3 and 9-11; SEQ ID NOs: 17-19 and SEQ ID NOs: 25-27; SEQ ID NOs: 33-35 and SEQ ID NOs: 41-43; SEQ ID NOs: 49-51 and SEQ ID NOs: 57-59; SEQ ID NOs: 65-67 and SEQ ID NOs: 73-75; SEQ ID NOs: 81-83 and SEQ ID NOs: 89-91; SEQ ID NOs: 97-99 and SEQ ID NOs: 105-107; or SEQ ID NOs: 113-115 and SEQ ID NOs: 121-123), followed by a linker such as a linker/hinge set forth in Example 12 or Example 15 (e.g., SEQ ID NO:135), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIGS. 48-63).

In some cases, a cell engager (e.g., a BiKE or a TriKE) targeting a PSCA polypeptide can be designed to include an IgG (e.g., IgG1) configuration having (a) a heavy chain comprising, consisting essentially of, or consisting of a heavy chain variable domain comprising a set of three CDRs provided herein (e.g., SEQ ID NOs: 1-3; SEQ ID NOs: 17-19; SEQ ID NOs: 33-35; SEQ ID NOs: 49-51; SEQ ID NOs: 65-67; SEQ ID NOs: 81-83; SEQ ID NOs: 97-99; or SEQ ID NOs: 113-115), an Ig hinge, and constant domains (e.g., CH1, CH2, and CH3 domains), (b) a light chain comprising, consisting essentially of, or consisting of a light chain variable domain comprising a set of three CDRs provided herein (e.g., SEQ ID NOs: 9-11; SEQ ID NOs: 25-27; SEQ ID NOs: 41-43; SEQ ID NOs: 57-59; SEQ ID NOs: 73-75; SEQ ID NOs: 89-91; SEQ ID NOs: 105-107; or SEQ ID NOs: 121-123), a constant domain (e.g., a kappa or lambda constant domain), and (c) an antigen binding domain having the ability to bind to a polypeptide expressed on the surface of a NK cell (e.g., an anti-human CD16a VH domain, an anti-human CD16a scFv, or an anti-human NKG2A scFv).

In one embodiment, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (i) a heavy chain variable domain having a CDR1 having the amino acid sequence set forth in SEQ ID NO:1 (or a variant of SEQ ID NO:1 with one, two, or three amino acid modifications), a CDR2 having the amino acid sequence set forth in SEQ ID NO:2 (or a variant of SEQ ID NO:2 with one, two, or three amino acid modifications), and a CDR3 having the amino acid sequence set forth in SEQ ID NO:3 (or a variant of SEQ ID NO:3 with one, two, or three amino acid modifications); and/or (ii) a light chain variable domain having a CDR1 having the amino acid sequence set forth in SEQ ID NO:9 (or a variant of SEQ ID NO:9 with one, two, or three amino acid modifications), a CDR2 having the amino acid sequence set forth in SEQ ID NO:10 (or a variant of SEQ ID NO: 10 with one, two, or three amino acid modifications), and a CDR3 having the amino acid sequence set forth SEQ ID NO: 11 (or a variant of SEQ ID NO:11 with one, two, or three amino acid modifications). An example of such an antigen binding fragment having these CDRs and the ability to bind to an PSCA polypeptide (e.g., a human PSCA polypeptide) includes, without limitation, the Fab set forth in FIGS. 2A and 2B.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) and (a) a heavy chain variable domain having a CDR1 having the amino acid sequence set forth in SEQ ID NO: 1 (or a variant of SEQ ID NO:1 with one, two, or three amino acid modifications), a CDR2 having the amino acid sequence set forth in SEQ ID NO:2 (or a variant of SEQ ID NO: 2 with one, two, or three amino acid modifications), and a CDR3 having the amino acid sequence set forth in SEQ ID NO:3 (or a variant of SEQ ID NO:3 with one, two, or three amino acid modifications) and/or (b) a light chain variable domain having a CDR1 having the amino acid sequence set forth in SEQ ID NO:9 (or a variant of SEQ ID NO:9 with one, two, or three amino acid modifications), a CDR2 having the amino acid sequence set forth in SEQ ID NO:10 (or a variant of SEQ ID NO:10 with one, two, or three amino acid modifications), and a CDR3 having the amino acid sequence set forth SEQ ID NO:11 (or a variant of SEQ ID NO:11 with one, two, or three amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO: 12 (or a variant of SEQ ID NO: 12 with one, two, three, four, five, six, seven, eight, nine, ten, or more amino acid modifications), a framework region 2 having the amino acid sequence set forth in SEQ ID NO:13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO:14 with one, two, three, four, five, six, seven, eight, nine, ten, or more amino acid modifications), and a framework region 4 having the amino acid sequence set forth in SEQ ID NO:15 (or a variant of SEQ ID NO:15 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIGS. 2A or 2B can be designed to include framework regions as set forth in FIGS. 2A and 2B or can be designed to include one or more framework regions from another antibody, antibody fragment, or antibody domain. For example, an Fab or a scFv can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having the amino acid set forth in SEQ ID NO:20, a framework region 1 having the amino acid set forth in SEQ ID NO:36, a framework region 1 having the amino acid set forth in SEQ ID NO:52, a framework region 1 having the amino acid set forth in SEQ ID NO:68, a framework region 1 having the amino acid set forth in SEQ ID NO:84, a framework region 1 having the amino acid set forth in SEQ ID NO:100, or a framework region 1 having the amino acid set forth in SEQ ID NO:116.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO:16. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO: 16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs: 1, 2, and 3, and/or (b) a light chain variable domain that includes an amino acid sequence having at least 90 percent identity to the amino acid sequence set forth in SEQ ID NO:16, provided that the light chain variable domain includes the amino acid sequences set forth in SEQ ID NOs: 9, 10, and 11. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs: 1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO: 16, provided that the light chain variable domain includes the amino acid sequences set forth in SEQ ID NOs: 9, 10, and 11.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:16 or the amino acid set forth in SEQ ID NO: 16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an PSCA polypeptide (e.g., a human PSCA polypeptide), can include a heavy chain variable domain having the amino acid sequence set forth in SEQ ID NO: 8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 1, 2, and 3, and can include a light chain variable domain having the amino acid sequence set forth in SEQ ID NO:16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 9, 10, and 11.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO: 2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO:11. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO: 1, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO: 1 include, without limitation, those set forth in Table 1.

TABLE 1
Exemplary CDR1s that consist essentially of the
amino acid sequence set forth in SEQ ID NO: 1.

| Sequence | SEQ ID NO: |
|---|---|
| EYYMS | 355 |
| DYYMT | 356 |
| EYYMT | 357 |
| DYYMH | 358 |
| DYYMA | 359 |
| EYYMH | 360 |
| EYYMA | 361 |
| EYYMD | 362 |
| DYYMD | 363 |
| EYYME | 364 |

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:2 include, without limitation, those set forth in Table 2.

TABLE 2
Exemplary CDR2s that consist essentially of the
amino acid sequence set forth in SEQ ID NO: 2.

| Sequence | SEQ ID NO: |
|---|---|
| YLSSSGSTIYYADSVKG | 365 |
| YLTSSGSTIYYADSVKG | 366 |
| YISSSGSTIYYAESVKG | 367 |
| YISTSGSTIYYAESVKG | 368 |
| YISSSGTTIYYAESVKG | 369 |
| YISSSGSTIYFAESVKG | 370 |
| YISSTGSTIYYADSVKG | 371 |
| YISTSGSTIYYADSVKG | 372 |
| YISSSGTTIYYADSVKG | 373 |
| YISSSGSTIYYADTVKG | 374 |

As used herein, a “CDR3 that consists essentially of the amino acid sequence set forth in SEQ ID NO:3” is a CDR3 that has zero, one, or two amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:3 include, without limitation, those set forth in Table 3.

TABLE 3
Exemplary CDR3s that consist essentially of the
amino acid sequence set forth in SEQ ID NO: 3.

| Sequence | SEQ ID NO: |
|---|---|
| PLKWGMDV | 375 |
| PLRWGMEV | 376 |
| PLRWGMDA | 377 |
| PLRWSMDV | 378 |
| PLKWGMDA | 379 |
| PLKWGMEV | 380 |
| PLRWGLDV | 381 |
| PVRWGMDV | 382 |
| PLRFGMDV | 383 |
| PVKWGMDV | 384 |

As used herein, a “CDR1 that consists essentially of the amino acid sequence set forth in SEQ ID NO:9” is a CDR1 that has zero, one, or two amino acid substitutions within SEQ ID NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO:9 include, without limitation, those set forth in Table 4.

TABLE 4
Exemplary CDR1s that consist essentially of the
amino acid sequence set forth in SEQ ID NO: 9.

| Sequence | SEQ ID NO: |
|---|---|
| KASQSISSYLN | 385 |
| RATQSISSYLN | 386 |
| RASQTISSYLN | 387 |
| RASQSITSYLN | 388 |
| RASQSISTYLN | 389 |
| KASQSISSYLD | 390 |
| RASESISSYLN | 391 |
| RASQSISSYMN | 392 |
| RTSQSISSYLN | 393 |
| RASQSLSSYLN | 394 |

As used herein, a “CDR2 that consists essentially of the amino acid sequence set forth in SEQ ID NO:10” is a CDR2 that has zero, one, or two amino acid substitutions within SEQ ID NO:10, that has zero, one, two, three, four, or five amino acid residues directly preceding SEQ ID NO: 10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:10 include, without limitation, those set forth in Table 5.

TABLE 5
Exemplary CDR2s that consist essentially of the
amino acid sequence set forth in SEQ ID NO: 10.

| Sequence | SEQ ID NO: |
|---|---|
| TASSLQS | 395 |
| ATSSLQS | 396 |
| AATSLQS | 397 |
| AASTLQS | 398 |
| AASSLES | 399 |
| AASSLQT | 400 |
| TASSLQT | 401 |
| AASSLQA | 402 |
| AASSMQS | 403 |
| ADSSLQS | 404 |

As used herein, a “CDR3 that consists essentially of the amino acid sequence set forth in SEQ ID NO:11” is a CDR3 that has zero, one, or two amino acid substitutions within SEQ ID NO:11, that has zero, one, two, three, four, or five amino acid residues directly preceding SEQ ID NO: 11, and/or that has zero, one, two, three, four, or five amino acid residues directly following SEQ ID NO:11, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:11 include, without limitation, those set forth in Table 6.

TABLE 6
Exemplary CDR3s that consist essentially of the
amino acid sequence set forth in SEQ ID NO: 11.

| Sequence | SEQ ID NO: |
|---|---|
| HESSSLPLT | 405 |
| HQTSSLPLT | 406 |
| HQSTSLPLT | 407 |
| HQSSTLPLT | 408 |
| HQSSSMPLT | 409 |
| HQSSSLPMT | 410 |
| HQSSSLPLS | 411 |
| TQSSSLPLT | 412 |
| HQSSSLPLD | 413 |
| HQSSSVPLT | 414 |

In another embodiment, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (i) a heavy chain variable domain having a CDR1 having the amino acid sequence set forth in SEQ ID NO:17 (or a variant of SEQ ID NO:17 with one, two, or three amino acid modifications), a CDR2 having the amino acid sequence set forth in SEQ ID NO:18 (or a variant of SEQ ID NO:18 with one, two, or three amino acid modifications), and a CDR3 having the amino acid sequence set forth in SEQ ID NO:19 (or a variant of SEQ ID NO: 19 with one, two, or three amino acid modifications); and/or (ii) a light chain variable domain having a CDR1 having the amino acid sequence set forth in SEQ ID NO:25 (or a variant of SEQ ID NO:25 with one, two, or three amino acid modifications), a CDR2 having the amino acid sequence set forth in SEQ ID NO:26 (or a variant of SEQ ID NO: 26 with one, two, or three amino acid modifications), and a CDR3 having the amino acid sequence set forth SEQ ID NO:27 (or a variant of SEQ ID NO:27 with one, two, or three amino acid modifications). An example of such an antigen binding fragment having these CDRs and the ability to bind to an PSCA polypeptide (e.g., a human PSCA polypeptide) includes, without limitation, the Fab set forth in FIGS. 3A and 3B.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) and (a) a heavy chain variable domain having a CDR1 having the amino acid sequence set forth in SEQ ID NO: 17 (or a variant of SEQ ID NO: 17 with one, two, or three amino acid modifications), a CDR2 having the amino acid sequence set forth in SEQ ID NO:18 (or a variant of SEQ ID NO: 18 with one, two, or three amino acid modifications), and a CDR3 having the amino acid sequence set forth in SEQ ID NO:19 (or a variant of SEQ ID NO:19 with one, two, or three amino acid modifications) and/or (b) a light chain variable domain having a CDR1 having the amino acid sequence set forth in SEQ ID NO:25 (or a variant of SEQ ID NO: 25 with one, two, or three amino acid modifications), a CDR2 having the amino acid sequence set forth in SEQ ID NO:26 (or a variant of SEQ ID NO:26 with one, two, or three amino acid modifications), and a CDR3 having the amino acid sequence set forth SEQ ID NO:27 (or a variant of SEQ ID NO:27 with one, two, or three amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:20 (or a variant of SEQ ID NO:20 with one, two, three, four, five, six, seven, eight, nine, ten, or more amino acid modifications), a framework region 2 having the amino acid sequence set forth in SEQ ID NO:21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO: 22 with one, two, three, four, five, six, seven, eight, nine, ten, or more amino acid modifications), and a framework region 4 having the amino acid sequence set forth in SEQ ID NO:23 (or a variant of SEQ ID NO:23 with one, two, three, four, five, six, seven, eight, nine, ten, or more amino acid modifications) and/or (b) a light chain variable domain that includes a framework region 1 having the amino acid sequence set forth in SEQ ID NO:28 (or a variant of SEQ ID NO:28 with one, two, three, four, five, six, seven, eight, nine, ten, or more amino acid modifications), a framework region 2 having the amino acid sequence set forth in SEQ ID NO:29 (or a variant of SEQ ID NO:29 with one, two, three, four, five, six, seven, eight, nine, ten, or more amino acid modifications), a framework region 3 having the amino acid sequence set forth in SEQ ID NO:30 (or a variant of SEQ ID NO:30 with one, two, three, four, five, six, seven, eight, nine, ten, or more amino acid modifications), and a framework region 4 having the amino acid sequence set forth in SEQ ID NO:31 (or a variant of SEQ ID NO:31 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIGS. 3A or 3B can be designed to include framework regions as set forth in FIGS. 3A and 3B or can be designed to include one or more framework regions from another antibody, antibody fragment, or antibody domain. For example, an Fab or a scFv can be designed to include the six CDRs set forth in FIGS. 3A and 3B and the framework regions set forth in FIGS. 3A and 3B except that framework region 1 having the amino acid set forth in SEQ ID NO:20 is replaced with a framework region 1 having the amino acid set forth in SEQ ID NO:4, a framework region 1 having the amino acid set forth in SEQ ID NO:36, a framework region 1 having the amino acid set forth in SEQ ID NO:52, a framework region 1 having the amino acid set forth in SEQ ID NO:68, a framework region 1 having the amino acid set forth in SEQ ID NO:84, a framework region 1 having the amino acid set forth in SEQ ID NO: 100, or a framework region 1 having the amino acid set forth in SEQ ID NO:116.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:24 and/or (b) a light chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24 and/or (b) a light chain variable domain that includes an amino acid sequence having at least 90, 91, 92, 93, 94, 95, 96, 97, 98, or 99 percent identity to the amino acid sequence set forth in SEQ ID NO: 32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24 and/or (b) a light chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs: 17, 18, and 19, and/or (b) a light chain variable domain that includes an amino acid sequence having at least 90 percent identity to the amino acid sequence set forth in SEQ ID NO:32, provided that the light chain variable domain includes the amino acid sequences set forth in SEQ ID NOs: 25, 26, and 27. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24, provided that the heavy chain variable domain includes the amino acid sequences set forth in SEQ ID NOs: 17, 18, and 19, and/or (b) a light chain variable domain that includes an amino acid sequence having at least 90, 91, 92, 93, 94, 95, 96, 97, 98, or 99 percent identity to the amino acid sequence set forth in SEQ ID NO: 32, provided that the light chain variable domain includes the amino acid sequences set forth in SEQ ID NOs: 25, 26, and 27.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:32 or the amino acid set forth in SEQ ID NO: 32 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an PSCA polypeptide (e.g., a human PSCA polypeptide), can include a heavy chain variable domain having the amino acid sequence set forth in SEQ ID NO: 24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 17, 18, and 19, and can include a light chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 25, 26, and 27.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain comprising (i) a CDR1 that comprises, consists essentially of, or consists of the amino acid sequence set forth in SEQ ID NO:17, (ii) a CDR2 that comprises, consists essentially of, or consists of the amino acid sequence set forth in SEQ ID NO: 18, and (iii) a CDR3 that comprises, consists essentially of, or consists of the amino acid sequence set forth in SEQ ID NO:19, and/or (b) a light chain variable domain comprising (i) a CDR1 that comprises, consists essentially of, or consists of the amino acid sequence set forth in SEQ ID NO:25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO:17” is a CDR1 that has zero, one, or two amino acid substitutions within SEQ ID NO:17, that has zero, one, two, three, four, or five amino acid residues directly preceding SEQ ID NO: 17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO: 17 include, without limitation, those set forth in Table 7.

TABLE 7
Exemplary CDR1s that consist essentially of the
amino acid sequence set forth in SEQ ID NO: 17.

| Sequence | SEQ ID NO: |
|---|---|
| EYYMS | 415 |
| DYYMT | 416 |
| EYYMT | 417 |
| DYYMH | 418 |
| DYYMA | 419 |
| EYYMH | 420 |
| EYYMA | 421 |
| EYYMD | 422 |
| DYYMD | 423 |
| EYYME | 424 |

As used herein, a “CDR2 that consists essentially of the amino acid sequence set forth in SEQ ID NO:18” is a CDR2 that has zero, one, or two amino acid substitutions within SEQ ID NO:18, that has zero, one, two, three, four, or five amino acid residues directly preceding SEQ ID NO:18, and/or that has zero, one, two, three, four, or five amino acid residues directly following SEQ ID NO:18, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO: 18 include, without limitation, those set forth in Table 8.

TABLE 8
Exemplary CDR2s that consist essentially of the
amino acid sequence set forth in SEQ ID NO: 18.

| Sequence | SEQ ID NO: |
|---|---|
| YLSSSGSTIYYADSVKG | 425 |
| YLTSSGSTIYYADSVKG | 426 |
| YISSSGSTIYYAESVKG | 427 |
| YISTSGSTIYYAESVKG | 428 |
| YISSSGTTIYYAESVKG | 429 |
| YISSSGSTIYFAESVKG | 430 |
| YISSTGSTIYYADSVKG | 431 |
| YISTSGSTIYYADSVKG | 432 |
| YISSSGTTIYYADSVKG | 433 |
| YISSSGSTIYYADTVKG | 434 |

As used herein, a “CDR3 that consists essentially of the amino acid sequence set forth in SEQ ID NO: 19” is a CDR3 that has zero, one, or two amino acid substitutions within SEQ ID NO:19, that has zero, one, two, three, four, or five amino acid residues directly preceding SEQ ID NO: 19, and/or that has zero, one, two, three, four, or five amino acid residues directly following SEQ ID NO:19, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO: 19 include, without limitation, those set forth in Table 9.

TABLE 9
Exemplary CDR3s that consist essentially of the
amino acid sequence set forth in SEQ ID NO: 19.

| Sequence | SEQ ID NO: |
|---|---|
| PLKWGMDV | 435 |
| PLRWGMEV | 436 |
| PLRWGMDA | 437 |
| PLRWSMDV | 438 |
| PLKWGMDA | 439 |
| PLKWGMEV | 440 |
| PLRWGLDV | 441 |
| PVRWGMDV | 442 |
| PLRFGMDV | 443 |
| PVKWGMDV | 444 |

As used herein, a “CDR1 that consists essentially of the amino acid sequence set forth in SEQ ID NO:25” is a CDR1 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO:25 include, without limitation, those set forth in Table 10.

TABLE 10
Exemplary CDR1s that consist essentially of the
amino acid sequence set forth in SEQ ID NO: 25.

| Sequence | SEQ ID NO: |
|---|---|
| KASQSISSYLN | 445 |
| RATQSISSYLN | 446 |
| RASQTISSYLN | 447 |
| RASQSITSYLN | 448 |
| RASQSISTYLN | 449 |
| KASQSISSYLD | 450 |
| RASESISSYLN | 451 |
| RASQSISSYMN | 452 |
| RTSQSISSYLN | 453 |
| RASQSLSSYLN | 454 |

As used herein, a “CDR2 that consists essentially of the amino acid sequence set forth in SEQ ID NO:26” is a CDR2 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:26 include, without limitation, those set forth in Table 11.

TABLE 11
Exemplary CDR2s that consist essentially of the
amino acid sequence set forth in SEQ ID NO: 26.

| Sequence | SEQ ID NO: |
|---|---|
| TASSLQS | 455 |
| ATSSLQS | 456 |
| AATSLQS | 457 |
| AASTLQS | 458 |
| AASSLES | 459 |
| AASSLQT | 460 |
| TASSLQT | 461 |
| AASSLQA | 462 |
| AASSMQS | 463 |
| ADSSLQS | 464 |

As used herein, a “CDR3 that consists essentially of the amino acid sequence set forth in SEQ ID NO:27” is a CDR3 that has zero, one, or two amino acid substitutions within SEQ ID NO:27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:27 include, without limitation, those set forth in Table 12.

TABLE 12
Exemplary CDR3s that consist essentially of the
amino acid sequence set forth in SEQ ID NO: 27.

| Sequence | SEQ ID NO: |
|---|---|
| HESSSLPLT | 465 |
| HQTSSLPLT | 466 |
| HQSTSLPLT | 467 |
| HQSSTLPLT | 468 |
| HQSSSMPLT | 469 |
| HQSSSLPMT | 470 |
| HQSSSLPLS | 471 |
| TQSSSLPLT | 472 |
| HQSSSLPLD | 473 |
| HQSSSVPLT | 474 |

In another embodiment, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (i) a heavy chain variable domain having a CDR1 having the amino acid sequence set forth in SEQ ID NO:33 (or a variant of SEQ ID NO:33 with one, two, or three amino acid modifications), a CDR2 having the amino acid sequence set forth in SEQ ID NO:34 (or a variant of SEQ ID NO:34 with one, two, or three amino acid modifications), and a CDR3 having the amino acid sequence set forth in SEQ ID NO:35 (or a variant of SEQ ID NO: 35 with one, two, or three amino acid modifications); and/or (ii) a light chain variable domain having a CDR1 having the amino acid sequence set forth in SEQ ID NO:41 (or a variant of SEQ ID NO:41 with one, two, or three amino acid modifications), a CDR2 having the amino acid sequence set forth in SEQ ID NO:42 (or a variant of SEQ ID NO: 42 with one, two, or three amino acid modifications), and a CDR3 having the amino acid sequence set forth SEQ ID NO:43 (or a variant of SEQ ID NO:43 with one, two, or three amino acid modifications). An example of such an antigen binding fragment having these CDRs and the ability to bind to an PSCA polypeptide (e.g., a human PSCA polypeptide) includes, without limitation, the Fab set forth in FIGS. 4A and 4B.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) and (a) a heavy chain variable domain having a CDR1 having the amino acid sequence set forth in SEQ ID NO: 33 (or a variant of SEQ ID NO:33 with one, two, or three amino acid modifications), a CDR2 having the amino acid sequence set forth in SEQ ID NO:34 (or a variant of SEQ ID NO: 34 with one, two, or three amino acid modifications), and a CDR3 having the amino acid sequence set forth in SEQ ID NO:35 (or a variant of SEQ ID NO:35 with one, two, or three amino acid modifications) and/or (b) a light chain variable domain having a CDR1 having the amino acid sequence set forth in SEQ ID NO:41 (or a variant of SEQ ID NO: 41 with one, two, or three amino acid modifications), a CDR2 having the amino acid sequence set forth in SEQ ID NO:42 (or a variant of SEQ ID NO:42 with one, two, or three amino acid modifications), and a CDR3 having the amino acid sequence set forth SEQ ID NO:43 (or a variant of SEQ ID NO:43 with one, two, or three amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:36 (or a variant of SEQ ID NO:36 with one, two, three, four, five, six, seven, eight, nine, ten, or more amino acid modifications), a framework region 2 having the amino acid sequence set forth in SEQ ID NO:37 (or a variant of SEQ ID NO:37 with one, two, three, four, five, six, seven, eight, nine, ten, or more amino acid modifications), a framework region 3 having the amino acid sequence set forth in SEQ ID NO:38 (or a variant of SEQ ID NO: 38 with one, two, three, four, five, six, seven, eight, nine, ten, or more amino acid modifications), and a framework region 4 having the amino acid sequence set forth in SEQ ID NO:39 (or a variant of SEQ ID NO:39 with one, two, three, four, five, six, seven, eight, nine, ten, or more amino acid modifications) and/or (b) a light chain variable domain that includes a framework region 1 having the amino acid sequence set forth in SEQ ID NO:44 (or a variant of SEQ ID NO:44 with one, two, three, four, five, six, seven, eight, nine, ten, or more amino acid modifications), a framework region 2 having the amino acid sequence set forth in SEQ ID NO:45 (or a variant of SEQ ID NO:45 with one, two, three, four, five, six, seven, eight, nine, ten, or more amino acid modifications), a framework region 3 having the amino acid sequence set forth in SEQ ID NO:46 (or a variant of SEQ ID NO:46 with one, two, three, four, five, six, seven, eight, nine, ten, or more amino acid modifications), and a framework region 4 having the amino acid sequence set forth in SEQ ID NO:47 (or a variant of SEQ ID NO:47 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIGS. 4A or 4B can be designed to include framework regions as set forth in FIGS. 4A and 4B or can be designed to include one or more framework regions from another antibody, antibody fragment, or antibody domain. For example, an Fab or a scFv can be designed to include the six CDRs set forth in FIGS. 4A and 4B and the framework regions set forth in FIGS. 4A and 4B except that framework region 1 having the amino acid set forth in SEQ ID NO:36 is replaced with a framework region 1 having the amino acid set forth in SEQ ID NO:4, a framework region 1 having the amino acid set forth in SEQ ID NO:20, a framework region 1 having the amino acid set forth in SEQ ID NO:52, a framework region 1 having the amino acid set forth in SEQ ID NO:68, a framework region 1 having the amino acid set forth in SEQ ID NO:84, a framework region 1 having the amino acid set forth in SEQ ID NO: 100, or a framework region 1 having the amino acid set forth in SEQ ID NO:116.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:40 and/or (b) a light chain variable domain that includes an amino acid sequence having at least 90 percent identity to the amino acid sequence set forth in SEQ ID NO:48. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:40 and/or (b) a light chain variable domain that includes an amino acid sequence having at least 90, 91, 92, 93, 94, 95, 96, 97, 98, or 99 percent identity to the amino acid sequence set forth in SEQ ID NO: 48. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:40 and/or (b) a light chain variable domain that includes an amino acid sequence having 100 percent identity to the amino acid sequence set forth in SEQ ID NO:48.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:40, provided that the heavy chain variable domain includes the amino acid sequences set forth in SEQ ID NOs: 33, 34, and 35, and/or (b) a light chain variable domain that includes an amino acid sequence having at least 90 percent identity to the amino acid sequence set forth in SEQ ID NO:48, provided that the light chain variable domain includes the amino acid sequences set forth in SEQ ID NOs: 41, 42, and 43. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:40, provided that the heavy chain variable domain includes the amino acid sequences set forth in SEQ ID NOs: 33, 34, and 35, and/or (b) a light chain variable domain that includes an amino acid sequence having at least 90, 91, 92, 93, 94, 95, 96, 97, 98, or 99 percent identity to the amino acid sequence set forth in SEQ ID NO: 48, provided that the light chain variable domain includes the amino acid sequences set forth in SEQ ID NOs: 41, 42, and 43.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain having the amino acid sequence set forth in SEQ ID NO:40 or the amino acid set forth in SEQ ID NO:40 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:48 or the amino acid set forth in SEQ ID NO: 48 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an PSCA polypeptide (e.g., a human PSCA polypeptide), can include a heavy chain variable domain having the amino acid sequence set forth in SEQ ID NO: 40 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 33, 34, and 35, and can include a light chain variable domain having the amino acid sequence set forth in SEQ ID NO:48 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 41, 42, and 43.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain comprising (i) a CDR1 that comprises, consists essentially of, or consists of the amino acid sequence set forth in SEQ ID NO:33, (ii) a CDR2 that comprises, consists essentially of, or consists of the amino acid sequence set forth in SEQ ID NO: 34, and (iii) a CDR3 that comprises, consists essentially of, or consists of the amino acid sequence set forth in SEQ ID NO:35, and/or (b) a light chain variable domain comprising (i) a CDR1 that comprises, consists essentially of, or consists of the amino acid sequence set forth in SEQ ID NO:41, (ii) a CDR2 that comprises, consists essentially of, or consists of the amino acid sequence set forth in SEQ ID NO:42, and (iii) a CDR3 that comprises, consists essentially of, or consists of the amino acid sequence set forth in SEQ ID NO:43. As used herein, a “CDR1 that consists essentially of the amino acid sequence set forth in SEQ ID NO:33” is a CDR1 that has zero, one, or two amino acid substitutions within SEQ ID NO:33, that has zero, one, two, three, four, or five amino acid residues directly preceding SEQ ID NO:33, and/or that has zero, one, two, three, four, or five amino acid residues directly following SEQ ID NO:33, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO:33 include, without limitation, those set forth in Table 13.

TABLE 13
Exemplary CDR1s that consist essentially of the
amino acid sequence set forth in SEQ ID NO: 33.

| Sequence | SEQ ID NO: |
|---|---|
| EYYMS | 475 |
| DYYMT | 476 |
| EYYMT | 477 |
| DYYMH | 478 |
| DYYMA | 479 |
| EYYMH | 480 |
| EYYMA | 481 |
| EYYMD | 482 |
| DYYMD | 483 |
| EYYME | 484 |

As used herein, a “CDR2 that consists essentially of the amino acid sequence set forth in SEQ ID NO:34” is a CDR2 that has zero, one, or two amino acid substitutions within SEQ ID NO:34, that has zero, one, two, three, four, or five amino acid residues directly preceding SEQ ID NO:34, and/or that has zero, one, two, three, four, or five amino acid residues directly following SEQ ID NO:34, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:34 include, without limitation, those set forth in Table 14.

TABLE 14
Exemplary CDR2s that consist essentially of the
amino acid sequence set forth in SEQ ID NO: 34.

| Sequence | SEQ ID NO: |
|---|---|
| YLSSSGSTIYYADSVKG | 485 |
| YLTSSGSTIYYADSVKG | 486 |
| YISSSGSTIYYAESVKG | 487 |
| YISTSGSTIYYAESVKG | 488 |
| YISSSGTTIYYAESVKG | 489 |
| YISSSGSTIYFAESVKG | 490 |
| YISSTGSTIYYADSVKG | 491 |
| YISTSGSTIYYADSVKG | 492 |
| YISSSGTTIYYADSVKG | 493 |
| YISSSGSTIYYADTVKG | 494 |

As used herein, a “CDR3 that consists essentially of the amino acid sequence set forth in SEQ ID NO:35” is a CDR3 that has zero, one, or two amino acid substitutions within SEQ ID NO:35, that has zero, one, two, three, four, or five amino acid residues directly preceding SEQ ID NO:35, and/or that has zero, one, two, three, four, or five amino acid residues directly following SEQ ID NO:35, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:35 include, without limitation, those set forth in Table 15.

TABLE 15
Exemplary CDR3s that consist essentially of the
amino acid sequence set forth in SEQ ID NO: 35.

| Sequence | SEQ ID NO: |
|---|---|
| PLKWGMDV | 495 |
| PLRWGMEV | 496 |
| PLRWGMDA | 497 |
| PLRWSMDV | 498 |
| PLKWGMDA | 499 |
| PLKWGMEV | 500 |
| PLRWGLDV | 501 |
| PVRWGMDV | 502 |
| PLRFGMDV | 503 |
| PVKWGMDV | 504 |

As used herein, a “CDR1 that consists essentially of the amino acid sequence set forth in SEQ ID NO:41” is a CDR1 that has zero, one, or two amino acid substitutions within SEQ ID NO:41, that has zero, one, two, three, four, or five amino acid residues directly preceding SEQ ID NO:41, and/or that has zero, one, two, three, four, or five amino acid residues directly following SEQ ID NO:41, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO:41 include, without limitation, those set forth in Table 16.

TABLE 16
Exemplary CDR1s that consist essentially of the
amino acid sequence set forth in SEQ ID NO: 41.

| Sequence | SEQ ID NO: |
|---|---|
| KASQSISSYLN | 505 |
| RATQSISSYLN | 506 |
| RASQTISSYLN | 507 |
| RASQSITSYLN | 508 |
| RASQSISTYLN | 509 |
| KASQSISSYLD | 510 |
| RASESISSYLN | 511 |
| RASQSISSYMN | 512 |
| RTSQSISSYLN | 513 |
| RASQSLSSYLN | 514 |

As used herein, a “CDR2 that consists essentially of the amino acid sequence set forth in SEQ ID NO:42” is a CDR2 that has zero, one, or two amino acid substitutions within SEQ ID NO:42, that has zero, one, two, three, four, or five amino acid residues directly preceding SEQ ID NO:42, and/or that has zero, one, two, three, four, or five amino acid residues directly following SEQ ID NO:42, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:42 include, without limitation, those set forth in Table 17.

TABLE 17
Exemplary CDR2s that consist essentially of the
amino acid sequence set forth in SEQ ID NO: 42.

| Sequence | SEQ ID NO: |
|---|---|
| TASSLQS | 515 |
| ATSSLQS | 516 |
| AATSLQS | 517 |
| AASTLQS | 518 |
| AASSLES | 519 |
| AASSLQT | 520 |
| TASSLQT | 521 |
| AASSLQA | 522 |
| AASSMQS | 523 |
| ADSSLQS | 524 |

As used herein, a “CDR3 that consists essentially of the amino acid sequence set forth in SEQ ID NO:43” is a CDR3 that has zero, one, or two amino acid substitutions within SEQ ID NO:43, that has zero, one, two, three, four, or five amino acid residues directly preceding SEQ ID NO:43, and/or that has zero, one, two, three, four, or five amino acid residues directly following SEQ ID NO:43, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:43 include, without limitation, those set forth in Table 18.

TABLE 18
Exemplary CDR3s that consist essentially of the
amino acid sequence set forth in SEQ ID NO: 43.

| Sequence | SEQ ID NO: |
|---|---|
| DQSNSLPLT | 525 |
| HESNSLPLT | 526 |
| HQTNSLPLT | 527 |
| HQSESLPLT | 528 |
| HQSNTLPLT | 529 |
| HQSNSMPLT | 530 |
| HQSNSLPMT | 531 |
| HQSNSLPLS | 532 |
| HQANSLPLT | 533 |
| HQSNALPLT | 534 |

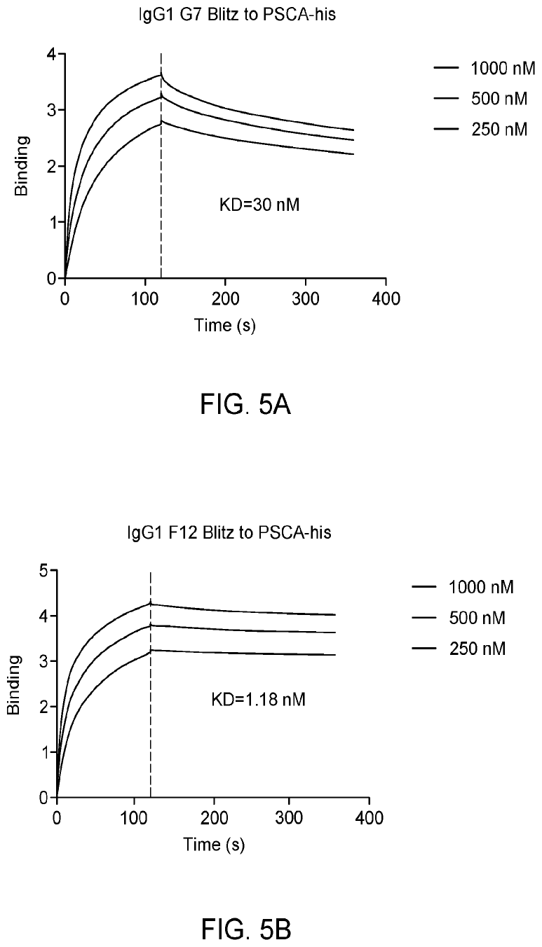

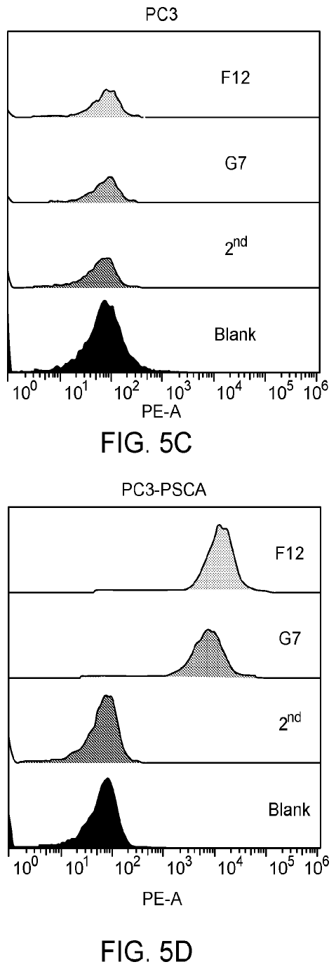

In another embodiment, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (i) a heavy chain variable domain having a CDR1 having the amino acid sequence set forth in SEQ ID NO:49 (or a variant of SEQ ID NO:49 with one, two, or three amino acid modifications), a CDR2 having the amino acid sequence set forth in SEQ ID NO:50 (or a variant of SEQ ID NO:50 with one, two, or three amino acid modifications), and a CDR3 having the amino acid sequence set forth in SEQ ID NO:51 (or a variant of SEQ ID NO: 51 with one, two, or three amino acid modifications); and/or (ii) a light chain variable domain having a CDR1 having the amino acid sequence set forth in SEQ ID NO:57 (or a variant of SEQ ID NO:57 with one, two, or three amino acid modifications), a CDR2 having the amino acid sequence set forth in SEQ ID NO:58 (or a variant of SEQ ID NO: 58 with one, two, or three amino acid modifications), and a CDR3 having the amino acid sequence set forth SEQ ID NO:59 (or a variant of SEQ ID NO:59 with one, two, or three amino acid modifications). An example of such an antigen binding fragment having these CDRs and the ability to bind to an PSCA polypeptide (e.g., a human PSCA polypeptide) includes, without limitation, the Fab set forth in FIGS. 5A and 5B.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) and (a) a heavy chain variable domain having a CDR1 having the amino acid sequence set forth in SEQ ID NO: 49 (or a variant of SEQ ID NO:49 with one, two, or three amino acid modifications), a CDR2 having the amino acid sequence set forth in SEQ ID NO:50 (or a variant of SEQ ID NO: 50 with one, two, or three amino acid modifications), and a CDR3 having the amino acid sequence set forth in SEQ ID NO:51 (or a variant of SEQ ID NO:51 with one, two, or three amino acid modifications) and/or (b) a light chain variable domain having a CDR1 having the amino acid sequence set forth in SEQ ID NO:57 (or a variant of SEQ ID NO: 57 with one, two, or three amino acid modifications), a CDR2 having the amino acid sequence set forth in SEQ ID NO:58 (or a variant of SEQ ID NO:58 with one, two, or three amino acid modifications), and a CDR3 having the amino acid sequence set forth SEQ ID NO:59 (or a variant of SEQ ID NO:59 with one, two, or three amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:52 (or a variant of SEQ ID NO:52 with one, two, three, four, five, six, seven, eight, nine, ten, or more amino acid modifications), a framework region 2 having the amino acid sequence set forth in SEQ ID NO:53 (or a variant of SEQ ID NO:53 with one, two, three, four, five, six, seven, eight, nine, ten, or more amino acid modifications), a framework region 3 having the amino acid sequence set forth in SEQ ID NO:54 (or a variant of SEQ ID NO: 54 with one, two, three, four, five, six, seven, eight, nine, ten, or more amino acid modifications), and a framework region 4 having the amino acid sequence set forth in SEQ ID NO:55 (or a variant of SEQ ID NO:55 with one, two, three, four, five, six, seven, eight, nine, ten, or more amino acid modifications) and/or (b) a light chain variable domain that includes a framework region 1 having the amino acid sequence set forth in SEQ ID NO:60 (or a variant of SEQ ID NO:60 with one, two, three, four, five, six, seven, eight, nine, ten, or more amino acid modifications), a framework region 2 having the amino acid sequence set forth in SEQ ID NO:61 (or a variant of SEQ ID NO:61 with one, two, three, four, five, six, seven, eight, nine, ten, or more amino acid modifications), a framework region 3 having the amino acid sequence set forth in SEQ ID NO:62 (or a variant of SEQ ID NO:62 with one, two, three, four, five, six, seven, eight, nine, ten, or more amino acid modifications), and a framework region 4 having the amino acid sequence set forth in SEQ ID NO:63 (or a variant of SEQ ID NO:63 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIGS. 5A or 5B can be designed to include framework regions as set forth in FIGS. 5A and 5B or can be designed to include one or more framework regions from another antibody, antibody fragment, or antibody domain. For example, an Fab or a scFv can be designed to include the six CDRs set forth in FIGS. 5A and 5B and the framework regions set forth in FIGS. 5A and 5B except that framework region 1 having the amino acid set forth in SEQ ID NO:52 is replaced with a framework region 1 having the amino acid set forth in SEQ ID NO:4, a framework region 1 having the amino acid set forth in SEQ ID NO:20, a framework region 1 having the amino acid set forth in SEQ ID NO:36, a framework region 1 having the amino acid set forth in SEQ ID NO:68, a framework region 1 having the amino acid set forth in SEQ ID NO:84, a framework region 1 having the amino acid set forth in SEQ ID NO: 100, or a framework region 1 having the amino acid set forth in SEQ ID NO:116.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:56 and/or (b) a light chain variable domain that includes an amino acid sequence having at least 90 percent identity to the amino acid sequence set forth in SEQ ID NO:64. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:56 and/or (b) a light chain variable domain that includes an amino acid sequence having at least 90, 91, 92, 93, 94, 95, 96, 97, 98, or 99 percent identity to the amino acid sequence set forth in SEQ ID NO: 64. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:56 and/or (b) a light chain variable domain that includes an amino acid sequence having 100 percent identity to the amino acid sequence set forth in SEQ ID NO:64.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:56, provided that the heavy chain variable domain includes the amino acid sequences set forth in SEQ ID NOs: 49, 50, and 51, and/or (b) a light chain variable domain that includes an amino acid sequence having at least 90 percent identity to the amino acid sequence set forth in SEQ ID NO:64, provided that the light chain variable domain includes the amino acid sequences set forth in SEQ ID NOs: 57, 58, and 59. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:56, provided that the heavy chain variable domain includes the amino acid sequences set forth in SEQ ID NOs: 49, 50, and 51, and/or (b) a light chain variable domain that includes an amino acid sequence having at least 90, 91, 92, 93, 94, 95, 96, 97, 98, or 99 percent identity to the amino acid sequence set forth in SEQ ID NO: 64, provided that the light chain variable domain includes the amino acid sequences set forth in SEQ ID NOs: 57, 58, and 59.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain having the amino acid sequence set forth in SEQ ID NO:56 or the amino acid set forth in SEQ ID NO:56 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:64 or the amino acid set forth in SEQ ID NO: 64 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an PSCA polypeptide (e.g., a human PSCA polypeptide), can include a heavy chain variable domain having the amino acid sequence set forth in SEQ ID NO: 56 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 49, 50, and 51, and can include a light chain variable domain having the amino acid sequence set forth in SEQ ID NO:64 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 57, 58, and 59.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain comprising (i) a CDR1 that comprises, consists essentially of, or consists of the amino acid sequence set forth in SEQ ID NO:49, (ii) a CDR2 that comprises, consists essentially of, or consists of the amino acid sequence set forth in SEQ ID NO: 50, and (iii) a CDR3 that comprises, consists essentially of, or consists of the amino acid sequence set forth in SEQ ID NO:51, and/or (b) a light chain variable domain comprising (i) a CDR1 that comprises, consists essentially of, or consists of the amino acid sequence set forth in SEQ ID NO:57, (ii) a CDR2 that comprises, consists essentially of, or consists of the amino acid sequence set forth in SEQ ID NO:58, and (iii) a CDR3 that comprises, consists essentially of, or consists of the amino acid sequence set forth in SEQ ID NO:59. As used herein, a “CDR1 that consists essentially of the amino acid sequence set forth in SEQ ID NO:49” is a CDR1 that has zero, one, or two amino acid substitutions within SEQ ID NO:49, that has zero, one, two, three, four, or five amino acid residues directly preceding SEQ ID NO:49, and/or that has zero, one, two, three, four, or five amino acid residues directly following SEQ ID NO:49, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO:49 include, without limitation, those set forth in Table 19.

TABLE 19
Exemplary CDR1s that consist essentially of the
amino acid sequence set forth in SEQ ID NO: 49.

| Sequence | SEQ ID NO: |
|---|---|
| EYYMS | 535 |
| DYYMT | 536 |
| EYYMT | 537 |
| DYYMH | 538 |
| DYYMA | 539 |
| EYYMH | 540 |
| EYYMA | 541 |
| EYYMD | 542 |
| DYYMD | 543 |
| EYYME | 544 |

As used herein, a “CDR2 that consists essentially of the amino acid sequence set forth in SEQ ID NO:50” is a CDR2 that has zero, one, or two amino acid substitutions within SEQ ID NO:50, that has zero, one, two, three, four, or five amino acid residues directly preceding SEQ ID NO:50, and/or that has zero, one, two, three, four, or five amino acid residues directly following SEQ ID NO:50, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:50 include, without limitation, those set forth in Table 20.

TABLE 20
Exemplary CDR2s that consist essentially of the
amino acid sequence set forth in SEQ ID NO: 50.

| Sequence | SEQ ID NO: |
|---|---|
| FISSSGSTIVYADSVKG | 545 |
| YMSSSGSTIVYADSVKG | 546 |
| YITSSGSTIVYADSVKG | 547 |
| YISTSGSTIVYADSVKG | 548 |
| YISSTGSTIVYADSVKG | 549 |
| YISSSGTTIVYADSVKG | 550 |
| YISSSGSTLVYADSVKG | 551 |
| YISSSGSTIVYAESVKG | 552 |
| YISSSGSTIVYADTVKG | 553 |
| YISSSGSTIVYADSVRG | 554 |

As used herein, a “CDR3 that consists essentially of the amino acid sequence set forth in SEQ ID NO:51” is a CDR3 that has zero, one, or two amino acid substitutions within SEQ ID NO:51, that has zero, one, two, three, four, or five amino acid residues directly preceding SEQ ID NO:51, and/or that has zero, one, two, three, four, or five amino acid residues directly following SEQ ID NO:51, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:51 include, without limitation, those set forth in Table 21.

TABLE 21
Exemplary CDR3s that consist essentially of the
amino acid sequence set forth in SEQ ID NO: 51.

| Sequence | SEQ ID NO: |
|---|---|
| PLRRGMDV | 555 |
| PMRRGMDV | 556 |
| PIKRGMDV | 557 |
| PIRKGMDV | 558 |
| PIRRAMDV | 559 |
| PIRRGLDV | 560 |
| PIRRGMEV | 561 |
| PIRRGMDA | 562 |
| PIRRSMDV | 563 |
| PIRRGMQV | 564 |

As used herein, a “CDR1 that consists essentially of the amino acid sequence set forth in SEQ ID NO:57” is a CDR1 that has zero, one, or two amino acid substitutions within SEQ ID NO:57, that has zero, one, two, three, four, or five amino acid residues directly preceding SEQ ID NO:57, and/or that has zero, one, two, three, four, or five amino acid residues directly following SEQ ID NO:57, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO:57 include, without limitation, those set forth in Table 22.

TABLE 22
Exemplary CDR1s that consist essentially of the
amino acid sequence set forth in SEQ ID NO: 57.

| Sequence | SEQ ID NO: |
|---|---|
| KASQSISSYLN | 565 |
| RATQSISSYLN | 566 |
| RASQTISSYLN | 567 |
| RASQSITSYLN | 568 |
| RASQSISTYLN | 569 |
| KASQSISSYLD | 570 |
| RASESISSYLN | 571 |
| RASQSISSYMN | 572 |
| RTSQSISSYLN | 573 |
| RASQSLSSYLN | 574 |

As used herein, a “CDR2 that consists essentially of the amino acid sequence set forth in SEQ ID NO:58” is a CDR2 that has zero, one, or two amino acid substitutions within SEQ ID NO:58, that has zero, one, two, three, four, or five amino acid residues directly preceding SEQ ID NO:58, and/or that has zero, one, two, three, four, or five amino acid residues directly following SEQ ID NO:58, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:58 include, without limitation, those set forth in Table 23.

TABLE 23
Exemplary CDR2s that consist essentially of the
amino acid sequence set forth in SEQ ID NO: 58.

| Sequence | SEQ ID NO: |
|---|---|
| TASSLQS | 575 |
| ATSSLQS | 576 |
| AATSLQS | 577 |
| AASTLQS | 578 |
| AASSLES | 579 |
| AASSLQT | 580 |
| TASSLQT | 581 |
| AASSLQA | 582 |
| AASSMQS | 583 |
| ADSSLQS | 584 |

As used herein, a “CDR3 that consists essentially of the amino acid sequence set forth in SEQ ID NO:59” is a CDR3 that has zero, one, or two amino acid substitutions within SEQ ID NO:59, that has zero, one, two, three, four, or five amino acid residues directly preceding SEQ ID NO:59, and/or that has zero, one, two, three, four, or five amino acid residues directly following SEQ ID NO:59, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:59 include, without limitation, those set forth in Table 24.

TABLE 24
Exemplary CDR3s that consist essentially of the
amino acid sequence set forth in SEQ ID NO: 59.

| Sequence | SEQ ID NO: |
|---|---|
| HESSSLPLT | 585 |
| HQTSSLPLT | 586 |
| HQSTSLPLT | 587 |
| HQSSTLPLT | 588 |
| HQSSSMPLT | 589 |
| HQSSSLPMT | 590 |
| HQSSSLPLS | 591 |
| TQSSSLPLT | 592 |
| HQSSSLPLD | 593 |
| HQSSSVPLT | 594 |

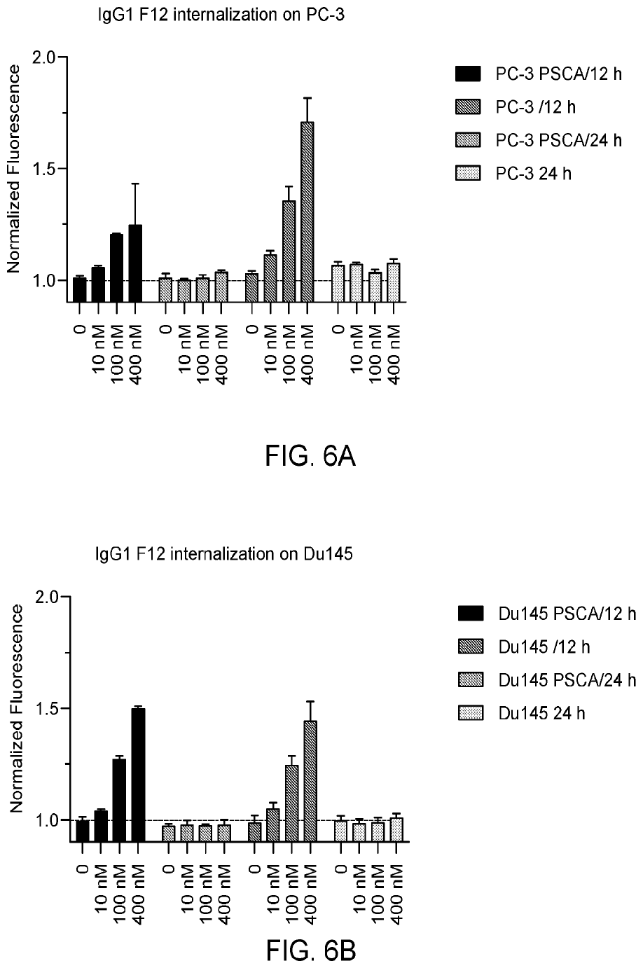

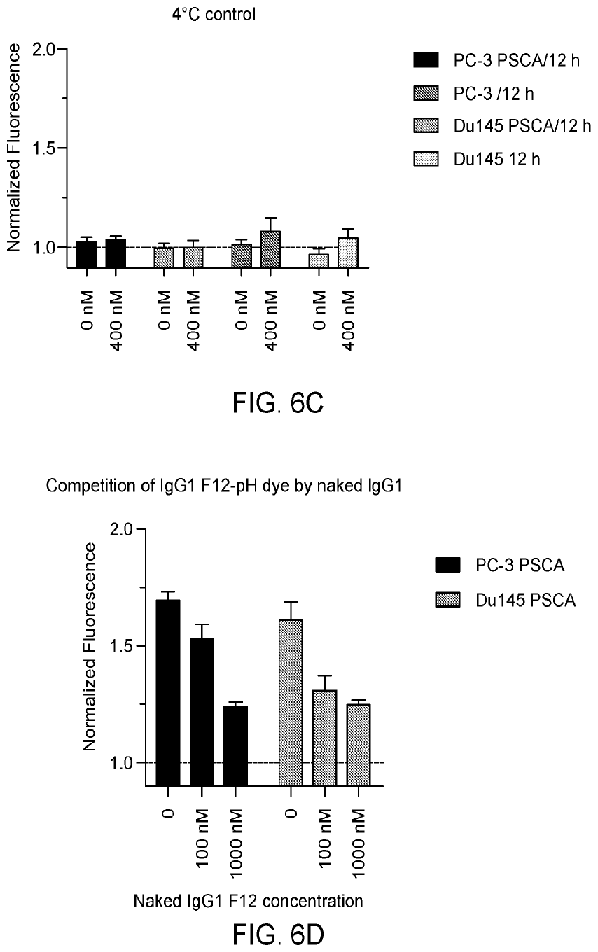

In another embodiment, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (i) a heavy chain variable domain having a CDR1 having the amino acid sequence set forth in SEQ ID NO:65 (or a variant of SEQ ID NO:65 with one, two, or three amino acid modifications), a CDR2 having the amino acid sequence set forth in SEQ ID NO:66 (or a variant of SEQ ID NO:66 with one, two, or three amino acid modifications), and a CDR3 having the amino acid sequence set forth in SEQ ID NO:67 (or a variant of SEQ ID NO: 67 with one, two, or three amino acid modifications); and/or (ii) a light chain variable domain having a CDR1 having the amino acid sequence set forth in SEQ ID NO:73 (or a variant of SEQ ID NO:73 with one, two, or three amino acid modifications), a CDR2 having the amino acid sequence set forth in SEQ ID NO:74 (or a variant of SEQ ID NO: 74 with one, two, or three amino acid modifications), and a CDR3 having the amino acid sequence set forth SEQ ID NO:75 (or a variant of SEQ ID NO:75 with one, two, or three amino acid modifications). An example of such an antigen binding fragment having these CDRs and the ability to bind to an PSCA polypeptide (e.g., a human PSCA polypeptide) includes, without limitation, the Fab set forth in FIGS. 6A and 6B.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) and (a) a heavy chain variable domain having a CDR1 having the amino acid sequence set forth in SEQ ID NO: 65 (or a variant of SEQ ID NO:65 with one, two, or three amino acid modifications), a CDR2 having the amino acid sequence set forth in SEQ ID NO:66 (or a variant of SEQ ID NO: 66 with one, two, or three amino acid modifications), and a CDR3 having the amino acid sequence set forth in SEQ ID NO:67 (or a variant of SEQ ID NO:67 with one, two, or three amino acid modifications) and/or (b) a light chain variable domain having a CDR1 having the amino acid sequence set forth in SEQ ID NO:73 (or a variant of SEQ ID NO: 73 with one, two, or three amino acid modifications), a CDR2 having the amino acid sequence set forth in SEQ ID NO:74 (or a variant of SEQ ID NO:74 with one, two, or three amino acid modifications), and a CDR3 having the amino acid sequence set forth SEQ ID NO:75 (or a variant of SEQ ID NO:75 with one, two, or three amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:68 (or a variant of SEQ ID NO:68 with one, two, three, four, five, six, seven, eight, nine, ten, or more amino acid modifications), a framework region 2 having the amino acid sequence set forth in SEQ ID NO:69 (or a variant of SEQ ID NO:69 with one, two, three, four, five, six, seven, eight, nine, ten, or more amino acid modifications), a framework region 3 having the amino acid sequence set forth in SEQ ID NO:70 (or a variant of SEQ ID NO: 70 with one, two, three, four, five, six, seven, eight, nine, ten, or more amino acid modifications), and a framework region 4 having the amino acid sequence set forth in SEQ ID NO:71 (or a variant of SEQ ID NO:71 with one, two, three, four, five, six, seven, eight, nine, ten, or more amino acid modifications) and/or (b) a light chain variable domain that includes a framework region 1 having the amino acid sequence set forth in SEQ ID NO:76 (or a variant of SEQ ID NO:76 with one, two, three, four, five, six, seven, eight, nine, ten, or more amino acid modifications), a framework region 2 having the amino acid sequence set forth in SEQ ID NO:77 (or a variant of SEQ ID NO:77 with one, two, three, four, five, six, seven, eight, nine, ten, or more amino acid modifications), a framework region 3 having the amino acid sequence set forth in SEQ ID NO:78 (or a variant of SEQ ID NO:78 with one, two, three, four, five, six, seven, eight, nine, ten, or more amino acid modifications), and a framework region 4 having the amino acid sequence set forth in SEQ ID NO:79 (or a variant of SEQ ID NO:79 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIGS. 6A or 6B can be designed to include framework regions as set forth in FIGS. 6A and 6B or can be designed to include one or more framework regions from another antibody, antibody fragment, or antibody domain. For example, an Fab or a scFv can be designed to include the six CDRs set forth in FIGS. 6A and 6B and the framework regions set forth in FIGS. 6A and 6B except that framework region 1 having the amino acid set forth in SEQ ID NO:68 is replaced with a framework region 1 having the amino acid set forth in SEQ ID NO:4, a framework region 1 having the amino acid set forth in SEQ ID NO:20, a framework region 1 having the amino acid set forth in SEQ ID NO:36, a framework region 1 having the amino acid set forth in SEQ ID NO:52, a framework region 1 having the amino acid set forth in SEQ ID NO:84, a framework region 1 having the amino acid set forth in SEQ ID NO: 100, or a framework region 1 having the amino acid set forth in SEQ ID NO:116.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:72 and/or (b) a light chain variable domain that includes an amino acid sequence having at least 90 percent identity to the amino acid sequence set forth in SEQ ID NO:80. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:72 and/or (b) a light chain variable domain that includes an amino acid sequence having at least 90, 91, 92, 93, 94, 95, 96, 97, 98, or 99 percent identity to the amino acid sequence set forth in SEQ ID NO: 80. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:72 and/or (b) a light chain variable domain that includes an amino acid sequence having 100 percent identity to the amino acid sequence set forth in SEQ ID NO:80.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain that includes an amino acid sequence having at least 90 percent identity to the amino acid sequence set forth in SEQ ID NO:72, provided that the heavy chain variable domain includes the amino acid sequences set forth in SEQ ID NOs: 65, 66, and 67, and/or (b) a light chain variable domain that includes an amino acid sequence having at least 90 percent identity to the amino acid sequence set forth in SEQ ID NO:80, provided that the light chain variable domain includes the amino acid sequences set forth in SEQ ID NOs: 73, 74, and 75. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:72, provided that the heavy chain variable domain includes the amino acid sequences set forth in SEQ ID NOs: 65, 66, and 67, and/or (b) a light chain variable domain that includes an amino acid sequence having at least 90, 91, 92, 93, 94, 95, 96, 97, 98, or 99 percent identity to the amino acid sequence set forth in SEQ ID NO: 80, provided that the light chain variable domain includes the amino acid sequences set forth in SEQ ID NOs: 73, 74, and 75.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain having the amino acid sequence set forth in SEQ ID NO: 72 or the amino acid set forth in SEQ ID NO:72 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:80 or the amino acid set forth in SEQ ID NO: 80 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to an PSCA polypeptide (e.g., a human PSCA polypeptide), can include a heavy chain variable domain having the amino acid sequence set forth in SEQ ID NO: 72 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 65, 66, and 67, and can include a light chain variable domain having the amino acid sequence set forth in SEQ ID NO:80 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 73, 74, and 75.

In some cases, a binder (e.g., an antibody, antigen binding fragment, antibody domain, a CAR, a cell engager, and/or an ADC) provided herein having the ability to bind to an PSCA polypeptide (e.g., a human PSCA polypeptide) can include (a) a heavy chain variable domain comprising (i) a CDR1 that comprises, consists essentially of, or consists of the amino acid sequence set forth in SEQ ID NO:65, (ii) a CDR2 that comprises, consists essentially of, or consists of the amino acid sequence set forth in SEQ ID NO: 66, and (iii) a CDR3 that comprises, consists essentially of, or consists of the amino acid sequence set forth in SEQ ID NO:67, and/or (b) a light chain variable domain comprising (i) a CDR1 that comprises, consists essentially of, or consists of the amino acid sequence set forth in SEQ ID NO:73, (ii) a CDR2 that comprises, consists essentially of, or consists of the amino acid sequence set forth in SEQ ID NO:74, and (iii) a CDR3 that comprises, consists essentially of, or consists of the amino acid sequence set forth in SEQ ID NO:75. As used herein, a “CDR1 that consists essentially of the amino acid sequence set forth in SEQ ID NO:65” is a CDR1 that has zero, one, or two amino acid substitutions within SEQ ID NO:65, that has zero, one, two, three, four, or five amino acid residues directly preceding SEQ ID NO:65, and/or that has zero, one, two, three, four, or five amino acid residues directly following SEQ ID NO:65, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO:65 include, without limitation, those set forth in Table 25.

TABLE 25
Exemplary CDR1s that consist essentially of the
amino acid sequence set forth in SEQ ID NO: 65.

| Sequence | SEQ ID NO: |
|---|---|
| EYYMS | 595 |
| DYYMT | 596 |
| EYYMT | 597 |
| DYYMH | 598 |
| DYYMA | 599 |
| EYYMH | 600 |
| EYYMA | 601 |
| EYYMD | 602 |
| DYYMD | 603 |
| EYYME | 604 |

As used herein, a “CDR2 that consists essentially of the amino acid sequence set forth in SEQ ID NO:66” is a CDR2 that has zero, one, or two amino acid substitutions within SEQ ID NO:66, that has zero, one, two, three, four, or five amino acid residues directly preceding SEQ ID NO:66, and/or that has zero, one, two, three, four, or five amino acid residues directly following SEQ ID NO:66, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:66 include, without limitation, those set forth in Table 26.

TABLE 26
Exemplary CDR2s that consist essentially of the
amino acid sequence set forth in SEQ ID NO: 66.

| Sequence | SEQ ID NO: |
|---|---|
| FISSSGSTINYADSVKG | 605 |
| YLSSSGSTINYADSVKG | 606 |
| YITSSGSTINYADSVKG | 607 |
| YISTSGSTINYADSVKG | 608 |
| YISSTGSTINYADSVKG | 609 |
| YISSSASTINYADSVKG | 610 |
| YISSSGTTINYADSVKG | 611 |
| YISSSGSTINYAESVKG | 612 |
| YISSSGSTINYADSVRG | 613 |
| YISSSGSTINYADSVKA | 614 |

As used herein, a “CDR3 that consists essentially of the amino acid sequence set forth in SEQ ID NO:67” is a CDR3 that has zero, one, or two amino acid substitutions within SEQ ID NO:67, that has zero, one, two, three, four, or five amino acid residues directly preceding SEQ ID NO:67, and/or that has zero, one, two, three, four, or five amino acid residues directly following SEQ ID NO:67, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:67 include, without limitation, those set forth in Table 27.

TABLE 27
Exemplary CDR3s that consist essentially of the
amino acid sequence set forth in SEQ ID NO: 67.

| Sequence | SEQ ID NO: |
|---|---|
| PLRWGMDV | 615 |
| PIKWGMDV | 616 |
| PIRFGMDV | 617 |
| PIRWAMDV | 618 |
| PIRWGLDV | 619 |
| PIRWGMEV | 620 |
| PIRWGMDA | 621 |
| PIRWSMDV | 622 |
| PMRWGMDV | 623 |
| PIRWGMEA | 624 |

As used herein, a “CDR1 that consists essentially of the amino acid sequence set forth in SEQ ID NO:73” is a CDR1 that has zero, one, or two amino acid substitutions within SEQ ID NO:73, that has zero, one, two, three, four, or five amino acid residues directly preceding SEQ ID NO:73, and/or that has zero, one, two, three, four, or five amino acid residues directly following SEQ ID NO:73, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR1 that consists essentially of the amino acid sequence set forth in SEQ ID NO:73 include, without limitation, those set forth in Table 28.

TABLE 28
Exemplary CDR1s that consist essentially of the
amino acid sequence set forth in SEQ ID NO: 73.

| Sequence | SEQ ID NO: |
|---|---|
| KASQSISSYLN | 625 |
| RATQSISSYLN | 626 |
| RASQTISSYLN | 627 |
| RASQSITSYLN | 628 |
| RASQSISTYLN | 629 |
| KASQSISSYLD | 630 |
| RASESISSYLN | 631 |
| RASQSISSYMN | 632 |
| RTSQSISSYLN | 633 |
| RASQSLSSYLN | 634 |

As used herein, a “CDR2 that consists essentially of the amino acid sequence set forth in SEQ ID NO:74” is a CDR2 that has zero, one, or two amino acid substitutions within SEQ ID NO:74, that has zero, one, two, three, four, or five amino acid residues directly preceding SEQ ID NO:74, and/or that has zero, one, two, three, four, or five amino acid residues directly following SEQ ID NO:74, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR2 that consists essentially of the amino acid sequence set forth in SEQ ID NO:74 include, without limitation, those set forth in Table 29.

TABLE 29
Exemplary CDR2s that consist essentially of the
amino acid sequence set forth in SEQ ID NO: 74.

| Sequence | SEQ ID NO: |
|---|---|
| TASSLQS | 635 |
| ATSSLQS | 636 |
| AATSLQS | 637 |
| AASTLQS | 638 |
| AASSLES | 639 |
| AASSLQT | 640 |
| TASSLQT | 641 |
| AASSLQA | 642 |
| AASSMQS | 643 |
| ADSSLQS | 644 |

As used herein, a “CDR3 that consists essentially of the amino acid sequence set forth in SEQ ID NO:75” is a CDR3 that has zero, one, or two amino acid substitutions within SEQ ID NO:75, that has zero, one, two, three, four, or five amino acid residues directly preceding SEQ ID NO:75, and/or that has zero, one, two, three, four, or five amino acid residues directly following SEQ ID NO:75, provided that the binder (e.g., an antibody, antigen binding fragment, antibody domain, a CAR, a cell engager, and/or an ADC) maintains its basic ability to bind to an PSCA polypeptide (e.g., a human PSCA polypeptide). Examples of a CDR3 that consists essentially of the amino acid sequence set forth in SEQ ID NO:75 include, without limitation, those set forth in Table 30.

TABLE 30
Exemplary CDR3s that consist essentially of the
amino acid sequence set forth in SEQ ID NO: 75.

| Sequence | SEQ ID NO: |
|---|---|
| HESSSLPLT | 645 |
| HQTSSLPLT | 646 |
| HQSTSLPLT | 647 |
| HQSSTLPLT | 648 |
| HQSSSMPLT | 649 |
| HQSSSLPMT | 650 |
| HQSSSLPLS | 651 |
| TQSSSLPLT | 652 |
| HQSSSLPLD | 653 |
| HQSSSVPLT | 654 |

In another embodiment, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (i) a heavy chain variable domain having a CDR1 having the amino acid sequence set forth in SEQ ID NO:81 (or a variant of SEQ ID NO:81 with one or two amino acid modifications), a CDR2 having the amino acid sequence set forth in SEQ ID NO:82 (or a variant of SEQ ID NO:82 with one or two amino acid modifications), and a CDR3 having the amino acid sequence set forth in SEQ ID NO:83 (or a variant of SEQ ID NO:83 with one or two amino acid modifications); and/or (ii) a light chain variable domain having a CDR1 having the amino acid sequence set forth in SEQ ID NO:89 (or a variant of SEQ ID NO: 89 with one or two amino acid modifications), a CDR2 having the amino acid sequence set forth in SEQ ID NO:90 (or a variant of SEQ ID NO:90 with one or two amino acid modifications), and a CDR3 having the amino acid sequence set forth SEQ ID NO: 91 (or a variant of SEQ ID NO:91 with one or two amino acid modifications). An example of such a binder having these CDRs and the ability to bind to a PSCA polypeptide (e.g., a human PSCA polypeptide) includes, without limitation, the scFv set forth in FIGS. 7A and 7B.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) and (a) a heavy chain variable domain having a CDR1 having the amino acid sequence set forth in SEQ ID NO: 81 (or a variant of SEQ ID NO:81 with one or two amino acid modifications), a CDR2 having the amino acid sequence set forth in SEQ ID NO:82 (or a variant of SEQ ID NO: 82 with one or two amino acid modifications), and a CDR3 having the amino acid sequence set forth in SEQ ID NO:83 (or a variant of SEQ ID NO:83 with one or two amino acid modifications) and/or (b) a light chain variable domain having a CDR1 having the amino acid sequence set forth in SEQ ID NO:89 (or a variant of SEQ ID NO: 89 with one or two amino acid modifications), a CDR2 having the amino acid sequence set forth in SEQ ID NO:90 (or a variant of SEQ ID NO:90 with one or two amino acid modifications), and a CDR3 having the amino acid sequence set forth SEQ ID NO: 91 (or a variant of SEQ ID NO:91 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:84 (or a variant of SEQ ID NO: 84 with one, two, three, four, five, six, seven, eight, nine, ten, or more amino acid modifications), a framework region 2 having the amino acid sequence set forth in SEQ ID NO: 85 (or a variant of SEQ ID NO:85 with one, two, three, four, five, six, seven, eight, nine, ten, or more amino acid modifications), a framework region 3 having the amino acid sequence set forth in SEQ ID NO:86 (or a variant of SEQ ID NO:86 with one, two, three, four, five, six, seven, eight, nine, ten, or more amino acid modifications), and a framework region 4 having the amino acid sequence set forth in SEQ ID NO:87 (or a variant of SEQ ID NO:87 with one, two, three, four, five, six, seven, eight, nine, ten, or more amino acid modifications) and/or (b) a light chain variable domain that includes a framework region 1 having the amino acid sequence set forth in SEQ ID NO:92 (or a variant of SEQ ID NO:92 with one, two, three, four, five, six, seven, eight, nine, ten, or more amino acid modifications), a framework region 2 having the amino acid sequence set forth in SEQ ID NO:93 (or a variant of SEQ ID NO:93 with one, two, three, four, five, six, seven, eight, nine, ten, or more amino acid modifications), a framework region 3 having the amino acid sequence set forth in SEQ ID NO:94 (or a variant of SEQ ID NO: 94 with one, two, three, four, five, six, seven, eight, nine, ten, or more amino acid modifications), and a framework region 4 having the amino acid sequence set forth in SEQ ID NO:95 (or a variant of SEQ ID NO:95 with one, two, three, four, five, six, seven, eight, nine, ten, or more amino acid modifications).

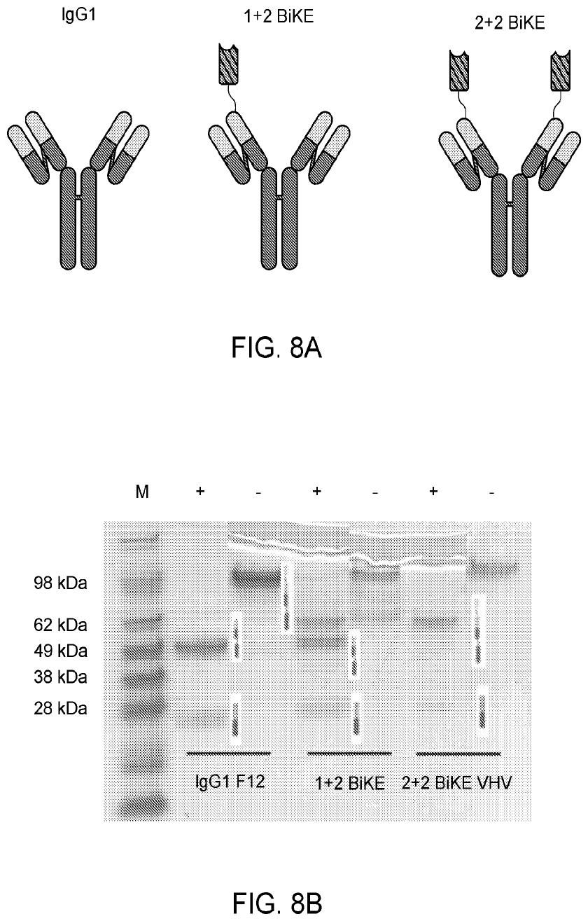

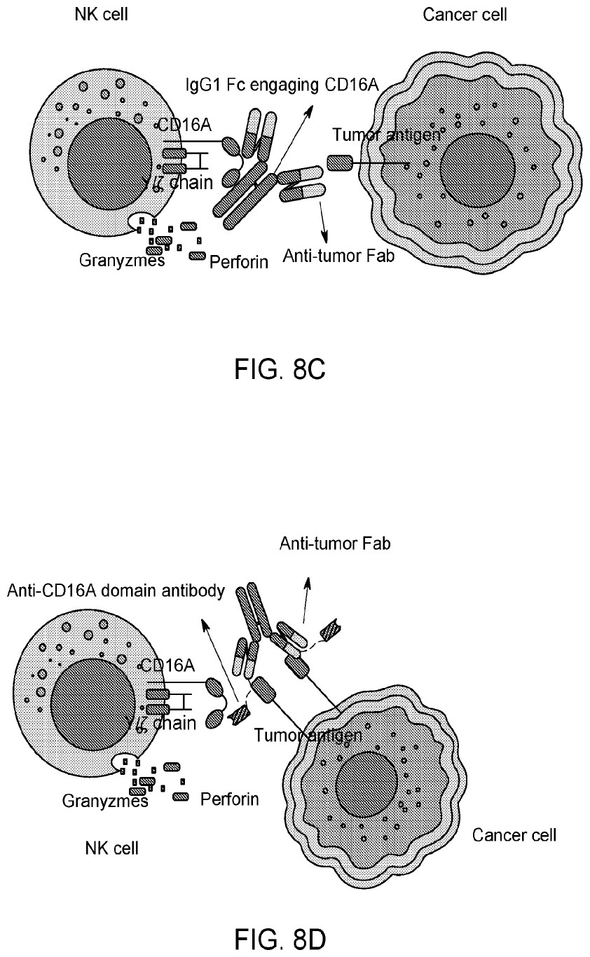

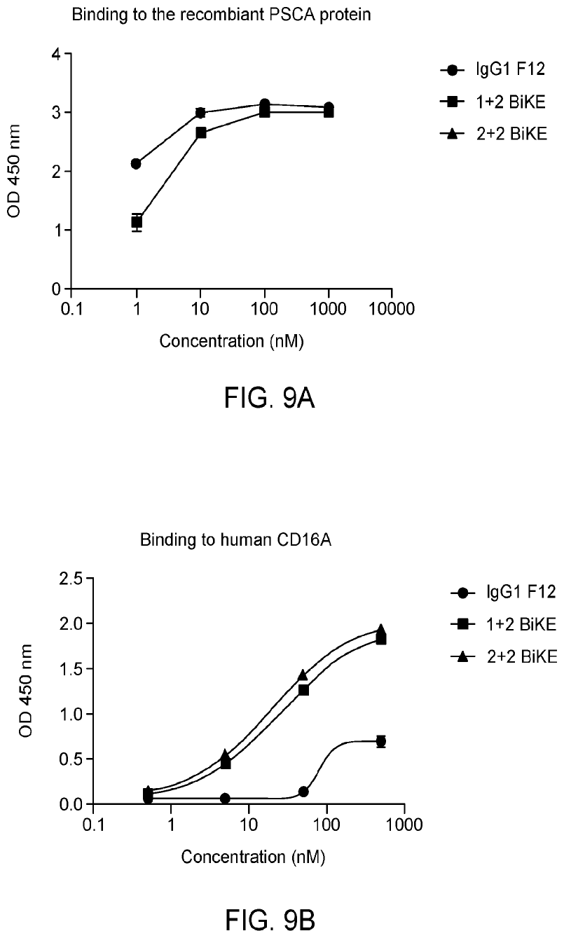

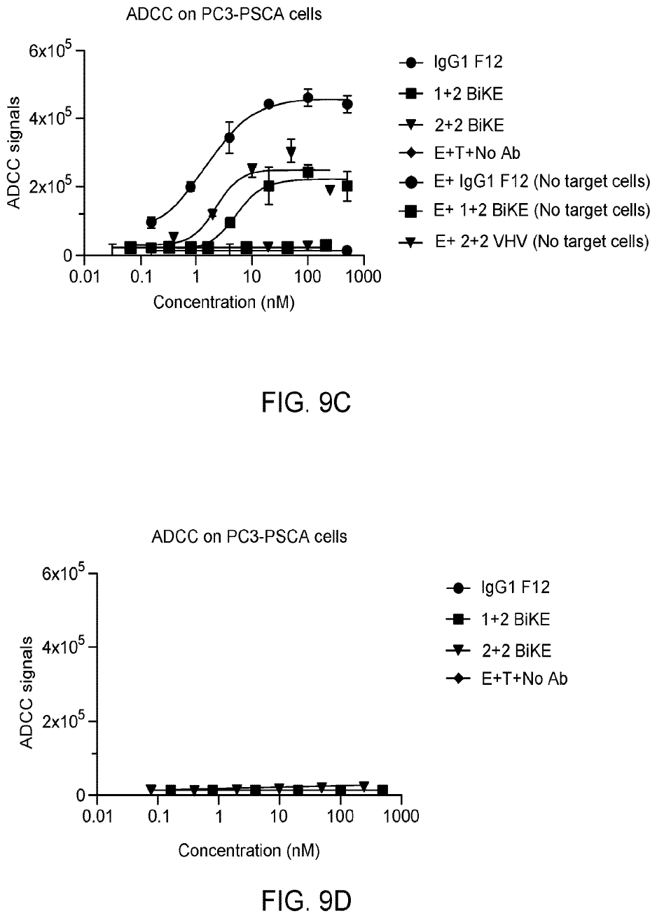

In some cases, a binder (e.g., an antibody, antigen binding fragment, antibody domain, a CAR, a cell engager, and/or an ADC) having any of the CDRs set forth in FIGS. 7A or 7B can be designed to include framework regions as set forth in FIGS. 7A and 7B or can be designed to include one or more framework regions from another antibody, antibody fragment, or antibody domain. For example, a scFv can be designed to include the six CDRs set forth in FIGS. 7A and 7B and the framework regions set forth in FIGS. 7A and 7B except that framework region 1 having the amino acid set forth in SEQ ID NO:84 is replaced with a framework region 1 having the amino acid set forth in SEQ ID NO:4 or a framework region 1 having the amino acid set forth in SEQ ID NO: 20 or a framework region 1 having the amino acid set forth in SEQ ID NO:36 or a framework region 1 having the amino acid set forth in SEQ ID NO:52 or a framework region 1 having the amino acid set forth in SEQ ID NO:68 or a framework region 1 having the amino acid set forth in SEQ ID NO: 100 or a framework region 1 having the amino acid set forth in SEQ ID NO:116. In some cases, an Fab can be designed to include the six CDRs set forth in FIGS. 7A and 7B and the framework regions set forth in FIGS. 7A and 7B. In some cases, an Fab can be designed to include the six CDRs set forth in FIGS. 7A and 7B and the framework regions set forth in FIGS. 7A and 7B except that framework region 1 having the amino acid set forth in SEQ ID NO:84 is replaced with a framework region 1 having the amino acid set forth in SEQ ID NO:4 or a framework region 1 having the amino acid set forth in SEQ ID NO:20 or a framework region 1 having the amino acid set forth in SEQ ID NO:36 or a framework region 1 having the amino acid set forth in SEQ ID NO:52 or a framework region 1 having the amino acid set forth in SEQ ID NO:68 or a framework region 1 having the amino acid set forth in SEQ ID NO: 100 or a framework region 1 having the amino acid set forth in SEQ ID NO: 116. In another example, a scFv can be designed to include the six CDRs set forth in FIGS. 7A and 7B and the framework regions set forth in FIGS. 8A and 8B or FIGS. 9A and 9B.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain that includes an amino acid sequence having at least 90 percent identity to the amino acid sequence set forth in SEQ ID NO:88 and/or (b) a light chain variable domain that includes an amino acid sequence having at least 90 percent identity to the amino acid sequence set forth in SEQ ID NO:96. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:88 and/or (b) a light chain variable domain that includes an amino acid sequence having at least 90, 91, 92, 93, 94, 95, 96, 97, 98, or 99 percent identity to the amino acid sequence set forth in SEQ ID NO: 96. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:88 and/or (b) a light chain variable domain that includes an amino acid sequence having 100 percent identity to the amino acid sequence set forth in SEQ ID NO:96.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain that includes an amino acid sequence having at least 90 percent identity to the amino acid sequence set forth in SEQ ID NO:88, provided that the heavy chain variable domain includes the amino acid sequences set forth in SEQ ID NOs: 81, 82, and 83, and/or (b) a light chain variable domain that includes an amino acid sequence having at least 90 percent identity to the amino acid sequence set forth in SEQ ID NO:96, provided that the light chain variable domain includes the amino acid sequences set forth in SEQ ID NOs: 89, 90, and 91. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:88, provided that the heavy chain variable domain includes the amino acid sequences set forth in SEQ ID NOs: 81, 82, and 83, and/or (b) a light chain variable domain that includes an amino acid sequence having at least 90, 91, 92, 93, 94, 95, 96, 97, 98, or 99 percent identity to the amino acid sequence set forth in SEQ ID NO: 96, provided that the light chain variable domain includes the amino acid sequences set forth in SEQ ID NOs: 89, 90, and 91.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain having the amino acid sequence set forth in SEQ ID NO:88 or the amino acid set forth in SEQ ID NO:88 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:96 or the amino acid set forth in SEQ ID NO: 96 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a PSCA polypeptide (e.g., a human PSCA polypeptide), can include a heavy chain variable domain having the amino acid sequence set forth in SEQ ID NO: 88 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 81, 82, and 83, and can include a light chain variable domain having the amino acid sequence set forth in SEQ ID NO:96 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 89, 90, and 91.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain comprising (i) a CDR1 that comprises, consists essentially of, or consists of the amino acid sequence set forth in SEQ ID NO:81, (ii) a CDR2 that comprises, consists essentially of, or consists of the amino acid sequence set forth in SEQ ID NO: 82, and (iii) a CDR3 that comprises, consists essentially of, or consists of the amino acid sequence set forth in SEQ ID NO:83, and/or (b) a light chain variable domain comprising (i) a CDR1 that comprises, consists essentially of, or consists of the amino acid sequence set forth in SEQ ID NO:89, (ii) a CDR2 that comprises, consists essentially of, or consists of the amino acid sequence set forth in SEQ ID NO:90, and (iii) a CDR3 that comprises, consists essentially of, or consists of the amino acid sequence set forth in SEQ ID NO:91. As used herein, a “CDR1 that consists essentially of the amino acid sequence set forth in SEQ ID NO:81” is a CDR1 that has zero, one, or two amino acid substitutions within SEQ ID NO:81, that has zero, one, two, three, four, or five amino acid residues directly preceding SEQ ID NO:81, and/or that has zero, one, two, three, four, or five amino acid residues directly following SEQ ID NO:81, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR1 that consists essentially of the amino acid sequence set forth in SEQ ID NO:81 include, without limitation, those set forth in Table 31.

TABLE 31
Exemplary CDR1s that consist essentially of the
amino acid sequence set forth in SEQ ID NO: 81.

| Sequence | SEQ ID NO: |
|---|---|
| TYAIS | 655 |
| SFAIS | 656 |
| SYTIS | 657 |
| SYALS | 658 |
| SYAMS | 659 |
| SYAIT | 660 |
| SYAIA | 661 |
| SYAID | 662 |
| EYAIT | 663 |
| SYDIS | 664 |

As used herein, a “CDR2 that consists essentially of the amino acid sequence set forth in SEQ ID NO:82” is a CDR2 that has zero, one, or two amino acid substitutions within SEQ ID NO:82, that has zero, one, two, three, four, or five amino acid residues directly preceding SEQ ID NO:82, and/or that has zero, one, two, three, four, or five amino acid residues directly following SEQ ID NO:82, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR2 that consists essentially of the amino acid sequence set forth in SEQ ID NO:82 include, without limitation, those set forth in Table 32.

TABLE 32
Exemplary CDR2s that consist essentially of the
amino acid sequence set forth in SEQ ID NO: 82.

| Sequence | SEQ ID NO: |
|---|---|
| AIIPIFGTANYAQKFQG | 665 |
| GILPIFGTANYAQKFQG | 666 |
| GIIPLFGTANYAQKFQG | 667 |
| GIIPIFATANYAQKFQG | 668 |
| GIIPIFGTAEYAQKFQG | 669 |
| GIIPIFGTANYANKFQG | 670 |
| GIIPIFGTANYAQRFQG | 671 |
| GIIPIFGTANYAQKFNG | 672 |
| GIIPIFGTANYAQKFQA | 673 |
| GIIPIFGTANYAQKYQG | 674 |

As used herein, a “CDR3 that consists essentially of the amino acid sequence set forth in SEQ ID NO:83” is a CDR3 that has zero, one, or two amino acid substitutions within SEQ ID NO:83, that has zero, one, two, three, four, or five amino acid residues directly preceding SEQ ID NO:83, and/or that has zero, one, two, three, four, or five amino acid residues directly following SEQ ID NO:83, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR3 that consists essentially of the amino acid sequence set forth in SEQ ID NO:83 include, without limitation, those set forth in Table 33.

TABLE 33
Exemplary CDR3s that consist essentially of the
amino acid sequence set forth in SEQ ID NO: 83.

| Sequence | SEQ ID NO: |
|---|---|
| DRIAVAGTNYYYGMDV | 675 |
| EKIAVAGTNYYYGMDV | 676 |
| ERLAVAGTNYYYGMDV | 677 |
| ERIALAGTNYYYGMDV | 678 |
| ERIAVAGSNYYYGMDV | 679 |
| ERIAVAGTDYYYGMDV | 680 |
| ERIAVAGTNYYYAMDV | 681 |
| ERIAVAGTNYYYGMEV | 682 |
| ERIAVAGTNYYYGMDA | 683 |
| ERIAVAGTNYYYGMDS | 684 |

As used herein, a “CDR1 that consists essentially of the amino acid sequence set forth in SEQ ID NO:89” is a CDR1 that has zero, one, or two amino acid substitutions within SEQ ID NO:89, that has zero, one, two, three, four, or five amino acid residues directly preceding SEQ ID NO:89, and/or that has zero, one, two, three, four, or five amino acid residues directly following SEQ ID NO:89, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR1 that consists essentially of the amino acid sequence set forth in SEQ ID NO:89 include, without limitation, those set forth in Table 34.

TABLE 34
Exemplary CDR1s that consist essentially of the
amino acid sequence set forth in SEQ ID NO: 89.

| Sequence | SEQ ID NO: |
|---|---|
| KASQSVSSSNLA | 685 |
| RSSQSVSSSNLA | 686 |
| RATQSVSSSNLA | 687 |
| RASNSVSSSNLA | 688 |
| RASQTVSSSNLA | 689 |
| RASQSASSSNLA | 690 |
| RASQSVTSSNLA | 691 |
| RASQSVSSSELA | 692 |
| RASQSVSSSNVA | 693 |
| RASQSVSSSNLG | 694 |

As used herein, a “CDR2 that consists essentially of the amino acid sequence set forth in SEQ ID NO:90” is a CDR2 that has zero, one, or two amino acid substitutions within SEQ ID NO:90, that has zero, one, two, three, four, or five amino acid residues directly preceding SEQ ID NO:90, and/or that has zero, one, two, three, four, or five amino acid residues directly following SEQ ID NO:90, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR2 that consists essentially of the amino acid sequence set forth in SEQ ID NO:90 include, without limitation, those set forth in Table 35.

TABLE 35
Exemplary CDR2s that consist essentially of the
amino acid sequence set forth in SEQ ID NO: 90.

| Sequence | SEQ ID NO: |
|---|---|
| AASTRAT | 695 |
| GGSTRAT | 696 |
| GATTRAT | 697 |
| GASSRAT | 698 |
| GASTKAT | 699 |
| GASTRGT | 700 |
| GASTRAS | 701 |
| GASTRAA | 702 |
| GASTRAD | 703 |
| GASTKGT | 704 |

As used herein, a “CDR3 that consists essentially of the amino acid sequence set forth in SEQ ID NO:91” is a CDR3 that has zero, one, or two amino acid substitutions within SEQ ID NO:91, that has zero, one, two, three, four, or five amino acid residues directly preceding SEQ ID NO:91, and/or that has zero, one, two, three, four, or five amino acid residues directly following SEQ ID NO:91, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR3 that consists essentially of the amino acid sequence set forth in SEQ ID NO:91 include, without limitation, those set forth in Table 36.

TABLE 36
Exemplary CDR3s that consist essentially of the
amino acid sequence set forth in SEQ ID NO: 91.

| Sequence | SEQ ID NO: |
|---|---|
| EQYGSSIT | 705 |
| QEYGSSIT | 706 |
| QQFGSSIT | 707 |
| QQYASSIT | 708 |
| QQYGTSIT | 709 |
| QQYGSTIT | 710 |
| QQYGSSLT | 711 |
| QQYGSSIS | 712 |
| QQYGSSIA | 713 |
| QNYGSSIT | 714 |

In another embodiment, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (i) a heavy chain variable domain having a CDR1 having the amino acid sequence set forth in SEQ ID NO:97 (or a variant of SEQ ID NO:97 with one or two amino acid modifications), a CDR2 having the amino acid sequence set forth in SEQ ID NO:98 (or a variant of SEQ ID NO:982 with one or two amino acid modifications), and a CDR3 having the amino acid sequence set forth in SEQ ID NO:99 (or a variant of SEQ ID NO: 99 with one or two amino acid modifications); and/or (ii) a light chain variable domain having a CDR1 having the amino acid sequence set forth in SEQ ID NO: 105 (or a variant of SEQ ID NO: 105 with one or two amino acid modifications), a CDR2 having the amino acid sequence set forth in SEQ ID NO:106 (or a variant of SEQ ID NO:106 with one or two amino acid modifications), and a CDR3 having the amino acid sequence set forth SEQ ID NO:107 (or a variant of SEQ ID NO:107 with one or two amino acid modifications). An example of such a binder having these CDRs and the ability to bind to a PSCA polypeptide (e.g., a human PSCA polypeptide) includes, without limitation, the scFv set forth in FIGS. 8A and 8B.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) and (a) a heavy chain variable domain having a CDR1 having the amino acid sequence set forth in SEQ ID NO: 97 (or a variant of SEQ ID NO:97 with one or two amino acid modifications), a CDR2 having the amino acid sequence set forth in SEQ ID NO:98 (or a variant of SEQ ID NO: 98 with one or two amino acid modifications), and a CDR3 having the amino acid sequence set forth in SEQ ID NO:99 (or a variant of SEQ ID NO:99 with one or two amino acid modifications) and/or (b) a light chain variable domain having a CDR1 having the amino acid sequence set forth in SEQ ID NO: 105 (or a variant of SEQ ID NO: 105 with one or two amino acid modifications), a CDR2 having the amino acid sequence set forth in SEQ ID NO: 106 (or a variant of SEQ ID NO:106 with one or two amino acid modifications), and a CDR3 having the amino acid sequence set forth SEQ ID NO: 107 (or a variant of SEQ ID NO:107 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO: 100 (or a variant of SEQ ID NO: 100 with one, two, three, four, five, six, seven, eight, nine, ten, or more amino acid modifications), a framework region 2 having the amino acid sequence set forth in SEQ ID NO: 101 (or a variant of SEQ ID NO:101 with one, two, three, four, five, six, seven, eight, nine, ten, or more amino acid modifications), a framework region 3 having the amino acid sequence set forth in SEQ ID NO:102 (or a variant of SEQ ID NO:102 with one, two, three, four, five, six, seven, eight, nine, ten, or more amino acid modifications), and a framework region 4 having the amino acid sequence set forth in SEQ ID NO:103 (or a variant of SEQ ID NO:103 with one, two, three, four, five, six, seven, eight, nine, ten, or more amino acid modifications) and/or (b) a light chain variable domain that includes a framework region 1 having the amino acid sequence set forth in SEQ ID NO: 108 (or a variant of SEQ ID NO:108 with one, two, three, four, five, six, seven, eight, nine, ten, or more amino acid modifications), a framework region 2 having the amino acid sequence set forth in SEQ ID NO: 109 (or a variant of SEQ ID NO:109 with one, two, three, four, five, six, seven, eight, nine, ten, or more amino acid modifications), a framework region 3 having the amino acid sequence set forth in SEQ ID NO:110 (or a variant of SEQ ID NO:110 with one, two, three, four, five, six, seven, eight, nine, ten, or more amino acid modifications), and a framework region 4 having the amino acid sequence set forth in SEQ ID NO:111 (or a variant of SEQ ID NO:111 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIGS. 8A or 8B can be designed to include framework regions as set forth in FIGS. 8A and 8B or can be designed to include one or more framework regions from another antibody, antibody fragment, or antibody domain. For example, a scFv can be designed to include the six CDRs set forth in FIGS. 8A and 8B and the framework regions set forth in FIGS. 8A and 8B except that framework region 1 having the amino acid set forth in SEQ ID NO:100 is replaced with a framework region 1 having the amino acid set forth in SEQ ID NO:4 or a framework region 1 having the amino acid set forth in SEQ ID NO: 20 or a framework region 1 having the amino acid set forth in SEQ ID NO:36 or a framework region 1 having the amino acid set forth in SEQ ID NO:52 or a framework region 1 having the amino acid set forth in SEQ ID NO:68 or a framework region 1 having the amino acid set forth in SEQ ID NO:84 or a framework region 1 having the amino acid set forth in SEQ ID NO:116. In some cases, an Fab can be designed to include the six CDRs set forth in FIGS. 8A and 8B and the framework regions set forth in FIGS. 8A and 8B. In some cases, an Fab can be designed to include the six CDRs set forth in FIGS. 8A and 8B and the framework regions set forth in FIGS. 8A and 8B except that framework region 1 having the amino acid set forth in SEQ ID NO:100 is replaced with a framework region 1 having the amino acid set forth in SEQ ID NO:4 or a framework region 1 having the amino acid set forth in SEQ ID NO:20 or a framework region 1 having the amino acid set forth in SEQ ID NO:36 or a framework region 1 having the amino acid set forth in SEQ ID NO:52 or a framework region 1 having the amino acid set forth in SEQ ID NO:68 or a framework region 1 having the amino acid set forth in SEQ ID NO:84 or a framework region 1 having the amino acid set forth in SEQ ID NO: 116. In another example, a scFv can be designed to include the six CDRs set forth in FIGS. 8A and 8B and the framework regions set forth in FIGS. 7A and 7B or FIGS. 9A and 9B.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain that includes an amino acid sequence having at least 90 percent identity to the amino acid sequence set forth in SEQ ID NO:104 and/or (b) a light chain variable domain that includes an amino acid sequence having at least 90 percent identity to the amino acid sequence set forth in SEQ ID NO:112. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:104 and/or (b) a light chain variable domain that includes an amino acid sequence having at least 90, 91, 92, 93, 94, 95, 96, 97, 98, or 99 percent identity to the amino acid sequence set forth in SEQ ID NO: 112. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:104 and/or (b) a light chain variable domain that includes an amino acid sequence having 100 percent identity to the amino acid sequence set forth in SEQ ID NO: 112.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain that includes an amino acid sequence having at least 90 percent identity to the amino acid sequence set forth in SEQ ID NO:104, provided that the heavy chain variable domain includes the amino acid sequences set forth in SEQ ID NOs: 97, 98, and 99, and/or (b) a light chain variable domain that includes an amino acid sequence having at least 90 percent identity to the amino acid sequence set forth in SEQ ID NO: 112, provided that the light chain variable domain includes the amino acid sequences set forth in SEQ ID NOs: 105, 106, and 107. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO: 104, provided that the heavy chain variable domain includes the amino acid sequences set forth in SEQ ID NOs: 97, 98, and 99, and/or (b) a light chain variable domain that includes an amino acid sequence having at least 90, 91, 92, 93, 94, 95, 96, 97, 98, or 99 percent identity to the amino acid sequence set forth in SEQ ID NO:112, provided that the light chain variable domain includes the amino acid sequences set forth in SEQ ID NOs: 105, 106, and 107.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain having the amino acid sequence set forth in SEQ ID NO: 104 or the amino acid set forth in SEQ ID NO: 104 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO: 112 or the amino acid set forth in SEQ ID NO: 112 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a PSCA polypeptide (e.g., a human PSCA polypeptide), can include a heavy chain variable domain having the amino acid sequence set forth in SEQ ID NO: 104 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 97, 98, and 99, and can include a light chain variable domain having the amino acid sequence set forth in SEQ ID NO: 112 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 105, 106, and 107.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain comprising (i) a CDR1 that comprises, consists essentially of, or consists of the amino acid sequence set forth in SEQ ID NO:97, (ii) a CDR2 that comprises, consists essentially of, or consists of the amino acid sequence set forth in SEQ ID NO: 98, and (iii) a CDR3 that comprises, consists essentially of, or consists of the amino acid sequence set forth in SEQ ID NO:99, and/or (b) a light chain variable domain comprising (i) a CDR1 that comprises, consists essentially of, or consists of the amino acid sequence set forth in SEQ ID NO:105, (ii) a CDR2 that comprises, consists essentially of, or consists of the amino acid sequence set forth in SEQ ID NO: 106, and (iii) a CDR3 that comprises, consists essentially of, or consists of the amino acid sequence set forth in SEQ ID NO:107. As used herein, a “CDR1 that consists essentially of the amino acid sequence set forth in SEQ ID NO:97” is a CDR1 that has zero, one, or two amino acid substitutions within SEQ ID NO:97, that has zero, one, two, three, four, or five amino acid residues directly preceding SEQ ID NO:97, and/or that has zero, one, two, three, four, or five amino acid residues directly following SEQ ID NO:97, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR1 that consists essentially of the amino acid sequence set forth in SEQ ID NO:97 include, without limitation, those set forth in Table 37.

TABLE 37
Exemplary CDR1s that consist essentially of the
amino acid sequence set forth in SEQ ID NO: 97.

| Sequence | SEQ ID NO: |
|---|---|
| AYYMH | 715 |
| GFYMH | 716 |
| GYFMH | 717 |
| GYYLH | 718 |
| GYYVH | 719 |
| GYYMD | 720 |
| GYYMS | 721 |
| GYYME | 722 |
| GYYMT | 723 |
| GYYMA | 724 |

As used herein, a “CDR2 that consists essentially of the amino acid sequence set forth in SEQ ID NO:98” is a CDR2 that has zero, one, or two amino acid substitutions within SEQ ID NO:98, that has zero, one, two, three, four, or five amino acid residues directly preceding SEQ ID NO:98, and/or that has zero, one, two, three, four, or five amino acid residues directly following SEQ ID NO:98, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR2 that consists essentially of the amino acid sequence set forth in SEQ ID NO:98 include, without limitation, those set forth in Table 38.

TABLE 38
Exemplary CDR2s that consist essentially of the
amino acid sequence set forth in SEQ ID NO: 98.

| Sequence | SEQ ID NO: |
|---|---|
| KINPNSGGTNYAQKFQG | 725 |
| RLNPNSGGTNYAQKFQG | 726 |
| RIDPNSGGTNYAQKFQG | 727 |
| RINPDSGGTNYAQKFQG | 728 |
| RINPNTGGTNYAQKFQG | 729 |
| RINPNSAGTNYAQKFQG | 730 |
| RINPNSGGTEYAQKFQG | 731 |
| RINPNSGGTNYSQKFQG | 732 |
| RINPNSGGTNYAQRFQG | 733 |
| RINPNSGGTNYAQKFNG | 734 |

As used herein, a “CDR3 that consists essentially of the amino acid sequence set forth in SEQ ID NO:99” is a CDR3 that has zero, one, or two amino acid substitutions within SEQ ID NO:99, that has zero, one, two, three, four, or five amino acid residues directly preceding SEQ ID NO:99, and/or that has zero, one, two, three, four, or five amino acid residues directly following SEQ ID NO:99, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR3 that consists essentially of the amino acid sequence set forth in SEQ ID NO:99 include, without limitation, those set forth in Table 39.

TABLE 39
Exemplary CDR3s that consist essentially of the
amino acid sequence set forth in SEQ ID NO: 99.

| Sequence | SEQ ID NO: |
|---|---|
| ATRHAFDI | 735 |
| GSRHAFDI | 736 |
| GTKHAFDI | 737 |
| GTRSAFDI | 738 |
| GTRHDFDI | 739 |
| GTRHAYDI | 740 |
| GTRHAFEI | 741 |
| GTRHAFDV | 742 |
| GTRHAFDL | 743 |
| GARHAFDI | 744 |

As used herein, a “CDR1 that consists essentially of the amino acid sequence set forth in SEQ ID NO: 105” is a CDR1 that has zero, one, or two amino acid substitutions within SEQ ID NO:105, that has zero, one, two, three, four, or five amino acid residues directly preceding SEQ ID NO: 105, and/or that has zero, one, two, three, four, or five amino acid residues directly following SEQ ID NO:105, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR1 that consists essentially of the amino acid sequence set forth in SEQ ID NO: 105 include, without limitation, those set forth in Table 40.

TABLE 40
Exemplary CDR1s that consist essentially of the
amino acid sequence set forth in SEQ ID NO: 105.

| Sequence | SEQ ID NO: |
|---|---|
| SRSSGSIASNYVQ | 745 |
| TKSSGSIASNYVQ | 746 |
| TRTSGSIASNYVQ | 747 |
| TRSTGSIASNYVQ | 748 |
| TRSSASIASNYVQ | 749 |
| TRSSGTIASNYVQ | 750 |
| TRSSGSLASNYVQ | 751 |
| TRSSGSIGSNYVQ | 752 |
| TRSSGSIASNYLQ | 753 |
| TRSSGSIASNYIQ | 754 |

As used herein, a “CDR2 that consists essentially of the amino acid sequence set forth in SEQ ID NO:106” is a CDR2 that has zero, one, or two amino acid substitutions within SEQ ID NO:106, that has zero, one, two, three, four, or five amino acid residues directly preceding SEQ ID NO: 106, and/or that has zero, one, two, three, four, or five amino acid residues directly following SEQ ID NO:106, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR2 that consists essentially of the amino acid sequence set forth in SEQ ID NO: 106 include, without limitation, those set forth in Table 41.

TABLE 41
Exemplary CDR2s that consist essentially of the
amino acid sequence set forth in SEQ ID NO: 106.

| Sequence | SEQ ID NO: |
|---|---|
| DDNQRPS | 755 |
| EENQRPS | 756 |
| EDQQRPS | 757 |
| EDNNRPS | 758 |
| EDNQKPS | 759 |
| EDNQRPT | 760 |
| EEDQRPS | 761 |
| EDNQKPT | 762 |
| NDNQRPS | 763 |
| EENQKPS | 764 |

As used herein, a “CDR3 that consists essentially of the amino acid sequence set forth in SEQ ID NO: 107” is a CDR3 that has zero, one, or two amino acid substitutions within SEQ ID NO:107, that has zero, one, two, three, four, or five amino acid residues directly preceding SEQ ID NO: 107, and/or that has zero, one, two, three, four, or five amino acid residues directly following SEQ ID NO:107, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR3 that consists essentially of the amino acid sequence set forth in SEQ ID NO: 107 include, without limitation, those set forth in Table 42.

TABLE 42
Exemplary CDR3s that consist essentially of the
amino acid sequence set forth in SEQ ID NO: 107.

| Sequence | SEQ ID NO: |
|---|---|
| NSYDSIVHVV | 765 |
| QTYDSIVHVV | 766 |
| QSFDSIVHVV | 767 |
| QSYESIVHVV | 768 |
| QSYDTIVHVV | 769 |
| QSYDSLVHVV | 770 |
| QSYDSIAHVV | 771 |
| QSYDSIVDVV | 772 |
| QSYDSIVHIV | 773 |
| QSYDSIVHVA | 774 |

In another embodiment, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (i) a heavy chain variable domain having a CDR1 having the amino acid sequence set forth in SEQ ID NO:113 (or a variant of SEQ ID NO:113 with one or two amino acid modifications), a CDR2 having the amino acid sequence set forth in SEQ ID NO: 114 (or a variant of SEQ ID NO:114 with one or two amino acid modifications), and a CDR3 having the amino acid sequence set forth in SEQ ID NO: 115 (or a variant of SEQ ID NO: 115 with one or two amino acid modifications); and/or (ii) a light chain variable domain having a CDR1 having the amino acid sequence set forth in SEQ ID NO:121 (or a variant of SEQ ID NO: 121 with one or two amino acid modifications), a CDR2 having the amino acid sequence set forth in SEQ ID NO: 122 (or a variant of SEQ ID NO:122 with one or two amino acid modifications), and a CDR3 having the amino acid sequence set forth SEQ ID NO:123 (or a variant of SEQ ID NO:123 with one or two amino acid modifications). An example of such a binder having these CDRs and the ability to bind to a PSCA polypeptide (e.g., a human PSCA polypeptide) includes, without limitation, the scFv set forth in FIGS. 9A and 9B.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) and (a) a heavy chain variable domain having a CDR1 having the amino acid sequence set forth in SEQ ID NO: 113 (or a variant of SEQ ID NO:113 with one or two amino acid modifications), a CDR2 having the amino acid sequence set forth in SEQ ID NO:114 (or a variant of SEQ ID NO: 114 with one or two amino acid modifications), and a CDR3 having the amino acid sequence set forth in SEQ ID NO:115 (or a variant of SEQ ID NO:115 with one or two amino acid modifications) and/or (b) a light chain variable domain having a CDR1 having the amino acid sequence set forth in SEQ ID NO:121 (or a variant of SEQ ID NO: 121 with one or two amino acid modifications), a CDR2 having the amino acid sequence set forth in SEQ ID NO:122 (or a variant of SEQ ID NO:122 with one or two amino acid modifications), and a CDR3 having the amino acid sequence set forth SEQ ID NO: 123 (or a variant of SEQ ID NO:123 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:116 (or a variant of SEQ ID NO: 116 with one, two, three, four, five, six, seven, eight, nine, ten, or more amino acid modifications), a framework region 2 having the amino acid sequence set forth in SEQ ID NO: 117 (or a variant of SEQ ID NO:117 with one, two, three, four, five, six, seven, eight, nine, ten, or more amino acid modifications), a framework region 3 having the amino acid sequence set forth in SEQ ID NO: 118 (or a variant of SEQ ID NO:118 with one, two, three, four, five, six, seven, eight, nine, ten, or more amino acid modifications), and a framework region 4 having the amino acid sequence set forth in SEQ ID NO:119 (or a variant of SEQ ID NO: 119 with one, two, three, four, five, six, seven, eight, nine, ten, or more amino acid modifications) and/or (b) a light chain variable domain that includes a framework region 1 having the amino acid sequence set forth in SEQ ID NO: 124 (or a variant of SEQ ID NO:124 with one, two, three, four, five, six, seven, eight, nine, ten, or more amino acid modifications), a framework region 2 having the amino acid sequence set forth in SEQ ID NO:125 (or a variant of SEQ ID NO:125 with one, two, three, four, five, six, seven, eight, nine, ten, or more amino acid modifications), a framework region 3 having the amino acid sequence set forth in SEQ ID NO:126 (or a variant of SEQ ID NO:126 with one, two, three, four, five, six, seven, eight, nine, ten, or more amino acid modifications), and a framework region 4 having the amino acid sequence set forth in SEQ ID NO: 127 (or a variant of SEQ ID NO: 127 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIGS. 9A or 9B can be designed to include framework regions as set forth in FIGS. 9A and 9B or can be designed to include one or more framework regions from another antibody, antibody fragment, or antibody domain. For example, a scFv can be designed to include the six CDRs set forth in FIGS. 9A and 9B and the framework regions set forth in FIGS. 9A and 9B except that framework region 1 having the amino acid set forth in SEQ ID NO:116 is replaced with a framework region 1 having the amino acid set forth in SEQ ID NO:4 or a framework region 1 having the amino acid set forth in SEQ ID NO: 20 or a framework region 1 having the amino acid set forth in SEQ ID NO:36 or a framework region 1 having the amino acid set forth in SEQ ID NO:52 or a framework region 1 having the amino acid set forth in SEQ ID NO:68 or a framework region 1 having the amino acid set forth in SEQ ID NO:84 or a framework region 1 having the amino acid set forth in SEQ ID NO:100. In some cases, an Fab can be designed to include the six CDRs set forth in FIGS. 9A and 9B and the framework regions set forth in FIGS. 9A and 9B. In some cases, an Fab can be designed to include the six CDRs set forth in FIGS. 9A and 9B and the framework regions set forth in FIGS. 9A and 9B except that framework region 1 having the amino acid set forth in SEQ ID NO:116 is replaced with a framework region 1 having the amino acid set forth in SEQ ID NO:4 or a framework region 1 having the amino acid set forth in SEQ ID NO:20 or a framework region 1 having the amino acid set forth in SEQ ID NO:36 or a framework region 1 having the amino acid set forth in SEQ ID NO:52 or a framework region 1 having the amino acid set forth in SEQ ID NO:68 or a framework region 1 having the amino acid set forth in SEQ ID NO:84 or a framework region 1 having the amino acid set forth in SEQ ID NO: 100. In another example, a scFv can be designed to include the six CDRs set forth in FIGS. 9A and 9B and the framework regions set forth in FIGS. 7A and 7B or FIGS. 8A and 8B.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain that includes an amino acid sequence having at least 90 percent identity to the amino acid sequence set forth in SEQ ID NO:120 and/or (b) a light chain variable domain that includes an amino acid sequence having at least 90 percent identity to the amino acid sequence set forth in SEQ ID NO:128. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:120 and/or (b) a light chain variable domain that includes an amino acid sequence having at least 90, 91, 92, 93, 94, 95, 96, 97, 98, or 99 percent identity to the amino acid sequence set forth in SEQ ID NO: 128. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:120 and/or (b) a light chain variable domain that includes an amino acid sequence having 100 percent identity to the amino acid sequence set forth in SEQ ID NO:128.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain that includes an amino acid sequence having at least 90 percent identity to the amino acid sequence set forth in SEQ ID NO:120, provided that the heavy chain variable domain includes the amino acid sequences set forth in SEQ ID NOs: 113, 114, and 115, and/or (b) a light chain variable domain that includes an amino acid sequence having at least 90 percent identity to the amino acid sequence set forth in SEQ ID NO: 128, provided that the light chain variable domain includes the amino acid sequences set forth in SEQ ID NOs: 121, 122, and 123. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO: 120, provided that the heavy chain variable domain includes the amino acid sequences set forth in SEQ ID NOs: 113, 114, and 115, and/or (b) a light chain variable domain that includes an amino acid sequence having at least 90, 91, 92, 93, 94, 95, 96, 97, 98, or 99 percent identity to the amino acid sequence set forth in SEQ ID NO:128, provided that the light chain variable domain includes the amino acid sequences set forth in SEQ ID NOs: 121, 122, and 123.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain having the amino acid sequence set forth in SEQ ID NO: 120 or the amino acid set forth in SEQ ID NO: 120 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO: 128 or the amino acid set forth in SEQ ID NO: 128 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a PSCA polypeptide (e.g., a human PSCA polypeptide), can include a heavy chain variable domain having the amino acid sequence set forth in SEQ ID NO: 120 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 113, 114, and 115, and can include a light chain variable domain having the amino acid sequence set forth in SEQ ID NO: 128 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs: 121, 122, and 123.

In some cases, a binder (e.g., an antibody, antigen binding fragment, antibody domain, a CAR, a cell engager, and/or an ADC) provided herein having the ability to bind to a PSCA polypeptide (e.g., a human PSCA polypeptide) can include (a) a heavy chain variable domain comprising (i) a CDR1 that comprises, consists essentially of, or consists of the amino acid sequence set forth in SEQ ID NO:113, (ii) a CDR2 that comprises, consists essentially of, or consists of the amino acid sequence set forth in SEQ ID NO: 114, and (iii) a CDR3 that comprises, consists essentially of, or consists of the amino acid sequence set forth in SEQ ID NO:115, and/or (b) a light chain variable domain comprising (i) a CDR1 that comprises, consists essentially of, or consists of the amino acid sequence set forth in SEQ ID NO: 121, (ii) a CDR2 that comprises, consists essentially of, or consists of the amino acid sequence set forth in SEQ ID NO:122, and (iii) a CDR3 that comprises, consists essentially of, or consists of the amino acid sequence set forth in SEQ ID NO:123. As used herein, a “CDR1 that consists essentially of the amino acid sequence set forth in SEQ ID NO:113” is a CDR1 that has zero, one, or two amino acid substitutions within SEQ ID NO:113, that has zero, one, two, three, four, or five amino acid residues directly preceding SEQ ID NO:113, and/or that has zero, one, two, three, four, or five amino acid residues directly following SEQ ID NO: 113, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR1 that consists essentially of the amino acid sequence set forth in SEQ ID NO:113 include, without limitation, those set forth in Table 43.

TABLE 43
Exemplary CDR1s that consist essentially of the
amino acid sequence set forth in SEQ ID NO: 113.

| Sequence | SEQ ID NO: |
|---|---|
| TYAIS | 775 |
| SFAIS | 776 |
| SYTIS | 777 |
| SYALS | 778 |
| SYAMS | 779 |
| SYAIT | 780 |
| SYAIA | 781 |
| SYAID | 782 |
| EYAIT | 783 |
| SYDIS | 784 |

As used herein, a “CDR2 that consists essentially of the amino acid sequence set forth in SEQ ID NO:114” is a CDR2 that has zero, one, or two amino acid substitutions within SEQ ID NO:114, that has zero, one, two, three, four, or five amino acid residues directly preceding SEQ ID NO: 114, and/or that has zero, one, two, three, four, or five amino acid residues directly following SEQ ID NO:114, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR2 that consists essentially of the amino acid sequence set forth in SEQ ID NO: 114 include, without limitation, those set forth in Table 44.

TABLE 44
Exemplary CDR2s that consist essentially of the
amino acid sequence set forth in SEQ ID NO: 114.

| Sequence | SEQ ID NO: |
|---|---|
| AIIPIFGTANYAQKFQG | 785 |
| GILPIFGTANYAQKFQG | 786 |
| GIIPLFGTANYAQKFQG | 787 |
| GIIPIFATANYAQKFQG | 788 |
| GIIPIFGTAEYAQKFQG | 789 |
| GIIPIFGTANYANKFQG | 790 |
| GIIPIFGTANYAQRFQG | 791 |
| GIIPIFGTANYAQKFNG | 792 |
| GIIPIFGTANYAQKFQA | 793 |
| GIIPIFGTANYAQKYQG | 794 |

As used herein, a “CDR3 that consists essentially of the amino acid sequence set forth in SEQ ID NO:115” is a CDR3 that has zero, one, or two amino acid substitutions within SEQ ID NO:115, that has zero, one, two, three, four, or five amino acid residues directly preceding SEQ ID NO: 115, and/or that has zero, one, two, three, four, or five amino acid residues directly following SEQ ID NO:115, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR3 that consists essentially of the amino acid sequence set forth in SEQ ID NO:115 include, without limitation, those set forth in Table 45.

TABLE 45
Exemplary CDR3s that consist essentially of the
amino acid sequence set forth in SEQ ID NO: 115.

| Sequence | SEQ ID NO: |
|---|---|
| EVIVGATSSGMDV | 795 |
| DIIVGATSSGMDV | 796 |
| DVLVGATSSGMDV | 797 |
| DVIAGATSSGMDV | 798 |
| DVIVGASSSGMDV | 799 |
| DVIVGATTSGMDV | 800 |
| DVIVGATSSAMDV | 801 |
| DVIVGATSSGMEV | 802 |
| DVIVGATSSGMDA | 803 |
| DVIVGATSSGMDL | 804 |

As used herein, a “CDR1 that consists essentially of the amino acid sequence set forth in SEQ ID NO:121” is a CDR1 that has zero, one, or two amino acid substitutions within SEQ ID NO:121, that has zero, one, two, three, four, or five amino acid residues directly preceding SEQ ID NO: 121, and/or that has zero, one, two, three, four, or five amino acid residues directly following SEQ ID NO:121, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR1 that consists essentially of the amino acid sequence set forth in SEQ ID NO:121 include, without limitation, those set forth in Table 46.

TABLE 46
Exemplary CDR1s that consist essentially of the
amino acid sequence set forth in SEQ ID NO: 121.

| Sequence | SEQ ID NO: |
|---|---|
| KASQSISRYLN | 805 |
| RGSQSISRYLN | 806 |
| RATQSISRYLN | 807 |
| RASESISRYLN | 808 |
| RASQTISRYLN | 809 |
| RASQSITRYLN | 810 |
| RASQSISKYLN | 811 |
| RASQSISRFLN | 812 |
| RASQSISRYIN | 813 |
| RASQSISRYLE | 814 |

As used herein, a “CDR2 that consists essentially of the amino acid sequence set forth in SEQ ID NO: 122” is a CDR2 that has zero, one, or two amino acid substitutions within SEQ ID NO:122, that has zero, one, two, three, four, or five amino acid residues directly preceding SEQ ID NO:122, and/or that has zero, one, two, three, four, or five amino acid residues directly following SEQ ID NO: 122, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR2 that consists essentially of the amino acid sequence set forth in SEQ ID NO:122 include, without limitation, those set forth in Table 47.

TABLE 47
Exemplary CDR2s that consist essentially of the
amino acid sequence set forth in SEQ ID NO: 122.

| Sequence | SEQ ID NO: |
|---|---|
| AASSLQS | 815 |
| GGSSLQS | 816 |
| GATSLQS | 817 |
| GASTLQS | 818 |
| GASSIQS | 819 |
| GASSAQS | 820 |
| GASSLES | 821 |
| GASSLQT | 822 |
| GASSLQD | 823 |
| GASSMQS | 824 |

As used herein, a “CDR3 that consists essentially of the amino acid sequence set forth in SEQ ID NO: 123” is a CDR3 that has zero, one, or two amino acid substitutions within SEQ ID NO:123, that has zero, one, two, three, four, or five amino acid residues directly preceding SEQ ID NO: 123, and/or that has zero, one, two, three, four, or five amino acid residues directly following SEQ ID NO:123, provided that the binder (e.g., an antibody, antigen binding fragment, antibody domain, a CAR, a cell engager, and/or an ADC) maintains its basic ability to bind to a PSCA polypeptide (e.g., a human PSCA polypeptide). Examples of a CDR3 that consists essentially of the amino acid sequence set forth in SEQ ID NO:123 include, without limitation, those set forth in Table 48.

TABLE 48
Exemplary CDR3s that consist essentially of the
amino acid sequence set forth in SEQ ID NO: 123.

| Sequence | SEQ ID NO: |
|---|---|
| NQSYSTPLT | 825 |
| QNSYSTPLT | 826 |
| QQTYSTPLT | 827 |
| QQSFSTPLT | 828 |
| QQSYTTPLT | 829 |
| QQSYSSPLT | 830 |
| QQSYSTPIT | 831 |
| QQSYSTPVT | 832 |
| QQSYSTPLS | 833 |
| QQSYSTPLA | 834 |

When designing a single chain antibody (e.g., a scFv) having a heavy chain variable domain and a light chain variable domain, the two regions can be directly connected or can be connected using any appropriate linker sequence. For example, a heavy chain variable domain can be connected to a light chain variable domain via a linker sequence. Examples of linker sequences that can be used to connect a heavy chain variable domain and a light chain variable domain to create a scFv include, without limitation, those linkers set forth in Example 12.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

The percent sequence identity between a particular amino acid or nucleic acid sequence and an amino acid or nucleic acid sequence referenced by a particular sequence identification number is determined as follows. First, an amino acid or nucleic acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q -1 -r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt-j c:\seq2.txt-p blastp -o c: \output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. A matched position refers to a position in which an identical nucleotide or amino acid residue occurs at the same position in aligned sequences. The percent sequence identity is determined by dividing the number of matches by the length of the sequence set forth in the identified sequence (e.g., SEQ ID NO: 8, SEQ ID NO: 16, or SEQ ID NO:24), followed by multiplying the resulting value by 100. For example, an amino acid sequence that has 100 matches when aligned with the sequence set forth in SEQ ID NO:8 is 85 percent identical to the sequence set forth in SEQ ID NO:8 (i.e., 100-117×100=85.47). It is noted that the percent sequence identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3:348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to a PSCA polypeptide (e.g., a human PSCA polypeptide) are further described in Table 49.

TABLE 49
Representative number of binders.

| | | SEQ ID NOs of | | | SEQ ID NOs of | |
|---|---|---|---|---|---|---|
| | SEQ ID NOs of | Heavy Chain | | SEQ ID NOs of | Light Chain | |
| | Heavy Chain | Variable | SEQ ID NO of | Light Chain | Variable | SEQ ID NO of |
| Clone # | Variable | Domain/Region | Heavy Chain | Variable | Domain/Region | Light Chain |
| (Antibody | Domain/Region | Framework | Variable | Domain/Region | Framework | Variable |
| type) | CDRs | Regions | Domain/Region | CDRs | Regions | Domain/Region |
| #1 (Fab) | 1, 2, 3 | 4, 5, 6, 7 | 8 | 9, 10, 11 | 12, 13, 14, 15 | 16 |
| #2 (Fab) | 17, 18, 19 | 20, 21, 22, 23 | 24 | 25, 26, 27 | 28, 29, 30, 31 | 32 |
| #3 (Fab) | 33, 34, 35 | 36, 37, 38, 39 | 40 | 41, 42, 43 | 44, 45, 46, 47 | 48 |
| #4 (Fab) | 49, 50, 51 | 52, 53, 54, 55 | 56 | 57, 58, 59 | 60, 61, 62, 63 | 64 |
| #5 (Fab) | 65, 66, 67 | 68, 69, 70, 71 | 72 | 73, 74, 75 | 76, 77, 78, 79 | 80 |
| #6 (scFv) | 81, 82, 83 | 84, 85, 86, 87 | 88 | 89, 90, 91 | 92, 93, 94, 95 | 96 |
| #7 (scFv) | 97, 98, 99 | 100, 101, 102, | 104 | 105, 106, 107 | 108, 109, 110, | 112 |
| | | 103 | | | 111 | |
| #8 (scFv) | 113, 114, 115 | 116, 117, 118, | 120 | 121, 122, 123 | 124, 125, 126, | 128 |
| | | 119 | | | 127 | |

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. Example 8 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae/casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansemila polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NSO cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a PSCA polypeptide (e.g., a human PSCA polypeptide). In some cases, a bispecific binder provided herein can be designed to bind to two different epitopes of the same PSCA polypeptide (e.g., a human PSCA polypeptide). In some cases, a bispecific binder provided herein can bind to a PSCA polypeptide (e.g., a human PSCA polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a PSCA polypeptide (e.g., a human PSCA polypeptide) and (b) at least one drug covalently linked directly or indirectly to that antigen binding domain. Any appropriate binder (e.g., an antibody, antigen binding fragment, and/or antibody domain) provided herein and having the ability to bind to a PSCA polypeptide (e.g., a human PSCA polypeptide) can be used as an antigen binding domain to make an ADC described herein. For example, any of the binders set forth in Table 49 can be used to make an ADC having the ability to bind to a PSCA polypeptide (e.g., a human PSCA polypeptide). Examples of drugs that can be used to make an ADC described herein include, without limitation, auristatins (e.g., monomethyl auristatin E (MMAE)), mertansine (DM-1), and pyrrolobenzodiazepine (PBD) dimers. Any appropriate ADC linker can be used to covalently attach one or more drugs to an antigen binding domain having the ability to bind to a PSCA polypeptide (e.g., a human PSCA polypeptide) to form an ADC provided herein. For example, cleavable or non-cleavable ADC linkers can be used to covalently attach one or more drugs to an antigen binding domain having the ability to bind to a PSCA polypeptide (e.g., a human PSCA polypeptide) to form an ADC provided herein. Examples of ADC linkers can be used to covalently attach one or more drugs to an antigen binding domain having the ability to bind to a PSCA polypeptide (e.g., a human PSCA polypeptide) to form an ADC provided herein include, without limitation, ADC disulfide linkers, ADC hydrazone linkers, ADC peptide linkers, ADC thioether linkers, and ADC PEG-containing linkers. In some cases, the methods and materials described elsewhere can be used to make an ADC having the ability to bind to a PSCA polypeptide (e.g., a human PSCA polypeptide) as described herein (see, e.g., International Patent Application Publication No. WO2018058001 and U.S. Pat. No. 8,436,147).

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a variable domain such as a variable domain as set forth in any one of FIGS. 2-9. In another example, an isolated nucleic acid molecule provided herein can include a nucleic acid sequence encoding a CAR or cell engager (e.g., a BiTE, BiKE, or TriKE) described herein. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokaryotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a PSCA polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a PSCA polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g., a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g., a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a PSCA polypeptide (e.g., a human PSCA polypeptide) can be formulated as a pharmaceutical composition for administration to a mammal (e.g., a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a PSCA polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a PSCA polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. Examples of cancers that can be treated as described herein include, without limitation, prostate cancer such as metastatic prostate cancer, bladder cancer, renal cell cancer, pancreatic cancer, and gastric cancer. In some cases, a mammal (e.g., a human) having a PSCA⁺ cancer (e.g., a PSCA⁺ prostate cancer or a PSCA⁺ metastatic prostate cancer) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), intratumorally (e.g., via an intratumoral injection), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a PSCA polypeptide (e.g., a human PSCA polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of a PSCA polypeptide (e.g., a human PSCA polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a PSCA polypeptide (e.g., a human PSCA polypeptide). In some cases, a binder (e.g., an antibody, antigen binding fragment, and/or antibody domain) provided herein can be immobilized, e.g., on a support, and retention of a PSCA polypeptide (e.g., a human PSCA polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a PSCA polypeptide (e.g., a human PSCA polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Obtaining Binders Having the Ability to Bind to a Human PSCA Polypeptide

Large phage displayed antibody libraries were panned and screened to identify binders that bind to a human PSCA polypeptide using the underlined amino acid sequence set forth in SEQ ID NO:129 as shown in Example 6. To identify such binders, a polypeptide having amino acid residues 12 to 87 of human PSCA polypeptide set forth in Example 6 (the 114 isoform) was fused to the AviTag sequence at the C-terminus of the PSCA sequence, and the PSCA-AviTag polypeptide was used for panning of human Fab and scFv phage-displayed libraries. Five Fabs (Clones #1, #2, #3, #4, and #5; FIGS. 2, 3, 4, 5, and 6) were identified, and three scFvs (Clones #6, #7, and 8 #; FIGS. 7, 8, and 9) were identified. Clones #4 and #5 were obtained by phage display based affinity maturation screening.

Binding affinity and specificity to a human PSCA polypeptide were tested using an ELISA and flow cytometry. Clones #4 and #5 exhibited high affinity binding to a human PSCA polypeptide having EC₅₀ values of 4 nM and 0.4 nM, respectively. Clone F12 (Clone #5) bound to PC-3 cells displaying human PSCA, but did not bind to PC-3 cells lacking expression of human PSCA, demonstrating that it can bind to PSCA polypeptides present on cells (see, e.g., FIGS. 4-6).

Specificity characterization and epitope mapping of the affinity enhanced Clone #5 was performed. Clone #5 binding to 5300 human membrane proteins was tested by the proteomic membrane array. Results showed Clone #5 only binds to PSCA without any discernable binding to other human proteins. Epitope mapping by the peptide array method was performed using Clone #5, in which the overlapping peptides consisting of 7-11 amino acids were printed on a chip followed by addition of Clone #5 and detection of antibody for observing binding. Results showed that Clone #5 binds to a DDSQDYY motif (SEQ ID NO: 383) in PSCA.

### Example 2—Designing ADCs from Binders Having the Ability to Bind to a Human PSCA Polypeptide

PSCA antibody F12 clone (Clone #5) was conjugated to MMAE through a valine-citrulline dipeptide linker by lysine-NHS coupling chemistry. The drug: antibody ratio (DAR) was determined to be two based on LS/MS. The ADC, F12-MMAE, exhibited specific cytotoxicity against PSCA overexpressing PC-3 cells, both in vitro and in a PC-3-PSCA xenografted mouse model, without obvious toxicity (e.g., no body weight loss) (FIG. 7).

### Example 3—Designing BiKEs from Binders Having the Ability to Bind to a Human PSCA Polypeptide

By combining a PSCA binder with an anti-CD16A VH domain antibody (VH7), BiKE molecules were generated based on the PSCA binder (FIG. 8). BiKEs with two arms were designed to bring NK cells and cancer cells into proximity, forming immunosynapses and enlisting NK cell ADCC against tumor cells. Two types of BiKEs were produced based on an IgG1 scaffold to achieve prolonged half-life. One type (designed as 1+2 BiKE) was produced with a monovalent anti-CD16A binder; and the other type was produced with a bivalent anti-CD16A (designed as 2+2 BiKE) to increase NK cell binding and reinforce the immunosynapses. The fine tuning of molecular architecture of BiKEs can minimize tonic activation of NK cells mediated by BiKEs in the absence of tumor cells. These BiKEs exhibited effective binding to the recombinant human PSCA and CD16A proteins, as tested by ELISA (FIG. 9). Their capability to activate NK cell ADCC was tested on the Promega ADCC reporter cells (Jurkat T-Luc cells). Results revealed that 2+2 BiKE was more potent than 1+2 BiKE (FIG. 9). These results demonstrate that BiKEs can be designed and used to direct the killing of cells expressing human PSCA such as PSCA⁺ cancer cells.

### Example 4—Designing CARs from Binders Having the Ability to Bind to a Human PSCA Polypeptide

Clones #1-#8 are used to make CARs having configurations as set forth in FIGS. 29-44.
Cells (e.g., T cells) designed to express such CARs are used to kill cells expressing human PSCA such as PSCA⁺ cancer cells.

### Example 5—Designing BiTEs from Binders Having the Ability to Bind to a Human PSCA Polypeptide

Clones #1-#8 are used to make BiTEs having configurations as set forth in FIGS. 64-71. Such BiTEs are used to direct T cells to kill cells expressing human PSCA such as PSCA⁺ cancer cells.

### Example 6—Exemplary PSCA Polypeptide Sequences

This Example depicts an amino acid sequence of an exemplary full length human PSCA polypeptide (SEQ ID NO:129) and an exemplary nucleic acid sequence encoding that full length human PSCA polypeptide (SEQ ID NO:130). An exemplary extracellular domain is underlined and was used to identify Clone #1, Clone #2, and Clone #3.

### Full Length Human PSCA:

(SEQ ID NO: 129)
MAGLALQPGTALLCYSCKAQVSNEDCLQVENCTQLGEQCWTARIRAVGL
LTVISKGCSLNCVDDSQDYYVGKKNITCCDTDLCNASGAHALQPAAAIL
ALLPALGLLLWGPGQL

### Nucleic Acid Encoding Full Length Human PSCA:

(SEQ ID NO: 130)
ATGGCAGGCTTGGCCCTGCAGCCAGGCACTGCCCTGCTGTGCTACTCCT
GCAAAGCCCAGGTGAGCAACGAGGACTGCCTGCAGGTGGAGAACTGCAC
CCAGCTGGGGGAGCAGTGCTGGACCGCGCGCATCCGCGCAGTTGGCCTC
CTGACCGTCATCAGCAAAGGCTGCAGCTTGAACTGCGTGGATGACTCAC
AGGACTACTACGTGGGCAAGAAGAACATCACGTGCTGTGACACCGACTT
GTGCAACGCCAGCGGGGCCCATGCCCTGCAGCCGGCTGCTGCCATCCTT
GCGCTGCTCCCTGCACTCGGCCTGCTGCTCTGGGGACCCGGCCAGCTCT
AG

### Example 7—Sequences of Exemplary Binders Having the Ability to Bind to a Human PSCA Polypeptide

This Example depicts the amino acid sequences of Fabs and scFvs having the ability to bind to a human PSCA polypeptide. The CDRs and framework sequences also are delineated.

Anti-PSCA Clone #1 (also referred to as G7) (Fab)
Heavy chain variable domain (without first constant domain and partial hinge):
QVQLVESGGGLVKPGGSLRLSCAASGFTFSDYYMSWIRQAPGKGLEWVSYISSS
GSTIYYADSVKGRFTISRDNAKNSLYLQMNSLRAEDTAVYYCARPLRWGMDV
WGQGTTVTVSS (SEQ ID NO: 8)
Framework Region 1 of heavy chain variable domain:
QVQLVESGGGLVKPGGSLRLSCAASGFTFS (SEQ ID NO: 4)
CDR1 of heavy chain variable domain:
DYYMS (SEQ ID NO: 1)
Framework Region 2 of heavy chain variable domain:
WIRQAPGKGLEWVS (SEQ ID NO: 5)
CDR2 of heavy chain variable domain:
YISSSGSTIYYADSVKG (SEQ ID NO: 2)
Framework Region 3 of heavy chain variable domain:
RFTISRDNAKNSLYLQMNSLRAEDTAVYYCAR (SEQ ID NO: 6)
CDR3 of heavy chain variable domain:
PLRWGMDV (SEQ ID NO: 3)
Framework Region 4 of heavy chain variable domain:
WGQGTTVTVSS (SEQ ID NO: 7)
Human IgG1 heavy chain constant domain 1 (CH1):
ASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKV (SEQ ID NO: 131)
Human immunoglobulin G1 hinge region (partial):
EPKSC (SEQ ID NO: 132)
Anti-PSCA Clone #1 (also referred to as G7) (Fab)
Light chain variable domain (without kappa/lambda constant domain):
DIQMTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIK (SEQ ID
NO: 16)
Framework Region 1 of light chain variable domain:
DIQMTQSPSSLSASVGDRVTITC (SEQ ID NO: 12)
CDR1 of light chain variable domain:
RASQSISSYLN (SEQ ID NO: 9)
Framework Region 2 of light chain variable domain:
WYQQKPGKAPKLLIY (SEQ ID NO: 13)
CDR2 of light chain variable domain:
AASSLQS (SEQ ID NO: 10)
Framework Region 3 of light chain variable domain:
GVPSRFSGSGSGTDFTLTISSLQPEDAATYYC (SEQ ID NO: 14)
CDR3 of light chain variable domain:
HQSSSLPLT (SEQ ID NO: 11)
Framework Region 4 of light chain variable domain:
FGGGTKVEIK (SEQ ID NO: 15)
Human kappa light chain constant domain:
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
(SEQ ID NO: 133)
Anti-PSCA Clone #2 (also referred to as F1) (Fab)
Heavy chain variable domain (without first constant domain and partial hinge):
QVQLVESGGGLVKPGGSLRLSCAASGFTFSDYYMSWIRQAPGKGLEWVSYISSS
GSTIYYADSVKGRFTISRDNAKNSLYLQMNSLRAEDTAVYYCARPLRWGMDV
WGQGTTVTVSS (SEQ ID NO: 24)
Framework Region 1 of heavy chain variable domain:
QVQLVESGGGLVKPGGSLRLSCAASGFTFS (SEQ ID NO: 20)
CDR1 of heavy chain variable domain:
DYYMS (SEQ ID NO: 17)
Framework Region 2 of heavy chain variable domain:
WIRQAPGKGLEWVS (SEQ ID NO: 21)
CDR2 of heavy chain variable domain:
YISSSGSTIYYADSVKG (SEQ ID NO: 18)
Framework Region 3 of heavy chain variable domain:
RFTISRDNAKNSLYLQMNSLRAEDTAVYYCAR (SEQ ID NO: 22)
CDR3 of heavy chain variable domain:
PLRWGMDV (SEQ ID NO: 19)
Framework Region 4 of heavy chain variable domain:
WGQGTTVTVSS (SEQ ID NO: 23)
Human IgG1 heavy chain constant domain 1 (CH1):
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKV (SEQ ID NO: 131)
Human immunoglobulin G1 hinge region (partial):
EPKSC (SEQ ID NO: 132)
Anti-PSCA Clone #2 (also referred to as F1) (Fab)
Light chain variable domain (without kappa/lambda constant domain):
DIQMTQSPSSLSASVGDRVTITCRASQSISSYLNWYQQKPGKAPKLLIYAASSLQ
SGVPSRFSGSGSGTDFTLTISSLQPEDFAVYYCHQSSSLPLTFGGGTKLEIK (SEQ
ID NO: 32)
Framework Region 1 of light chain variable domain:
DIQMTQSPSSLSASVGDRVTITC (SEQ ID NO: 28)
CDR1 of light chain variable domain:
RASQSISSYLN (SEQ ID NO: 25)
Framework Region 2 of light chain variable domain:
WYQQKPGKAPKLLIY (SEQ ID NO: 29)
CDR2 of light chain variable domain:
AASSLQS (SEQ ID NO: 26)
Framework Region 3 of light chain variable domain:
GVPSRFSGSGSGTDFTLTISSLQPEDFAVYYC (SEQ ID NO: 30)
CDR3 of light chain variable domain:
HQSSSLPLT (SEQ ID NO: 27)
Framework Region 4 of light chain variable domain:
FGGGTKLEIK (SEQ ID NO: 31)
Human kappa light chain constant domain:
RTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
(SEQ ID NO: 133)
Anti-PSCA Clone #3 (also referred to as C10) (Fab)
Heavy chain variable domain (without first constant domain and partial hinge):
QVQLVESGGGLVKPGGSLRLSCAASGFTFSDYYMSWIRQAPGKGLEWVSYISSS
GSTIYYADSVKGRFTISRDNAKNSLYLQMNSLRAEDTAVYYCARPLRWGMDV
WGQGTTVTVSS (SEQ ID NO: 40)
Framework Region 1 of heavy chain variable domain:
QVQLVESGGGLVKPGGSLRLSCAASGFTFS (SEQ ID NO: 36)
CDR1 of heavy chain variable domain:
DYYMS (SEQ ID NO: 33)
Framework Region 2 of heavy chain variable domain:
WIRQAPGKGLEWVS (SEQ ID NO: 37)
CDR2 of heavy chain variable domain:
YISSSGSTIYYADSVKG (SEQ ID NO: 34)
Framework Region 3 of heavy chain variable domain:
RFTISRDNAKNSLYLQMNSLRAEDTAVYYCAR (SEQ ID NO: 38)
CDR3 of heavy chain variable domain:
PLRWGMDV (SEQ ID NO: 35)
Framework Region 4 of heavy chain variable domain:
WGQGTTVTVSS (SEQ ID NO: 39)
Human IgG1 heavy chain constant domain 1 (CH1):
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKV (SEQ ID NO: 131)
Human immunoglobulin G1 hinge region (partial):
EPKSC (SEQ ID NO: 132)
Anti-PSCA Clone #3 (also referred to as C10) (Fab)
Light chain variable domain (without kappa/lambda constant domain):
DIQMTQSPSSLSASVGDRVTITCRASQSISSYLNWYQQKPGKAPKLLIYAASSLQ
SGVPSRFSGSGSGTDFTLTISSLQPEDVAVYYCHQSNSLPLTFGGGTKVEIK (SEQ
ID NO: 48)
Framework Region 1 of light chain variable domain:
DIQMTQSPSSLSASVGDRVTITC (SEQ ID NO: 44)
CDR1 of light chain variable domain:
RASQSISSYLN (SEQ ID NO: 41)
Framework Region 2 of light chain variable domain:
WYQQKPGKAPKLLIY (SEQ ID NO: 45)
CDR2 of light chain variable domain:
AASSLQS (SEQ ID NO: 42)
Framework Region 3 of light chain variable domain:
GVPSRFSGSGSGTDFTLTISSLQPEDVAVYYC (SEQ ID NO: 46)
CDR3 of light chain variable domain:
HQSNSLPLT (SEQ ID NO: 43)
Framework Region 4 of light chain variable domain:
FGGGTKVEIK (SEQ ID NO: 47)
Human kappa light chain constant domain:
RTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
(SEQ ID NO: 133)
Anti-PSCA Clone #4 (also referred to as F2) (Fab)
Heavy chain variable domain (without first constant domain and partial hinge):
QVQLVESGGGLVKPGGSLRLSCAASGFTFSDYYMSWIRQAPGKGLEWVSYISSS
GSTIVYADSVKGRFTISRDNAKNSLYLQMNSLRAEDTAVYYCARPIRRGMDVW
GQGTTVTVSS (SEQ ID NO: 56)
Framework Region 1 of heavy chain variable domain:
QVQLVESGGGLVKPGGSLRLSCAASGFTFS (SEQ ID NO: 52)
CDR1 of heavy chain variable domain:
DYYMS (SEQ ID NO: 49)
Framework Region 2 of heavy chain variable domain:
WIRQAPGKGLEWVS (SEQ ID NO: 53)
CDR2 of heavy chain variable domain:
YISSSGSTIVYADSVKG (SEQ ID NO: 50)
Framework Region 3 of heavy chain variable domain:
RFTISRDNAKNSLYLQMNSLRAEDTAVYYCAR (SEQ ID NO: 54)
CDR3 of heavy chain variable domain:
PIRRGMDV (SEQ ID NO: 51)
Framework Region 4 of heavy chain variable domain:
WGQGTTVTVSS (SEQ ID NO: 55)
Human IgG1 heavy chain constant domain 1 (CH1):
ASTRGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKV (SEQ ID NO: 134)
Human immunoglobulin G1 hinge region (partial):
EPKSC (SEQ ID NO: 132)
Anti-PSCA Clone #4 (also referred to as F2) (Fab)
Light chain variable domain (without kappa/lambda constant domain):
DIQMTQSPSSLSASVGDRVTITCRASQSISSYLNWYQQKPGKAPKLLIYAASSLQ
SGVPSRFSGSGSGTDFTLTISSLQPEDAATYYCHQSSSLPLTFGGGTKVEIK (SEQ
ID NO: 64)
Framework Region 1 of light chain variable domain:
DIQMTQSPSSLSASVGDRVTITC (SEQ ID NO: 60)
CDR1 of light chain variable domain:
RASQSISSYLN (SEQ ID NO: 57)
Framework Region 2 of light chain variable domain:
WYQQKPGKAPKLLIY (SEQ ID NO: 61)
CDR2 of light chain variable domain:
AASSLQS (SEQ ID NO: 58)
Framework Region 3 of light chain variable domain:
GVPSRFSGSGSGTDFTLTISSLQPEDAATYYC (SEQ ID NO: 62)
CDR3 of light chain variable domain:
HQSSSLPLT (SEQ ID NO: 59)
Framework Region 4 of light chain variable domain:
FGGGTKVEIK (SEQ ID NO: 63)
Human kappa light chain constant domain:
RTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
(SEQ ID NO: 133)
Anti-PSCA Clone #5 (also referred to as F12) (Fab)
Heavy chain variable domain (without first constant domain and partial hinge):
QVQLVESGGGLVKPGGSLRLSCAASGFTFSDYYMSWIRQAPGKGLEWVSYISSS
GSTINYADSVKGRFTISRDNAKNSLYLQMNSLRAEDTAVYYCARPIRWGMDV
WGQGTTVTVSS (SEQ ID NO: 72)
Framework Region 1 of heavy chain variable domain:
QVQLVESGGGLVKPGGSLRLSCAASGFTFS (SEQ ID NO: 68)
CDR1 of heavy chain variable domain:
DYYMS (SEQ ID NO: 65)
Framework Region 2 of heavy chain variable domain:
WIRQAPGKGLEWVS (SEQ ID NO: 69)
CDR2 of heavy chain variable domain:
YISSSGSTINYADSVKG (SEQ ID NO: 66)
Framework Region 3 of heavy chain variable domain:
RFTISRDNAKNSLYLQMNSLRAEDTAVYYCAR (SEQ ID NO: 70)
CDR3 of heavy chain variable domain:
PIRWGMDV (SEQ ID NO: 67)
Framework Region 4 of heavy chain variable domain:
WGQGTTVTVSS (SEQ ID NO: 71)
Human IgGl heavy chain constant domain 1 (CH1):
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKV (SEQ ID NO: 131)
Human immunoglobulin G1 hinge region (partial):
EPKSC (SEQ ID NO: 132)
Anti-PSCA Clone #5 (also referred to as F12) (Fab)
Light chain variable domain (without kappa/lambda constant domain):
DIQMTQSPSSLSASVGDRVTITCRASQSISSYLNWYQQKPGKAPKLLIYAASSLQ
SGVPSRFSGSGSGTDFTLTISSLQPEDAATYYCHQSSSLPLTFGGGTKVEIK (SEQ
ID NO: 80)
Framework Region 1 of light chain variable domain:
DIQMTQSPSSLSASVGDRVTITC (SEQ ID NO: 76)
CDR1 of light chain variable domain:
RASQSISSYLN (SEQ ID NO: 73)
Framework Region 2 of light chain variable domain:
WYQQKPGKAPKLLIY (SEQ ID NO: 77)
CDR2 of light chain variable domain:
AASSLQS (SEQ ID NO: 74)
Framework Region 3 of light chain variable domain:
GVPSRFSGSGSGTDFTLTISSLQPEDAATYYC (SEQ ID NO: 78)
CDR3 of light chain variable domain:
HQSSSLPLT (SEQ ID NO: 75)
Framework Region 4 of light chain variable domain:
FGGGTKVEIK (SEQ ID NO: 79)
Human kappa light chain constant domain:
RTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
(SEQ ID NO: 133)
Anti-PSCA Clone #6 (also referred to as SD10) (scFv)
Heavy chain:
EVQLVQSGAEVKKPGSSVKVSCKASGGTFSSYAISWVRQAPGQGLEWMGGIIPI
FGTANYAQKFQGRVTITADESTSTAYMELSSLRSDDTAVYYCARERIAVAGTNY
YYGMDVWGQGTLVTVSS (SEQ ID NO: 88)
Framework Region 1 of heavy chain variable domain:
EVQLVQSGAEVKKPGSSVKVSCKASGGTFS (SEQ ID NO: 84)
CDR1 of heavy chain variable domain:
SYAIS (SEQ ID NO: 81)
Framework Region 2 of heavy chain variable domain:
WVRQAPGQGLEWMG (SEQ ID NO: 85)
CDR2 of heavy chain variable domain:
GIIPIFGTANYAQKFQG (SEQ ID NO: 82)
Framework Region 3 of heavy chain variable domain:
RVTITADESTSTAYMELSSLRSDDTAVYYCAR (SEQ ID NO: 86)
CDR3 of heavy chain variable domain:
ERIAVAGTNYYYGMDV (SEQ ID NO: 83)
Framework Region 4 of heavy chain variable domain:
WGQGTLVTVSS (SEQ ID NO: 87)
Linker
GGGGSGGGGSGGGGS (SEQ ID NO: 135)
Anti-PSCA Clone #6 (also referred to as SD10) (scFv)
Light chain:
EIVLTQSPATLSVSPGERATLSCRASQSVSSSNLAWYQQKPGQAPRLLIYGASTRA
TGIPARFSGTGSGTDFTLTISRLEPEDFAVYYCQQYGSSITFGQGTRLEIK (SEQ ID
NO: 96)
Framework Region 1 of light chain variable domain:
EIVLTQSPATLSVSPGERATLSC (SEQ ID NO: 92)
CDR1 of light chain variable domain:
RASQSVSSSNLA (SEQ ID NO: 89)
Framework Region 2 of light chain variable domain:
WYQQKPGQAPRLLIY (SEQ ID NO: 93)
CDR2 of light chain variable domain:
GASTRAT (SEQ ID NO: 90)
Framework Region 3 of light chain variable domain:
GIPARFSGTGSGTDFTLTISRLEPEDFAVYYC (SEQ ID NO: 94)
CDR3 of light chain variable domain:
QQYGSSIT (SEQ ID NO: 91)
Framework Region 4 of light chain variable domain:
FGQGTRLEIK (SEQ ID NO: 95)
Anti-PSCA Clone #7 (also referred to as SH8) (scFv)
Heavy chain:
EVQLVESGAEVKKPGASVKVSCKASGYTFTGYYMHWVRQAPGQGLVWMGRIN
PNSGGTNYAQKFQGRVTMTRDTSISTAYMELSRLRSDDTAVYYCAFGTRHAFDI
WGQGTMVTVSS (SEQ ID NO: 104)
Framework Region 1 of heavy chain variable domain:
EVQLVESGAEVKKPGASVKVSCKASGYTFT (SEQ ID NO: 100)
CDR1 of heavy chain variable domain:
GYYMH (SEQ ID NO: 97)
Framework Region 2 of heavy chain variable domain:
WVRQAPGQGLVWMG (SEQ ID NO: 101)
CDR2 of heavy chain variable domain:
RINPNSGGTNYAQKFQG (SEQ ID NO: 98)
Framework Region 3 of heavy chain variable domain:
RVTMTRDTSISTAYMELSRLRSDDTAVYYCAF (SEQ ID NO: 102)
CDR3 of heavy chain variable domain:
GTRHAFDI (SEQ ID NO: 99)
Framework Region 4 of heavy chain variable domain:
WGQGTMVTVSS (SEQ ID NO: 103)
Linker
GGGGSGGGGSGGGGS (SEQ ID NO: 135)
Anti-PSCA Clone #7 (also referred to as SH8) (scFv)
Light chain:
NFMLTQPHSVSESPGKTVTISCTRSSGSIASNYVQWYQQRPGSAPTTVIYEDNQR
PSGVPDRFSGSIDSSSNSASLTISGLKTEDEADYYCQSYDSIVHVVFGGGTKVTVL
G (SEQ ID NO: 112)
Framework Region 1 of light chain variable domain:
NFMLTQPHSVSESPGKTVTISC (SEQ ID NO: 108)
CDR1 of light chain variable domain:
TRSSGSIASNYVQ (SEQ ID NO: 105)
Framework Region 2 of light chain variable domain:
WYQQRPGSAPTTVIY (SEQ ID NO: 109)
CDR2 of light chain variable domain:
EDNQRPS (SEQ ID NO: 106)
Framework Region 3 of light chain variable domain:
GVPDRFSGSIDSSSNSASLTISGLKTEDEADYYC (SEQ ID NO: 110)
CDR3 of light chain variable domain:
QSYDSIVHVV (SEQ ID NO: 107)
Framework Region 4 of light chain variable domain:
FGGGTKVTVLG (SEQ ID NO: 111)
Anti-PSCA Clone #8 (also referred to as SE8) (scFv)
Heavy chain:
EVQLVQSGAEVKKPGASVKVSCKASGGTFSSYAISWVRQAPGQGLEWMGGIIPI
FGTANYAQKFQGRVTITADKSTSTAYMELSSLRSEDTAVYYCARDVIVGATSSG
MDVWGQGTTVTVSS (SEQ ID NO: 120)
Framework Region 1 of heavy chain variable domain:
EVQLVQSGAEVKKPGASVKVSCKASGGTFS (SEQ ID NO: 116)
CDR1 of heavy chain variable domain:
SYAIS (SEQ ID NO: 113)
Framework Region 2 of heavy chain variable domain:
WVRQAPGQGLEWMG (SEQ ID NO: 117)
CDR2 of heavy chain variable domain:
GIIPIFGTANYAQKFQG (SEQ ID NO: 114)
Framework Region 3 of heavy chain variable domain:
RVTITADKSTSTAYMELSSLRSEDTAVYYCAR (SEQ ID NO: 118)
CDR3 of heavy chain variable domain:
DVIVGATSSGMDV (SEQ ID NO: 115)
Framework Region 4 of heavy chain variable domain:
WGQGTTVTVSS (SEQ ID NO: 119)
Linker
GGGGSGGGGSGGGGS (SEQ ID NO: 135)
Anti-PSCA Clone #8 (also referred to as SE8) (scFv)
Light chain:
DIQMTQSPSSVSASVGDRVTITCRASQSISRYLNWYQQKPGKAPKLLIYGASSLQ
SGVPSRFSGSGFGTDFTLTISSLQPEDFATYYCQQSYSTPLTFGQGTKLEIK (SEQ
ID NO: 128)
Framework Region 1 of light chain variable domain:
DIQMTQSPSSVSASVGDRVTITC (SEQ ID NO: 124)
CDR1 of light chain variable domain:
RASQSISRYLN (SEQ ID NO: 121)
Framework Region 2 of light chain variable domain:
WYQQKPGKAPKLLIY (SEQ ID NO: 125)
CDR2 of light chain variable domain:
GASSLQS (SEQ ID NO: 122)
Framework Region 3 of light chain variable domain:
GVPSRFSGSGFGTDFTLTISSLQPEDFATYYC (SEQ ID NO: 126)
CDR3 of light chain variable domain:
QQSYSTPLT (SEQ ID NO: 123)
Framework Region 4 of light chain variable domain:
FGQGTKLEIK (SEQ ID NO: 127)

### Example 8—Sequences Encoding Exemplary Binders Having the Ability to Bind to a Human PSCA Polypeptide

This Example depicts the nucleic acid sequences encoding the indicated Fabs of Clones #1-#5 and scFvs of Clones #6- #8.

Clone #1
Nucleic acid encoding SEQ ID NO: 8, SEQ ID NO: 131, and SEQ ID NO: 132:
(SEQ ID NO: 136)
CAAGTTCAATTAGTGGAGAGCGGCGGCGGATTGGTTAAACCGGGGGGCAGTC
TGCGTTTAAGCTGCGCAGCTTCCGGGTTTACTTTTTCGGATTACTATATGAGCT
GGATTCGTCAGGCTCCCGGTAAAGGGTTGGAATGGGTTAGCTACATCTCAAGT
TCCGGCTCTACAATCTACTATGCAGATTCCGTGAAAGGGCGCTTCACCATCTCT
CGTGACAATGCAAAGAACAGTTTATACTTACAAATGAACTCGCTGCGTGCGGA
AGATACGGCTGTGTATTACTGTGCGAGACCCCTCCGGTGGGGTATGGACGTCT
GGGGCCAAGGGACCACGGTCACCGTCTCCTCAGCGTCGACCAAAGGGCCCAG
TGTCTTTCCGTTAGCTCCGTCTTCCAAAAGTACGTCGGGCGGCACGGCGGCAC
TTGGCTGCTTAGTCAAGGACTACTTTCCCGAACCCGTTACTGTCTCTTGGAATA
GTGGGGCTTTGACCAGTGGGGTTCATACCTTTCCAGCCGTACTTCAGTCTTCA
GGGCTGTATAGTTTAAGTTCAGTGGTTACAGTGCCCTCTTCATCTTTAGGGACC
CAAACCTATATCTGCAACGTCAATCATAAGCCTTCAAATACCAAGGTTGACAA
AAAGGTGGAGCCCAAATCTTGT
Nucleic acid encoding SEQ ID NO: 16 and SEQ ID NO: 133:
(SEQ ID NO: 137)
GATATTCAAATGACCCAGTCTCCTTCTTCCTTGAGCGCGTCTGTGGGAGACCGT
GTAACTATCACGTGTCGTGCATCCCAAAGCATTTCGAGTTACTTGAACTGGTAT
CAACAAAAGCCTGGTAAAGCGCCCAAACTGTTGATCTATGCTGCCAGTAGTCT
TCAATCTGGTGTGCCCTCGCGCTTCTCAGGTTCGGGGTCAGGAACGGACTTTA
CCCTGACCATTAGCTCTCTGCAACCGGAAGATGCTGCAACGTATTACTGTCATC
AGAGTAGTAGTTTACCGCTCACTTTCGGCGGAGGGACCAAGGTGGAAATCAA
ACGTACGGTGGCTGCTCCGTCGGTGTTTATCTTTCCACCGAGTGACGAACAAC
TGAAGTCGGGGACTGCCAGCGTTGTTTGCCTTCTGAACAATTTTTACCCCCGC
GAGGCGAAGGTTCAGTGGAAAGTCGATAATGCGTTACAGAGCGGGAATAGCC
AGGAAAGTGTTACTGAACAGGATAGTAAAGACAGCACTTACAGCTTATCGTCT
ACACTTACTCTGAGTAAAGCGGATTATGAAAAGCATAAGGTGTACGCTTGCGA
GGTTACACATCAAGGGTTATCTTCGCCAGTCACGAAGAGCTTTAATCGCGGCG
AGTGC
Clone #2
Nucleic acid encoding SEQ ID NO: 24, SEQ ID NO: 131, and SEQ ID NO: 132:
(SEQ ID NO: 138)
CAAGTTCAATTAGTGGAGAGCGGCGGCGGATTGGTTAAACCGGGGGGCAGTC
TGCGTTTAAGCTGCGCAGCTTCCGGGTTTACTTTTTCGGATTACTATATGAGCT
GGATTCGTCAGGCTCCCGGTAAAGGGTTGGAATGGGTTAGCTACATCTCAAGT
TCCGGCTCTACAATCTACTATGCAGATTCCGTGAAAGGGCGCTTCACCATCTCT
CGTGACAATGCAAAGAACAGTTTATACTTACAAATGAACTCGCTGCGTGCGGA
AGATACGGCCGTGTATTACTGTGCGAGACCCCTCCGGTGGGGTATGGACGTCT
GGGGCCAAGGGACCACGGTCACCGTCTCCTCAGCGTCGACCAAAGGGCCCAG
TGTCTTTCCGTTAGCTCCGTCTTCCAAAAGTACGTCGGGCGGCACGGCGGCAC
TTGGCTGCTTAGTCAAGGACTACTTTCCCGAACCCGTTACTGTCTCTTGGAATA
GTGGGGCTTTGACCAGTGGGGTTCATACCTTTCCAGCCGTACTTCAGTCTTCA
GGGCTGTATAGTTTAAGTTCAGTGGTTACAGTGCCCTCTTCATCTTTAGGGACC
CAAACCTATATCTGCAACGTCAATCATAAGCCTTCAAATACCAAGGTTGACAA
AAAGGTGGAGCCCAAATCTTGT
Nucleic acid encoding SEQ ID NO: 32 and SEQ ID NO: 133:
(SEQ ID NO: 139)
GATATTCAAATGACCCAGTCTCCTTCTTCCTTGAGCGCGTCTGTGGGAGACCGT
GTAACTATCACGTGTCGTGCATCCCAAAGCATTTCGAGTTACTTGAACTGGTAT
CAACAAAAGCCTGGTAAAGCGCCCAAACTGTTGATCTATGCTGCCAGTAGTCT
TCAATCTGGTGTGCCCTCGCGCTTCTCAGGTTCGGGGTCAGGAACGGACTTTA
CCCTGACCATTAGCTCTCTGCAACCGGAAGATTTTGCAGTGTATTACTGTCATC
AGAGTAGTAGTTTACCCCTCACTTTCGGCGGAGGGACCAAACTGGAGATCAA
ACGTACGGTGGCTGCTCCGTCGGTGTTTATCTTTCCACCGAGTGACGAACAAC
TGAAGTCGGGGACTGCCAGCGTTGTTTGCCTTCTGAACAATTTTTACCCCCGC
GAGGCGAAGGTTCAGTGGAAAGTCGATAATGCGTTACAGAGCGGGAATAGCC
AGGAAAGTGTTACTGAACAGGATAGTAAAGACAGCACTTACAGCTTATCGTCT
ACACTTACTCTGAGTAAAGCGGATTATGAAAAGCATAAGGTGTACGCTTGCGA
GGTTACACATCAAGGGTTATCTTCGCCAGTCACGAAGAGCTTTAATCGCGGCG
AGTGC
Clone #3
Nucleic acid encoding SEQ ID NO: 40, SEQ ID NO: 131, and SEQ ID NO: 132:
(SEQ ID NO: 140)
CAAGTTCAATTAGTGGAGAGCGGCGGCGGATTGGTTAAACCGGGGGGCAGTC
TGCGTTTAAGCTGCGCAGCTTCCGGGTTTACTTTTTCGGATTACTATATGAGCT
GGATTCGTCAGGCTCCCGGTAAAGGGTTGGAATGGGTTAGCTACATCTCAAGT
TCCGGCTCTACAATCTACTATGCAGATTCCGTGAAAGGGCGCTTCACCATCTCT
CGTGACAATGCAAAGAACAGTTTATACTTACAAATGAACTCGCTGCGTGCGGA
AGATACGGCTGTGTATTACTGTGCGAGACCCCTCCGGTGGGGTATGGACGTCT
GGGGCCAAGGGACCACGGTCACTGTCTCCTCAGCGTCGACCAAAGGGCCCAG
TGTCTTTCCGTTAGCTCCGTCTTCCAAAAGTACGTCGGGCGGCACGGCGGCAC
TTGGCTGCTTAGTCAAGGACTACTTTCCCGAACCCGTTACTGTCTCTTGGAATA
GTGGGGCTTTGACCAGTGGGGTTCATACCTTTCCAGCCGTACTTCAGTCTTCA
GGGCTGTATAGTTTAAGTTCAGTGGTTACAGTGCCCTCTTCATCTTTAGGGACC
CAAACCTATATCTGCAACGTCAATCATAAGCCTTCAAATACCAAGGTTGACAA
AAAGGTGGAGCCCAAATCTTGT
Nucleic acid encoding SEQ ID NO: 48 and SEQ ID NO: 133:
(SEQ ID NO: 141)
GATATTCAAATGACCCAGTCTCCTTCTTCCTTGAGCGCGTCTGTGGGAGACCGT
GTAACTATCACGTGTCGTGCATCCCAAAGCATTTCGAGTTACTTGAACTGGTAT
CAACAAAAGCCTGGTAAAGCGCCCAAACTGTTGATCTATGCTGCCAGTAGTCT
TCAATCTGGTGTGCCCTCGCGCTTCTCAGGTTCGGGGTCAGGAACGGACTTTA
CCCTGACCATTAGCTCTCTGCAACCGGAAGATGTGGCAGTTTATTACTGTCATC
AGAGTAATAGTTTACCGCTCACTTTCGGCGGAGGGACCAAAGTGGAGATCAA
ACGTACGGTGGCTGCTCCGTCGGTGTTTATCTTTCCACCGAGTGACGAACAAC
TGAAGTCGGGGACTGCCAGCGTTGTTTGCCTTCTGAACAATTTTTACCCCCGC
GAGGCGAAGGTTCAGTGGAAAGTCGATAATGCGTTACAGAGCGGGAATAGCC
AGGAAAGTGTTACTGAACAGGATAGTAAAGACAGCACTTACAGCTTATCGTCT
ACACTTACTCTGAGTAAAGCGGATTATGAAAAGCATAAGGTGTACGCTTGCGA
GGTTACACATCAAGGGTTATCTTCGCCAGTCACGAAGAGCTTTAATCGCGGCG
AGTGC
Clone #4
Nucleic acid encoding SEQ ID NO: 56, SEQ ID NO: 131, and SEQ ID NO: 132:
(SEQ ID NO: 142)
CAAGTTCAATTAGTGGAGAGCGGCGGCGGATTGGTTAAACCGGGGGGCAGTC
TGCGTTTAAGCTGCGCAGCTTCCGGGTTTACTTTTTCGGATTACTATATGAGCT
GGATTCGTCAGGCTCCCGGTAAAGGGTTGGAATGGGTTAGCTACATCTCAAGT
TCCGGCTCTACAATCGTCTATGCAGATTCCGTGAAAGGGCGCTTCACCATCTCT
CGTGACAATGCAAAGAACAGTTTATACTTACAAATGAACTCGCTGCGTGCGGA
AGATACGGCTGTGTATTACTGTGCGAGACCCATCCGGAGGGGTATGGACGTCT
GGGGCCAAGGGACCACGGTCACCGTCTCCTCAGCGTCGACCAGAGGGCCCAG
TGTCTTTCCGTTAGCTCCGTCTTCCAAAAGTACGTCGGGCGGCACGGCGGCAC
TTGGCTGCTTAGTCAAGGACTACTTTCCCGAACCCGTTACTGTCTCTTGGAATA
GTGGGGCTTTGACCAGTGGGGTTCATACCTTTCCAGCCGTACTTCAGTCTTCA
GGGCTGTATAGTTTAAGTTCAGTGGTTACAGTGCCCTCTTCATCTTTAGGGACC
CAAACCTATATCTGCAACGTCAATCATAAGCCTTCAAATACCAAGGTTGACAA
AAAGGTGGAGCCCAAATCTTGT
Nucleic acid encoding SEQ ID NO: 64 and SEQ ID NO: 133:
(SEQ ID NO: 143)
GATATTCAAATGACCCAGTCTCCTTCTTCCTTGAGCGCGTCTGTGGGAGACCGT
GTAACTATCACGTGTCGTGCATCCCAAAGCATTTCGAGTTACTTGAACTGGTAT
CAACAAAAGCCTGGTAAAGCGCCCAAACTGTTGATCTATGCTGCCAGTAGTCT
TCAATCTGGTGTGCCCTCGCGCTTCTCAGGTTCGGGGTCAGGAACGGACTTTA
CCCTGACCATTAGCTCTCTGCAACCGGAAGATGCTGCAACGTATTACTGTCATC
AGAGTAGTAGTTTACCGCTCACTTTCGGCGGAGGGACCAAGGTGGAAATCAA
ACGTACGGTGGCTGCTCCGTCGGTGTTTATCTTTCCACCGAGTGACGAACAAC
TGAAGTCGGGGACTGCCAGCGTTGTTTGCCTTCTGAACAATTTTTACCCCCGC
GAGGCGAAGGTTCAGTGGAAAGTCGATAATGCGTTACAGAGCGGGAATAGCC
AGGAAAGTGTTACTGAACAGGATAGTAAAGACAGCACTTACAGCTTATCGTCT
ACACTTACTCTGAGTAAAGCGGATTATGAAAAGCATAAGGTGTACGCTTGCGA
GGTTACACATCAAGGGTTATCTTCGCCAGTCACGAAGAGCTTTAATCGCGGCG
AGTGC
Clone #5
Nucleic acid encoding SEQ ID NO: 72, SEQ ID NO: 131, and SEQ ID NO: 132:
(SEQ ID NO: 144)
CAAGTTCAATTAGTGGAGAGCGGCGGCGGATTGGTTAAACCGGGGGGCAGTC
TGCGTTTAAGCTGCGCAGCTTCCGGGTTTACTTTTTCGGATTACTATATGAGCT
GGATTCGTCAGGCTCCCGGTAAAGGGTTGGAATGGGTTAGCTACATCTCAAGT
TCCGGCTCTACAATCAACTATGCAGATTCCGTGAAAGGGCGCTTCACCATCTCT
CGTGACAATGCAAAGAACAGTTTATACTTACAAATGAACTCGCTGCGTGCGGA
AGATACGGCTGTGTATTACTGTGCGAGACCCATCCGGTGGGGTATGGACGTCT
GGGGCCAAGGGACCACGGTCACCGTCTCCTCAGCGTCGACCAAAGGGCCCAG
TGTCTTTCCGTTAGCTCCGTCTTCCAAAAGTACGTCGGGCGGCACGGCGGCAC
TTGGCTGCTTAGTCAAGGACTACTTTCCCGAACCCGTTACTGTCTCTTGGAATA
GTGGGGCTTTGACCAGTGGGGTTCATACCTTTCCAGCCGTACTTCAGTCTTCA
GGGCTGTATAGTTTAAGTTCAGTGGTTACAGTGCCCTCTTCATCTTTAGGGACC
CAAACCTATATCTGCAACGTCAATCATAAGCCTTCAAATACCAAGGTTGACAA
AAAGGTGGAGCCCAAATCTTGT
Nucleic acid encoding SEQ ID NO: 80 and SEQ ID NO: 133:
(SEQ ID NO: 145)
GATATTCAAATGACCCAGTCTCCTTCTTCCTTGAGCGCGTCTGTGGGAGACCGT
GTAACTATCACGTGTCGTGCATCCCAAAGCATTTCGAGTTACTTGAACTGGTAT
CAACAAAAGCCTGGTAAAGCGCCCAAACTGTTGATCTATGCTGCCAGTAGTCT
TCAATCTGGTGTGCCCTCGCGCTTCTCAGGTTCGGGGTCAGGAACGGACTTTA
CCCTGACCATTAGCTCTCTGCAACCGGAAGATGCTGCAACGTATTACTGTCATC
AGAGTAGTAGTTTACCGCTCACTTTCGGCGGAGGGACCAAGGTGGAAATCAA
ACGTACGGTGGCTGCTCCGTCGGTGTTTATCTTTCCACCGAGTGACGAACAAC
TGAAGTCGGGGACTGCCAGCGTTGTTTGCCTTCTGAACAATTTTTACCCCCGC
GAGGCGAAGGTTCAGTGGAAAGTCGATAATGCGTTACAGAGCGGGAATAGCC
AGGAAAGTGTTACTGAACAGGATAGTAAAGACAGCACTTACAGCTTATCGTCT
ACACTTACTCTGAGTAAAGCGGATTATGAAAAGCATAAGGTGTACGCTTGCGA
GGTTACACATCAAGGGTTATCTTCGCCAGTCACGAAGAGCTTTAATCGCGGCG
AGTGC
Clone #6
Nucleic acid encoding scFv that includes SEQ ID NO: 88 and SEQ ID NO: 96:
(SEQ ID NO: 146)
GAGGTCCAGCTGGTGCAGTCTGGGGCTGAGGTGAAGAAGCCTGGGTCCTCGG
TGAAGGTCTCTTGCAAGGCTTCTGGAGGCACCTTCAGCAGCTATGCTATCAGC
TGGGTGCGACAGGCCCCTGGACAAGGGCTTGAGTGGATGGGAGGGATCATCC
CTATCTTTGGTACAGCAAACTACGCACAGAAGTTCCAGGGCAGAGTCACGATT
ACCGCGGACGAATCCACGAGCACAGCCTACATGGAGCTGAGCAGCCTGAGAT
CTGACGACACGGCCGTGTATTACTGTGCGAGAGAGCGTATAGCAGTGGCTGGT
ACTAACTACTACTACGGTATGGACGTCTGGGGCCAAGGCACCCTGGTCACCGT
CTCCTCAGGAGGTGGCGGGTCTGGTGGGGGCGGAAGCGGTGGTGGCGGATCC
GAAATTGTGTTGACACAGTCTCCAGCCACCCTGTCTGTGTCTCCAGGGGAAAG
AGCCACCCTCTCCTGCAGGGCCAGTCAGAGTGTTAGCAGCAGCAACTTAGCC
TGGTACCAGCAGAAACCTGGCCAGGCTCCCAGGCTCCTCATCTATGGTGCATC
CACCAGGGCCACTGGTATCCCAGCCAGGTTCAGTGGCACTGGGTCTGGGACA
GACTTCACTCTCACCATCAGCAGGCTGGAGCCTGAAGATTTTGCAGTGTATTA
CTGTCAGCAGTATGGTAGCTCGATCACCTTCGGCCAAGGGACACGACTAGAGA
TTAAA
Clone #7
Nucleic acid encoding scFv that includes SEQ ID NO: 104 and SEQ ID NO: 112:
(SEQ ID NO: 147)
GAGGTGCAGCTGGTGGAGTCCGGGGCTGAGGTGAAGAAGCCTGGGGCCTCA
GTGAAGGTCTCCTGCAAGGCTTCTGGATACACCTTCACCGGCTACTATATGCAC
TGGGTGCGACAGGCCCCTGGACAAGGGCTTGTGTGGATGGGACGGATCAACC
CTAACAGTGGTGGCACAAACTATGCACAGAAGTTTCAGGGCAGGGTCACCAT
GACCAGGGACACGTCCATCAGCACAGCCTACATGGAGCTGAGCAGGCTGAGA
TCTGACGACACGGCCGTGTATTACTGTGCGTTCGGGACGCGCCATGCTTTTGAT
ATCTGGGGCCAAGGGACAATGGTCACCGTCTCTTCAGGAGGTGGCGGGTCTG
GTGGGGGCGGAAGCGGTGGTGGCGGATCCAATTTTATGCTGACTCAGCCCCAC
TCTGTGTCGGAGTCCCCGGGAAAGACGGTAACCATCTCCTGCACCCGCAGTAG
TGGCTCCATTGCCAGCAACTATGTGCAGTGGTACCAGCAGCGCCCGGGCAGTG
CCCCCACCACTGTGATCTATGAGGATAACCAAAGACCCTCTGGGGTCCCTGAT
CGGTTCTCTGGCTCCATCGACAGCTCCTCCAACTCTGCCTCCCTCACCATCTCT
GGACTGAAGACTGAGGACGAGGCTGACTACTACTGTCAGTCTTATGATAGCAT
CGTCCATGTGGTGTTCGGCGGAGGGACCAAGGTCACCGTCCTAGGT
Clone #8
Nucleic acid encoding scFv that includes SEQ ID NO: 120 and SEQ ID NO: 128:
(SEQ ID NO: 148)
GAGGTGCAGCTGGTGCAGTCTGGGGCTGAGGTGAAGAAGCCTGGGGCCTCAG
TGAAGGTCTCCTGCAAGGCTTCTGGAGGCACCTTCAGCAGCTATGCTATCAGC
TGGGTGCGACAGGCCCCTGGACAAGGGCTTGAGTGGATGGGAGGGATCATCC
CTATCTTTGGTACAGCAAACTACGCACAGAAGTTCCAGGGCAGAGTCACGATT
ACCGCGGACAAATCCACGAGCACAGCCTACATGGAGCTGAGCAGCCTGAGAT
CTGAGGACACGGCCGTGTATTACTGTGCGAGAGACGTTATAGTGGGAGCTACT
AGCTCGGGTATGGACGTCTGGGGCCAAGGGACCACGGTCACCGTCTCCTCAG
GAGGTGGCGGGTCTGGTGGGGGCGGAAGCGGTGGTGGCGGATCCGACATCCA
GATGACCCAGTCTCCATCTTCCGTGTCTGCATCTGTAGGAGACAGAGTCACCA
TCACTTGCCGGGCAAGTCAGAGCATTAGCAGATATTTAAATTGGTATCAGCAG
AAACCAGGGAAAGCCCCTAAGCTCCTGATCTATGGTGCATCCAGTTTGCAAAG
TGGGGTCCCATCAAGGTTCAGTGGCAGTGGATTTGGGACAGATTTCACTCTCA
CCATCAGCAGTCTGCAACCTGAAGATTTTGCAACTTACTACTGTCAACAGAGT
TACAGTACCCCTCTGACGTTCGGCCAAGGGACCAAGCTGGAAATCAAA (SEQ

### Example 9—Exemplary Ig Sequences

This Example depicts the structure of an exemplary Ig and provides the amino acid and nucleic acid sequences of exemplary hinge, CH2, and CH3 regions/domains.

Exemplary Ig (e.g., IgG1) structure
Heavy chain: Heavy Chain Variable Domain + CH1 +
Hinge + CH2 + CH3
Light chain: Light Chain Variable Domain +
Constant Light (Kappa or Lambda)
Human immunoglobulin G1 hinge region:
(SEQ ID NO: 149)
EPKSCDKTHTCPPCP
(SEQ ID NO: 150)
GAGCCCAAATCTTGTGACAAAACTCACACATGCCCACCGTGCCCA
Human immunoglobulin G1 heavy chain constant
domains 2 and 3 (CH2—CH3):
(SEQ ID NO: 151)
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNV
FSCSVMHEALHNHYTQKSLSLSPGK
(SEQ ID NO: 152)
GCACCTGAACTCCTGGGGGGACCGTCAGTCTTCCTCTTCCCCCCAAAA
CCCAAGGACACCCTCATGATCTCCCGGACCCCTGAGGTCACATGCGTG
GTGGTGGACGTGAGCCACGAAGACCCTGAGGTCAAGTTCAACTGGTAC
GTGGACGGCGTGGAGGTGCATAATGCCAAGACAAAGCCGCGGGAGGAG
CAGTACAACAGCACGTACCGTGTGGTCAGCGTCCTCACCGTCCTGCAC
CAGGACTGGCTGAATGGCAAGGAGTACAAGTGCAAGGTCTCCAACAAA
GCCCTCCCAGCCCCCATCGAGAAAACCATCTCCAAAGCCAAAGGGCAG
CCCCGAGAACCACAGGTGTACACCCTGCCCCCATCCCGGGATGAGCTG
ACCAAGAACCAGGTCAGCCTGACCTGCCTGGTCAAAGGCTTCTATCCC
AGCGACATCGCCGTGGAGTGGGAGAGCAATGGGCAGCCGGAGAACAAC
TACAAGACCACGCCTCCCGTGCTGGACTCCGACGGCTCCTTCTTCCTC
TACAGCAAGCTCACCGTGGACAAGAGCAGGTGGCAGCAGGGGAACGTC
TTCTCATGCTCCGTGATGCATGAGGCTCTGCACAACCACTACACGCAG
AAGAGCCTCTCCCTGTCTCCGGGTAAA

### Example 10—Structures of Exemplary scFv's

This Example depicts the structure of exemplary scFv's.

Exemplary scFv Structure:
Heavy Chain Variable Domain/Region+Linker+Light Chain Variable Domain/Region
   Exemplary scFv Structure:
Light Chain Variable Domain/Region+Linker+Heavy Chain Variable Domain/Region

### Example 11—Sequences of Exemplary scFv's

This Example depicts the amino acid sequences of exemplary heavy chain variable domains and exemplary light chain variable domains of exemplary scFvs. The CDRs and framework sequences of each also are delineated. An exemplary linker amino acid sequence such as a linker amino acid sequence set forth in Example 12 can be used to link the heavy chain variable domain and the light chain variable domain together to form a scFv.

Variable Heavy Region structure:
Framework region (FR) 1 + CDR1 + FR2 + CDR2 + FR3 + CDR3 + FR4
Framework Region 1 of Variable Heavy Region:
(SEQ ID NO: 154)
EVQLVESGGGLVQPGGSLRLSCAASAFYFT
Framework Region 2 of Variable Heavy Region:
(SEQ ID NO: 155)
WVRQAPGQGLEWVS
Framework Region 3 of Variable Heavy Region:
(SEQ ID NO: 156)
RFTISRDNSKNTLYLQMNSLRAEDTAMYYCAR
Framework Region 4 of Variable Heavy Region:
(SEQ ID NO: 157)
WGQGTLVTVSS
Variable Light Region structure:
FR1 + CDR1 + FR2 + CDR2 + FR3 + CDR3 + FR4
Framework Region 1 of Variable Light Region:
(SEQ ID NO: 159)
VPVLTQPHSVSESPGKTVTISCTRS
Framework Region 2 of Variable Light Region:
(SEQ ID NO: 160)
VQWYQQRPGSSPTTVIY
Framework Region 3 of Variable Light Region:
(SEQ ID NO: 161)
QRPSGVPDRFSGSIDMSSNSASLTISGLRTEDEGDYYC
Framework Region 4 of Variable Light Region:
(SEQ ID NO: 162)
FGGGTQLTVLG
Exemplary scFv structures
Variable Heavy Region structure:
FR1 + CDR1 + FR2 + CDR2 + FR3 + CDR3 + FR4
Framework Region 1 of Variable Heavy Region:
(SEQ ID NO: 164)
EVQLVESGGGLVQPGGSLRLSCAASAFYFT
Framework Region 2 of Variable Heavy Region:
(SEQ ID NO: 165)
WVRQAPGQGLERVS
Framework Region 3 of Variable Heavy Region:
(SEQ ID NO: 166)
RFTISRDNSKNTLYLEMNSLRAEDTAMYYCAR
Framework Region 4 of Variable Heavy Region:
(SEQ ID NO: 167)
WGQGTLVTVSS
Variable Light Region structure:
FR1 + CDR1 + FR2 + CDR2 + FR3 + CDR3 + FR4
Framework Region 1 of Variable Light Region:
(SEQ ID NO: 169)
QSALTQPASVSGSPGQSITISCTRA
Framework Region 2 of Variable Light Region:
(SEQ ID NO: 170)
VSWYQQHPGKAPKLMIY
Framework Region 3 of Variable Light Region:
(SEQ ID NO: 171)
NRPSGVSNRFSGSKSGNTASLTISGLQAEDEADYYC
Framework Region 4 of Variable Light Region:
(SEQ ID NO: 172)
FGGGTQLTVLG
Variable Heavy Region structure:
FR1 + CDR1 + FR2 + CDR2 + FR3 + CDR3 + FR4
Framework Region 1 of Variable Heavy Region:
(SEQ ID NO: 174)
EVQLVESGGGLVQPGGSLRLSCAASYFDFD
Framework Region 2 of Variable Heavy Region:
(SEQ ID NO: 175)
WVRQAPGQGLEWVS
Framework Region 3 of Variable Heavy Region:
(SEQ ID NO: 176)
RFTISRDNSKNTLYLQMNSLRAEDTAVYYCAR
Framework Region 4 of Variable Heavy Region:
(SEQ ID NO: 177)
WGQGTLVTVSS
Variable Light Region structure:
FR1 + CDR1 + FR2 + CDR2 + FR3 + CDR3 + FR4
Framework Region 1 of Variable Light Region:
(SEQ ID NO: 179)
DIQMTQSPSSLSASVGDRVTITC
Framework Region 2 of Variable Light Region:
(SEQ ID NO: 180)
WYQQKPGKAPKLLIY
Framework Region 3 of Variable Light Region:
(SEQ ID NO: 181)
GVPSRFSGSGSGTDFTLTISSLQPEDFGVYYC
Framework Region 4 of Variable Light Region:
(SEQ ID NO: 182)
FGQGTRLEIK
Variable Heavy Region structure:
FR1 + CDR1 + FR2 + CDR2 + FR3 + CDR3 + FR4
Framework Region 1 of Variable Heavy Region:
(SEQ ID NO: 184)
EVQLVESGGGLVQPGGSLRLSCAASYLDFD
Framework Region 2 of Variable Heavy Region:
(SEQ ID NO: 185)
WFRQAPGQGLEWVS
Framework Region 3 of Variable Heavy Region:
(SEQ ID NO: 186)
RFTISRDNSQNTLYLQMNSLRAEDTAVYYCAR
Framework Region 4 of Variable Heavy Region:
(SEQ ID NO: 187)
WGQGTLVTVSS
Variable Light Region structure:
FR1 + CDR1 + FR2 + CDR2 + FR3 + CDR3 + FR4
Framework Region 1 of Variable Light Region:
(SEQ ID NO: 189)
DIQMTQSPSSLSASVGDRVTITC
Framework Region 2 of Variable Light Region:
(SEQ ID NO: 190)
WYQQKPGKAPKLLIY
Framework Region 3 of Variable Light Region:
(SEQ ID NO: 191)
GVPSRFSGSGSGTDFTLTISSLQPEDVGVYYC
Framework Region 4 of Variable Light Region:
(SEQ ID NO: 192)
FGGGTKLEIK
Variable Heavy Region structure:
FR1 + CDR1 + FR2 + CDR2 + FR3 + CDR3 + FR4
Framework Region 1 of heavy chain variable domain:
(SEQ ID NO: 4)
QVQLVESGGGLVKPGGSLRLSCAASGFTFS
Framework Region 2 of heavy chain variable domain:
(SEQ ID NO: 5)
WIRQAPGKGLEWVS
Framework Region 3 of heavy chain variable domain:
(SEQ ID NO: 6)
RFTISRDNAKNSLYLQMNSLRAEDTAVYYCAR
Framework Region 4 of heavy chain variable domain:
(SEQ ID NO: 7)
WGQGTTVTVSS
Variable Light Region structure:
FR1 + CDR1 + FR2 + CDR2 + FR3 + CDR3 + FR4
Framework Region 1 of light chain variable domain:
(SEQ ID NO: 12)
DIQMTQSPSSLSASVGDRVTITC
Framework Region 2 of light chain variable domain:
(SEQ ID NO: 13)
WYQQKPGKAPKLLIY
Framework Region 3 of light chain variable domain:
(SEQ ID NO: 14)
GVPSRFSGSGSGTDFTLTISSLQPEDAATYYC
Framework Region 4 of light chain variable domain:
(SEQ ID NO: 15)
FGGGTKVEIK
Variable Heavy Region structure:
FR1 + CDR1 + FR2 + CDR2 + FR3 + CDR3 + FR4
Framework Region 1 of heavy chain variable domain:
(SEQ ID NO: 20)
QVQLVESGGGLVKPGGSLRLSCAASGFTFS
Framework Region 2 of heavy chain variable domain:
(SEQ ID NO: 21)
WIRQAPGKGLEWVS
Framework Region 3 of heavy chain variable domain:
(SEQ ID NO: 22)
RFTISRDNAKNSLYLQMNSLRAEDTAVYYCAR
Framework Region 4 of heavy chain variable domain:
(SEQ ID NO: 23)
WGQGTTVTVSS
Variable Light Region structure:
FR1 + CDR1 + FR2 + CDR2 + FR3 + CDR3 + FR4
Framework Region 1 of light chain variable domain:
(SEQ ID NO: 28)
DIQMTQSPSSLSASVGDRVTITC
Framework Region 2 of light chain variable domain:
(SEQ ID NO: 29)
WYQQKPGKAPKLLIY
Framework Region 3 of light chain variable domain:
(SEQ ID NO: 30)
GVPSRFSGSGSGTDFTLTISSLQPEDFAVYYC
Framework Region 4 of light chain variable domain:
(SEQ ID NO: 31)
FGGGTKLEIK
Variable Heavy Region structure:
FR1 + CDR1 + FR2 + CDR2 + FR3 + CDR3 + FR4
Framework Region 1 of heavy chain variable domain:
(SEQ ID NO: 36)
QVQLVESGGGLVKPGGSLRLSCAASGFTFS
Framework Region 2 of heavy chain variable domain:
(SEQ ID NO: 37)
WIRQAPGKGLEWVS
Framework Region 3 of heavy chain variable domain:
(SEQ ID NO: 38)
RFTISRDNAKNSLYLQMNSLRAEDTAVYYCAR
Framework Region 4 of heavy chain variable domain:
(SEQ ID NO: 39)
WGQGTTVTVSS
Variable Light Region structure:
FR1 + CDR1 + FR2 + CDR2 + FR3 + CDR3 + FR4
Framework Region 1 of light chain variable domain:
(SEQ ID NO: 44)
DIQMTQSPSSLSASVGDRVTITC
Framework Region 2 of light chain variable domain:
(SEQ ID NO: 45)
WYQQKPGKAPKLLIY
Framework Region 3 of light chain variable domain:
(SEQ ID NO: 46)
GVPSRFSGSGSGTDFTLTISSLQPEDVAVYYC
Framework Region 4 of light chain variable domain:
(SEQ ID NO: 47)
FGGGTKVEIK
Variable Heavy Region structure:
FR1 + CDR1 + FR2 + CDR2 + FR3 + CDR3 + FR4
Framework Region 1 of heavy chain variable domain:
(SEQ ID NO: 52)
QVQLVESGGGLVKPGGSLRLSCAASGFTFS
Framework Region 2 of heavy chain variable domain:
(SEQ ID NO: 53)
WIRQAPGKGLEWVS
Framework Region 3 of heavy chain variable domain:
(SEQ ID NO: 54)
RFTISRDNAKNSLYLQMNSLRAEDTAVYYCAR
Framework Region 4 of heavy chain variable domain:
(SEQ ID NO: 55)
WGQGTTVTVSS
Variable Light Region structure:
FR1 + CDR1 + FR2 + CDR2 + FR3 + CDR3 + FR4
Framework Region 1 of light chain variable domain:
(SEQ ID NO: 60)
DIQMTQSPSSLSASVGDRVTITC
Framework Region 2 of light chain variable domain:
(SEQ ID NO: 61)
WYQQKPGKAPKLLIY
Framework Region 3 of light chain variable domain:
(SEQ ID NO: 62)
GVPSRFSGSGSGTDFTLTISSLQPEDAATYYC
Framework Region 4 of light chain variable domain:
(SEQ ID NO: 63)
FGGGTKVEIK
Variable Heavy Region structure:
FR1 + CDR1 + FR2 + CDR2 + FR3 + CDR3 + FR4
Framework Region 1 of heavy chain variable domain:
(SEQ ID NO: 68)
QVQLVESGGGLVKPGGSLRLSCAASGFTFS
Framework Region 2 of heavy chain variable domain:
(SEQ ID NO: 69)
WIRQAPGKGLEWVS
Framework Region 3 of heavy chain variable domain:
(SEQ ID NO: 70)
RFTISRDNAKNSLYLQMNSLRAEDTAVYYCAR
Framework Region 4 of heavy chain variable domain:
(SEQ ID NO: 71)
WGQGTTVTVSS
Variable Light Region structure:
FR1 + CDR1 + FR2 + CDR2 + FR3 + CDR3 + FR4
Framework Region 1 of light chain variable domain:
(SEQ ID NO: 76)
DIQMTQSPSSLSASVGDRVTITC
Framework Region 2 of light chain variable domain:
(SEQ ID NO: 77)
WYQQKPGKAPKLLIY
Framework Region 3 of light chain variable domain:
(SEQ ID NO: 78)
GVPSRFSGSGSGTDFTLTISSLQPEDAATYYC
Framework Region 4 of light chain variable domain:
(SEQ ID NO: 79)
FGGGTKVEIK

### Example 12—Exemplary Linkers

This Example depicts exemplary linker amino acid sequences that can be used to link a heavy chain variable domain and a light chain variable domain together to form a scFv. These linker sequences also can be used to create CARs and cell engagers.

a)
(SEQ ID NO: 203)
GGGGSGGSGSGGGGS
b)
(SEQ ID NO: 204)
GGGGSGGSGSSGGGS
c)
(SEQ ID NO: 135)
GGGGSGGGGSGGGGS
d)
(SEQ ID NO: 205)
GGGGSGSGASSGGGS
e)
(SEQ ID NO: 206)
GGGGSGSGASGGGGS
f)
(SEQ ID NO: 207)
SGGGSGGGGSGGGGS
g)
(SEQ ID NO: 208)
GSTSGSGKPGSGEGSTKG
h)
(SEQ ID NO: 209)
PNGASQSSSASHTGSAPGS
i)
(SEQ ID NO: 210)
PNGASNSGSAPDTSSAPGS
j)
(SEQ ID NO: 211)
PNGASHSGSAPNTSSAPGS
k)
(SEQ ID NO: 212)
PNGASESGSASKTSSASGS
l)
(SEQ ID NO: 213)
PNGASNSGSAPKTGSASGS
m)
(SEQ ID NO: 214)
PNGASKSGSASQTSSAPGS
n)
(SEQ ID NO: 215)
PNGASHSSSASQTGSAPGS
o)
(SEQ ID NO: 216)
PNGASKRSAPGS
p)
(SEQ ID NO: 217)
PNGASHSGSAPHTSSASGS
q)
(SEQ ID NO: 218)
RGRGRGRGRSRGGGS
r)
(SEQ ID NO: 219)
SHGGSHGGGSGGGGS
s)
(SEQ ID NO: 220)
GQAGR
t)
(SEQ ID NO: 221)
GGGSGGGG
u)
(SEQ ID NO: 222)
SGGGG
v)
(SEQ ID NO: 223)
GGGGS
w)
(SEQ ID NO: 224)
GGGGSGGGGSGGGGSGGGGSGGGGSGGGGS
x)
(SEQ ID NO: 225)
GGGGGSGGGGSGGGGS
y)
(SEQ ID NO: 226)
GGGGGSGGGGSGGGGSGGGGSGGGGS
z)
(SEQ ID NO: 227)
GGGGSGGGGSGGGGSGGGGSGGGGS
a1)
(SEQ ID NO: 228)
AAA
b1)
(SEQ ID NO: 229)
VEGGSGGSGGSGGSGGVD
c1)
(SEQ ID NO: 839)
GSGSSGSGSSGSGSS
d1)
(SEQ ID NO: 840)
GSGSSG

### Example 13—Structures of Exemplary CARs

This Example depicts structures of exemplary CARs.

### Exemplary CAR Structures:

Signal Peptide+scFv+(Linker/Hinge)ₙ₁+Transmembrane Domain+(Intracellular Signaling Domain)ₙ₂
   n1=0, 1,2, or 3
   n2=1, 2, 3, 4, or 5 with or without a linker located between consecutive intracellular signaling domains
Signal Peptide+scFv+Linker+Hinge+Transmembrane Domain+Intracellular Signaling Domain+Intracellular Signaling Domain+Linker+Intracellular Signaling Domain
Signal Peptide+scFv+Linker+Hinge+Transmembrane Domain+Intracellular Signaling Domain+Linker+Intracellular Signaling Domain
Signal Peptide+scFv+Hinge+Transmembrane Domain+Intracellular Signaling Domain+Intracellular Signaling Domain+Linker+Intracellular Signaling Domain
Signal Peptide+scFv+Hinge+Transmembrane Domain+Intracellular Signaling Domain+Intracellular Signaling Domain+Intracellular Signaling Domain
Signal Peptide+scFv+Linker+Transmembrane Domain+Intracellular Signaling Domain+Intracellular Signaling Domain+Intracellular Signaling Domain

### Exemplary CAR Structures:

Signal Peptide+VH Domain+(Linker/Hinge)ₙ₁+Transmembrane Domain+(Intracellular Signaling Domain)ₙ₂
   n1=0, 1,2, or 3
   n2=1, 2, 3, 4, or 5 with or without a linker located between consecutive intracellular signaling domains
Signal Peptide+VH Domain+Linker+Hinge+Transmembrane Domain+Intracellular Signaling Domain+Intracellular Signaling Domain+Linker+Intracellular Signaling Domain
Signal Peptide+VH Domain+Linker+Hinge+Transmembrane Domain+Intracellular Signaling Domain+Linker+Intracellular Signaling Domain
Signal Peptide+VH Domain+Hinge+Transmembrane Domain+Intracellular Signaling Domain+Intracellular Signaling Domain+Linker+Intracellular Signaling Domain
Signal Peptide+VH Domain+Hinge+Transmembrane Domain+Intracellular Signaling Domain+Intracellular Signaling Domain+Intracellular Signaling Domain
Signal Peptide+VH Domain+Linker+Transmembrane Domain+Intracellular Signaling Domain+Intracellular Signaling Domain+Intracellular Signaling Domain

### Example 14—Exemplary Signal Peptides

This Example depicts the amino acid sequences of exemplary signal peptides that can be used to design a CAR.

Exemplary signal peptides for CARS
Exemplary human IGKV1-39-derived signal peptide:
(SEQ ID NO: 230)
MDMRVPAQLLGLLLLWLRGARC
(SEQ ID NO: 231)
ATGGATATGAGGGTCCCCGCACAATTGCTCGGTCTCCTTCTGCTTTGGCT
CCGGGGCGCGCGGTGC
Exemplary human IGKV1-16-derived signal peptide:
(SEQ ID NO: 232)
MDMRVLAQLLGLLLLCFPGARC
Exemplary human IGKV3-11-derived signal peptide:
(SEQ ID NO: 233)
MEAPAQLLFLLLLWLPDTTG
Exemplary human IGKV4-1-derived signal peptide:
(SEQ ID NO: 234)
MVLQTQVFISLLLWISGAYG
Exemplary human IGKV6-21-derived signal peptide:
(SEQ ID NO: 235)
MLPSQLIGFLLLWVPASRG
Exemplary human IGKV1-33-derived signal peptide:
(SEQ ID NO: 236)
MDMRVPAQLLGLLLLWLSGARC

### Example 15—Exemplary Hinges

This Example depicts the amino acid sequences of exemplary hinges that can be used to design a CAR.

Exemplary hinges for CARs (in some cases,
these hinges can be used a linkers)
Exemplary human IgG1-derived hinge:
(SEQ ID NO: 237)
EPKSCDKTHTCPPCP
Exemplary human IgG2-derived hinge:
(SEQ ID NO: 238)
DKTHTCPPCPAPPVA
Exemplary human IgG4-derived hinges:
(SEQ ID NO: 239)
ESKYGPPCPPCP
(SEQ ID NO: 240)
ESKYGPPCPSCP
Exemplary human CD8α-derived hinge:
(SEQ ID NO: 241)
KPTTTPAPRPPTPAPTIASQPLSLRPEACRPAAGGAVHTRGLDFACDIY
(SEQ ID NO: 242)
TTTPAPRPPTPAPTIASQPLSLRPEACRPAAGGAVHTRGLDFACDIY
(SEQ ID NO: 243)
TTTPAPRPPTPAPTIASQPLSLRPEACRPAAGGAVHTRGLDFACD
Exemplary human CD28-derived hinges:
(SEQ ID NO: 244)
IEVMYPPPYLDNERSNGTIIHVKGKHLCPSPLFPGPSKP
(SEQ ID NO: 245)
GAAAIEVMYPPPYLDNEKSNGTIIHVKGKHLCPSPLFPGPSKP

### Example 16—Exemplary Transmembrane Domains

This Example depicts the amino acid sequences of exemplary transmembrane domains that can be used to design a CAR.

Exemplary transmembrane domains for CARs
Exemplary human CD3ζ transmembrane domain:
(SEQ ID NO: 246)
LCYLLDGILFIYGVILTALFL
Exemplary human CD4 transmembrane domain:
(SEQ ID NO: 247)
MALIVLGGVAGLLLFIGLGIFF
Exemplary human CD8α transmembrane domains:
(SEQ ID NO: 836)
IYIWAPLAGTCGVLLLSLVIT
(SEQ ID NO: 249)
IYIWAPLAGTCGVLLLSLVITLYC
(SEQ ID NO: 250)
IWAPLAGTCGVLLLSLVITLYC
(SEQ ID NO: 251)
IWAPLAGTCGVLLLSLVIT
Exemplary human CD28 transmembrane domain:
(SEQ ID NO: 252)
FWVLVVVGGVLACYSLLVTVAFIIFWV
Exemplary human CD278 transmembrane domain:
(SEQ ID NO: 253)
FWLPIGCAAFVVVCILGCILI

### Example 17—Exemplary Intracellular Signaling Domains

This Example depicts the amino acid sequences of exemplary intracellular signaling domains that can be used to design a CAR.

Exemplary intracellular signaling domains for CARS
Exemplary human CD3ζ intracellular signaling
domains:
(SEQ ID NO: 254)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR
(SEQ ID NO: 255)
RVKFSRSADAPAYKQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR
(SEQ ID NO: 256)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
Exemplary human 4-1BB (CD137) intracellular
signaling domains:
(SEQ ID NO: 257)
KRGRKKLLYIFKQPFMRPVQTTQEEDGCSCRFPEEEEGGCEL
(SEQ ID NO: 258)
KRGRKKLLYIFKQPFMRPVQTTQEEDGCCRFPEEEE
Exemplary human CD28 intracellular signaling
domain:
(SEQ ID NO: 259)
RSKRSRLLHSDYMNMTPRRPGPTRKHYQPYAPPRDFAAYRS
Exemplary human OX40 (CD134) intracellular
signaling domain:
(SEQ ID NO: 260)
ALYLLRRDQRLPPDAHKPPGGGSFRTPIQEEQADAHSTLAKI
Exemplary human CD278 intracellular signaling
domain:
(SEQ ID NO: 261)
CWLTKKKYSSSVHDPNGEYMFMRAVNTAKKSRLTDVTL
Exemplary human DAP10 intracellular signaling
domain:
(SEQ ID NO: 262)
LCARPRRSPAQEDGKVYINMPGRG
Exemplary human DAP12 intracellular signaling
domain:
(SEQ ID NO: 263)
YFLGRLVPRGRGAAEAATRKQRITETESPYQELQGQRSDVYSDLNTQRPY
YK
Exemplary human CD27 intracellular signaling
domain:
(SEQ ID NO: 264)
QRRKYRSNKGESPVEPAEPCHYSCPREEEGSTIPIQEDYRKPEPACSP

### Example 18—Exemplary CARs

This Example depicts the structures and sequences of exemplary CARs having the ability to bind to a human PSCA polypeptide.

The various components of each CAR (e.g., domains and linkers) are provided and delineated.

A CAR designed using CDRs of Clone #1 (CAR Clone #1A):
CD8a Signal Peptide + scFv using CDRs of Clone #1 + CD8a Hinge + CD8a
Transmembrane Domain + 4-1BB Intracellular Signaling Domain + CD3ζ
Intracellular Signaling Domain
(SEQ ID NO: 265)
MALPVTALLLPLALLLHAARPQVQLVESGGGLVKPGGSLRLSCAASGFTFSDY
YMSWIRQAPGKGLEWVSYISSSGSTIYYADSVKGRFTISRDNAKNSLYLQMN
SLRAEDTAVYYCARPLRWGMDVWGQGTTVTVSSGGGGSGGGGSGGGGSDI
QMTQSPSSLSASVGDRVTITCRASQSISSYLNWYQQKPGKAPKLLIYAASSLQ
SGVPSRFSGSGSGTDFTLTISSLQPEDAATYYCHQSSSLPLTFGGGTKVEIKTT
TPAPRPPTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
A CAR designed using CDRs of Clone #2 (CAR Clone #2A):
CD8a Signal Peptide + scFv using CDRs of Clone #2 + CD8a Hinge + CD8a
Transmembrane Domain + 4-1BB Intracellular Signaling Domain + CD3ζ
Intracellular Signaling Domain
(SEQ ID NO: 266)
MALPVTALLLPLALLLHAARPQVQLVESGGGLVKPGGSLRLSCAASGFTFSDY
YMSWIRQAPGKGLEWVSYISSSGSTIYYADSVKGRFTISRDNAKNSLYLQMN
SLRAEDTAVYYCARPLRWGMDVWGQGTTVTVSSGGGGSGGGGSGGGGSDI
QMTQSPSSLSASVGDRVTITCRASQSISSYLNWYQQKPGKAPKLLIYAASSLQ
SGVPSRFSGSGSGTDFTLTISSLQPEDFAVYYCHQSSSLPLTFGGGTKLEIKTTT
PAPRPPTPAPTIASQPLSLRPEACRPAAGGAVHTRGLDFACDIYIWAPLAGTCGVL
LLSLVITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR
A CAR designed using CDRs of Clone #3 (CAR Clone #3A):
CD8a Signal Peptide + scFv using CDRs of Clone #3 + CD8a Hinge + CD8a
Transmembrane Domain + 4-1BB Intracellular Signaling Domain + CD3ζ
Intracellular Signaling Domain
(SEQ ID NO: 267)
MALPVTALLLPLALLLHAARPQVQLVESGGGLVKPGGSLRLSCAASGFTFSDY
YMSWIRQAPGKGLEWVSYISSSGSTIYYADSVKGRFTISRDNAKNSLYLQMN
SLRAEDTAVYYCARPLRWGMDVWGQGTTVTVSSGGGGSGGGGSGGGGSDI
QMTQSPSSLSASVGDRVTITCRASQSISSYLNWYQQKPGKAPKLLIYAASSLQ
SGVPSRFSGSGSGTDFTLTISSLQPEDVAVYYCHQSNSLPLTFGGGTKVEIKTT
TPAPRPPTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
A CAR designed using CDRs of Clone #4 (CAR Clone #4A):
CD8a Signal Peptide + scFv using CDRs of Clone #4 + CD8a Hinge + CD8a
Transmembrane Domain + 4-1BB Intracellular Signaling Domain + CD3ζ
Intracellular Signaling Domain
(SEQ ID NO: 268)
MALPVTALLLPLALLLHAARPQVQLVESGGGLVKPGGSLRLSCAASGFTFSDY
YMSWIRQAPGKGLEWVSYISSSGSTIVYADSVKGRFTISRDNAKNSLYLQMN
SLRAEDTAVYYCARPIRRGMDVWGQGTTVTVSSGGGGSGGGGSGGGGSDIQ
MTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIKTTT
PAPRPPTPAPTIASQPLSLRPEACRPAAGGAVHTRGLDFACDIYIWAPLAGTCGVL
LLSLVITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR
A CAR designed using CDRs of Clone #5 (CAR Clone #5A):
CD8a Signal Peptide + scFv using CDRs of Clone #5 + CD8a Hinge + CD8a
Transmembrane Domain + 4-1BB Intracellular Signaling Domain + CD3ζ
Intracellular Signaling Domain
(SEQ ID NO: 269)
MALPVTALLLPLALLLHAARPQVQLVESGGGLVKPGGSLRLSCAASGFTFSDY
YMSWIRQAPGKGLEWVSYISSSGSTINYADSVKGRFTISRDNAKNSLYLQMN
SLRAEDTAVYYCARPIRWGMDVWGQGTTVTVSSGGGGSGGGGSGGGGSDI
QMTQSPSSLSASVGDRVTITCRASQSISSYLNWYQQKPGKAPKLLIYAASSLQ
SGVPSRFSGSGSGTDFTLTISSLQPEDAATYYCHQSSSLPLTFGGGTKVEIKTT
TPAPRPPTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
A CAR designed using CDRs of Clone #6 (CAR Clone #6A):
CD8a Signal Peptide + Clone #6 scFv + CD8a Hinge + CD8a Transmembrane
Domain + 4-1BB Intracellular Signaling Domain + CD3ζ Intracellular
Signaling Domain
(SEQ ID NO: 270)
MALPVTALLLPLALLLHAARPEVQLVQSGAEVKKPGSSVKVSCKASGGTFSSY
AISWVRQAPGQGLEWMGGIIPIFGTANYAQKFQGRVTITADESTSTAYMELSS
LRSDDTAVYYCARERIAVAGTNYYYGMDVWGQGTLVTVSSGGGGSGGGGS
GGGGSEIVLTQSPATLSVSPGERATLSCRASQSVSSSNLAWYQQKPGQAPRLL
IYGASTRATGIPARFSGTGSGTDFTLTISRLEPEDFAVYYCQQYGSSITFGQGT
RLEIKTTTPAPRPPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR
A CAR designed using CDRs of Clone #7 (CAR Clone #7A):
CD8a Signal Peptide + Clone #7 scFv + CD8a Hinge + CD8a Transmembrane
Domain + 4-1BB Intracellular Signaling Domain + CD3ζ Intracellular
Signaling Domain
(SEQ ID NO: 271)
MALPVTALLLPLALLLHAARPEVQLVESGAEVKKPGASVKVSCKASGYTFTGY
YMHWVRQAPGQGLVWMGRINPNSGGTNYAQKFQGRVTMTRDTSISTAYME
LSRLRSDDTAVYYCAFGTRHAFDIWGQGTMVTVSSGGGGSGGGGSGGGGS
NFMLTQPHSVSESPGKTVTISCTRSSGSIASNYVQWYQQRPGSAPTTVIYEDN
QRPSGVPDRFSGSIDSSSNSASLTISGLKTEDEADYYCQSYDSIVHVVFGGGTK
VTVLGTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMG
GKPRRKNPQEGLYNELQKDKMAEAYSEIGMKGERRRGKGHIDGLYQGLSTA
TKDTYDALHMQALPPR
A CAR designed using CDRs of Clone #8 (CAR Clone #8A):
CD8a Signal Peptide + Clone #8 scFv + CD8a Hinge + CD8a Transmembrane
Domain + 4-1BB Intracellular Signaling Domain + CD3ζ Intracellular
Signaling Domain
(SEQ ID NO: 272)
MALPVTALLLPLALLLHAARPEVQLVQSGAEVKKPGASVKVSCKASGGTFSSY
AISWVRQAPGQGLEWMGGIIPIFGTANYAQKFQGRVTITADKSTSTAYMELS
SLRSEDTAVYYCARDVIVGATSSGMDVWGQGTTVTVSSGGGGSGGGGSGGG
GSDIQMTQSPSSVSASVGDRVTITCRASQSISRYLNWYQQKPGKAPKLLIYGA
SSLQSGVPSRFSGSGFGTDFTLTISSLQPEDFATYYCQQSYSTPLTFGQGTKLE
IKTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCE
LRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
A CAR designed using CDRs of Clone #1 (CAR Clone #1B):
CD8a Signal Peptide + scFv using CDRs of Clone #1 + CD28 Hinge, CD28
Transmembrane Domain, and CD28 Intracellular Signaling Domain + CD3ζ
Intracellular Signaling Domain
(SEQ ID NO: 273)
MALPVTALLLPLALLLHAARPQVQLVESGGGLVKPGGSLRLSCAASGFTFSDY
YMSWIRQAPGKGLEWVSYISSSGSTIYYADSVKGRFTISRDNAKNSLYLQMN
SLRAEDTAVYYCARPLRWGMDVWGQGTTVTVSSGGGGGGGGSGGGGSDI
QMTQSPSSLSASVGDRVTITCRASQSISSYLNWYQQKPGKAPKLLIYAASSLQ
SGVPSRESGSGSGTDFTLTISSLQPEDAATYYCHQSSSLPLTFGGGTKVEIKGA
AAIEVMYPPPYLDNEKSNGTIIHVKGKHLCPSPLFPGPSKPFWVLVVVGGVLACY
SLLVTVAFIIFWVRSKRSRLLHSDYMNMTPRRPGPTRKHYQPYAPPRDFAAYRSR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR
A CAR designed using CDRs of Clone #2 (CAR Clone #2B):
CD8a Signal Peptide + scFv using CDRs of Clone #2 + CD28 Hinge, CD28
Transmembrane Domain, and CD28 Intracellular Signaling Domain + CD3ζ
Intracellular Signaling Domain
(SEQ ID NO: 274)
MALPVTALLLPLALLLHAARPQVQLVESGGGLVKPGGSLRLSCAASGFTFSDY
YMSWIRQAPGKGLEWVSYISSSGSTIYYADSVKGRFTISRDNAKNSLYLQMN
SLRAEDTAVYYCARPLRWGMDVWGQGTTVTVSSGGGGSGGGGSGGGGSDI
QMTQSPSSLSASVGDRVTITCRASQSISSYLNWYQQKPGKAPKLLIYAASSLQ
SGVPSRFSGSGSGTDFTLTISSLQPEDFAVYYCHQSSSLPLTFGGGTKLEIKGA
AAIEVMYPPPYLDNEKSNGTIIHVKGKHLCPSPLFPGPSKPFWVLVVVGGVLACY
SLLVTVAFIIFWVRSKRSRLLHSDYMNMTPRRPGPTRKHYQPYAPPRDFAAYRSR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR
A CAR designed using CDRs of Clone #3 (CAR Clone #3B):
CD8a Signal Peptide + scFv using CDRs of Clone #3 + CD28 Hinge, CD28
Transmembrane Domain, and CD28 Intracellular Signaling Domain + CD3ζ
Intracellular Signaling Domain
(SEQ ID NO: 275)
MALPVTALLLPLALLLHAARPQVQLVESGGGLVKPGGSLRLSCAASGFTFSDY
YMSWIRQAPGKGLEWVSYISSSGSTIYYADSVKGRFTISRDNAKNSLYLQMN
SLRAEDTAVYYCARPLRWGMDVWGQGTTVTVSSGGGGSGGGGSGGGGSDI
QMTQSPSSLSASVGDRVTITCRASQSISSYLNWYQQKPGKAPKLLIYAASSLQ
SGVPSRFSGSGSGTDFTLTISSLQPEDVAVYYCHQSNSLPLTFGGGTKVEIKGA
AAIEVMYPPPYLDNEKSNGTIIHVKGKHLCPSPLFPGPSKPFWVLVVVGGVLACY
SLLVTVAFIIFWVRSKRSRLLHSDYMNMTPRRPGPTRKHYQPYAPPRDFAAYRSR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR
A CAR designed using CDRs of Clone #4 (CAR Clone #4B):
CD8a Signal Peptide + scFv using CDRs of Clone #4 + CD28 Hinge, CD28
Transmembrane Domain, and CD28 Intracellular Signaling Domain + CD3ζ
Intracellular Signaling Domain
(SEQ ID NO: 276)
MALPVTALLLPLALLLHAARPQVQLVESGGGLVKPGGSLRLSCAASGFTFSDY
YMSWIRQAPGKGLEWVSYISSSGSTIVYADSVKGRFTISRDNAKNSLYLQMN
SLRAEDTAVYYCARPIRRGMDVWGQGTTVTVSSGGGGSGGGGSGGGGSDIQ
MTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIKGAA
AIEVMYPPPYLDNEKSNGTIIHVKGKHLCPSPLFPGPSKPFWVLVVVGGVLACYS
LLVTVAFIIFWVRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
A CAR designed using CDRs of Clone #5 (CAR Clone #5B):
CD8a Signal Peptide + scFv using CDRs of Clone #5 + CD28 Hinge, CD28
Transmembrane Domain, and CD28 Intracellular Signaling Domain + CD3ζ
Intracellular Signaling Domain
(SEQ ID NO: 277)
MALPVTALLLPLALLLHAARPQVQLVESGGGLVKPGGSLRLSCAASGFTFSDY
YMSWIRQAPGKGLEWVSYISSSGSTINYADSVKGRFTISRDNAKNSLYLQMN
SLRAEDTAVYYCARPIRWGMDVWGQGTTVTVSSGGGGSGGGGSGGGGSDI
QMTQSPSSLSASVGDRVTITCRASQSISSYLNWYQQKPGKAPKLLIYAASSLQ
SGVPSRFSGSGSGTDFTLTISSLQPEDAATYYCHQSSSLPLTFGGGTKVEIKGA
AAIEVMYPPPYLDNEKSNGTIIHVKGKHLCPSPLFPGPSKPFWVLVVVGGVLACY
SLLVTVAFIIFWVRSKRSRLLHSDYMNMTPRRPGPTRKHYQPYAPPRDFAAYRSR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR
A CAR designed using CDRs of Clone #6 (CAR Clone #6B):
CD8a Signal Peptide + Clone #6 scFv + CD28 Hinge, CD28 Transmembrane Domain,
and CD28 Intracellular Signaling Domain + CD3ζ Intracellular Signaling Domain
(SEQ ID NO: 278)
MALPVTALLLPLALLLHAARPEVQLVQSGAEVKKPGSSVKVSCKASGGTFSSY
AISWVRQAPGQGLEWMGGIIPIFGTANYAQKFQGRVTITADESTSTAYMELSS
LRSDDTAVYYCARERIAVAGTNYYYGMDVWGQGTLVTVSSGGGGSGGGGS
GGGGSEIVLTQSPATLSVSPGERATLSCRASQSVSSSNLAWYQQKPGQAPRLL
IYGASTRATGIPARFSGTGSGTDFTLTISRLEPEDFAVYYCQQYGSSITFGQGT
RLEIKGAAAIEVMYPPPYLDNEKSNGTIIHVKGKHLCPSPLFPGPSKPFWVLVVV
GGVLACYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR
A CAR designed using CDRs of Clone #7 (CAR Clone #7B):
CD8a Signal Peptide + Clone #7 scFv + CD28 Hinge, CD28 Transmembrane Domain,
and CD28 Intracellular Signaling Domain + CD3ζ Intracellular Signaling Domain
(SEQ ID NO: 279)
MALPVTALLLPLALLLHAARPEVQLVESGAEVKKPGASVKVSCKASGYTFTGY
YMHWVRQAPGQGLVWMGRINPNSGGTNYAQKFQGRVTMTRDTSISTAYME
LSRLRSDDTAVYYCAFGTRHAFDIWGQGTMVTVSSGGGGSGGGGSGGGGS
NFMLTQPHSVSESPGKTVTISCTRSSGSIASNYVQWYQQRPGSAPTTVIYEDN
QRPSGVPDRFSGSIDSSSNSASLTISGLKTEDEADYYCQSYDSIVHVVFGGGTK
VTVLGGAAAIEVMYPPPYLDNEKSNGTIIHVKGKHLCPSPLFPGPSKPFWVLVVV
GGVLACYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR
A CAR designed using CDRs of Clone #8 (CAR Clone #8B):
CD8a Signal Peptide + Clone #8 scFv + CD28 Hinge, CD28 Transmembrane Domain,
and CD28 Intracellular Signaling Domain + CD3ζ Intracellular Signaling Domain
(SEQ ID NO: 280)
MALPVTALLLPLALLLHAARPEVQLVQSGAEVKKPGASVKVSCKASGGTFSSY
AISWVRQAPGQGLEWMGGIIPIFGTANYAQKFQGRVTITADKSTSTAYMELS
SLRSEDTAVYYCARDVIVGATSSGMDVWGQGTTVTVSSGGGGSGGGGSGGG
GSDIQMTQSPSSVSASVGDRVTITCRASQSISRYLNWYQQKPGKAPKLLIYGA
SSLQSGVPSRFSGSGFGTDFTLTISSLQPEDFATYYCQQSYSTPLTFGQGTKLE
IKGAAAIEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAPPRDFAA
YRSRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR

### Example 19—Exemplary Components that can be Used to Design BiTEs

This Example depicts exemplary components that can be used to design cell engagers that bind to T cells.

Exemplary linkers that can be used to design cell engagers
that bind to T cells
An amino acid sequence of a linker sequence (SEQ ID NO: 135;
nucleic acid sequence of the linker is underlined in SEQ ID
NO: 282) can be followed by an gOKT3-7 scFv sequence, which
can be attached to a light chain as shown in FIG. 3. Also
depicted is a nucleic acid sequence encoding that linker and
gOKT3-7 scFv.
Linker + gOKT3-7 (anti-CD3 scFv)
(SEQ ID NO: 281)
GGGGSGGGGSGGGGSQVQLVQSGGGVVQPGRSLRLSCKASGYTFTRYTMHWV
RQAPGKGLEWIGYINPSRGYTNYNQKVKDRFTISRDNSKNTAFLQMDSLRPEDT
GVYFCARYYDDHYCLDYWGQGTPVTVSSGGGGSGGGGSGGGGSGGGGSGGGG
SGGGGSDIQMTQSPSSLSASVGDRVTITCSASSSVSYMNWYQQTPGKAPKRWIY
DTSKLASGVPSRFSGSGSGTDYTFTISSLQPEDIATYYCQQWSSNPFTFGQGTKLQ
ITR
(SEQ ID NO: 282)
GGAGGTGGCGGGTCTGGTGGAGGCGGAAGCGGTGGTGGCGGATCCCAGGTC
CAACTGGTGCAAAGTGGAGGCGGCGTAGTACAGCCCGGTCGGTCCCTGCGGC
TCAGCTGTAAAGCAAGTGGGTACACTTTCACCAGATATACAATGCACTGGGT
TAGACAGGCCCCCGGCAAGGGTCTTGAATGGATAGGATATATCAACCCATCC
CGCGGATATACAAACTACAATCAGAAGGTGAAGGACCGGTTCACGATTAGTA
GAGACAACTCCAAAAACACTGCGTTCCTTCAAATGGACTCATTGCGACCCGA
GGACACTGGAGTGTATTTCTGTGCCAGGTATTATGACGACCATTATTGCCTGG
ACTACTGGGGGCAAGGCACACCGGTTACGGTATCTTCCGGAGGTGGCGGGTC
TGGTGGAGGCGGAAGCGGTGGTGGCGGATCCGGGGGAGGAGGCTCTGGCGG
AGGCGGATCAGGGGGTGGAGGTTCCGACATTCAAATGACACAATCCCCTAGT
TCTTTGTCTGCGTCCGTGGGGGATCGGGTAACAATCACTTGCTCTGCATCAAG
CAGTGTTTCTTATATGAACTGGTATCAGCAGACACCCGGTAAGGCCCCAAAA
CGATGGATTTATGACACGAGTAAACTCGCATCCGGGGTGCCTTCACGCTTCTC
CGGTTCTGGTTCCGGCACGGATTACACTTTCACCATCAGTAGCCTGCAACCTG
AGGACATTGCAACTTATTACTGTCAACAATGGAGCAGTAATCCATTCACGTTC
GGCCAAGGGACAAAACTGCAGATTACTAGG
Exemplary antigen binding domains that bind to T cells
Exemplary anti-human CD3 scFv (clone OKT3):
Heavy chain:
(SEQ ID NO: 837)
QVQLVQSGGGVVQPGRSLRLSCKASGYTFTRYTMHWVRQAPGKGLEWIGYINP
SRGYTNYNQKVKDRFTISRDNSKNTAFLQMDSLRPEDTGVYFCARYYDDHYCL
DYWGQGTPVTVSS
Light chain:
(SEQ ID NO: 283)
DIQMTQSPSSLSASVGDRVTITCSASSSVSYMNWYQQTPGKAPKRWIYDTSKLAS
GVPSRFSGSGSGTDYTFTISSLQPEDIATYYCQQWSSNPFTFGQGTKLQITR
Exemplary anti-human CD3 scFv (clone UCHT1):
Heavy chain:
(SEQ ID NO: 284)
EVQLQQSGPELVKPGASMKISCKASGYSFTGYTMNWVKQSHGKNLEWMGLINP
YKGVSTYNQKFKDKATLTVDKSSSTAYMELLSLTSEDSAVYYCARSGYYGDSDW
YFDVWGQGTTLTVFS
Light chain:
(SEQ ID NO: 285)
MDIQMTQTTSSLSASLGDRVTISCRASQDIRNYLNWYQQKPDGTVKLLIYYTSRL
HSGVPSKFSGSGSGTDYSLTISNLEQEDIATYFCQQGNTLPWTFAGGTKLEIK

### Example 20—Exemplary Components that can be Used to Design BiKEs

This Example depicts exemplary components that can be used to design cell engagers that bind to NK cells.

Exemplary antigen binding domains that bind to NK cells
VH7:
(SEQ ID NO: 835)
EVQLVESGGGLVQPGGSLRLSCAASGFTFSNYGMSWIRQAPGKGLEWIGSIYYSG
STIYNPSLKSLVTISRDNSKNTLYLQMNSLRAEDTATYYCARESIDYWGQGTLVTV
SS
Exemplary anti-human CD16a scFv:
Heavy chain:
(SEQ ID NO: 286)
EVQLVESGGGVVRPGGSLRLSCAASGFTFDDYGMSWVRQAPGKGLEWVSGINW
NGGSTGYADSVKGRFTISRDNAKNSLYLQMNSLRAEDTAVYYCARGRSLLFDYW
GQGTLVTVSR
Light chain:
(SEQ ID NO: 287)
SSELTQDPAVSVALGQTVRITCQGDSLRSYYASWYQQKPGQAPVLVIYGKNNRPS
GIPDRFSGSSSGNTASLTITGAQAEDEADYYCNSRDSSGNHVVFGGGTKLTVG
Exemplary anti-human NKG2A scFv:
Heavy chain:
(SEQ ID NO: 288)
QVQLVQSGAEVKKPGASVKVSCKASGYTFTSYWMNWVRQAPGQGLEWMGRID
PYDSETHYAQKLQGRVTMTTDTSTSTAYMELRSLRSDDTAVYYCARGGYDFDVG
TLYWFFDVWGQGTTVTVSS
Light chain:
(SEQ ID NO: 289)
DIQMTQSPSSLSASVGDRVTITCRASENIYSYLAWYQQKPGKAPKLLIYNAKTLA
EGVPSRFSGSGSGTDFTLTISSLQPEDFATYYCQHHYGTPRTFGGGTKVEIK
Exemplary anti-human NKG2A scFv:
Heavy chain:
(SEQ ID NO: 290)
EVQLVESGGGLVKPGGSLRLSCAASGFTFSSYAMSWVRQAPGKGLEWVSEISS
GGSYTYYADSVKGRFTISRDNAKNSLYLQMNSLRAEDTAVYYCARHGDYPRFF
DVWGQGTTVTVSS
Light chain:
(SEQ ID NO: 291)
EIVLTQSPATLSLSPGERATLSCSASSSVSSYIYWYQQKPGQAPRLLIYLTSNLASGI
PARFSGSGSGTDFTLTISSLEPEDFAVYYCQQWSGNPYTFGQGTKLEIK
Exemplary anti-human NKG2D scFv:
Heavy chain:
(SEQ ID NO: 292)
QVQLVESGGGLVKPGGSLRLSCAASGFTFSSYGMHWVRQAPGKGLEWVAFIRYD
GSNKYYADSVKGRFTISRDNSKNTLYLQMNSLRAEDTAVYYCAKDRGLGDGTYF
DYWGQGTTVTVSS
Light chain:
(SEQ ID NO: 293)
QSALTQPASVSGSPGQSITISCSGSSSNIGNNAVNWYQQLPGKAPKLLIYYDDLLPS
GVSDRFSGSKSGTSAFLAISGLQSEDEADYYCAAWDDSLNGPVFGGGTKLTVL
Exemplary anti-human NKp30 scFv:
Alternative heavy chains with underline indicating the differences:
(SEQ ID NO: 294)
QVQLVQSGAEVKKPGASVKVSCKASGHTFTSYFMHWVRQAPGQGLEWMGIINP
SDDYANYAQKFQGRVTMTRDTSTSTVYMELSSLRSEDTAVYYCATAIFDYWGQG
TLVTVSS
or
(SEQ ID NO: 295)
QVQLVQSGAEVKKPGASVKVSCKASGHTFTSYFMHWVRQAPGQGLEWMGIINP
SDDYANYAQKFQGRVTMTRDTSTSTVYMELSSLRSEDTAVYYCATAIFDYWGQG
TPVTVSS
Light chain:
(SEQ ID NO: 296)
DIQMTQSPSSLSASVGDRVTITCRASQSISSYLNWYQQKPGKAPKLLIYAASSLQS
GVPSRFSGSGSGTDFTLTISSLQPEDFATYYCQQSYSTPLTFGGGTKVEIK
Exemplary anti-human NKp46 scFv
Heavy chain:
(SEQ ID NO: 297)
EIQLQQSGAELVKPGASVKLSCTASGFNIKDTYFHWVKQRPEQGLEWIGRIDPAN
GNTKYDPKFHDKATIIADISSNTAYLQFSSLTSEDTAVYYCAANRYGYWGQGTTLT
VSS
Light chain:
(SEQ ID NO: 298)
DIVMTQAAPSIPVTPGESVSISCRSSKSLLYINGNTHLFWFLQRPGQSPQLLIYRMS
NLASGVPDRFSGSGSGTAFTLRISRVEAEDVGVYYCMQHLEYPFTFGSGTKLEIK
Exemplary anti-human NKp46 scFv
Heavy chain:
(SEQ ID NO: 299)
QVQLQQSGPELVKPGASVKMSCKASGYTFTDYVINWGKQRSGQGLEWIGEIYPG
SGTNYYNEKFKAKATLTADKSSNIAYMQLSSLTSEDSAVYFCARRGRYGLYAMDY
WGQGTSVTVSS
Light chain:
(SEQ ID NO: 300)
DIQMTQTTSSLSASLGDRVTISCRASQDISNYLNWYQQKPDGTVKLLIYYTSRLHS
GVPSRFSGSGSGTDYSLTINNLEQEDIATYFCQQGNTRPWTFGGGTKLEIK
Exemplary anti-human CD16a VH domain:
(SEQ ID NO: 301)
EVQLVESGGGLVQPGGSLRLSCAASGFTFSNYGMSWVRQAPGKGLEWIGSIYYS
GSTNYNPSLKSLVTISRDNSKNTLYLQMNSLRAEDTATYYCARESIDYWGQGTLV
TVSS

### Example 21—Exemplary BiKEs that Bind to PSCA and NK Cells

This Example depicts the structures and sequences of exemplary BiKEs designed to include an IgG having the CDRs of clones #1-#8.

The various components of each BiKE (e.g., domains and linkers) are provided and delineated.

Exemplary 1 + 2 BiKE that binds to PSCA and NK cells (designated BiKE #1A):
Anti-CD16 VH7 Binder + Linker + Variable Domain of Heavy Chain of Clone #1 +
CH1 + Hinge + CH2 + CH3 (hole)
(SEQ ID NO: 302)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGGGGGSQVQLVESGGGLVKPGGSLRLSCAASGFTFSDYY
MSWIRQAPGKGLEWVSYISSSGSTIYYADSVKGRFTISRDNAKNSLYLQMNSLRA
EDTAVYYCARPLRWGMDVWGQGTTVTVSSASTKGPSVFPLAPSSKSTSGGTA
ALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVC
TLPPSRDELTKNQVSLSCAVKGFYPSDIAVEWESNGQPENNYKTTPPVLDSDGSF
FLVSKLTVDKSRWQQGNVFSCSVMHEALHNRFTQKSLSLSPGK
Variable Domain of Heavy Chain of Clone #1 + CH1 + Hinge + CH2 + CH3 (knob)
(SEQ ID NO: 303)
QVQLVESGGGLVKPGGSLRLSCAASGFTFSDYYMSWIRQAPGKGLEWVSYISSS
GSTIYYADSVKGRFTISRDNAKNSLYLQMNSLRAEDTAVYYCARPLRWGMDVW
GQGTTVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNG
KEYKCKVSNKALGAPIEKTISKAKGQPREPQVYTLPPCRDELTKNQVSLWCLV
KGFYPSDIAVEWESNGQPENNYKTTPPVLDSDGSFFLYSKLTVDKSRWQQGNVF
SCSVMHEALHNHYTQKSLSLSPGK
Variable Domain of Light Chain of Clone #1 + Constant Domain of Light Chain
(SEQ ID NO: 304)
DIQMTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Exemplary 1 + 2 BiKE that binds to PSCA and NK cells (designated BiKE #2A):
Anti-CD16 VH7 Binder + Linker + Variable Domain of Heavy Chain of Clone #2 +
CH1 + Hinge + CH2 + CH3 (hole)
(SEQ ID NO: 305)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSQVQLVESGGGLVKPGGSLRLSCAASGFTFSDYY
MSWIRQAPGKGLEWVSYISSSGSTIYYADSVKGRFTISRDNAKNSLYLQMNSLRA
EDTAVYYCARPLRWGMDVWGQGTTVTVSSASTKGPSVFPLAPSSKSTSGGTA
ALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVC
TLPPSRDELTKNQVSLSCAVKGFYPSDIAVEWESNGQPENNYKTTPPVLDSDGSF
FLVSKLTVDKSRWQQGNVFSCSVMHEALHNRFTQKSLSLSPGK
Variable Domain of Heavy Chain of Clone #2 + CH1 + Hinge + CH2 + CH3 (knob)
(SEQ ID NO: 306)
QVQLVESGGGLVKPGGSLRLSCAASGFTFSDYYMSWIRQAPGKGLEWVSYISSS
GSTIYYADSVKGRFTISRDNAKNSLYLQMNSLRAEDTAVYYCARPLRWGMDVW
GQGTTVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNG
KEYKCKVSNKALGAPIEKTISKAKGQPREPQVYTLPPCRDELTKNQVSLWCLV
KGFYPSDIAVEWESNGQPENNYKTTPPVLDSDGSFFLYSKLTVDKSRWQQGNVF
SCSVMHEALHNHYTQKSLSLSPGK
Variable Domain of Light Chain of Clone #2 + Constant Domain of Light Chain
(SEQ ID NO: 307)
DIQMTQSPSSLSASVGDRVTITCRASQSISSYLNWYQQKPGKAPKLLIYAASSLQS
GVPSRFSGSGSGTDFTLTISSLQPEDFAVYYCHQSSSLPLTFGGGTKLEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Exemplary 1 + 2 BiKE that binds to PSCA and NK cells (designated BiKE #3A):
Anti-CD16 VH7 Binder + Linker + Variable Domain of Heavy Chain of Clone #3 +
CH1 + Hinge + CH2 + CH3 (hole)
(SEQ ID NO: 308)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSQVQLVESGGGLVKPGGSLRLSCAASGFTFSDYY
MSWIRQAPGKGLEWVSYISSSGSTIYYADSVKGRFTISRDNAKNSLYLQMNSLRA
EDTAVYYCARPLRWGMDVWGQGTTVTVSSASTKGPSVFPLAPSSKSTSGGTA
ALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVC
TLPPSRDELTKNQVSLSCAVKGFYPSDIAVEWESNGQPENNYKTTPPVLDSDGSF
FLVSKLTVDKSRWQQGNVFSCSVMHEALHNRFTQKSLSLSPGK
Variable Domain of Heavy Chain of Clone #3 + CH1 + Hinge + CH2 + CH3 (knob)
(SEQ ID NO: 309)
QVQLVESGGGLVKPGGSLRLSCAASGFTFSDYYMSWIRQAPGKGLEWVSYISSS
GSTIYYADSVKGRFTISRDNAKNSLYLQMNSLRAEDTAVYYCARPLRWGMDVW
GQGTTVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNG
KEYKCKVSNKALGAPIEKTISKAKGQPREPQVYTLPPCRDELTKNQVSLWCLV
KGFYPSDIAVEWESNGQPENNYKTTPPVLDSDGSFFLYSKLTVDKSRWQQGNVF
SCSVMHEALHNHYTQKSLSLSPGK
Variable Domain of Light Chain of Clone #3 + Constant Domain of Light Chain
(SEQ ID NO: 310)
DIQMTQSPSSLSASVGDRVTITCRASQSISSYLNWYQQKPGKAPKLLIYAASSLQS
GVPSRFSGSGSGTDFTLTISSLQPEDVAVYYCHQSNSLPLTFGGGTKVEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFFNRGEC
Exemplary 1 + 2 BiKE that binds to PSCA and NK cells (designated BiKE #4A):
Anti-CD16 VH7 Binder + Linker + Variable Domain of Heavy Chain of Clone #4 +
CH1 + Hinge + CH2 + CH3 (hole)
(SEQ ID NO: 311)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGGGGGSGGGGSQVQLVESGGGLVKPGGSLRLSCAASGFTFSDYY
MSWIRQAPGKGLEWVSYISSSGSTIVYADSVKGRFTISRDNAKNSLYLQMNSLRA
EDTAVYYCARPIRRGMDVWGQGTTVTVSSASTKGPSVFPLAPSSKSTSGGTAA
LGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNS
TYRVVSVLTVLHQDWLNGKEYKCKVSNKALGAPIEKTISKAKGQPREPQVC
TLPPSRDELTKNQVSLSCAVKGFYPSDIAVEWESNGQPENNYKTTPPVLDSDGSF
FLVSKLTVDKSRWQQGNVFSCSVMHEALHNRFTQKSLSLSPGK
Variable Domain of Heavy Chain of Clone #4 + CH1 + Hinge + CH2 + CH3 (knob)
(SEQ ID NO: 312)
QVQLVESGGGLVKPGGSLRLSCAASGFTFSDYYMSWIRQAPGKGLEWVSYISSS
GSTIVYADSVKGRFTISRDNAKNSLYLQMNSLRAEDTAVYYCARPIRRGMDVW
GQGTTVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNG
KEYKCKVSNKALGAPIEKTISKAKGQPREPQVYTLPPCRDELTKNQVSLWCLV
KGFYPSDIAVEWESNGQPENNYKTTPPVLDSDGSFFLYSKLTVDKSRWQQGNVF
SCSVMHEALHNHYTQKSLSLSPGK
Variable Domain of Light Chain of Clone #4 + Constant Domain of Light Chain
(SEQ ID NO: 313)
DIQMTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Exemplary 1 + 2 BiKE that binds to PSCA and NK cells (designated BiKE #5A):
Anti-CD16 VH7 Binder + Linker + Variable Domain of Heavy Chain of Clone #5 +
CH1 + Hinge + CH2 + CH3 (hole)
(SEQ ID NO: 314)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSQVQLVESGGGLVKPGGSLRLSCAASGFTFSDYY
MSWIRQAPGKGLEWVSYISSSGSTINYADSVKGRFTISRDNAKNSLYLQMNSLRA
EDTAVYYCARPIRWGMDVWGQGTTVTVSSASTKGPSVFPLAPSSKSTSGGTAA
LGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNS
TYRVVSVLTVLHQDWLNGKEYKCKVSNKALGAPIEKTISKAKGQPREPQVC
TLPPSRDELTKNQVSLSCAVKGFYPSDIAVEWESNGQPENNYKTTPPVLDSDGSF
FLVSKLTVDKSRWQQGNVFSCSVMHEALHNRFTQKSLSLSPGK
Variable Domain of Heavy Chain of Clone #5 + CH1 + Hinge + CH2 + CH3 (knob)
(SEQ ID NO: 315)
QVQLVESGGGLVKPGGSLRLSCAASGFTFSDYYMSWIRQAPGKGLEWVSYISSS
GSTINYADSVKGRFTISRDNAKNSLYLQMNSLRAEDTAVYYCARPIRWGMDVW
GQGTTVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNG
KEYKCKVSNKALGAPIEKTISKAKGQPREPQVYTLPPCRDELTKNQVSLWCLV
KGFYPSDIAVEWESNGQPENNYKTTPPVLDSDGSFFLYSKLTVDKSRWQQGNVF
SCSVMHEALHNHYTQKSLSLSPGK
Variable Domain of Light Chain of Clone #5 + Constant Domain of Light Chain
(SEQ ID NO: 316)
DIQMTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Nucleic acid encoding SEQ ID NO: 314:
(SEQ ID NO: 317)
GAGGTGCAGCTGGTGGAGTCTGGGGGAGGCTTGGTACAGCCTGGAGGGTCCC
TGAGACTCTCCTGTGCAGCCTCTGGATTCACCTTCAGTAACTATGGTATGAGC
TGGATCCGCCAGGCTCCAGGGAAGGGGCTAGAGTGGATTGGGAGTATCTATT
ACAGTGGGAGCACCATCTACAACCCCTCCCTCAAGAGTCTAGTCACCATCTC
CAGAGACAATTCCAAGAACACGCTGTATCTGCAAATGAACAGCCTGAGAGCC
GAGGACACAGCCACGTATTACTGTGCGAGAGAGAGTATTGACTACTGGGGCC
AGGGAACCCTGGTCACCGTCTCCTCAAGTGGAGGGGGTGGCAGCGGTGGCGG
GGGAAGTGGCGGTGGAGGGTCGCAAGTTCAATTAGTGGAGAGCGGCGGCGG
ATTGGTTAAACCGGGGGGCAGTCTGCGTTTAAGCTGCGCAGCTTCCGGGTTTA
CTTTTTCGGATTACTATATGAGCTGGATTCGTCAGGCTCCCGGTAAAGGGTTG
GAATGGGTTAGCTACATCTCAAGTTCCGGCTCTACAATCAACTATGCAGATTC
CGTGAAAGGGCGCTTCACCATCTCTCGTGACAATGCAAAGAACAGTTTATAC
TTACAAATGAACTCGCTGCGTGCGGAAGATACGGCTGTGTATTACTGTGCGA
GACCCATCCGGTGGGGTATGGACGTCTGGGGCCAAGGGACCACGGTCACCGT
CTCCTCAGCGTCGACCAAAGGGCCCAGTGTCTTTCCGTTAGCTCCGTCTTCCA
AAAGTACGTCGGGCGGCACGGCGGCACTTGGCTGCTTAGTCAAGGACTACTT
TCCCGAACCCGTTACTGTCTCTTGGAATAGTGGGGCTTTGACCAGTGGGGTTC
ATACCTTTCCAGCCGTACTTCAGTCTTCAGGGCTGTATAGTTTAAGTTCAGTG
GTTACAGTGCCCTCTTCATCTTTAGGGACCCAAACCTATATCTGCAACGTCAA
TCATAAGCCTTCAAATACCAAGGTTGACAAAAAGGTGGAGCCCAAATCTTGT
GACAAAACTCACACATGCCCACCGTGCCCAGCACCTGAAGCCGCGGGGGGA
CCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAA
AGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCC
AACAAAGCCCTCGGAGCCCCCATCGAGAAAACCATCTCCAAAGCCAAAGGG
CAGCCCCGAGAACCACAGGTGTGCACCCTGCCCCCATCCCGGGATGAGCTGA
CCAAGAACCAGGTCAGCCTGAGCTGCGCGGTCAAAGGCTTCTATCCCAGCGA
CATCGCCGTGGAGTGGGAGAGCAATGGGCAGCCGGAGAACAACTACAAGAC
CACGCCTCCCGTGCTGGACTCCGACGGCTCCTTCTTCCTCGTCAGCAAGCTCA
CCGTGGACAAGAGCAGGTGGCAGCAGGGGAACGTCTTCTCATGCTCCGTGAT
GCATGAGGCTCTGCACAACCGCTTCACGCAGAAGAGCCTCTCCCTGTCTCCG
GGTAAA
Nucleic acid encoding SEQ ID NO: 315:
(SEQ ID NO: 318)
CAAGTTCAATTAGTGGAGAGCGGCGGCGGATTGGTTAAACCGGGGGGCAGTC
TGCGTTTAAGCTGCGCAGCTTCCGGGTTTACTTTTTCGGATTACTATATGAGC
TGGATTCGTCAGGCTCCCGGTAAAGGGTTGGAATGGGTTAGCTACATCTCAA
GTTCCGGCTCTACAATCAACTATGCAGATTCCGTGAAAGGGCGCTTCACCATC
TCTCGTGACAATGCAAAGAACAGTTTATACTTACAAATGAACTCGCTGCGTG
CGGAAGATACGGCTGTGTATTACTGTGCGAGACCCATCCGGTGGGGTATGGA
CGTCTGGGGCCAAGGGACCACGGTCACCGTCTCCTCAGCGTCGACCAAAGGG
CCCAGTGTCTTTCCGTTAGCTCCGTCTTCCAAAAGTACGTCGGGCGGCACGGC
GGCACTTGGCTGCTTAGTCAAGGACTACTTTCCCGAACCCGTTACTGTCTCTT
GGAATAGTGGGGCTTTGACCAGTGGGGTTCATACCTTTCCAGCCGTACTTCAG
TCTTCAGGGCTGTATAGTTTAAGTTCAGTGGTTACAGTGCCCTCTTCATCTTTA
GGGACCCAAACCTATATCTGCAACGTCAATCATAAGCCTTCAAATACCAAGG
TTGACAAAAAGGTGGAGCCCAAATCTTGTGACAAAACTCACACATGCCCACC
GTGCCCAGCACCTGAAGCCGCGGGGGGACCGTCAGTCTTCCTCTTCCCCCCA
AAACCCAAGGACACCCTCATGATCTCCCGGACCCCTGAGGTCACATGCGTGG
TGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGCTG
AATGGCAAGGAGTACAAGTGCAAGGTCTCCAACAAAGCCCTCGGAGCCCCC
ATCGAGAAAACCATCTCCAAAGCCAAAGGGCAGCCCCGAGAACCACAGGTG
TACACCCTGCCCCCATGCCGGGATGAGCTGACCAAGAACCAGGTCAGCCTGT
GGTGCCTGGTCAAAGGCTTCTATCCCAGCGACATCGCCGTGGAGTGGGAGAG
CAATGGGCAGCCGGAGAACAACTACAAGACCACGCCTCCCGTGCTGGACTCC
GACGGCTCCTTCTTCCTCTACAGCAAGCTCACCGTGGACAAGAGCAGGTGGC
AGCAGGGGAACGTCTTCTCATGCTCCGTGATGCATGAGGCTCTGCACAACCA
CTACACGCAGAAGAGCCTCTCCCTGTCTCCGGGTAAA
Nucleic acid encoding SEQ ID NO: 316:
(SEQ ID NO: 319)
GATATTCAAATGACCCAGTCTCCTTCTTCCTTGAGCGCGTCTGTGGGAGACCG
TGTAACTATCACGTGTCGTGCATCCCAAAGCATTTCGAGTTACTTGAACTGGT
ATCAACAAAAGCCTGGTAAAGCGCCCAAACTGTTGATCTATGCTGCCAGTAG
TCTTCAATCTGGTGTGCCCTCGCGCTTCTCAGGTTCGGGGTCAGGAACGGACT
TTACCCTGACCATTAGCTCTCTGCAACCGGAAGATGCTGCAACGTATTACTGT
CATCAGAGTAGTAGTTTACCGCTCACTTTCGGCGGAGGGACCAAGGTGGAAA
TCAAACGTACGGTGGCTGCTCCGTCGGTGTTTATCTTTCCACCGAGTGACGAA
CAACTGAAGTCGGGGACTGCCAGCGTTGTTTGCCTTCTGAACAATTTTTACCC
CCGCGAGGCGAAGGTTCAGTGGAAAGTCGATAATGCGTTACAGAGCGGGAA
TAGCCAGGAAAGTGTTACTGAACAGGATAGTAAAGACAGCACTTACAGCTTA
TCGTCTACACTTACTCTGAGTAAAGCGGATTATGAAAAGCATAAGGTGTACG
CTTGCGAGGTTACACATCAAGGGTTATCTTCGCCAGTCACGAAGAGCTTTAAT
CGCGGCGAGTGC
Exemplary 1 + 2 BiKE that binds to PSCA and NK cells (designated BiKE #6A):
Anti-CD16 VH7 Binder + Linker + Variable Domain of Heavy Chain of Clone #6 +
CH1 + Hinge + CH2 + CH3 (hole)
(SEQ ID NO: 320)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSEVQLVQSGAEVKKPGSSVKVSCKASGGTFSSYA
ISWVRQAPGQGLEWMGGIIPIFGTANYAQKFQGRVTITADESTSTAYMELSSLRS
DDTAVYYCARERIAVAGTNYYYGMDVWGQGTLVTVSSASTKGPSVFPLAPSS
KSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHNAK
TKPREEQYNSTYRVVSVLTVLHQDWLNGKEYKCKVSNKALGAPIEKTISKA
KGQPREPQVCTLPPSRDELTKNQVSLSCAVKGFYPSDIAVEWESNGQPENNYKT
TPPVLDSDGSFFLVSKLTVDKSRWQQGNVFSCSVMHEALHNRFTQKSLSLSPGK
Variable Domain of Heavy Chain of Clone #6 + CH1 + Hinge + CH2 + CH3 (knob)
(SEQ ID NO: 321)
EVQLVQSGAEVKKPGSSVKVSCKASGGTFSSYAISWVRQAPGQGLEWMGGIIPIF
GTANYAQKFQGRVTITADESTSTAYMELSSLRSDDTAVYYCARERIAVAGTNYY
YGMDVWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKP
SNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALGAPIEKTISKAKGQPREPQVYTLPPCRDELTKNQ
VSLWCLVKGFYPSDIAVEWESNGQPENNYKTTPPVLDSDGSFFLYSKLTVDKSR
WQQGNVFSCSVMHEALHNHYTQKSLSLSPGK
Variable Domain of Light Chain of Clone #6 + Constant Domain of Light Chain
(SEQ ID NO: 322)
EIVLTQSPATLSVSPGERATLSCRASQSVSSSNLAWYQQKPGQAPRLLIYGASTRA
TGIPARFSGTGSGTDFTLTISRLEPEDFAVYYCQQYGSSITFGQGTRLEIKRTVAAP
SVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Exemplary 1 + 2 BiKE that binds to PSCA and NK cells (designated BiKE #7A):
Anti-CD16 VH7 Binder + Linker + Variable Domain of Heavy Chain of Clone #7 +
CH1 + Hinge + CH2 + CH3 (hole)
(SEQ ID NO: 323)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSEVQLVESGAEVKKPGASVKVSCKASGYTFTGY
YMHWVRQAPGQGLVWMGRINPNSGGTNYAQKFQGRVTMTRDTSISTAYMELS
RLRSDDTAVYYCAFGTRHAFDIWGQGTMVTVSSASTKGPSVFPLAPSSKSTSG
GTAALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKPREE
QYNSTYRVVSVLTVLHQDWLNGKEYKCKVSNKALGAPIEKTISKAKGQPRE
PQVCTLPPSRDELTKNQVSLSCAVKGFYPSDIAVEWESNGQPENNYKTTPPVLDS
DGSFFLVSKLTVDKSRWQQGNVFSCSVMHEALHNRFTQKSLSLSPGK
Variable Domain of Heavy Chain of Clone #7 + CH1 + Hinge + CH2 + CH3 (knob)
(SEQ ID NO: 324)
EVQLVESGAEVKKPGASVKVSCKASGYTFTGYYMHWVRQAPGQGLVWMGRIN
PNSGGTNYAQKFQGRVTMTRDTSISTAYMELSRLRSDDTAVYYCAFGTRHAFDI
WGQGTMVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALGAPIEKTISKAKGQPREPQVYTLPPCRDELTKNQVSLWC
LVKGFYPSDIAVEWESNGQPENNYKTTPPVLDSDGSFFLYSKLTVDKSRWQQGN
VFSCSVMHEALHNHYTQKSLSLSPGK
Variable Domain of Light Chain of Clone #7 + Constant Domain of Light Chain
(SEQ ID NO: 325)
NFMLTQPHSVSESPGKTVTISCTRSSGSIASNYVQWYQQRPGSAPTTVIYEDNQRP
SGVPDRFSGSIDSSSNSASLTISGLKTEDEADYYCQSYDSIVHVVFGGGTKVTVLG
QPKAAPSVTLFPPSSEELQANKATLVCLISDFYPGAVTVAWKADSSPVKAGV
ETTTPSKQSNNKYAASSYLSLTPEQWKSHRSYSCQVTHEGSTVEKTVAPTEC
S
Exemplary 1 + 2 BiKE that binds to PSCA and NK cells (designated BiKE #8A):
Anti-CD16 VH7 Binder + Linker + Variable Domain of Heavy Chain of Clone #8 +
CH1 + Hinge + CH2 + CH3 (hole)
(SEQ ID NO: 326)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSEVQLVQSGAEVKKPGASVKVSCKASGGTFSSY
AISWVRQAPGQGLEWMGGIIPIFGTANYAQKFQGRVTITADKSTSTAYMELSSLR
SEDTAVYYCARDVIVGATSSGMDVWGQGTTVTVSSASTKGPSVFPLAPSSKSTS
GGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPRE
EQYNSTYRVVSVLTVLHQDWLNGKEYKCKVSNKALGAPIEKTISKAKGQPR
EPQVCTLPPSRDELTKNQVSLSCAVKGFYPSDIAVEWESNGQPENNYKTTPPVLD
SDGSFFLVSKLTVDKSRWQQGNVFSCSVMHEALHNRFTQKSLSLSPGK
Variable Domain of Heavy Chain of Clone #8 + CH1 + Hinge + CH2 + CH3 (knob)
(SEQ ID NO: 327)
EVQLVQSGAEVKKPGASVKVSCKASGGTFSSYAISWVRQAPGQGLEWMGGIIPI
FGTANYAQKFQGRVTITADKSTSTAYMELSSLRSEDTAVYYCARDVIVGATSSG
MDVWGQGTTVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSN
TKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALGAPIEKTISKAKGQPREPQVYTLPPCRDELTKNQV
SLWCLVKGFYPSDIAVEWESNGQPENNYKTTPPVLDSDGSFFLYSKLTVDKSRW
QQGNVFSCSVMHEALHNHYTQKSLSLSPGK
Variable Domain of Light Chain of Clone #8 + Constant Domain of Light Chain
(SEQ ID NO: 328)
DIQMTQSPSSVSASVGDRVTITCRASQSISRYLNWYQQKPGKAPKLLIYGASSLQS
GVPSRFSGSGFGTDFTLTISSLQPEDFATYYCQQSYSTPLTFGQGTKLEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Exemplary 2 + 2 BiKE that binds to PSCA and NK cells (designated BiKE #1B):
Anti-CD16 VH7 Binder + Linker + Variable Domain of Heavy Chain of Clone #1 +
CH1 + Hinge + CH2 + CH3
(SEQ ID NO: 329)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSQVQLVESGGGLVKPGGSLRLSCAASGFTFSDYY
MSWIRQAPGKGLEWVSYISSSGSTIYYADSVKGRFTISRDNAKNSLYLQMNSLRA
EDTAVYYCARPLRWGMDVWGQGTTVTVSSASTKGPSVFPLAPSSKSTSGGTA
ALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQV
YTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
Variable Domain of Light Chain of Clone #1 + Constant Domain of Light Chain
(SEQ ID NO: 330)
DIQMTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Exemplary 2 + 2 BiKE that binds to PSCA and NK cells (designated BiKE #2B):
Anti-CD16 VH7 Binder + Linker + Variable Domain of Heavy Chain of Clone #2 +
CH1 + Hinge + CH2 + CH3
(SEQ ID NO: 331)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSQVQLVESGGGLVKPGGSLRLSCAASGFTFSDYY
MSWIRQAPGKGLEWVSYISSSGSTIYYADSVKGRFTISRDNAKNSLYLQMNSLRA
EDTAVYYCARPLRWGMDVWGQGTTVTVSSASTKGPSVFPLAPSSKSTSGGTA
ALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQV
YTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
Variable Domain of Light Chain of Clone #2 + Constant Domain of Light Chain
(SEQ ID NO: 332)
DIQMTQSPSSLSASVGDRVTITCRASQSISSYLNWYQQKPGKAPKLLIYAASSLQS
GVPSRFSGSGSGTDFTLTISSLQPEDFAVYYCHQSSSLPLTFGGGTKLEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Exemplary 2 + 2 BiKE that binds to PSCA and NK cells (designated BiKE #3B):
Anti-CD16 VH7 Binder + Linker + Variable Domain of Heavy Chain of Clone #3 +
CH1 + Hinge + CH2 + CH3
(SEQ ID NO: 333)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSQVQLVESGGGLVKPGGSLRLSCAASGFTFSDYY
MSWIRQAPGKGLEWVSYISSSGSTIYYADSVKGRFTISRDNAKNSLYLQMNSLRA
EDTAVYYCARPLRWGMDVWGQGTTVTVSSASTKGPSVFPLAPSSKSTSGGTA
ALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQV
YTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
Variable Domain of Light Chain of Clone #3 + Constant Domain of Light Chain
(SEQ ID NO: 334)
DIQMTQSPSSLSASVGDRVTITCRASQSISSYLNWYQQKPGKAPKLLIYAASSLQS
GVPSRFSGSGSGTDFTLTISSLQPEDVAVYYCHQSNSLPLTFGGGTKVEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Exemplary 2 + 2 BiKE that binds to PSCA and NK cells (designated BiKE #4B):
Anti-CD16 VH7 Binder + Linker + Variable Domain of Heavy Chain of Clone #4 +
CH1 + Hinge + CH2 + CH3
(SEQ ID NO: 335)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSQVQLVESGGGLVKPGGSLRLSCAASGFTFSDYY
MSWIRQAPGKGLEWVSYISSSGSTIVYADSVKGRFTISRDNAKNSLYLQMNSLRA
EDTAVYYCARPIRRGMDVWGQGTTVTVSSASTKGPSVFPLAPSSKSTSGGTAA
LGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNS
TYRVVSVLTVLHQDWLNGKEYKCKVSNKALGAPIEKTISKAKGQPREPQVY
TLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
Variable Domain of Light Chain of Clone #4 + Constant Domain of Light Chain
(SEQ ID NO: 336)
DIQMTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Exemplary 2 + 2 BiKE that binds to PSCA and NK cells (designated BiKE #5B):
Anti-CD16 VH7 Binder + Linker + Variable Domain of Heavy Chain of Clone #5 +
CH1 + Hinge + CH2 + CH3
(SEQ ID NO: 337)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSQVQLVESGGGLVKPGGSLRLSCAASGFTFSDYY
MSWIRQAPGKGLEWVSYISSSGSTINYADSVKGRFTISRDNAKNSLYLQMNSLRA
EDTAVYYCARPIRWGMDVWGQGTTVTVSSASTKGPSVFPLAPSSKSTSGGTAA
LGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNS
TYRVVSVLTVLHQDWLNGKEYKCKVSNKALGAPIEKTISKAKGQPREPQVY
TLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
Variable Domain of Light Chain of Clone #5 + Constant Domain of Light Chain
(SEQ ID NO: 338)
DIQMTQSPSSLSASVGDRVTITCRASQSISSYLNWYQQKPGKAPKLLIYAASSLQS
GVPSRFSGSGSGTDFTLTISSLQPEDAATYYCHQSSSLPLTFGGGTKVEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Nucleic acid encoding SEQ ID NO: 337:
(SEQ ID NO: 339)
GAGGTGCAGCTGGTGGAGTCTGGGGGAGGCTTGGTACAGCCTGGAGGGTCCC
TGAGACTCTCCTGTGCAGCCTCTGGATTCACCTTCAGTAACTATGGTATGAGC
TGGATCCGCCAGGCTCCAGGGAAGGGGCTAGAGTGGATTGGGAGTATCTATT
ACAGTGGGAGCACCATCTACAACCCCTCCCTCAAGAGTCTAGTCACCATCTC
CAGAGACAATTCCAAGAACACGCTGTATCTGCAAATGAACAGCCTGAGAGCC
GAGGACACAGCCACGTATTACTGTGCGAGAGAGAGTATTGACTACTGGGGCC
AGGGAACCCTGGTCACCGTCTCCTCAAGTGGAGGGGGTGGCAGCGGTGGCGG
GGGAAGTGGCGGTGGAGGGTCGCAAGTTCAATTAGTGGAGAGCGGCGGCGG
ATTGGTTAAACCGGGGGGCAGTCTGCGTTTAAGCTGCGCAGCTTCCGGGTTTA
CTTTTTCGGATTACTATATGAGCTGGATTCGTCAGGCTCCCGGTAAAGGGTTG
GAATGGGTTAGCTACATCTCAAGTTCCGGCTCTACAATCAACTATGCAGATTC
CGTGAAAGGGCGCTTCACCATCTCTCGTGACAATGCAAAGAACAGTTTATAC
TTACAAATGAACTCGCTGCGTGCGGAAGATACGGCTGTGTATTACTGTGCGA
GACCCATCCGGTGGGGTATGGACGTCTGGGGCCAAGGGACCACGGTCACCGT
CTCCTCAGCGTCGACCAAAGGGCCCAGTGTCTTTCCGTTAGCTCCGTCTTCCA
AAAGTACGTCGGGCGGCACGGCGGCACTTGGCTGCTTAGTCAAGGACTACTT
TCCCGAACCCGTTACTGTCTCTTGGAATAGTGGGGCTTTGACCAGTGGGGTTC
ATACCTTTCCAGCCGTACTTCAGTCTTCAGGGCTGTATAGTTTAAGTTCAGTG
GTTACAGTGCCCTCTTCATCTTTAGGGACCCAAACCTATATCTGCAACGTCAA
TCATAAGCCTTCAAATACCAAGGTTGACAAAAAGGTGGAGCCCAAATCTTGT
GACAAAACTCACACATGCCCACCGTGCCCAGCACCTGAAGCCGCGGGGGGA
CCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAA
AGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCC
AACAAAGCCCTCGGAGCCCCCATCGAGAAAACCATCTCCAAAGCCAAAGGG
CAGCCCCGAGAACCACAGGTGTACACCCTGCCCCCATCCCGGGATGAGCTGA
CCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGAC
CACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTCCG
GGTAAA
Nucleic acid encoding SEQ ID NO: 338:
(SEQ ID NO: 340)
GATATTCAAATGACCCAGTCTCCTTCTTCCTTGAGCGCGTCTGTGGGAGACCG
TGTAACTATCACGTGTCGTGCATCCCAAAGCATTTCGAGTTACTTGAACTGGT
ATCAACAAAAGCCTGGTAAAGCGCCCAAACTGTTGATCTATGCTGCCAGTAG
TCTTCAATCTGGTGTGCCCTCGCGCTTCTCAGGTTCGGGGTCAGGAACGGACT
TTACCCTGACCATTAGCTCTCTGCAACCGGAAGATGCTGCAACGTATTACTGT
CATCAGAGTAGTAGTTTACCGCTCACTTTCGGCGGAGGGACCAAGGTGGAAA
TCAAACGTACGGTGGCTGCTCCGTCGGTGTTTATCTTTCCACCGAGTGACGAA
CAACTGAAGTCGGGGACTGCCAGCGTTGTTTGCCTTCTGAACAATTTTTACCC
CCGCGAGGCGAAGGTTCAGTGGAAAGTCGATAATGCGTTACAGAGCGGGAA
TAGCCAGGAAAGTGTTACTGAACAGGATAGTAAAGACAGCACTTACAGCTTA
TCGTCTACACTTACTCTGAGTAAAGCGGATTATGAAAAGCATAAGGTGTACG
CTTGCGAGGTTACACATCAAGGGTTATCTTCGCCAGTCACGAAGAGCTTTAAT
CGCGGCGAGTGC
Exemplary 2 + 2 BiKE that binds to PSCA and NK cells (designated BiKE #6B):
Anti-CD16 VH7 Binder + Linker + Variable Domain of Heavy Chain of Clone #6 +
CH1 + Hinge + CH2 + CH3
(SEQ ID NO: 341)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSEVQLVQSGAEVKKPGSSVKVSCKASGGTFSSYA
ISWVRQAPGQGLEWMGGIIPIFGTANYAQKFQGRVTITADESTSTAYMELSSLRS
DDTAVYYCARERIAVAGTNYYYGMDVWGQGTLVTVSSASTKGPSVFPLAPSS
KSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHNAK
TKPREEQYNSTYRVVSVLTVLHQDWLNGKEYKCKVSNKALGAPIEKTISKA
KGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGK
Variable Domain of Light Chain of Clone #6 + Constant Domain of Light Chain
(SEQ ID NO: 342)
EIVLTQSPATLSVSPGERATLSCRASQSVSSSNLAWYQQKPGQAPRLLIYGASTRA
TGIPARFSGTGSGTDFTLTISRLEPEDFAVYYCQQYGSSITFGQGTRLEIKRTVAAP
SVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Exemplary 2 + 2 BiKE that binds to PSCA and NK cells (designated BiKE #7B):
Anti-CD16 VH7 Binder + Linker + Variable Domain of Heavy Chain of Clone #7 +
CH1 + Hinge + CH2 + CH3
(SEQ ID NO: 343)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSEVQLVESGAEVKKPGASVKVSCKASGYTFTGY
YMHWVRQAPGQGLVWMGRINPNSGGTNYAQKFQGRVTMTRDTSISTAYMELS
RLRSDDTAVYYCAFGTRHAFDIWGQGTMVTVSSASTKGPSVFPLAPSSKSTSG
GTAALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKPREE
QYNSTYRVVSVLTVLHQDWLNGKEYKCKVSNKALGAPIEKTISKAKGQPRE
PQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Variable Domain of Light Chain of Clone #7 + Constant Domain of Light Chain
(SEQ ID NO: 344)
NFMLTQPHSVSESPGKTVTISCTRSSGSIASNYVQWYQQRPGSAPTTVIYEDNQRP
SGVPDRFSGSIDSSSNSASLTISGLKTEDEADYYCQSYDSIVHVVFGGGTKVTVLG
QPKAAPSVTLFPPSSEELQANKATLVCLISDFYPGAVTVAWKADSSPVKAGV
ETTTPSKQSNNKYAASSYLSLTPEQWKSHRSYSCQVTHEGSTVEKTVAPTEC
S
Exemplary 2 + 2 BiKE that binds to PSCA and NK cells (designated BiKE #8B):
Anti-CD16 VH7 Binder + Linker + Variable Domain of Heavy Chain of Clone #8 +
CH1 + Hinge + CH2 + CH3
(SEQ ID NO: 345)
EVQLVESGGGLVQPGGSLRLSCAASGFTFSNYGMSWIRQAPGKGLEWIGSIYYS
GSTIYNPSLKSLVTISRDNSKNTLYLQMNSLRAEDTATYYCARESIDYWGQGTLV
TVSSSGGGGSGGGGSGGGGSEVQLVQSGAEVKKPGASVKVSCKASGGTFSSY
AISWVRQAPGQGLEWMGGIIPIFGTANYAQKFQGRVTITADKSTSTAYMELSSLR
SEDTAVYYCARDVIVGATSSGMDVWGQGTTVTVSSASTKGPSVFPLAPSSKSTS
GGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPRE
EQYNSTYRVVSVLTVLHQDWLNGKEYKCKVSNKALGAPIEKTISKAKGQPR
EPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
Variable Domain of Light Chain of Clone #8 + Constant Domain of Light Chain
(SEQ ID NO: 346)
DIQMTQSPSSVSASVGDRVTITCRASQSISRYLNWYQQKPGKAPKLLIYGASSLQS
GVPSRFSGSGFGTDFTLTISSLQPEDFATYYCQQSYSTPLTFGQGTKLEIKRTVAA
PSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC

### Example 22—Exemplary BiTEs that Bind to PSCA and T Cells

This Example depicts the structures and sequences of exemplary BiTEs designed to include the CDRs of clones #1-#8.

The various components of each BiTE (e.g., domains and linkers) are provided and delineated.

Exemplary BiTE that binds to PSCA and T cells
Signal peptide + scFv using CDRs of Clone #1 + (GSGSS)3 linker (SEQ ID NO: 839) +
gOKT3-7 (anti-CD3 scFv) + GSGSSG linker (SEQ ID NO: 840) + hIgG1 Fc
(SEQ ID NO: 347)
MTRLTVLALLAGLLASSRAQVQLVESGGGLVKPGGSLRLSCAASGFTFSDYYM
SWIRQAPGKGLEWVSYISSSGSTIYYADSVKGRFTISRDNAKNSLYLQMNSLRAE
DTAVYYCARPLRWGMDVWGQGTTVTVSSGGGGSGGGGSGGGGSDIQMTQSPS
SLSASVGDRVTITCRASQSISSYLNWYQQKPGKAPKLLIYAASSLQSGVPSRFSGS
GSGTDFTLTISSLQPEDAATYYCHQSSSLPLTFGGGTKVEIKGSGSSGSGSSGSGS
SQVQLVQSGGGVVQPGRSLRLSCKASGYTFTRYTMHWVRQAPGKGLEWIGYIN
PSRGYTNYNQKVKDRFTISRDNSKNTAFLQMDSLRPEDTGVYFCARYYDDHYC
LDYWGQGTPVTVSSGGGGSGGGGSGGGGSGGGGSGGGGSGGGGSDIQMTQSPS
SLSASVGDRVTITCSASSSVSYMNWYQQTPGKAPKRWIYDTSKLASGVPSRFSGS
GSGTDYTFTISSLQPEDIATYYCQQWSSNPFTFGQGTKLQITRGSGSSGDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLS
PGK
Exemplary BiTE that binds to PSCA and T cells
Signal peptide + scFv using CDRs of Clone #2 + (GSGSS)3 linker (SEQ ID NO: 839) +
gOKT3-7 (anti-CD3 scFv) + GSGSSG linker (SEQ ID NO: 840) + hIgG1 Fc
(SEQ ID NO: 348)
MTRLTVLALLAGLLASSRAQVQLVESGGGLVKPGGSLRLSCAASGFTFSDYYM
SWIRQAPGKGLEWVSYISSSGSTIYYADSVKGRFTISRDNAKNSLYLQMNSLRAE
DTAVYYCARPLRWGMDVWGQGTTVTVSSGGGGSGGGGSGGGGSDIQMTQSPS
SLSASVGDRVTITCRASQSISSYLNWYQQKPGKAPKLLIYAASSLQSGVPSRFSGS
GSGTDFTLTISSLQPEDFAVYYCHQSSSLPLTFGGGTKLEIKGSGSSGSGSSGSGS
SQVQLVQSGGGVVQPGRSLRLSCKASGYTFTRYTMHWVRQAPGKGLEWIGYIN
PSRGYTNYNQKVKDRFTISRDNSKNTAFLQMDSLRPEDTGVYFCARYYDDHYC
LDYWGQGTPVTVSSGGGGSGGGGSGGGGSGGGGSGGGGSGGGGSDIQMTQSPS
SLSASVGDRVTITCSASSSVSYMNWYQQTPGKAPKRWIYDTSKLASGVPSRFSGS
GSGTDYTFTISSLQPEDIATYYCQQWSSNPFTFGQGTKLQITRGSGSSGDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLS
PGK
Exemplary BiTE that binds to PSCA and T cells
Signal peptide + scFv using CDRs of Clone #3 + (GSGSS)3 linker (SEQ ID NO: 839) +
gOKT3-7 (anti-CD3 scFv) + GSGSSG linker (SEQ ID NO: 840) + hIgG1 Fc
(SEQ ID NO: 349)
MTRLTVLALLAGLLASSRAQVQLVESGGGLVKPGGSLRLSCAASGFTFSDYYM
SWIRQAPGKGLEWVSYISSSGSTIYYADSVKGRFTISRDNAKNSLYLQMNSLRAE
DTAVYYCARPLRWGMDVWGQGTTVTVSSGGGGSGGGGSGGGGSDIQMTQSPS
SLSASVGDRVTITCRASQSISSYLNWYQQKPGKAPKLLIYAASSLQSGVPSRFSGS
GSGTDFTLTISSLQPEDVAVYYCHQSNSLPLTFGGGTKVEIKGSGSSGSGSSGSGS
SQVQLVQSGGGVVQPGRSLRLSCKASGYTFTRYTMHWVRQAPGKGLEWIGYIN
PSRGYTNYNQKVKDRFTISRDNSKNTAFLQMDSLRPEDTGVYFCARYYDDHYC
LDYWGQGTPVTVSSGGGGSGGGGSGGGGSGGGGSGGGGSGGGGSDIQMTQSPS
SLSASVGDRVTITCSASSSVSYMNWYQQTPGKAPKRWIYDTSKLASGVPSRFSGS
GSGTDYTFTISSLQPEDIATYYCQQWSSNPFTFGQGTKLQITRGSGSSGDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLS
PGK
Exemplary BiTE that binds to PSCA and T cells
Signal peptide + scFv using CDRs of Clone #4 + (GSGSS)3 linker (SEQ ID NO: 839) +
gOKT3-7 (anti-CD3 scFv) + GSGSSG linker (SEQ ID NO: 840) + hIgG1 Fc
(SEQ ID NO: 350)
MTRLTVLALLAGLLASSRAQVQLVESGGGLVKPGGSLRLSCAASGFTFSDYYM
SWIRQAPGKGLEWVSYISSSGSTIVYADSVKGRFTISRDNAKNSLYLQMNSLRAE
DTAVYYCARPIRRGMDVWGQGTTVTVSSGGGGSGGGGSGGGGSDIQMTQSPSS
LSASVGDRVTITCRASQSISSYLNWYQQKPGKAPKLLIYAASSLQSGVPSRFSGSG
SGTDFTLTISSLQPEDAATYYCHQSSSLPLTFGGGTKVEIKGSGSSGSGSSGSGSS
QVQLVQSGGGVVQPGRSLRLSCKASGYTFTRYTMHWVRQAPGKGLEWIGYINP
SRGYTNYNQKVKDRFTISRDNSKNTAFLQMDSLRPEDIGVYFCARYYDDHYCL
DYWGQGTPVTVSSGGGGSGGGGSGGGGSGGGGSGGGGSGGGGSDIQMTQSPSS
LSASVGDRVTITCSASSSVSYMNWYQQTPGKAPKRWIYDTSKLASGVPSRFSGS
GSGTDYTFTISSLQPEDIATYYCQQWSSNPFTFGQGTKLQITRGSGSSGDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALP APIEKTI
SKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLS
PGK
Exemplary BiTE that binds to PSCA and T cells
Signal peptide + scFv using CDRs of Clone #5 + (GSGSS)3 linker (SEQ ID NO: 839) +
gOKT3-7 (anti-CD3 scFv) + GSGSSG linker (SEQ ID NO: 840) + hIgG1 Fc
(SEQ ID NO: 351)
MTRLTVLALLAGLLASSRAQVQLVESGGGLVKPGGSLRLSCAASGFTFSDYYM
SWIRQAPGKGLEWVSYISSSGSTINYADSVKGRFTISRDNAKNSLYLQMNSLRAE
DTAVYYCARPIRWGMDVWGQGTTVTVSSGGGGSGGGGSGGGGSDIQMTQSPSS
LSASVGDRVTITCRASQSISSYLNWYQQKPGKAPKLLIYAASSLQSGVPSRFSGSG
SGTDFTLTISSLQPEDAATYYCHQSSSLPLTFGGGTKVEIKGSGSSGSGSSGSGSS
QVQLVQSGGGVVQPGRSLRLSCKASGYTFTRYTMHWVRQAPGKGLEWIGYINP
SRGYTNYNQKVKDRFTISRDNSKNTAFLQMDSLRPEDTGVYFCARYYDDHYCL
DYWGQGTPVTVSSGGGGSGGGGSGGGGSGGGGSGGGGSGGGGSDIQMTQSPSS
LSASVGDRVTITCSASSSVSYMNWYQQTPGKAPKRWIYDTSKLASGVPSRFSGS
GSGTDYTFTISSLQPEDIATYYCQQWSSNPFTFGQGTKLQITRGSGSSGDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLS
PGK
Exemplary BiTE that binds to PSCA and T cells
Signal peptide + Clone #6 scFv + (GSGSS)3 linker (SEQ ID NO: 839) + gOKT3-7
(anti-CD3 scFv) + GSGSSG linker (SEQ ID NO: 840) + hIgG1 Fc
(SEQ ID NO: 352)
MTRLTVLALLAGLLASSRAEVQLVQSGAEVKKPGSSVKVSCKASGGTFSSYAIS
WVRQAPGQGLEWMGGIIPIFGTANYAQKFQGRVTITADESTSTAYMELSSLRSD
DTAVYYCARERIAVAGTNYYYGMDVWGQGTLVTVSSGGGGSGGGGSGGGGSE
IVLTQSPATLSVSPGERATLSCRASQSVSSSNLAWYQQKPGQAPRLLIYGASTRAT
GIPARFSGTGSGTDFTLTISRLEPEDFAVYYCQQYGSSITFGQGTRLEIKGSGSSGS
GSSGSGSSQVQLVQSGGGVVQPGRSLRLSCKASGYTFTRYTMHWVRQAPGKGL
EWIGYINPSRGYTNYNQKVKDRFTISRDNSKNTAFLQMDSLRPEDTGVYFCARY
YDDHYCLDYWGQGTPVTVSSGGGGSGGGGSGGGGSGGGGSGGGGSGGGGSDI
QMTQSPSSLSASVGDRVTITCSASSSVSYMNWYQQTPGKAPKRWIYDTSKLASG
VPSRFSGSGSGTDYTFTISSLQPEDIATYYCQQWSSNPFTFGQGTKLQITRGSGSS
GDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNH
YTQKSLSLSPGK
Exemplary BiTE that binds to PSCA and T cells
Signal peptide + Clone #7 scFv + (GSGSS)3 linker (SEQ ID NO: 839) + gOKT3-7
(anti-CD3 scFv) + GSGSSG linker (SEQ ID NO: 840) + hIgG1 Fc
(SEQ ID NO: 353)
MTRLTVLALLAGLLASSRAEVQLVESGAEVKKPGASVKVSCKASGYTFTGYY
MHWVRQAPGQGLVWMGRINPNSGGTNYAQKFQGRVTMTRDTSISTAYMELSR
LRSDDTAVYYCAFGTRHAFDIWGQGTMVTVSSGGGGSGGGGSGGGGSNFMLT
QPHSVSESPGKTVTISCTRSSGSIASNYVQWYQQRPGSAPTTVIYEDNQRPSGVPD
RFSGSIDSSSNSASLTISGLKTEDEADYYCQSYDSIVHVVFGGGTKVTVLGGSGSS
GSGSSGSGSSQVQLVQSGGGVVQPGRSLRLSCKASGYTFTRYTMHWVRQAPGK
GLEWIGYINPSRGYTNYNQKVKDRFTISRDNSKNTAFLQMDSLRPEDTGVYFCA
RYYDDHYCLDYWGQGTPVTVSSGGGGSGGGGSGGGGSGGGGSGGGGSGGGGS
DIQMTQSPSSLSASVGDRVTITCSASSSVSYMNWYQQTPGKAPKRWIYDTSKLAS
GVPSRFSGSGSGTDYTFTISSLQPEDIATYYCQQWSSNPFTFGQGTKLQITRGSGS
SGDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNH
YTQKSLSLSPGK
Exemplary BiTE that binds to PSCA and T cells
Signal peptide + Clone #8 scFv + (GSGSS)3 linker (SEQ ID NO: 839) + gOKT3-7
(anti-CD3 scFv) + GSGSSG linker (SEQ ID NO: 840) + hIgG1 Fc
(SEQ ID NO: 354)
MTRLTVLALLAGLLASSRAEVQLVQSGAEVKKPGASVKVSCKASGGTFSSYAI
SWVRQAPGQGLEWMGGIIPIFGTANYAQKFQGRVTITADKSTSTAYMELSSLRSE
DTAVYYCARDVIVGATSSGMDVWGQGTTVTVSSGGGGSGGGGSGGGGSDIQM
TQSPSSVSASVGDRVTITCRASQSISRYLNWYQQKPGKAPKLLIYGASSLQSGVPS
RFSGSGFGTDFTLTISSLQPEDFATYYCQQSYSTPLTFGQGTKLEIKGSGSSGSGSS
GSGSSQVQLVQSGGGVVQPGRSLRLSCKASGYTFTRYTMHWVRQAPGKGLEWI
GYINPSRGYTNYNQKVKDRFTISRDNSKNTAFLQMDSLRPEDTGVYFCARYYDD
HYCLDYWGQGTPVTVSSGGGGSGGGGSGGGGSGGGGSGGGGSGGGGSDIQMT
QSPSSLSASVGDRVTITCSASSSVSYMNWYQQTPGKAPKRWIYDTSKLASGVPSR
FSGSGSGTDYTFTISSLQPEDIATYYCQQWSSNPFTFGQGTKLQITRGSGSSGDKT
HTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQK
SLSLSPGK

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO: 1 with one, two, or three amino acid additions, deletions, or substitutions);
(ii) a heavy chain variable domain or region comprising the amino acid sequences set forth in SEQ ID NO: 17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO:105 (or SEQ ID NO:105 with one, two, or three amino acid additions, deletions, or substitutions), SEQ ID NO: 106 (or SEQ ID NO:106 with one, two, or three amino acid additions, deletions, or substitutions), and SEQ ID NO:107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO:113 with one, two, or three amino acid additions, deletions, or substitutions), SEQ ID NO:114 (or SEQ ID NO:114 with one, two, or three amino acid additions, deletions, or substitutions), and SEQ ID NO:115 (or SEQ ID NO:115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO:122 (or SEQ ID NO:122 with one, two, or three amino acid additions, deletions, or substitutions), and SEQ ID NO:123 (or SEQ ID NO:123 with one, two, or three amino acid additions, deletions, or substitutions).

2-12. (canceled)

13. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO:10 with one, two, or three amino acid additions, deletions, or substitutions), and SEQ ID NO:11 (or SEQ ID NO: 1 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO:105 (or SEQ ID NO:105 with one, two, or three amino acid additions, deletions, or substitutions), SEQ ID NO:106 (or SEQ ID NO:106 with one, two, or three amino acid additions, deletions, or substitutions), and SEQ ID NO:107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO:113 with one, two, or three amino acid additions, deletions, or substitutions), SEQ ID NO:114 (or SEQ ID NO:114 with one, two, or three amino acid additions, deletions, or substitutions), and SEQ ID NO:115 (or SEQ ID NO:115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO: 122 (or SEQ ID NO:122 with one, two, or three amino acid additions, deletions, or substitutions), and SEQ ID NO:123 (or SEQ ID NO:123 with one, two, or three amino acid additions, deletions, or substitutions).

14-36. (canceled)

37. A chimeric antigen receptor comprising an antigen binding domain, a hinge, a transmembrane domain, and one or more signaling domains, wherein said antigen binding domain comprises an antibody or an antigen-binding fragment comprising:
(i) a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO:11 (or SEQ ID NO: 1 with one, two, or three amino acid additions, deletions, or substitutions);
(ii) a heavy chain variable domain or region comprising the amino acid sequences set forth in SEQ ID NO: 17 (or SEQ ID NO: 17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO: 19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO: 106 (or SEQ ID NO:106 with one, two, or three amino acid additions, deletions, or substitutions), and SEQ ID NO:107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO: 113 with one, two, or three amino acid additions, deletions, or substitutions), SEQ ID NO:114 (or SEQ ID NO:114 with one, two, or three amino acid additions, deletions, or substitutions), and SEQ ID NO: 115 (or SEQ ID NO: 115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO: 122 (or SEQ ID NO:122 with one, two, or three amino acid additions, deletions, or substitutions), and SEQ ID NO: 123 (or SEQ ID NO:123 with one, two, or three amino acid additions, deletions, or substitutions).

38-43. (canceled)

44. A cell engager comprising a first antigen binding domain, a linker, and a second antigen binding domain, wherein said first antigen binding domain comprises an antibody or an antigen-binding fragment comprising:
(i) a heavy chain variable domain or region comprising the amino acid sequences set forth in SEQ ID NO:1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO: 10 with one, two, or three amino acid additions, deletions, or substitutions), and SEQ ID NO:11 (or SEQ ID NO: 1 with one, two, or three amino acid additions, deletions, or substitutions);
(ii) a heavy chain variable domain or region comprising the amino acid sequences set forth in SEQ ID NO: 17 (or SEQ ID NO: 17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO: 19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO: 106 (or SEQ ID NO:106 with one, two, or three amino acid additions, deletions, or substitutions), and SEQ ID NO:107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO: 113 with one, two, or three amino acid additions, deletions, or substitutions), SEQ ID NO:114 (or SEQ ID NO:114 with one, two, or three amino acid additions, deletions, or substitutions), and SEQ ID NO: 115 (or SEQ ID NO: 115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO: 122 (or SEQ ID NO:122 with one, two, or three amino acid additions, deletions, or substitutions), and SEQ ID NO: 123 (or SEQ ID NO:123 with one, two, or three amino acid additions, deletions, or substitutions).

45-57. (canceled)

58. A nucleic acid comprising a nucleic acid sequence encoding at least part of an antibody or an antigen-binding fragment comprising:
(i) a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO:11 (or SEQ ID NO: 1 with one, two, or three amino acid additions, deletions, or substitutions);
(ii) a heavy chain variable domain or region comprising the amino acid sequences set forth in SEQ ID NO:17 (or SEQ ID NO: 17 with one, two, or three amino acid additions, deletions, or substitutions), SEQ ID NO: 18 (or SEQ ID NO:18 with one, two, or three amino acid additions, deletions, or substitutions), and SEQ ID NO: 19 (or SEQ ID NO: 19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO: 106 (or SEQ ID NO:106 with one, two, or three amino acid additions, deletions, or substitutions), and SEQ ID NO: 107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO:113 with one, two, or three amino acid additions, deletions, or substitutions), SEQ ID NO:114 (or SEQ ID NO: 114 with one, two, or three amino acid additions, deletions, or substitutions), and SEQ ID NO:115 (or SEQ ID NO: 115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO:122 (or SEQ ID NO:122 with one, two, or three amino acid additions, deletions, or substitutions), and SEQ ID NO:123 (or SEQ ID NO:123 with one, two, or three amino acid additions, deletions, or substitutions).

59-65. (canceled)

66. A host cell that expresses (a) a chimeric antigen receptor comprising an antigen binding domain, a hinge, a transmembrane domain, and one or more signaling domains, wherein said antigen binding domain comprises an antibody or an antigen-binding fragment or (b) a cell engager comprising a first antigen binding domain, a linker, and a second antigen binding domain, wherein said first antigen binding domain comprises an antibody or an antigen-binding fragment,
wherein said antibody or an antigen-binding fragment comprises:
(i) a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 1 with one, two, or three amino acid additions, deletions, or substitutions);
(ii) a heavy chain variable domain or region comprising the amino acid sequences set forth in SEQ ID NO: 17 (or SEQ ID NO: 17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO: 19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO: 106 (or SEQ ID NO:106 with one, two, or three amino acid additions, deletions, or substitutions), and SEQ ID NO:107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO:113 with one, two, or three amino acid additions, deletions, or substitutions), SEQ ID NO:114 (or SEQ ID NO:114 with one, two, or three amino acid additions, deletions, or substitutions), and SEQ ID NO:115 (or SEQ ID NO:115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO: 122 (or SEQ ID NO:122 with one, two, or three amino acid additions, deletions, or substitutions), and SEQ ID NO:123 (or SEQ ID NO:123 with one, two, or three amino acid additions, deletions, or substitutions).

67. The host cell of claim 66, wherein said host cell is a T cell, stem cell, or NK cell.

68. An antibody-drug conjugate (ADC) comprising an antigen binging domain covalently linked to a drug, wherein said antigen binging domain comprises an antibody or an antigen binding fragment comprising:
(i) a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO: 11 (or SEQ ID NO: 1 with one, two, or three amino acid additions, deletions, or substitutions);
(ii) a heavy chain variable domain or region comprising the amino acid sequences set forth in SEQ ID NO: 17 (or SEQ ID NO: 17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO: 19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO: 106 (or SEQ ID NO:106 with one, two, or three amino acid additions, deletions, or substitutions), and SEQ ID NO:107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO:113 with one, two, or three amino acid additions, deletions, or substitutions), SEQ ID NO:114 (or SEQ ID NO:114 with one, two, or three amino acid additions, deletions, or substitutions), and SEQ ID NO:115 (or SEQ ID NO:115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO: 122 (or SEQ ID NO:122 with one, two, or three amino acid additions, deletions, or substitutions), and SEQ ID NO:123 (or SEQ ID NO:123 with one, two, or three amino acid additions, deletions, or substitutions).

69-70. (canceled)

71. A composition comprising (a) an antibody, (b) an antigen binding fragment, (c) a host cell that expresses a chimeric antigen receptor comprising an antigen binding domain, a hinge, a transmembrane domain, and one or more signaling domains, wherein said antigen binding domain comprises said antibody or said antigen-binding fragment, (d) a host cell that expresses a cell engager comprising a first antigen binding domain, a linker, and a second antigen binding domain, wherein said first antigen binding domain comprises said antibody or said antigen-binding fragment, (e) said cell engager, or (f) an antibody-drug conjugate (ADC) comprising an antigen binging domain covalently linked to a drug, wherein said antigen binging domain comprises said antibody or said antigen binding fragment,
wherein said antibody or said antigen binding fragment comprises:
(i) a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO:10 with one, two, or three amino acid additions, deletions, or substitutions), and SEQ ID NO:11 (or SEQ ID NO: 1 with one, two, or three amino acid additions, deletions, or substitutions);
(ii) a heavy chain variable domain or region comprising the amino acid sequences set forth in SEQ ID NO: 17 (or SEQ ID NO: 17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO: 106 (or SEQ ID NO:106 with one, two, or three amino acid additions, deletions, or substitutions), and SEQ ID NO: 107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO: 113 with one, two, or three amino acid additions, deletions, or substitutions), SEQ ID NO:114 (or SEQ ID NO:114 with one, two, or three amino acid additions, deletions, or substitutions), and SEQ ID NO:115 (or SEQ ID NO:115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO: 122 (or SEQ ID NO:122 with one, two, or three amino acid additions, deletions, or substitutions), and SEQ ID NO:123 (or SEQ ID NO:123 with one, two, or three amino acid additions, deletions, or substitutions).

72-77. (canceled)

78. The composition of claim 71, wherein said composition comprises a checkpoint inhibitor.

79. The composition of claim 78, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

80. A method of treating a mammal having cancer, wherein said method comprises administering, to said mammal, a composition comprising (a) an antibody, (b) an antigen binding fragment, (c) a host cell that expresses a chimeric antigen receptor comprising an antigen binding domain, a hinge, a transmembrane domain, and one or more signaling domains, wherein said antigen binding domain comprises said antibody or said antigen-binding fragment, (d) a host cell that expresses a cell engager comprising a first antigen binding domain, a linker, and a second antigen binding domain, wherein said first antigen binding domain comprises said antibody or said antigen-binding fragment, (e) said cell engager, or (f) an antibody-drug conjugate (ADC) comprising an antigen binging domain covalently linked to a drug, wherein said antigen binging domain comprises said antibody or said antigen binding fragment,
wherein said antibody or said antigen binding fragment comprises:
(i) a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions) and a light chain variable domain or region comprising the amino acid sequences set forth in SEQ ID NO:9 (or SEQ ID NO:9 with one, two, or three amino acid additions, deletions, or substitutions), SEQ ID NO: 10 (or SEQ ID NO: 10 with one, two, or three amino acid additions, deletions, or substitutions), and SEQ ID NO:11 (or SEQ ID NO: 1 with one, two, or three amino acid additions, deletions, or substitutions);
(ii) a heavy chain variable domain or region comprising the amino acid sequences set forth in SEQ ID NO:17 (or SEQ ID NO: 17 with one, two, or three amino acid additions, deletions, or substitutions), SEQ ID NO: 18 (or SEQ ID NO:18 with one, two, or three amino acid additions, deletions, or substitutions), and SEQ ID NO: 19 (or SEQ ID NO: 19 with one, two, or three amino acid additions, deletions, or substitutions) and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO: 26 with one, two, or three amino acid additions, deletions, or substitutions), and SEQ ID NO: 27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions) and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO: 42 with one, two, or three amino acid additions, deletions, or substitutions), and SEQ ID NO: 43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions) and a light chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO: 58 with one, two, or three amino acid additions, deletions, or substitutions), and SEQ ID NO: 59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions) and a light chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO: 74 with one, two, or three amino acid additions, deletions, or substitutions), and SEQ ID NO: 75 (or SEQ ID NO:75 with one, two, or three amino acid additions, deletions, or substitutions);
(vi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions) and a light chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO: 90 with one, two, or three amino acid additions, deletions, or substitutions), and SEQ ID NO: 91 (or SEQ ID NO:91 with one, two, or three amino acid additions, deletions, or substitutions);
(vii) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions) and a light chain variable domain or region comprising the amino acid sequences set forth in SEQ ID NO: 105 (or SEQ ID NO:105 with one, two, or three amino acid additions, deletions, or substitutions), SEQ ID NO: 106 (or SEQ ID NO:106 with one, two, or three amino acid additions, deletions, or substitutions), and SEQ ID NO: 107 (or SEQ ID NO:107 with one, two, or three amino acid additions, deletions, or substitutions); or
(viii) a heavy chain variable domain or region comprising the amino acid sequences set forth in SEQ ID NO: 113 (or SEQ ID NO:113 with one, two, or three amino acid additions, deletions, or substitutions), SEQ ID NO:114 (or SEQ ID NO: 114 with one, two, or three amino acid additions, deletions, or substitutions), and SEQ ID NO:115 (or SEQ ID NO: 115 with one, two, or three amino acid additions, deletions, or substitutions) and a light chain variable domain or region comprising the amino acid sequences set forth in SEQ ID NO: 121 (or SEQ ID NO:121 with one, two, or three amino acid additions, deletions, or substitutions), SEQ ID NO:122 (or SEQ ID NO:122 with one, two, or three amino acid additions, deletions, or substitutions), and SEQ ID NO:123 (or SEQ ID NO:123 with one, two, or three amino acid additions, deletions, or substitutions).

81. The method of claim 80, wherein said mammal is a human.

82. The method of claim 80, wherein said cancer is a PSCA⁺ cancer.

83. The method of claim 82, wherein said PSCA⁺ cancer is selected from the group consisting of PSCA⁺ prostate cancer, PSCA⁺ metastatic prostate cancer, PSCA⁺ bladder cancer, PSCA⁺ renal cell cancer, and PSCA⁺ pancreatic cancer.

84. (canceled)

85. The method of claim 80, wherein said method comprises
administering, to said mammal, a composition comprising a checkpoint inhibitor.

86-100. (canceled)
